SEQUENCE LISTING (91 sequences)

SEQ ID NO: 1 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 2 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 3 - DNA, 852 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcggaacctgcagatgacagcagcttctcgctgtcccaggaggttctccggcacctgcggcaggaggaaaaggaagaggttactgtgggcagcttga

SEQ ID NO: 4 - protein, 283 aa, Homo sapiens [sequence omitted]

SEQ ID NO: 5 - DNA, 783 bp, Homo sapiens
atgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 6 - protein, 260 aa, Homo sapiens
MLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 7 - DNA, 735 bp, Homo sapiens
ctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccatgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 8 - protein, 244 aa, Homo sapiens
LKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 9 - DNA, 945 bp, Homo sapiens
atgggctcgcaggccctgcccccggggcccatgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctatagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtag

SEQ ID NO: 10 - protein, 314 aa, Homo sapiens
MGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 11 - DNA, 915 bp, Homo sapiens
atgcagaccctcatctttttcgacatggaggccactggcttgcccttctcccagcccaaggtcacggagctgtgcctgctggctgtccacagatgtgccctggagagcccccccacctctcaggggccacctcccacagttcctccaccaccgcgtgtggtagacaagctctccctgtgtgtggctccggggaaggcctgcagccctgcagccagcgagatcacaggtctgagcacagctgtgctggcagcgcatgggcgtcaatgttttgatgacaacctggccaacctgctcctagccttcctgcggcgccagccacagccctggtgcctggtggcacacaatggtgaccgctacgacttccccctgctccaagcagagctggctatgctgggcctcaccagtgctctggatggtgccttctgtgtggatagcatcactgcgctgaaggccctggagcgagcaagcagcccctcagaacacggcccaaggaagagctatagcctaggcagcatctacactcgcctgtatgggcagtcccctccagactcgcacacggctgagggtgatgtcctggccctgctcagcatctgtcagtggagaccacaggccctgctgcggtgggtggatgctcacgccaggcctttcggcaccatcaggcccatgtatggggtcacagcctctgctaggaccaagccaagaccatctgctgtcacaaccactgcacacctggccacaaccaggaacactagtcccagccttggagagagcaggggtaccaaggatcttcctccagtgaaggaccctggagccctatccagggaggggctgctggccccactgggtctgctggccatcctgaccttggcagtagccacactgtatggactatccctggccacacctggggagtag

SEQ ID NO: 12 - protein, 304 aa, Homo sapiens
MQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 13 - protein, 369 aa, Homo sapiens
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 14 - DNA, 5592 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 15 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 16 - DNA, 5451 bp, Homo sapiens
atgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 17 - protein, 1816 aa, Homo sapiens
MLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 18 - DNA, 2280 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 19 - protein, 759 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 20 - DNA, 2100 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagggtgtccacccaattgtggttgtgcagccagatgcctggacagaggacaatggcttccatgcaattgggcagatgtgtga

SEQ ID NO: 21 - protein, 699 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGEAASGCESETSVSEDCSGLSSQSDILTTQQRDTMQHNLIKLQQEMAELEAVLEQHGSQPSNSYPSIISDSSALEDLRNPEQSTSEKVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTGCPPNCGCAARCLDRGQWLPCNWADV

SEQ ID NO: 22 - DNA, 5655 bp, Homo sapiens
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagattcgcatatacatggccaaaggaacaactccatgttttctaaaaggcctagagaacatatatcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 23 - protein, 1884 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKDSHIHGQRNNSMFSKRPREHISVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 24 - DNA, 10257 bp, Homo sapiens
atgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaa

SEQ ID NO: 25 - protein, 3418 aa, Homo sapiens [sequence omitted]

SEQ ID NO: 26 - DNA, 1881 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtga

SEQ ID NO: 27 - protein, 626 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 28 - DNA, 1776 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtga

SEQ ID NO: 29 - protein, 591 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 30 - DNA, 1770 bp, Homo sapiens
atgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcagtctcccaccagagcctcccactga

SEQ ID NO: 31 - protein, 589 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQSPTRASH

SEQ ID NO: 32 - DNA, 1083 bp, Homo sapiens
atgatcccgctgctgctggcagcgctgctgtgcgtccccgccggggccctgacctgctacggggactccgggcagcctgtagactggttcgtggtctacaagctgccagctcttagagggtccggggaggcggcgcagagagggctgcagtacaagtatctggacgagagctccggaggctggcgggacggcagggcactcatcaacagcccggagggggccgtgggccgaagcctgcagccgctgtaccggagcaacaccagccagctcgccttcctgctctacaatgaccaaccgcctcaacccagcaaggctcaggactcttccatgcgtgggcacacgaagggtgtcctgctccttgaccacgatgggggcttctggctggtccacagtgtacctaacttccctccaccggcctcctctgctgcatacagctggcctcatagcgcctgtacctacgggcagaccctgctctgtgtgtcttttcccttcgctcagttctcgaagatgggcaagcagctgacctacacctacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccctctaactgctcggatatctggcaggttctgaatgtgaaccagatagctttccctggaccagccggcccaagcttcaacagcacagaggaccactccaaatggtgcgtgtccccaaaagggccctggacctgcgtgggtgacatgaatcggaaccagggagaggagcaacggggtgggggcacactgtgtgcccagctgccagccctctggaaagccttccagccgctggtgaagaactaccagccctgtaatggcatggccaggaagcccagcagagcttataagatctaa

SEQ ID NO: 33 - protein, 360 aa, Homo sapiens
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 34 - DNA, 1029 bp, Homo sapiens
tgctacggggactccgggcagcctgtagactggttcgtggtctacaagctgccagctcttagagggtccggggaggcggcgcagagagggctgcagtacaagtatctggacgagagctccggaggctggcgggacggcagggcactcatcaacagcccggagggggccgtgggccgaagcctgcagccgctgtaccggagcaacaccagccagctcgccttcctgctctacaatgaccaaccgcctcaacccagcaaggctcaggactcttccatgcgtgggcacacgaagggtgtcctgctccttgaccacgatgggggcttctggctggtccacagtgtacctaacttccctccaccggcctcctctgctgcatacagctggcctcatagcgcctgtacctacgggcagaccctgctctgtgtgtcttttcccttcgctcagttctcgaagatgggcaagcagctgacctacacctacccctgggtctataactaccagctggaagggatctttgcccaggaattccccgacttggagaatgtggtcaagggccaccacgttagccaagaaccctggaacagcagcatcacactcacatcccaggccggggctgttttccagagctttgccaagttcagcaaatttggagatgacctgtactccggctggttggcagcagcccttggtaccaacctgcaggtccagttctggcacaaaactgtaggcatcctgccctctaactgctcggatatctggcaggttctgaatgtgaaccagatagctttccctggaccagccggcccaagcttcaacagcacagaggaccactccaaatggtgcgtgtccccaaaagggccctggacctgcgtgggtgacatgaatcggaaccagggagaggagcaacggggtgggggcacactgtgtgcccagctgccagccctctggaaagccttccagccgctggtgaagaactaccagccctgtaatggcatggccaggaagcccagcagagcttataagatctaa

SEQ ID NO: 35 - protein, 342 aa, Homo sapiens
CYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 36 - DNA, 4254 bp, Homo sapiens
atggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgatttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcccaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 37 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 38 - DNA, 3861 bp, Homo sapiens
atggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgatttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 39 - protein, 1286 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 40 - DNA, 3129 bp, Homo sapiens
atggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcccaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagctcgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcataa

SEQ ID NO: 41 - protein, 1042 aa, Homo sapiens
MEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 42 - DNA, 3045 bp, Homo sapiens
atggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaa

SEQ ID NO: 43 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 44 - DNA, 1851 bp, Homo sapiens
atggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 45 - protein, 616 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 46 - DNA, 1812 bp, Homo sapiens
atgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaagccggactactttagttctgtggccacagtggtgtatcttcgcaaagagaactgcatgtaccaagcctgcccgactcaggactgcaataagaaagtgattgatcaacagaatggattgtaccgctgtgagaagtgcgacaccgaatttcccaatttcaagtaccgcatgatcctgtcagtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 47 - protein, 603 aa, Homo sapiens
MQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 48 - DNA, 1674 bp, Homo sapiens
atggtcggccaactgagcgagggggccattgcggccatcatgcagaagggggatacaaacataaagcccatcctccaagtcatcaacatccgtcccattactacggggaatagtccgccgcgttatcgactgctcatgagtgatggattgaacactctatcctctttcatgttggcgacacagttgaaccctctcgtggaggaagaacaattgtccagcaactgtgtatgccagattcacagatttattgtgaacactctgaaagacggaaggagagtagttatcttgatggaattagaagttttgaagtcagctgaagcagttggagtgaagattggcaatccagtgccctataatgaaggactcgggcagccgcaagtagctcctccagcgccagcagccagcccagcagcaagcagcaggccccagccgcagaatggaagctcgggaatgggttctactgtttctaaggcttatggtgcttcaaagacatttggaaaagctgcaggtcccagcctgtcacacacttctgggggaacacagtccaaagtggtgcccattgccagcctcactccttaccagtccaagtggaccatttgtgctcgtgttaccaacaaaagtcagatccgtacctggagcaactcccgaggggaagggaagcttttctccctagaactggttgacgaaagtggtgaaatccgagctacagctttcaatgagcaagtggacaagttctttcctcttattgaagtgaacaaggtgtattatttctcgaaaggcaccctgaagattgctaacaagcagttcacagctgttaaaaatgactacgagatgaccttcaataacgagacttccgtcatgccctgtgaggacgaccatcatttacctacggttcagtttgatttcacggggattgatgacctcgagaacaagtcgaaagactcacttgtagacatcatcgggatctgcaagagctatgaagacgccactaaaatcacagtgaggtctaacaacagagaagttgccaagaggaatatctacttgatggacacatccgggaaggtggtgactgctacactgtggggggaagatgctgataaatttgatggttctagacagcccgtgttggctatcaaaggagcccgagtctctgatttcggtggacggagcctctccgtgctgtcttcaagcactatcattgcgaatcctgacatcccagaggcctataagcttcgtggatggtttgacgcagaaggacaagccttagatggtgtttccatctctgatctaaagagcggcggagtcggagggagtaacaccaactggaaaaccttgtatgaggtcaaatccgagaacctgggccaaggcgacaaggtaaatattgcagattttcaagagaatcagtgggtgacttgtttccaggagtctgctgaagctatccttggacaaaatgctgcttatcttggggaattaaaagacaagaatgaacaggcatttgaagaagttttccagaatgccaacttccgatctttcatattcagagtcagggtcaaagtggagacctacaacgacgagtctcgaattaaggccactgtgatggacgtgaagcccgtggactacagagagtatggccgaaggctggtcatgagcatcaggagaagtgcattgatgtga

SEQ ID NO: 49 - protein, 557 aa, Homo sapiens
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 50 - DNA, 1020 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactga

SEQ ID NO: 51 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 52 - DNA, 1023 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctgacggagtctcgctctgttgccaggctggagtgcaatagcgtgatcttggtctactgcaccctccgcctctcaggttcaagtgattctcctgcctcagcctcccgagtagttgggactacaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactga

SEQ ID NO: 53 - protein, 340 aa, Homo sapiens
MAMQMQLEANADTSVEEESFGPQPISRLEQCGINANDVKKLEEAGFHTVEAVAYAPKKELINIKGISEAKADKILTESRSVARLECNSVILVYCTLRLSGSSDSPASASRVVGTTGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 54 - DNA, 843 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagattgtatctgaggaaaggaagaggggaaaccagaatctgcaaaatctacgactctccctgtcttcctga

SEQ ID NO: 55 - protein, 280 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEIVSEERKRGNQNLQNLRLSLSS

SEQ ID NO: 56 - DNA, 1659 bp, Homo sapiens
atggcggccccggtccgcctgggccggaagcgcccgctgcctgcctgtcccaacccgctcttcgttcgctggctgaccgagtggcgggacgaggcgacccgcagcaggcgccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgctcgaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggcccaggagaccaatcctagagacccagcagcaaaccctggggagttggtactggatcacattgtggagcgcaagcgactggatgacctttgcagcagcatcatcgacggccgcttccgggagcagaagttccggctgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgacgcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctga

SEQ ID NO: 57 - protein, 552 aa, Homo sapiens
MAAPVRLGRKRPLPACPNPLFVRWLTEWRDEATRSRRRTRFVFQKALRSLRRYPLPLRSGKEAKILQHFGDGLCRMLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSARPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPAANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 58 - DNA, 1656 bp, Homo sapiens
atggcggccccggtccgcctgggccggaagcgcccgctgcctgcctgtcccaacccgctcttcgttcgctggctgaccgagtggcgggacgaggcgacccgcagcaggcgccgcacgcgcttcgtatttcagaaggcgctgcgttccctccgacggtacccactgccgctgcgcagcgggaaggaagctaagatcctacagcacttcggagacgggctctgccggatgctggacgagcggctgcagcggcaccgaacatcgggcggtgaccatgccccggactcaccatctggagagaacagtccagccccgcaggggcgacttgcggaagtccaggactcttccatgccagttcctgcccagcccaaagcgggaggctctggcagctactggccagctcggcactcaggagcccgagtgatactgctggtgctctaccgggagcacctgaatcctaatggtcaccacttcttaaccaaggaggagctgctgcagaggtgtgctcagaagtcccccagggtagcccctgggagtgctcgaccctggccagccctccgctccctccttcacaggaacctggtcctcaggacacaccagccagccaggtactcattgaccccagagggcctggagctggcccagaagttggccgagtcagaaggcctgagcttgctgaatgtgggcatcgggcccaaggagccccctggggaggagacagcagtgccaggagcagcttcagcagagcttgccagtgaagcaggggtccagcagcagccactggagctgaggcctggagagtacagggtgctgttgtgtgtggacattggcgagacccgggggggcgggcacaggccggagctgctccgagagctacagcggctgcacgtgacccacacggtgcgcaagctgcacgttggagattttgtgtgggtggcccaggagaccaatcctagagacccagcaaaccctggggagttggtactggatcacattgtggagcgcaagcgactggatgacctttgcagcagcatcatcgacggccgcttccgggagcagaagttccggctgaagcgctgtggtctggagcgccgggtatacctggtggaagagcatggttccgtccacaacctcagccttcctgagagcacactgctgcaggctgtcaccaacactcaggtcattgatggcttttttgtgaagcgcacagcagacattaaggagtcagccgcctacctggccctcttgacgcggggcctgcagagactctaccagggccacaccctacgcagccgcccctggggaacccctgggaaccctgaatcaggggccatgacctctccaaaccctctctgctcactcctcaccttcagtgacttcaacgcaggagccatcaagaataaggcccagtcggtgcgagaagtgtttgcccggcagctgatgcaggtgcgcggagtgagtggggagaaggcagcagccctggtggatcgatacagcacccctgccagcctcctggccgcctatgatgcctgtgccacccccaaggaacaagagacactgctgagcaccattaagtgtgggcgtctacagaggaatctggggcctgctctgagcaggaccttatcccagctctactgcagctacggccccttgacctga

SEQ ID NO: 59 - protein, 551 aa, Homo sapiens
MAAPVRLGRKRPLPACPNPLFVRWLTEWRDEATRSRRRTRFVFQKALRSLRRYPLPLRSGKEAKILQHFGDGLCRMLDERLQRHRTSGGDHAPDSPSGENSPAPQGRLAEVQDSSMPVPAQPKAGGSGSYWPARHSGARVILLVLYREHLNPNGHHFLTKEELLQRCAQKSPRVAPGSARPWPALRSLLHRNLVLRTHQPARYSLTPEGLELAQKLAESEGLSLLNVGIGPKEPPGEETAVPGAASAELASEAGVQQQPLELRPGEYRVLLCVDIGETRGGGHRPELLRELQRLHVTHTVRKLHVGDFVWVAQETNPRDPANPGELVLDHIVERKRLDDLCSSIIDGRFREQKFRLKRCGLERRVYLVEEHGSVHNLSLPESTLLQAVTNTQVIDGFFVKRTADIKESAAYLALLTRGLQRLYQGHTLRSRPWGTPGNPESGAMTSPNPLCSLLTFSDFNAGAIKNKAQSVREVFARQLMQVRGVSGEKAAALVDRYSTPASLLAAYDACATPKEQETLLSTIKCGRLQRNLGPALSRTLSQLYCSYGPLT

SEQ ID NO: 60 - DNA, 2358 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacagttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 61 - protein, 785 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 62 - DNA, 2190 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaaaagaaaaaaatcaaccaaagaaaaggctggacccaaagggagtaaggtgtccgaggaacagactcagcctccctctcctgcaggagccggcatgtccacagccatgggccgttccccatctcccaagacctcattgtcagctccacccaacagttcttcaactgagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 63 - protein, 729 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQKRKKSTKEKAGPKGSKVSEEQTQPPSPAGAGMSTAMGRSPSPKTSLSAPPNSSSTENPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 64 - DNA, 2190 bp, Homo sapiens
atgggaaaaaaatacaagaacattgttctactaaaaggattagaggtcatcaatgattatcattttagaatggttaagtccttactgagcaacgatttaaaacttaatttaaaaatgagagaagagtatgacaaaattcagattgctgacttgatggaagaaaagttccgaggtgatgctggtttgggcaaactaataaaaattttcgaagatataccaacgcttgaagacctggctgaaactcttaaaaaagaaaagttaaaagtaaaaggaccagccctatcaagaaagaggaagaaggaagtggatgctacttcacctgcaccctccacaagcagcactgtcaaaactgaaggagcagaggcaactcctggagctcagaacccgaaaacagtggccaaatgtcaggtaactcccagaagaaatgttctccaaaaacgcccagtgatagtgaaggtactgagtacaacaaagccatttgaatatgagaccccagaaatggagaaaaaaataatgtttcatgctacagtggctacacagacacagttcttccatgtgaaggttttaaacaccagcttgaaggagaaattcaatggaaagaaaatcatcatcatatcagattatttggaatatgatagtctcctagaggtcaatgaagaatctactgtatctgaagctggtcctaaccaaacgtttgaggttccaaataaaatcatcaacagagcaaaggaaactctgaagattgatattcttcacaaacaagcttcaggaaatattgtatatggggtatttatgctacataagaaaacagtaaatcagaagaccacaatctacgaaattcaggatgatagaggaaaaatggatgtagtggggacaggacaatgtcacaatatcccctgtgaagaaggagataagctccaacttttctgctttcgacttagaaaaaagaaccagatgtcaaaactgatttcagaaatgcatagttttatccagataaagaaaaaaacaaacccgagaaacaatgaccccaagagcatgaagctaccccaggaacagcgtcagcttccatatccttcagaggccagcacaaccttccctgagagccatcttcggactcctcagatgccaccaacaactccatccagcagtttcttcaccaagaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgaaaagtgaagacacaatctccaaaatgaatgacttcatgaggatgcagatactgaaggaagggagtcattttccaggaccgttcatgaccagcataggcccagctgagagccatccccacactcctcagatgcctccatcaacaccaagcagcagtttcttaaccacgttgaaaccaagactgaagactgaacctgaagaagtttccatagaagacagtgcccagagtgacctcaaagaagtgatggtgctgaacgcaacagaatcatttgtatatgagcccaaagagcagaagaaaatgtttcatgccacagtggcaactgagaatgaagtcttccgagtgaaggtttttaatattgacctaaaggagaagttcaccccaaagaagatcattgccatagcaaattatgtttgccgcaatgggttcctggaggtatatcctttcacacttgtggctgatgtgaatgctgaccgaaacatggagatcccaaaaggattgattagaagtgccagcgtaactcctaaaatcaatcagctttgctcacaaactaaaggaagttttgtgaatggggtgtttgaggtacataagaaaaatgtaaggggtgaattcacttattatgaaatacaagataatacagggaagatggaagtggtggtgcatggacgactgaccacaatcaactgtgaggaaggagataaactgaaactcacctgctttgaattggcaccgaaaagtgggaataccggggagttgagatctgtaattcatagtcacatcaaggtcatcaagaccaggaaaaacaagaaagacatactcaatcctgattcaagtatggaaacttcaccagactttttcttctaa

SEQ ID NO: 65 - protein, 729 aa, Homo sapiens
MGKKYKNIVLLKGLEVINDYHFRMVKSLLSNDLKLNLKMREEYDKIQIADLMEEKFRGDAGLGKLIKIFEDIPTLEDLAETLKKEKLKVKGPALSRKRKKEVDATSPAPSTSSTVKTEGAEATPGAQNPKTVAKCQVTPRRNVLQKRPVIVKVLSTTKPFEYETPEMEKKIMFHATVATQTQFFHVKVLNTSLKEKFNGKKIIIISDYLEYDSLLEVNEESTVSEAGPNQTFEVPNKIINRAKETLKIDILHKQASGNIVYGVFMLHKKTVNQKTTIYEIQDDRGKMDVVGTGQCHNIPCEEGDKLQLFCFRLRKKNQMSKLISEMHSFIQIKKKTNPRNNDPKSMKLPQEQRQLPYPSEASTTFPESHLRTPQMPPTTPSSSFFTKKSEDTISKMNDFMRMQILKEGSHFPGPFMTSIGPAESHPHTPQMPPSTPSSSFLTTKSEDTISKMNDFMRMQILKEGSHFPGPFMTSIGPAESHPHTPQMPPSTPSSSFLTTLKPRLKTEPEEVSIEDSAQSDLKEVMVLNATESFVYEPKEQKKMFHATVATENEVFRVKVFNIDLKEKFTPKKIIAIANYVCRNGFLEVYPFTLVADVNADRNMEIPKGLIRSASVTPKINQLCSQTKGSFVNGVFEVHKKNVRGEFTYYEIQDNTGKMEVVVHGRLTTINCEEGDKLKLTCFELAPKSGNTGELRSVIHSHIKVIKTRKNKKDILNPDSSMETSPDFFF

SEQ ID NO: 66 - DNA, 1569 bp, Homo sapiens
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 67 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 68 - DNA, 1869 bp, Homo sapiens
atggaggagtcggaacccgaacggaagcgggctcgcaccgacgaggtgcctgccggaggaagccgctccgaggcggaagatgaggacgacgaggactacgtgccctatgtgccgttacggcagcgccggcagctactgctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 69 - protein, 622 aa, Homo sapiens
MEESEPERKRARTDEVPAGGSRSEAEDEDDEDYVPYVPLRQRRQLLLQKLLQRRRKGAAEEEQQDSGSEPRGDEDDIPLGPQSNVSLLDQHQHLKEKAEARKESAKEKQLKEEEKILESVAEGRALMSVKEMAKGITYDDPIKTSWTPPRYVLSMSEERHERVRKKYHILVEGDGIPPPIKSFKEMKFPAAILRGLKKKGIHHPTPIQIQGIPTILSGRDMIGIAFTGSGKTLVFTLPVIMFCLEQEKRLPFSKREGPYGLIICPSRELARQTHGILEYYCRLLQEDSSPLLRCALCIGGMSVKEQMETIRHGVHMMVATPGRLMDLLQKKMVSLDICRYLALDEADRMIDMGFEGDIRTIFSYFKGQRQTLLFSATMPKKIQNFAKSALVKPVTINVGRAGAASLDVIQEVEYVKEEAKMVYLLECLQKTPPPVLIFAEKKADVDAIHEYLLLKGVEAVAIHGGKDQEERTKAIEAFREGKKDVLVATDVASKGLDFPAIQHVINYDMPEEIENYVHRIGRTGRSGNTGIATTFINKACDESVLMDLKALLLEAKQKVPPVLQVLHCGDESMLDIGGERGCAFCGGLGHRITDCPKLEAMQTKQVSNIGRKDYLAHSSMDF

SEQ ID NO: 70 - DNA, 1491 bp, Homo sapiens
atgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 71 - protein, 496 aa, Homo sapiens
MSVKEMAKGITYDDPIKTSWTPPRYVLSMSEERHERVRKKYHILVEGDGIPPPIKSFKEMKFPAAILRGLKKKGIHHPTPIQIQGIPTILSGRDMIGIAFTGSGKTLVFTLPVIMFCLEQEKRLPFSKREGPYGLIICPSRELARQTHGILEYYCRLLQEDSSPLLRCALCIGGMSVKEQMETIRHGVHMMVATPGRLMDLLQKKMVSLDICRYLALDEADRMIDMGFEGDIRTIFSYFKGQRQTLLFSATMPKKIQNFAKSALVKPVTINVGRAGAASLDVIQEVEYVKEEAKMVYLLECLQKTPPPVLIFAEKKADVDAIHEYLLLKGVEAVAIHGGKDQEERTKAIEAFREGKKDVLVATDVASKGLDFPAIQHVINYDMPEEIENYVHRIGRTGRSGNTGIATTFINKACDESVLMDLKALLLEAKQKVPPVLQVLHCGDESMLDIGGERGCAFCGGLGHRITDCPKLEAMQTKQVSNIGRKDYLAHSSMDF

SEQ ID NO: 72 - DNA, 2541 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaa

SEQ ID NO: 73 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 74 - DNA, 2538 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaa

SEQ ID NO: 75 - protein, 845 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 76 - DNA, 2412 bp, Homo sapiens
atggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaagaggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctacgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaattctga

SEQ ID NO: 77 - protein, 803 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 78 - DNA, 3183 bp, Homo sapiens
atggagcagctgaacgaactggagctgctgatggagaagagtttttgggaggaggcggagctgccggcggagctatttcagaagaaagtggtagcttcctttccaagaacagttctgagcacaggaatggataaccggtacctggtgttggcagtcaatactgtacagaacaaagagggaaactgtgaaaagcgcctggtcatcactgcttcacagtcactagaaaataaagaactatgcatccttaggaatgactggtgttctgttccagtagagccaggagatatcattcatttggagggagactgcacatctgacacttggataatagataaagattttggatatttgattctgtatccagacatgctgatttctggcaccagcatagccagtagtattcgatgtatgagaagagctgtcctgagtgaaacttttaggagctctgatccagccacacgccaaatgctaattggtacggttctccatgaggtgtttcaaaaagccataaataatagctttgccccagaaaagctacaagaacttgcttttcaaacaattcaagaaataagacatttgaaggaaatgtaccgcttaaatctaagtcaagatgaaataaaacaagaagtagaggactatcttccttcgttttgtaaatgggcaggagatttcatgcataaaaacacttcgactgacttccctcagatgcagctctctctgccaagtgataatagtaaggataattcaacatgtaacattgaagtcgtgaaaccaatggatattgaagaaagcatttggtcccctaggtttggattgaaaggcaaaatagatgttacagttggtgtgaaaatacatcgagggtataaaacaaaatacaagataatgccgctggaacttaaaactggcaaagaatcaaattctattgaacaccgtagtcaggttgttctgtacactctactaagccaagagagaagagctgatccagaggctggcttgcttctctacctcaagactggtcagatgtaccctgtgcctgccaaccatctagataaaagagaattattaaagctaagaaaccagatggcattctcattgtttcaccgtattagcaaatctgctactagacagaagacacagcttgcttctttgccacaaataattgaggaagagaaaacttgtaaatattgttcacaaattggcaattgtgctctttatagcagagcagttgaacaacagatggattgtagttcagtcccaattgtgatgctgcccaaaatagaagaagaaacccagcatctgaagcaaacacacttagaatatttcagcctttggtgtctaatgttaaccctggagtcacaatcgaaggataataaaaagaatcaccaaaatatctggctaatgcctgcttcggaaatggagaagagtggcagttgcattggaaacctgattagaatggaacatgtaaagatagtttgtgatgggcaatatttacataatttccaatgtaaacatggtgccatacctgtcacaaatctaatggcaggtgacagagttattgtaagtggagaagaaaggtcactgtttgctttgtctagaggatatgtgaaggagattaacatgacaacagtaacttgtttattagacagaaacttgtcggtccttccagaatcaactttgttcagattagaccaagaagaaaaaaattgtgatatagataccccattaggaaatctttccaaattgatggaaaacacgtttgtcagcaaaaaacttcgagatttaattattgactttcgtgaacctcagtttatatcctaccttagttctgttcttccacatgatgcaaaggatacagttgcctgcattctaaagggtttgaataagcctcagaggcaagcgatgaaaaaggtacttctttcaaaagactacacactcatcgtgggtatgcctgggacaggaaaaacaactacgatatgtactctcgtaagaattctctacgcctgtggttttagcgttttgttgaccagctatacacactctgctgttgacaatattcttttgaagttagccaagtttaaaataggatttttgcgtttgggtcagattcagaaggttcatccagctatccagcaatttacagagcaagaaatttgcagatcaaagtccattaaatccttagctcttctagaagaactctacaatagtcaacttatagttgcaacaacatgtatgggaataaaccatccaatattttcccgtaaaatttttgatttttgtattgtggatgaagcctctcaaattagccaaccaatttgtctgggccccctttttttttcacggagatttgtgttagtgggggaccatcagcagcttcctcccctggtgctaaaccgtgaagcaagagctcttggcatgagtgaaagcttattcaagaggctggagcagaataagagtgctgttgtacagttaaccgtgcagtacagaatgaacagtaaaattatgtccttaagtaataagctgacctatgagggcaagctggagtgtggatcagacaaagtggccaatgcagtgataaacctacgtcactttaaagatgtgaagctggaactggaattttatgctgactattctgataatccttggttgatgggagtatttgaacccaacaatcctgtttgtttccttaatacagacaaggttccagcgccagaacaagttgaaaaaggtggtgtgagcaatgtaacagaagccaaactcatagttttcctaacctccatttttgttaaggctggatgcagtccctctgatattggtattattgcaccgtacaggcagcaattaaagatcatcaatgatttattggcacgttctattgggatggtcgaagttaatacagtagacaaataccaaggaagggacaaaagtattgtcctagtatcttttgttagaagtaataaggatggaactgttggtgaactcttgaaagattggcgacgtcttaatgttgctataaccagagccaaacataaactgattcttctggggtgtgtgccctcactaaattgctatcctcctttggagaagctgcttaatcatttaaactcagaaaaattaatcattgatcttccatcaagagaacatgaaagtctttgccacatattgggtgactttcaaagagaataa

SEQ ID NO: 79 - protein, 1060 aa, Homo sapiens
MEQLNELELLMEKSFWEEAELPAELFQKKVVASFPRTVLSTGMDNRYLVLAVNTVQNKEGNCEKRLVITASQSLENKELCILRNDWCSVPVEPGDIIHLEGDCTSDTWIIDKDFGYLILYPDMLISGTSIASSIRCMRRAVLSETFRSSDPATRQMLIGTVLHEVFQKAINNSFAPEKLQELAFQTIQEIRHLKEMYRLNLSQDEIKQEVEDYLPSFCKWAGDFMHKNTSTDFPQMQLSLPSDNSKDNSTCNIEVVKPMDIEESIWSPRFGLKGKIDVTVGVKIHRGYKTKYKIMPLELKTGKESNSIEHRSQVVLYTLLSQERRADPEAGLLLYLKTGQMYPVPANHLDKRELLKLRNQMAFSLFHRISKSATRQKTQLASLPQIIEEEKTCKYCSQIGNCALYSRAVEQQMDCSSVPIVMLPKIEEETQHLKQTHLEYFSLWCLMLTLESQSKDNKKNHQNIWLMPASEMEKSGSCIGNLIRMEHVKIVCDGQYLHNFQCKHGAIPVTNLMAGDRVIVSGEERSLFALSRGYVKEINMTTVTCLLDRNLSVLPESTLFRLDQEEKNCDIDTPLGNLSKLMENTFVSKKLRDLIIDFREPQFISYLSSVLPHDAKDTVACILKGLNKPQRQAMKKVLLSKDYTLIVGMPGTGKTTTICTLVRILYACGFSVLLTSYTHSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLIIDLPSREHESLCHILGDFQRE

SEQ ID NO: 80 - DNA, 2694 bp, Homo sapiens
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagagagactagcttgcaaaattttcctcatattgaggtggttcggaaaaaagaggagagaagaaaactgcttgggcacacgtgtaaggaatgtgaaatttattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatag

SEQ ID NO: 81 - protein, 897 aa, Homo sapiens
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDERETSLQNFPHIEVVRKKEERRKLLGHTCKECEIYYADMPAEEREKKLASCSRHRFRYIPPNTPENFWEVGFPSTQTCMERGYIKEDLDPCPRPKRRQPYNAIFSPKGKEQKT

SEQ ID NO: 82 - DNA, 2604 bp, Homo sapiens
atgaacatctcgggaagcagctgtggaagccctaactctgcagatacatctagtgactttaaggacctttggacaaaactaaaagaatgtcatgatagagaagtacaaggtttacaagtaaaagtaaccaagctaaaacaggaacgaatcttagatgcacaaagactagaagaattcttcaccaaaaatcaacagctgagggaacagcagaaagtccttcatgaaaccattaaagttttagaagatcggttaagagcaggcttatgtgatcgctgtgcagtaactgaagaacatatgcggaaaaaacagcaagagtttgaaaatatccggcagcagaatcttaaacttattacagaacttatgaatgaaaggaatactctacaggaagaaaataaaaagctttctgaacaactccagcagaaaattgagaatgatcaacagcatcaagcagctgagcttgaatgtgaggaagacgttattccagattcaccgataacagccttctcattttctggcgttaaccggctacgaagaaaggagaacccccatgtccgatacatagaacaaacacatactaaattggagcactctgtgtgtgcaaatgaaatgagaaaagtttccaagtcttcaactcatccacaacataatcctaatgaaaatgaaattctagtagctgacacttatgaccaaagtcaatctccaatggccaaagcacatggaacaagcagctatacccctgataagtcatcttttaatttagctacagttgttgctgaaacacttggacttggtgttcaagaagaatctgaaactcaaggtcccatgagcccccttggtgatgagctctaccactgtctggaaggaaatcacaagaaacagccttttgaggaatctacaagaaatactgaagatagtttaagattttcagattctacttcaaagactcctcctcaagaagaattacctactcgagtgtcatctcctgtatttggagctacctctagtatcaaaagtggtttagatttgaatacaagtttgtccccttctcttttacagcctgggaaaaaaaaacatctgaaaacactcccttttagcaacacttgtatatctagattagaaaaaactagatcaaaatctgaagatagtgcccttttcacacatcacagtcttgggtctgaagtgaacaagatcattatccagtcatctaataaacagatacttataaataaaaatataagtgaatccctaggtgaacagaataggactgagtacggtaaagattctaacactgataaacatttggagcccctgaaatcattgggaggccgaacatccaaaaggaagaaaactgaggaagaaagtgaacatgaagtaagctgcccccaagcttcttttgataaagaaaatgctttcccttttccaatggataatcagttttccatgaatggagactgtgtgatggataaacctctggatctgtctgatcgattttcagctattcagcgtcaagagaaaagccaaggaagtgagacttctaaaaacaaatttaggcaagtgactctttatgaggctttgaagaccattccaaagggcttttcctcaagccgtaaggcctcagatggcaactgcacgttgcccaaagattccccaggggagccctgttcacaggaatgcatcatccttcagcccttgaataaatgctctccagacaataaaccatcattacaaataaaagaagaaaatgctgtctttaaaattcctctacgtccacgtgaaagtttggagactgagaatgttttagatgacataaagagtgctggttctcatgagccaataaaaatacaaaccaggtcagaccatggaggatgtgaacttgcatcagttcttcagttaaatccatgtagaactggtaaaataaagtctctacaaaacaaccaagatgtatcctttgaaaatatccagtggagtatagatccgggagcagacctttctcagtataaaatggatgttactgtaatagatacaaaggatggcagtcagtcaaaattaggaggagagacagtggacatggactgtacattggttagtgaaaccgttctcttaaaaatgaagaagcaagagcagaagggagaaaaaagttcaaatgaagaaagaaaaatgaatgatagcttggaagatatgtttgatcggacaacacatgaagagtatgaatcctgtttggcagacagtttctcccaagcagcagatgaagaggaggaattgtctactgccacaaagaaactacacactcatggtgataaacaagacaaagtcaagcagaaagcgtttgtggagccgtattttaaaggtgatgaaagtattatgcagatatgccagcagaagaaagagaaaagaaattggcttcctgctcaagacaccgattccgctacattccacccaacacaccagagaatttttgggaagttggttttccttccactcagacttgtatggaaagaggttatattaaggaagatcttgatccttgtcctcgtccaaaaagacgtcagccttacaacgcaatattttctccaaaaggcaaggagcagaagacatagacgttga

SEQ ID NO: 83 - protein, 867 aa, Homo sapiens
MNISGSSCGSPNSADTSSDFKDLWTKLKECHDREVQGLQVKVTKLKQERILDAQRLEEFFTKNQQLREQQKVLHETIKVLEDRLRAGLCDRCAVTEEHMRKKQQEFENIRQQNLKLITELMNERNTLQEENKKLSEQLQQKIENDQQHQAAELECEEDVIPDSPITAFSFSGVNRLRRKENPHVRYIEQTHTKLEHSVCANEMRKVSKSSTHPQHNPNENEILVADTYDQSQSPMAKAHGTSSYTPDKSSFNLATVVAETLGLGVQEESETQGPMSPLGDELYHCLEGNHKKQPFEESTRNTEDSLRFSDSTSKTPPQEELPTRVSSPVFGATSSIKSGLDLNTSLSPSLLQPGKKKHLKTLPFSNTCISRLEKTRSKSEDSALFTHHSLGSEVNKIIIQSSNKQILINKNISESLGEQNRTEYGKDSNTDKHLEPLKSLGGRTSKRKKTEEESEHEVSCPQASFDKENAFPFPMDNQFSMNGDCVMDKPLDLSDRFSAIQRQEKSQGSETSKNKFRQVTLYEALKTIPKGFSSSRKASDGNCTLPKDSPGEPCSQECIILQPLNKCSPDNKPSLQIKEENAVFKIPLRPRESLETENVLDDIKSAGSHEPIKIQTRSDHGGCELASVLQLNPCRTGKIKSLQNNQDVSFENIQWSIDPGADLSQYKMDVTVIDTKDGSQSKLGGETVDMDCTLVSETVLLKMKKQEQKGEKSSNEERKMNDSLEDMFDRTTHEEYESCLADSFSQAADEEEELSTATKKLHTHGDKQDKVKQKAFVEPYFKGDESIMQICQQKKEKRNWLPAQDTDSATFHPTHQRIFGKLVFLPLRLVWKEVILRKILILVLVQKDVSLTTQYFLQKARSRRHRR

SEQ ID NO: 84 - DNA, 2127 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 85 - protein, 708 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 86 - DNA, 2043 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 87 - protein, 680 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 88 - DNA, 2124 bp, Homo sapiens
atgagtactgcagatgcacttgatgatgaaaacacatttaaaatattagttgcaacagatattcatcttggatttatggagaaagatgcagtcagaggaaatgatacgtttgtaacactcgatgaaattttaagacttgcccaggaaaatgaagtggattttattttgttaggtggtgatctttttcatgaaaataagccctcaaggaaaacattacatacctgcctcgagttattaagaaaatattgtatgggtgatcggcctgtccagtttgaaattctcagtgatcagtcagtcaactttggttttagtaagtttccatgggtgaactatcaagatggcaacctcaacatttcaattccagtgtttagtattcatggcaatcatgacgatcccacaggggcagatgcactttgtgccttggacattttaagttgtgctggatttgtaaatcactttggacgttcaatgtctgtggagaagatagacattagtccggttttgcttcaaaaaggaagcacaaagattgcgctatatggtttaggatccattccagatgaaaggctctatcgaatgtttgtcaataaaaaagtaacaatgttgagaccaaaggaagatgagaactcttggtttaacttatttgtgattcatcagaacaggagtaaacatggaagtactaacttcattccagaacaatttttggatgacttcattgatcttgttatctggggccatgaacatgagtgtaaaatagctccaaccaaaaatgaacaacagctgttttatatctcacaacctggaagctcagtggttacttctctttccccaggagaagctgtaaagaaacatgttggtttgctgcgtattaaagggaggaagatgaatatgcataaaattcctcttcacacagtgcggcagtttttcatggaggatattgttctagctaatcatccagacatttttaacccagataatcctaaagtaacccaagccatacaaagcttctgtttggagaagattgaagaaatgcttgaaaatgctgaacgggaacgtctgggtaattctcaccagccagagaagcctcttgtacgactgcgagtggactatagtggaggttttgaacctttcagtgttcttcgctttagccagaaatttgtggatcgggtagctaatccaaaagacattatccattttttcaggcatagagaacaaaaggaaaaaacaggagaagagatcaactttgggaaacttatcacaaagccttcagaaggaacaactttaagggtagaagatcttgtaaaacagtactttcaaaccgcagagaagaatgtgcagctctcactgctaacagaaagagggatgggtgaagcagtacaagaatttgtggacaaggaggagaaagatgccattgaggaattagtgaaataccagttggaaaaaacacagcgatttcttaaagaacgtcatattgatgccctcgaagacaaaatcgatgaggaggtacgtcgtttcagagaaaccagacaaaaaaatactaatgaagaagatgatgaagtccgtgaggctatgaccagggccagagcactcagatctcagtcagaggagtctgcttctgcctttagtgctgatgaccttatgagtatagatttagcagaacagatggctaatgactctgatgatagcatctcagcagcaaccaacaaaggaagaggccgaggaagaggtcgaagaggtggaagagggcagaattcagcatcgagaggagggtctcaaagaggaagagacactggtctggagacttctacccgtagcaggaactcaaagactgctgtgtcagcatctagaaatatgtctattatagatgcctttaaatctacaagacagcagccttcccgaaatgtcactactaagaattattcagaggtgattgaggtagatgaatcagatgtggaagaagacatttttcctaccacttcaaagacagatcaaaggtggtccagcacatcatccagcaaaatcatgtcccagagtcaagtatcgaaaggggttgattttgaatcaagtgaggatgatgatgatgatccttttatgaacactagttctttaagaagaaatagaagataa

SEQ ID NO: 89 - protein, 707 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRDTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 90 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 91 - DNA, 12915 bp, Homo sapiens
aggtagctgcgtggctaacggagaaaagaagccgtggccgcgggaggaggcgagaggagtcgggatctgcgctgcagccaccgccgcggttgatactactttgaccttccgagtgcagtgacagtgatgtgtgttctgaaattgtgaaccatgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtgatcttcagtatatgaattaccctttcattcagcctttagaaattatattttagcctttatttttaacctgccaacatactttaagtagggattaatatttaagtgaactattgtgggtttttttgaatgttggttttaatacttgatttaatcaccactcaaaaatgttttgatggtcttaaggaacatctctgctttcactctttagaaataatggtcattcgggctgggcgcagcggctcacgcctgtaatcccagcactttgggaggccgaggtgagcggatcacaaggtcaggagttcgagaccagcctggccaagagaccagcctggccagtatggtgaaaccctgtctctactaaaaatacaaaaattagccgagcatggtggcgggcacctgtaatcccagctactcgagaggctgaggcaggagaatctcttgaacctgggaggtgaaggttgctgtgggccaaaatcatgccattgcactccagcctgggtgacaagagcgaaactccatctcaaaaaaaaaaaaaaaaaaacagaaacgtatttggatttttcctagtaagatcactcagtgttactaaataatgaagttgttatggagaacaaatttcaaagacacagttagtgtagttactatttttttaagtgtgtattaaaacttctcattctattctctttatcttttaagcccttctgtactgtccatgtatgttatctttctgtgataacttcatagattgccttctagttcatgaattctcttgtcagatgtatataatctcttttaccctatccattgggcttcttctttcagaaattgtttttcatttctaattatgcatcatttttcagatctctgtttcttgatgtcatttttaatgtttttttaatgttttttatgtcactaattattttaaatgtctgtacttgatagacactgtaatagttctattaaatttagttcctgctgtttatatctgttgatttttgtatttgataggctgttcatccagttttgtctttttgaaaagtgagtttattttcagcaaggctttatctatgggaatcttgagtgtctgtttatgtcatattcccagggctgttgctgcacacaagcccattcttattttaatttcttggctttagggtttccatacctgaagtgtagcataaatactgataggagatttcccaggccaaggcaaacacacttcctcctcatctccttgtgctagtgggcagaatatttgattgatgcctttttcactgagagtataagcttccatgtgtcccacctttatggcaggggtggaaggaggtacatttaattcccactgcctgcctttggcaagccctgggttctttgctccccatatagatgtctaagctaaaagccgtgggttaatgagactggcaaattgttccaggacagctacagcatcagctcacatattcacctctctggtttttcattcccctcatttttttctgagacagagtcttgctctgtcacccaggctggagtgcagtggcatgatctcagctcactgaaacctctgcctcctgggttcaagcaattctcctgcctcagcctcccgagtagctgggactacaggcgtgtgccaacacgcccggctaattttttgtatttttattagagacggagtttcaccgtgttagccaggatggtctcgatcgcttgacctcgtgatccaccctcctcggcctcccaaagtgctgggattacaggtgtgagccaccgcgcccggcctcattcccctcatttttgaccgtaaggatttcccctttcttgtaagttctgctatgtatttaaaagaatgttttctacattttatccagcatttctctgtgttctgttggaagggaagggcttaggtatctagtttgatacataggtagaagtggaacatttctctgtcccccagctgtcatcatataagataaacatcagataaaaagccacctgaaagtaaaactactgactcgtgtattagtgagtataatctcttctccatccttaggaaaatgttcatcccagctgcggagattaacaaatgggtgattgagctttctcctcgtatttggaccttgaaggttatataaatttttttcttatgaagagttggcatttctttttattgccaatggcaggcactcattcatatttgatctcctcaccttcccctcccctaaaaccaatctccagaactttttggactataaatttcttggtttgacttctggagaactgttcagaatattactttgcatttcaaattacaaacttaccttggtgtatctttttcttacaagctgcctaaatgaatatttggtatatattggtagttttattactatagtaaatcaaggaaatgcagtaaacttaaaatgtctttaagaaagccctgaaatcttcatgggtgaaattagaaattatcaactagataatagtatagataaatgaatttgtagctaattcttgctagttgttgcatccagagagctttgaataacatcattaatctactctttagccttgcatggtatgctatgaggctcctgttctgttcaagtattctaatcaatggctttgaaaagtttatcaaatttacatacagatcacaagcctaggagaaataactaattcacagatgacagaattaagattataaaagattttttttttgtaattttagtagagacagggttgccattgtattccagccttggcgacagagcaagactctgcctcaaaaaaaaaaaaaaaaaggttttggcaagctggaactctttctgcaaatgactaagatagaaaactgccaaggacaaatgaggagtagttagattttgaaaatattaatcatagaatagttgttgtatgctaagtcactgacccatattatgtacagcatttctgatctttactttgcaagattagtgatactatcccaatacactgctggagaaatcagaatttggagaaataagttgtccaaggcaagaagatagtaaattataagtacaagtgtaatatggacagtatctaacttgaaaagatttcaggcgaaaagaatctggggtttgccagtcagttgctcaaaaggtcaatgaaaaccaaatagtgaagctatcagagaagctaataaattatagactgcttgaacagttgtgtccagattaagggagataatagctttcccaccctactttgtgcaggtcatacctccccaaagtgtttacctaatcagtaggttcacaaactcttggtcattatagtatatgcctaaaatgtatgcacttaggaatgctaaaaatttaaatatggtctaaagcaaataaaagcaaagaggaaaaactttggacagcgtaaagactagaatagtcttttaaaaagaaagccagtatattggtttgaaatatagagatgtgtcccaatttcaagtattttaattgcaccttaatgaaattatctattttctatagattttagtactattgaatgtattactttactgttacctgaatttattataaagtgtttttgaataaataattctaaaagcata

(19) United States
(12) Patent Application Publication
(10) Pub. No.: US 2024/0060982 A1
(43) Pub. Date: Feb. 22, 2024

(54) METHODS OF TREATING CANCER

(71) Applicant: IFM Due, Inc., Boston, MA (US)

(72) Inventors: Gary Glick, Ann Arbor, MI (US); Anthony William Opipari, Jr., Dexter, MI (US); Hans Martin Seidel, Concord, MA (US)

(21) Appl. No.: 18/266,180

(22) PCT Filed: Dec. 21, 2021
(86) PCT No.: PCT/US2021/064638

§ 371 Date: Jun. 8, 2023

Related U.S. Application Data
(60) Provisional application No. 63/129,229, filed on Dec. 22, 2020.

(51) Int. Cl. G01N 33/574; A61K 45/06

(52) U.S. Cl. CPC G01N 33/57484; A61K 45/06; G01N 2800/52

(57) ABSTRACT

Provided herein are methods of treating a subject, such as a subject that has cancer, that include administering a therapeutically effective amount of a STING antagonist or a cGAS inhibitor or a pharmaceutically acceptable salt, solvate, or co-crystal thereof to a subject identified as having decreased ATM level and/or activity in a tumor sample obtained from the subject as compared to a reference level.

### CROSS REFERENCE TO RELATED APPLICATIONS

This application claims the benefit of U.S. Provisional Application Ser. No. 63/129,229, filed on Dec. 22, 2020, which is incorporated herein by reference in its entirety.

### TECHNICAL FIELD

The present disclosure relates to, in part, methods of treating a subject, e.g., a subject having cancer, which include administration of a STING antagonist or a cGAS inhibitor.

### BACKGROUND

The cGAS/STING (cyclic GMP-AMP Synthase/Stimulator of Interferon Genes) pathway is a component of inflammatory signaling pathways. When DNA is present in the cytosol of a cell, cGAS binds it and generates 2′-5′ cyclic GMP-AMP (cGAMP).

Activated by cGAMP, STING induces the phosphorylation of and nuclear translocation of interferon (IFN) regulatory factors (IRFs). As transcription factors, IRFs regulate the expression of genes, including the type I IFNs, which regulate the activity of the immune system.

The presence of DNA in the cytosol of a cell can sometimes be the result of an infection. In some cases, the presence of DNA in the cytosol of a cell can be the result of DNA damage in the nucleus of a cell or in the mitochondria of a cell. In some instances, the cytosolic DNA is degraded or modified by enzymes to prevent activation of the cGAS/STING pathway.

### SUMMARY

The present disclosure is based on the discovery that cancer cells having decreased ATM level and/or activity are more sensitive to treatment with a STING antagonist or a cGAS inhibitor, e.g., than cells that do not have decreased ATM level and/or activity.

Provided herein are methods of treating a subject in need thereof that include: (a) identifying a subject having a cancer cell having decreased ATM level and/or activity in a tumor sample obtained from the subject as compared to a reference level; and (b) administering a treatment comprising a therapeutically effective amount of a STING antagonist or a cGAS inhibitor, or a pharmaceutically acceptable salt, solvate, or co-crystal thereof to the identified subject.

Also provided herein are methods of treating a subject in need thereof that include administering a treatment comprising a therapeutically effective amount of a STING antagonist or a cGAS inhibitor, or a pharmaceutically acceptable salt, solvate, or co-crystal thereof to a subject identified as having a cancer cell having decreased ATM level and/or activity in a tumor sample obtained from the subject as compared to a reference level.

Also provided herein are methods of selecting a treatment for a subject in need thereof that include: (a) identifying a subject having a cancer cell having decreased ATM level and/or activity in a tumor sample obtained from the subject as compared to a reference level; and (b) selecting for the identified subject a treatment comprising a therapeutically effective amount of a STING antagonist or a cGAS inhibitor, or a pharmaceutically acceptable salt, solvate, or co-crystal thereof.

Also provided herein are methods of selecting a treatment for a subject in need thereof that include selecting a treatment comprising a therapeutically effective amount of a STING antagonist or cGAS inhibitor, or a pharmaceutically acceptable salt, solvate, or co-crystal thereof for a subject identified as having a cancer cell having decreased ATM level and/or activity in a tumor sample obtained from the subject as compared to a reference level.

Also provided herein are methods of selecting a subject for treatment that include: (a) identifying a subject having a cancer cell having decreased ATM level and/or activity in a tumor sample obtained from the subject as compared to a reference level; and (b) selecting the identified subject for treatment with a therapeutically effective amount of a STING antagonist or a cGAS inhibitor, or a pharmaceutically acceptable salt, solvate, or co-crystal thereof.

Also provided herein are methods of selecting a subject for participation in a clinical trial that include: (a) identifying a subject having a cancer cell having decreased ATM level and/or activity in a tumor sample obtained from the subject as compared to a reference level; and (b) selecting the identified subject for participation in a clinical trial that comprises administration of a treatment comprising a therapeutically effective amount of a STING antagonist or a cGAS inhibitor, or a pharmaceutically acceptable salt, solvate, or co-crystal thereof.

Also provided herein are methods of selecting a subject for participation in a clinical trial that include selecting a subject identified as having a cancer cell having decreased ATM level and/or activity in a tumor sample obtained from the subject as compared to a reference level, for participation in a clinical trial that comprises administration of a treatment comprising a therapeutically effective amount of a STING antagonist or a cGAS inhibitor, or a pharmaceutically acceptable salt, solvate, or co-crystal thereof.

Also provided herein are methods of predicting a subject's responsiveness to a STING antagonist or cGAS inhibitor that include: (a) determining that a subject has a cancer cell having decreased ATM level and/or activity in a tumor sample obtained from the subject as compared to a reference level; and (b) identifying that the subject determined to have decreased ATM expression and/or activity in a tumor sample obtained from the subject as compared to a reference level, in step (a) has an increased likelihood of being responsive to treatment with a STING antagonist or a cGAS inhibitor.

Also provided herein are methods of predicting a subject's responsiveness to a STING antagonist or cGAS inhibitor that include identifying a subject determined to have a cancer cell having decreased ATM level and/or activity in a tumor sample obtained from the subject as compared to a reference level, as having an increased likelihood of being responsive to treatment with a STING antagonist or a cGAS inhibitor.

In some embodiments of any of the methods described herein, the subject is identified having a cancer cell having both (i) decreased ATM level and/or activity and (ii) increased cGAS/STING signaling pathway activity, as compared to a reference level; and optionally wherein the subject is identified as having an elevated level of cGAMP in a serum or tumor sample obtained from the subject as compared to a reference level.

In some embodiments of any of the methods described herein, the decreased ATM level and/or activity is a result of loss of one or both alleles of an ATM gene in the subject. In some embodiments of any of the methods described herein, the decreased ATM level and/or activity is a result of a mutation in one or both alleles of an ATM gene in the subject.

In some embodiments of any of the methods described herein, the method further includes administering the selected treatment to the subject. In some embodiments of any of the methods described herein, the method further includes administering a therapeutically effective amount of a STING antagonist or a cGAS inhibitor to a subject identified as having an increased likelihood of being responsive to treatment with a STING antagonist or a cGAS inhibitor.

In some embodiments of any of the methods described herein, the subject has been diagnosed or identified as having a cancer, such as a cancer selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments of any of the methods described herein, the subject has been diagnosed or identified as having a cancer, such as a cancer selected from the group consisting of: renal clear cell carcinoma, uveal melanoma, tongue squamous cell carcinoma, breast cancer, and skin cancer.

In some embodiments of any of the methods described herein, the STING antagonist is a compound of any one of Formulas I-XXIV or Formulas M1-M6, or a pharmaceutically acceptable salt, solvate, or co-crystal thereof. In some embodiments of any of the methods described herein, the STING antagonist or the cGAS inhibitor is a compound selected from the group consisting of the compounds in Tables C1-C2, or a pharmaceutically acceptable salt, solvate, or co-crystal thereof.

As used herein, the term “STING antagonist” is an agent that decreases one or both of (i) the activity of STING (e.g., any of the exemplary activities of STING described herein) (e.g., as compared to the level of STING activity in the absence of the agent) and (ii) the expression level of STING in a mammalian cell (e.g., using any of the exemplary methods of detection described herein) (e.g., as compared to the expression level of STING in a mammalian cell not contacted with the agent). Non-limiting examples of STING antagonists are described herein.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

As used herein, the term “ATM” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

As used herein, the term “cGAS inhibitor” is an agent that decreases one or both of (i) the activity of cGAS (e.g., any of the exemplary activities of cGAS described herein) (e.g., as compared to the level of cGAS activity in the absence of the agent) and (ii) the expression level of cGAS in a mammalian cell (e.g., using any of the exemplary methods of detection described herein) (e.g., as compared to the expression level of cGAS in a mammalian cell not contacted with the agent). Non-limiting examples of cGAS inhibitors are described herein.

As used herein, the term “cGAS” is meant to include, without limitation, nucleic acids, polynucleotides, oligonucleotides, sense and antisense polynucleotide strands, complementary sequences, peptides, polypeptides, proteins, homologous and/or orthologous cGAS molecules, isoforms, precursors, mutants, variants, derivatives, splice variants, alleles, different species, and active fragments thereof.

The term “acceptable” with respect to a formulation, composition, or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a STING antagonist or cGAS inhibitor being administered that will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a STING antagonist or cGAS inhibitor disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts not specifically limited as far as it can be used in medicaments. Examples of a salt that the compounds described herein from with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a STING antagonist or cGAS inhibitor with other chemical components (referred to collectively herein as “excipients”), such as carriers, stabilizers, diluents, dispersing agents, suspending agents, and/or thickening agents. The pharmaceutical composition facilitates administration of the STING antagonist or cGAS inhibitor to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human. In some embodiments of any of the methods described herein, the subject is 1 year old or older, 2 years old or older, 4 years old or older, 5 years old or older, 10 years old or older, 12 years old or older, 13 years old or older, 15 years old or older, 16 years old or older, 18 years old or older, 20 years old or older, 25 years old or older, 30 years old or older, 35 years old or older, 40 years old or older, 45 years old or older, 50 years old or older, 55 years old or older, 60 years old or older, 65 years old or older, 70 years old or older, 75 years old or older, 80 years old or older, 85 years old or older, 90 years old or older, 95 years old or older, 100 years old or older, or 105 years old or older,

In some embodiments of any of the methods described herein, the subject has been previously diagnosed or identified as having a disease associated with STING activity (e.g., a cancer, e.g., any of the exemplary types of cancer described herein). In some embodiments of any of the methods described herein, the subject is suspected of having a cancer (e.g., any of the exemplary cancers described herein). In some embodiments of any of the methods described herein, the subject is presenting with one or more (e.g., two, three, four, or five) symptoms of a cancer (e.g., any of the exemplary cancers described herein).

In some embodiments of any of the methods described herein, the subject is a participant in a clinical trial. In some embodiments of any of the methods described herein, the subject has been previously administered a pharmaceutical composition and the different pharmaceutical composition was determined not to be therapeutically effective.

The term “administration” or “administering” refers to a method of providing a dosage of a pharmaceutical composition or a compound to an invertebrate or a vertebrate, including a fish, a bird and a mammal (e.g., a human). In some aspects, administration is performed, e.g., orally, intravenously, subcutaneously, intranasally, transdermally, intraperitoneally, intramuscularly, intrapulmonarilly, intralymphatic, topically, intraocularly, vaginally, rectally, intrathecally, or intracystically. The method of administration can depend on various factors, e.g., the site of the disease, the severity of the disease, and the components of the pharmaceutical composition.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread, or worsening of a disease, disorder or condition or of one or more symptoms thereof.

The phrase “an elevated level” or “an increased level” as used herein can be an increase or 1.1× to 100×, or higher (such as up to 200×) e.g., as compared to a reference level (e.g., any of the exemplary reference levels described herein). In some aspects, “an elevated level” or “an increased level” can be an increase of at least 1% (e.g., at least 2%, at least 4, at least 6%, at least 8%, at least 10%, at least 12%, at least 14%, at least 16%, at least 18%, at least 20%, at least 22%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 120%, at least 130%, at least 140%, at least 150%, at least 160%, at least 170%, at least 180%, at least 190%, at least 200%, at least 220%, at least 250%, at least 280%, at least 300%, at least 320%, at least 350%, at least 380%, at least 400%, at least 420%, at least 450%, at least 480%, at least 500%, at least 600%, at least 700%, at least 800%, at least 900%, or at least 1000%), e.g., as compared to a reference level (e.g., any of the exemplary reference levels described herein).

The phrase “a decreased level” as used herein can be a decrease of at least 1% (e.g., at least 2%, at least 4, at least 6%, at least 8%, at least 10%, at least 12%, at least 14%, at least 16%, at least 18%, at least 20%, at least 22%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, e.g., as compared to a reference level (e.g., any of the exemplary reference levels described herein).

The phrase “decreased ATM level” means a decrease in the level of ATM protein and/or ATM mRNA in a mammalian cell. For example, a decrease in the level of ATM can be a result of an ATM gene loss (at one or both alleles), a mutation in a regulatory region of an ATM gene that results in decreased transcription of an ATM gene as compared to the wildtype ATM gene, a mutation in an ATM gene that results in decreased translation of an ATM mRNA as compared to the wildtype ATM gene, and/or a mutation in an ATM gene that results in the production of an ATM protein that has decreased stability and/or half-life in a mammalian cell as compared to the wildtype ATM gene.

In some embodiments of any of the methods described herein can include determining the level of expression of an mRNA or a protein encoded by ATM. In some embodiments, a decreased level and/or activity of ATM can be determined by detection of a loss-of-function ATM mutation, an ATM gene deletion, one or more amino acid deletions in a protein encoded by an ATM gene, one or more amino acid insertions in a protein encoded by an ATM gene, and/or one or more amino acid substitutions in a protein encoded by an ATM gene.

The phrase “protein activity” (or “activity” of a particular protein) means one or more activities of the protein (e.g., enzymatic activity, localization activity, binding activity (e.g., binding another protein or binding a non-protein (e.g., a nucleic acid)). A decrease in activity of a protein in a mammalian cell can be, e.g., the result of an amino acid deletion in the protein, an amino acid insertion, or an amino acid substitution in the protein, e.g., as compared to the wildtype protein. In some cases, an increase in activity of a protein in a mammalian cell can be, e.g., the result of gene amplification or an activating amino acid substitution in the protein, e.g., as compared to the wildtype protein.

The phrase “ATM activity” means a direct activity of ATM in a mammalian cell (e.g., DNA damage-inducible protein kinase activity); or downstream signaling activity of ATM activity in a mammalian cell. For example, a decrease in ATM activity in a mammalian cell can be the result of, e.g., ATM gene loss (e.g., at one or both alleles), one or more nucleotide substitutions, deletions, and/or insertions in an ATM gene, one or more amino acid deletions, substitutions, insertions, truncations, or other modifications in an ATM protein, or one or more post-translational modifications to an ATM protein that alter its activity, localization, or function.

The term “increased STING pathway activity” means an increase in direct activity of STING in a mammalian cell (e.g., translocation of STING from the endoplasmic reticulum to the perinuclear area, or activation of TBK1 (TANK Binding Kinase 1); or an increase in upstream activity or a mutation (e.g., any of the exemplary mutations or single nucleotide polymorphisms described herein) in a mammalian cell that results in increased STING pathway activity in the mammalian cell (e.g., decreased level or activity of one or more of BRCA1, BRCA2, SAMHD1, DNASE2, BLM, PARP1, RPA1, and RAD51 (e.g., as compared to any of the exemplary reference levels described herein) or increased level or activity of one or more of MUS81, IFI16, cGAS, DDX41, EXO1, DNA2, RBBP8, and MRE11 (e.g., as compared to any of the exemplary reference levels described herein).

A decreased level or activity of one or more of BRCA1, BRCA2, SAMHD1, DNASE2, BLM, PARP1, RPA1, and RAD51 (e.g., in a cancer cell) can be caused by any mechanism.

In some embodiments, a decreased level or activity of BRCA1 can be a result of a frameshift mutation in a BRCA1 gene (e.g., an E111Gfs*3 frameshift insertion). In some embodiments, a decreased level or activity of BRCA1 can be a result of a BRCA1 gene loss (e.g., loss of one allele of BRCA1 or loss of both alleles of BRCA1). In some embodiments, a decreased level or activity of BRCA1 can be a result of one or more amino acid deletions in a protein encoded by a BRCA1 gene. In some embodiments, a decreased level or activity of BRCA1 in a can be a result of one or more inactivating amino acid substitutions in a protein encoded by a BRCA1 gene.

In some embodiments, a decreased level or activity of a BRCA2 gene can be result of a frameshift mutation in a BRCA2 gene (e.g., a N1784Kfs*3 frameshift insertion). In some embodiments, a decreased level or activity of BRCA2 can be a result of BRCA2 gene loss (e.g., loss of one allele of BRCA2 or loss of both alleles of BRCA2). In some embodiments, a decreased level or activity of BRCA2 can be a result of one or more amino acid deletions in a protein encoded by a BRCA2 gene. In some embodiments, a decreased level or activity of BRCA2 can be a result of one or more inactivating amino acid substitutions in a protein encoded by a BRCA2 gene.

In some embodiments, a decreased level or activity of SAMHD1 can be a result of one or more inactivating amino acid substitutions in a protein encoded by a SAMHD1 gene (e.g., a V133I amino acid substitution). In some embodiments, a decreased level or activity of SAMHD1 can be a result of gene loss (e.g., loss of one allele of SAMHD1 or loss of both alleles of SAMHD1). In some embodiments, a decreased level or activity of SAMHD1 can be a result of one or more amino acid deletions in a protein encoded by a SAMHD1 gene.

In some embodiments, a decreased level or activity of DNASE2 can be a result of one or more inactivating mutations in a protein encoded by a DNASE2 gene (e.g., a R314W amino acid substitution). In some embodiments, a decreased level or activity of DNASE2 can be a result of DNASE2 gene loss (e.g., loss of one allele of DNASE2 or loss of both alleles of DNASE2). In some embodiments, a decreased level or activity of DNASE2 can be a result of one or more amino acid deletions in a protein encoded by a DNASE2 gene.

In some embodiments, a decreased level or activity of BLM can be a result of a frameshift mutation in a BLM gene (e.g., a N515Mfs*16 frameshift deletion). In some embodiments, a decreased level or activity of BLM can be a result of BLM gene loss (e.g., loss of one allele of BLM or loss of both alleles of BLM). In some embodiments, a decreased level or activity of BLM can be a result of one or more amino acid deletions in a protein encoded by a BLM gene. In some embodiments, a decreased level or activity of BLM can be a result of one or more inactivating amino acid substitutions in a protein encoded by a BLM gene.

In some embodiments, a decreased level or activity of PARP1 can be a result of a frameshift mutation in a PARP1 gene (e.g., a S507Afs*17 frameshift deletion). In some embodiments, a decreased level or activity of PARP1 can be a result of gene loss (e.g., loss of one allele of PARP1 or loss of both alleles of PARP1). In some embodiments, a decreased level or activity of PARP1 can be a result of one or more amino acid deletions in a protein encoded by a PARP1 gene. In some embodiments, a decreased level or activity of PARP1 can be a result of one or more inactivating amino acid substitutions in a protein encoded by a PARP1 gene.

In some embodiments, a decreased level or activity of RPA1 can be a result of a mutation that results in aberrant RPA mRNA splicing (e.g., a X12 splice mutation). In some embodiments, a decreased level or activity of RPA1 can be a result of RPA1 gene loss (e.g., loss of one allele of RPA1 or loss of both alleles of RPA1). In some embodiments, a decreased level or activity of RPA1 can be a result of one or more amino acid deletions in a protein encoded by a RPA1 gene. In some embodiments, a decreased level or activity of RPA1 can be a result of one or more inactivating amino acid substitutions in a protein encoded by a RPA1 gene.

In some embodiments, a decreased level or activity of RAD51 can be a result of one or more inactivating mutations in a protein encoded by a RAD51 gene (e.g., a R254* mutation). In some embodiments, a decreased level or activity of RAD51 can be a result of RAD51 gene loss (e.g., loss of one allele of RAD51 or loss of both alleles of RAD51). In some embodiments, a decreased level or activity of RAD51 can be a result of one or more amino acid deletions in a protein encoded by a RAD51 gene.

An increased level or activity of one or more of MUS81, IFI16, cGAS, DDX41, EXO1, DNA2, RBBP8, or MRE11 (e.g., in a cancer cell) can be caused by any mechanism.

In some embodiments, an increased level or activity of MUS81 can be a result of MUS81 gene amplification. In some embodiments, an increased level or activity of MUS81 can be a result of one or more activating amino acid substitutions in a protein encoded by a MUS81 gene.

In some embodiments, an increased level or activity of IFI16 can be a result of IFI16 gene amplification. In some embodiments, an increased level or activity of IFI16 can be a result of one or more activating amino acid substitutions in a protein encoded by an IFI16 gene.

In some embodiments, an increased level or activity of cGAS can be a result of cGAS gene amplification. In some embodiments, an increased level or activity of cGAS can be a result of one or more activating amino acid substitutions in a protein encoded by a cGAS gene.

In some embodiments, an increased level or activity of DDX41 can be a result of DDX41 gene amplification. In some embodiments, an increased level or activity of DDX41 can be a result of one or more activating amino acid substitutions in a protein encoded by a DDX41 gene.

In some embodiments, an increased level or activity of EXO1 can be a result of EXO1 gene amplification. In some embodiments, an increased level or activity of EXO1 can be a result of one or more activating amino acid substitutions in a protein encoded by an EXO1 gene.

In some embodiments, an increased level or activity of DNA2 can be a result of DNA2 gene amplification. In some embodiments, an increased level or activity of DNA2 can be a result of one or more activating amino acid substitutions in a protein encoded by a DNA2 gene.

In some embodiments, an increased level or activity of RBBP8 (also called CtIP) can be a result of RBBP8 gene amplification. In some embodiments, an increased level or activity of RBBP8 can be a result of one or more activating amino acid substitutions in a protein encoded by a RBBP8 gene.

In some embodiments, an increased level or activity of MRE11 can be a result of MRE11 gene amplification. In some embodiments, an increased level or activity of MRE11 can be a result of one or more activating amino acid substitutions in a protein encoded by a MRE11 gene.

Non-limiting examples of human protein and human cDNA sequences for STING, TREX1, BRCA1, BRCA2, SAMHD1, DNASE2, BLM, PARP1, RPA1, RAD51, MUS81, IFI16, cGAS, DDX41, EXO1, DNA2, RBBP8 (CtIP), and MRE11 are shown below (SEQ ID NOs.: 1-89). It will be understood that other natural variants of these sequences can exist, and it will be understood that the name of a gene can be used to refer to the gene or to its protein product.

| SEQUENCE NAME | SEQ ID NO: |
|---|---|
| Human STING cDNA, Variant 1 | 1 |
| Human STING Protein, Variant 1 | 2 |
| Human STING cDNA, Variant 2 | 3 |
| Human STING Protein, Variant 2 | 4 |
| Human STING cDNA, Variant 3 Precursor | 5 |
| HUMAN STING Protein, Variant 3 Precursor | 6 |
| Human STING cDNA, Variant 3 Mature Sequence | 7 |
| HUMAN STING Protein, Variant 3 Mature Sequence | 8 |
| Human TREX1 cDNA Sequence, Variant 1 | 9 |
| Human TREX1 Protein Sequence, Variant 1 | 10 |
| Human TREX1 cDNA Sequence, Variant 2 | 11 |
| Human TREX1 Protein Sequence, Variant 2 | 12 |
| Human TREX Protein Sequence, Variant 3 | 13 |
| Human BRCA1 cDNA Sequence, Variant 1 | 14 |
| Human BRCA1 Protein Sequence, Variant 1 | 15 |
| Human BRCA1 cDNA Sequence, Variant 2 | 16 |
| Human BRCA1 Protein Sequence, Variant 2 | 17 |
| Human BRCA1 cDNA Sequence, Variant 3 | 18 |
| Human BRCA1 Protein Sequence, Variant 3 | 19 |
| Human BRCA1 cDNA Sequence, Variant 4 | 20 |
| Human BRCA1 Protein Sequence, Variant 4 | 21 |
| Human BRCA1 cDNA Sequence, Variant 5 | 22 |
| Human BRCA1 Protein Sequence, Variant 5 | 23 |
| Human BRCA2 cDNA Sequence | 24 |
| Human BRCA2 Protein Sequence | 25 |
| Human SAMHD1 cDNA Sequence, Variant 1 | 26 |
| Human SAMHD1 Protein Sequence, Variant 1 | 27 |
| Human SAMHD1 cDNA Sequence, Variant 2 | 28 |
| Human SAMHD1 Protein Sequence, Variant 2 | 29 |
| Human SAMHD1 cDNA Sequence, Variant 3 | 30 |
| Human SAMHD1 Protein Sequence, Variant 3 | 31 |
| Human DNASE2 Precursor cDNA Sequence | 32 |
| Human DNASE2 Precursor Protein Sequence | 33 |
| Human DNASE2 Mature cDNA Sequence | 34 |
| Human DNASE2 Mature Protein Sequence | 35 |
| Human BLM cDNA Sequence, Variant 1 | 36 |
| Human BLM Protein Sequence, Variant 1 | 37 |
| Human BLM cDNA Sequence, Variant 2 | 38 |
| Human BLM Protein Sequence, Variant 2 | 39 |
| Human BLM cDNA Sequence, Variant 3 | 40 |
| HUMAN BLM Protein Sequence, Variant 3 | 41 |
| Human PARP1 cDNA sequence | 42 |
| Human PARP protein sequence | 43 |
| Human RPA1 cDNA Sequence, Variant 1 | 44 |
| Human RPA1 Protein Sequence, Variant 1 | 45 |
| Human RPA1 cDNA Sequence, Variant 2 | 46 |
| HUMAN RPA1 Protein Sequence, Variant 2 | 47 |
| Human RPA1 cDNA Sequence, Variant 3 | 48 |
| HUMAN RPA1 Protein Sequence, Variant 3 | 49 |
| Human RAD51 cDNA Sequence, Variant 1 | 50 |
| Human RAD51 Protein Sequence, Variant 1 | 51 |
| Human RAD51 cDNA Sequence, Variant 2 | 52 |
| Human RAD51 Protein Sequence, Variant 2 | 53 |
| Human RAD51 cDNA Sequence, Variant 3 | 54 |
| Human RAD51 Protein Sequence, Variant 3 | 55 |
| Human MUS81 cDNA Sequence, Variant 1 | 56 |
| HUMAN MUS81 Protein Sequence, Variant 1 | 57 |
| Human MUS81 cDNA Sequence, Variant 2 | 58 |
| Human MUS81 Protein Sequence, Variant 2 | 59 |
| Human IFI16 cDNA Sequence, Variant 1 | 60 |
| HUMAN IFI16 Protein Sequence, Variant 1 | 61 |
| Human IFI16 cDNA Sequence, Variant 2 | 62 |
| Human IFI16 Protein Sequence, Variant 2 | 63 |
| Human IFI16 cDNA Sequence, Variant 3 | 64 |
| Human IFI16 Protein Sequence, Variant 3 | 65 |
| Human cGAS cDNA Sequence | 66 |
| Human cGAS Protein Sequence | 67 |
| Human DDX41 cDNA Sequence, Variant 1 | 68 |
| Human DDX41 Protein Sequence, Variant 1 | 69 |
| Human DDX41 cDNA Sequence, Variant 2 | 70 |
| HUMAN DDX41 Protein Sequence, Variant 2 | 71 |
| Human EXO1 cDNA Sequence, Variant 1 | 72 |
| Human EXO1 Protein Sequence, Variant 1 | 73 |
| Human EXO cDNA Sequence, Variant 2 | 74 |
| HUMAN EXO Protein Sequence, Variant 2 | 75 |
| Human EXO cDNA Sequence, Variant 3 | 76 |
| Human EXO Protein Sequence, Variant 3 | 77 |
| Human DNA2 cDNA Sequence | 78 |
| Human DNA2 Protein Sequence | 79 |
| Human RBBP8 cDNA Sequence, Variant 1 | 80 |
| Human RBBP8 Protein Sequence, Variant 1 | 81 |
| Human RBBP8 cDNA Sequence, Variant 2 | 82 |
| Human RBBP8 Protein Sequence, Variant 2 | 83 |
| Human MRE11 cDNA Sequence, Variant 1 | 84 |
| Human MRE11 Protein Sequence, Variant 1 | 85 |
| Human MRE11 cDNA Sequence, Variant 2 | 86 |
| Human MRE11 Protein Sequence, Variant 2 | 87 |
| Human MRE11 cDNA Sequence, Variant 3 | 88 |
| Human MRE11 Protein Sequence, Variant 3 | 89 |

Some embodiments of any of the methods described herein include determining the level of expression of a mRNA or a protein encoded by of one or more of STING, TREX1, BRCA1, BRCA2, SAMHD1, DNASE2, BLM, PARP1, RPA1, RAD51, MUS81, IFI16, cGAS, DDX41, EXO1, DNA2, RBBP8 (CtIP), and MRE11. In some examples of any of the methods described herein, increased STING or cGAS signaling activity can include, e.g., detecting a decreased level of a mRNA or a protein encoded by one or more of BRCA1, BRCA2, SAMHD1, DNASE2, BLM, PARP1, RPA1, and RAD51, and/or detecting an increased level of a mRNA or protein encoded by one or more of STING, MUS81, IFI16, cGAS, DDX41, EXO1, DNA2, RBBP8 (CtIP), and MRE11 in a mammalian cell (e.g., as compared to any of the exemplary reference levels described herein).

Some embodiments of any of the methods described herein, an increased cGAS/STING signaling activity can be determined by detecting of a gain-of-function mutation (e.g., a gene amplification or one or more activating amino acid substitutions in a protein encoded by one or more of MUS81, IFI16, cGAS, DDX41, EXO1, DNA2, RBBP8 (CtIP), and MRE1); a gene deletion of one or more of BRCA1, BRCA2, SAMHD1, DNASE2, BLM, PARP1, RPA1, and RAD51; one or more amino acid deletions in a protein encoded by one or more of BRCA1, BRCA2, SAMHD1, DNASE2, BLM, PARP1, RPA1, and RAD51; one or more inactivating amino acid mutations in a protein encoded by one or more of BRCA1, BRCA2, SAMHD1, DNASE2, BLM, PARP1, RPA1, or RAD51; or a frameshift mutation in one or more of BRCA1, BRCA2, SAMHD1, DNASE2, BLM, PARP1, RPA1, and RAD51.

Methods of detecting a level of each of these exemplary cGAS/STING signaling pathway activities are described herein. Additional examples of cGAS/STING signaling pathway activities are known in the art, as well as methods for detecting a level of the same.

As used herein, “gain-of-function mutation” refers to one or more nucleotide substitutions, deletions, and/or insertions in a gene that results in the production of a protein encoded by the gene that has one or more increased activities in a mammalian cell as compared to the version of the protein encoded by the corresponding wildtype gene. In some embodiments, a gain-of-function mutation can be a gene amplification or one or more activating amino acid substitutions in a protein encoded by one or more of MUS81, IFI16, cGAS, DDX41, EXO1, DNA2, RBBP8 (CtIP), STING, and MRE1.

As used herein, “loss-of-function mutation” refers to one or more nucleotide substitutions, deletions, and/or insertions in a gene that results in: a decrease in the level of expression of the encoded protein as compared to the level of the expression by the corresponding wildtype gene, and/or the expression of a protein encoded gene that has one or more decreased activities in a mammalian cell as compared to the version of the protein encoded by the corresponding wildtype gene. In some embodiments, a loss-of-function mutation can be a gene deletion, one or more amino acid deletions in a protein encoded by a gene, or one or more inactivating amino acid substitutions in a protein encoded by a gene.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, containing the indicated number of carbon atoms. For example, C1-10 indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclic rings include five-membered, six membered, and seven-membered heterocyclic rings.

The term “cycloalkyl” as used herein includes an aromatic or nonaromatic cyclic hydrocarbon radical having 3 to 10 carbons, such as 3 to 8 carbons, such as 3 to 7 carbons, wherein the cycloalkyl group which may be optionally substituted. Examples of cycloalkyls include five membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to an aromatic or nonaromatic 5-8 membered monocyclic, 8-12 membered bicyclic, or 11-14 membered tricyclic ring system radical having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkyls include five-membered, six-membered, and seven-membered heterocyclic rings. Examples include piperazinyl, pyrrolidinyl, dioxanyl, morpholinyl, tetrahydrofuranyl, and the like.

The term “hydroxy” refers to an OH group.

The term “amino” refers to an NH2 group.

The term “oxo” refers to O. By way of example, substitution of a CH2 a group with oxo gives a C═O group.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

Other features and advantages of the invention will be apparent from the following detailed description and FIGURES, and from the claims.

### DETAILED DESCRIPTION

The present invention is based on the discovery that cancer cells having decreased ATM level and/or activity are more sensitive to treatment with a STING antagonist or cGAS inhibitor. In view of these discoveries, provided herein are methods of treating a subject in need thereof with a treatment including a STING antagonist or cGAS inhibitor, methods of selecting a treatment for a subject in need thereof, where the treatment includes a STING antagonist or cGAS inhibitor, methods of selecting a subject for treatment with a STING antagonist or cGAS inhibitor, methods of selecting a subject for participation in a clinical trial with a STING antagonist or cGAS inhibitor, and methods of predicting a subject's responsiveness to a STING antagonist or cGAS inhibitor (e.g., a compound of any one of Formulas I-XXIV or Formulas M1-M6 or a compound shown in any one of Tables C1-C2).

Non-liming aspects of these methods are described below, and can be used in any combination without limitation. Additional aspects of these methods are known in the art.

### ATM

Ataxia-telangiectasia mutated kinase (ATM) is a DNA damage-inducible protein kinase that is recruited and activated by DNA double-strand breaks. ATM phosphorylates substrates (e.g., p53, CHK2, BRCA1, NBS1, and H2AX) participating in DNA damage response initiating activation of the DNA damage checkpoint, leading to cell cycle arrest, DNA repair, or apoptosis.

The ATM gene expresses the ATM protein that is located primarily in the nucleus of cells, where it helps control the rate of cell growth and division. The ATM protein also plays an important role in the normal development and activity of several body systems, including the nervous system and the immune system. Additionally, the ATM protein assists cells in recognizing damaged or broken DNA strands. DNA can be damaged by agents such as toxic chemicals or radiation, and breaks in DNA strands also occur naturally when chromosomes exchange genetic material during cell division. The ATM protein coordinates DNA repair by activating enzymes that fix the broken strands, while efficient repair of damaged DNA strands helps maintain the stability of the cell's genetic information.

ATM being a central component of the DNA repair machinery, dysfunction in the ATM gene results in non-canonical activation of STING. It has been shown that ATM plays an important role in the assembly of DNA damage-induced STING complex, wherein the molecular mechanism of double-strand break recognition by ATM and the subsequent phosphorylation of p53 provide a molecular link between DNA damage and STING complex formation (see, e.g., Dunphy et al., Molecular Cell 71:745-760, 2018). Furthermore, loss of ATM activity in cells leads to accumulation of cytosolic DNA and results in activation of the cGAS-STING pathway and cytokine production.

A decreased level or activity of ATM can be caused by any mechanism. Several mutations have been linked to inactivation of ATM. For example, mutations in the MRN subunit NBS1 and/or subunit Mre11 can result in decreased ATM activation. In some embodiments, heterozygous ATM mutations (e.g., germline ATM missense mutation 7271T>G, or IVD10-6T>G) can result in a decreased level of ATM activity (e.g., Bernstein et al., Br. J. Cancer 89(8):1513-1516, 2003). In some embodiments, amino acid substitutions in an ATM protein (e.g., G138R, V245A, or R2912K) can result in a decrease in one or more activities of an ATM protein (see, e.g., Squatrito et al., Cancer Cell 18(6), 2010). Additional examples of mutations in an ATM gene that result in a decrease in the level and/or activity of ATM include 8307G>A, 8140C>T, 1339C>T, 1240C>T, 9170G>C, and 8711A>G (see, e.g., Gilad et al., Human Molecular Genetics 5(4):433, 1996).

In some embodiments, a decreased level and/or activity of ATM can be a result of a deletion mutation in an ATM gene (e.g., 8578del3 deletion). In some embodiments, a decreased level and/or activity of ATM can be a result of an insertion mutation in an ATM gene (e.g., 5319ins9 insertion). In some embodiments, a decreased level and/or activity of ATM can be a result of ATM gene loss (e.g., loss of one allele of ATM or loss of both alleles of ATM). In some embodiments, a decreased level and/or activity of ATM can be a result of one or more amino acid deletions in a protein encoded by an ATM gene. In some embodiments, a decreased level and/or activity of ATM can be a result of one or more inactivating amino acid substitutions in a protein encoded by an ATM gene. In some embodiments, a decreased level and/or activity of ATM can be a result of one or more amino acid insertions in a protein encoded by an ATM gene.

In some embodiments, a decrease in the level of ATM can be the result of mutation in a regulatory region of an ATM gene (e.g., that results in a decrease in transcription of the ATM gene and/or a decrease in translation of an mRNA encoded by the ATM gene).

In some embodiments, a mutation (e.g., any of the exemplary types of mutations described herein) is present in both alleles of the ATM gene in the cancer cell. In some embodiments, a mutation (e.g., any of the exemplary types of mutations described herein) is present in one allele of the ATM gene in the cancer cell. In some embodiments, a mutation in an ATM gene results the production of a truncated and non-functional version of an ATM protein.

A sequence of an exemplary wildtype human ATM protein is SEQ ID NO: 90. A sequence of an exemplary wildtype human ATM cDNA is SEQ ID NO: 91.

### Methods of Treating

Provided herein are methods of treating a subject (e.g., any of the exemplary subjects described herein) in need thereof that include: (a) identifying a subject having a cell (e.g., a cancer cell) having decreased ATM level and/or activity (e.g., a decrease of 1% to about 99%, 1% to about 95%, 1% to about 90%, 1% to about 80%, 1% to about 70%, 1% to about 60%, 1% to about 50%, 1% to about 40%, 1% to about 30%, 1% to about 20%, 1% to about 10%, about 5% to about 99%, about 5% to about 90%, about 5% to about 80%, about 5% to about 70%, about 5% to about 60%, about 5% to about 55%, about 5% to about 50%, about 5% to about 40%, about 5% to about 30%, about 5% to about 20%, about 5% to about 10%, about 10% to about 99%, about 10% to about 90%, about 10% to about 80%, about 10% to about 70%, about 10% to about 60%, about 10% to about 50%, about 10% to about 40%, about 10% to about 30%, about 10% to about 20%, about 20% to about 99%, about 20% to about 90%, about 20% to about 80%, about 20% to about 70%, about 20% to about 60%, about 20% to about 50%, about 20% to about 40%, about 20% to about 30%, about 30% to about 99%, about 30% to about 90%, about 30% to about 80%, about 30% to about 70%, about 30% to about 60%, about 30% to about 50%, about 30% to about 40%, about 40% to about 99%, about 40% to about 90%, about 40% to about 80%, about 40% to about 70%, about 40% to about 60%, about 40% to about 50%, about 50% to about 99%, about 50% to about 90%, about 50% to about 80%, about 50% to about 70%, about 50% to about 60%, about 60% to about 99%, about 60% to about 90%, about 60% to about 80%, about 60% to about 70%, about 70% to about 99%, about 70% to about 90%, about 70% to about 80%, about 80% to about 99%, about 80% to about 90%, or about 90% to about 99%) or any subranges of this range described herein) (e.g., as compared to a reference level); and (b) administering a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof to the identified subject.

Also provided herein are methods of treating a subject (e.g., any of the exemplary subjects described herein) in need thereof that include: administering a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof to a subject identified as having a cell (e.g., a cancer cell) having decreased ATM level and/or activity (e.g., a decrease of about 1% to about 99%, or any subranges of this range described herein) (e.g., as compared to a reference level) in a tumor sample.

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

In some embodiments, the subject is further identified as having an elevated level of cGAMP in a serum or tumor sample obtained from the subject (e.g., an increase of between 1% and 1000%, or any of the subranges of this range described herein) (e.g., as compared to a reference level). In some embodiments, the subject is further identified as having a cancer cell having increased cGAS/STING signaling pathway activity (e.g., an increase of between 1% and 1000%, or any of the subranges of this range described herein) (e.g., as compared to a reference level).

In some embodiments, the subject has been diagnosed or identified as having a cancer. In some embodiments, the cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments the cancer is selected from the group consisting of: renal clear cell carcinoma, uveal melanoma, tongue squamous cell carcinoma, breast cancer, and skin cancer.

In some embodiments of any of the methods described herein, the STING antagonist or cGAS inhibitor is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof, with the proviso that in embodiments related to a gain of function mutation in STING, a cGAS inhibitor is not employed in a method described herein.

In some embodiments of any of the methods of treatment described herein, the method can result in a decreased risk (e.g., a 1% to a 99% decrease, or any of the subranges of this range described herein) of developing a comorbidity in the subject (e.g., as compared to the risk of developing a comorbidity in a subject having cancer cells having a similar decreased ATM level and/or activity and/or increased cGAS/STING signaling pathway activity, but administered a different treatment or a placebo).

Additional exemplary aspects that can be used or incorporated in these methods are described herein.

### Methods of Selecting a Treatment for a Subject

Provided herein are methods of selecting a treatment for a subject (e.g., any of the exemplary subjects described herein) in need thereof that include: (a) identifying a subject having a cell (e.g., a cancer cell) having decreased ATM level and/or activity (e.g., a decrease of about 1% to about 99%, or any subranges of this range described herein) (e.g., as compared to a reference level) in a tumor sample; and (b) selecting for the identified subject a treatment comprising a therapeutically effective amount of a STING antagonist or cGAS inhibitor (e.g., any of the exemplary STING antagonists or cGAS inhibitor described herein) or a pharmaceutically acceptable salt, solvate, or co-crystal thereof.

Provided herein are methods of selecting a treatment for a subject (e.g., any of the exemplary subjects described herein) in need thereof that include: selecting a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof for a subject identified as having a cell (e.g., a cancer cell) having decreased ATM level and/or activity (e.g., a decrease of about 1% to about 99%, or any subranges of this range described herein) (e.g., as compared to a reference level) in a tumor sample.

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

In some embodiments, the subject is further identified as having an elevated level of cGAMP in a serum or tumor sample obtained from the subject (e.g., an increase of between 1% and 1000%, or any of the subranges of this range described herein) (e.g., as compared to a reference level). In some embodiments, the subject is further identified as having a cancer cell having increased cGAS/STING signaling pathway activity (e.g., an increase of between 1% and 1000%, or any of the subranges of this range described herein) (e.g., as compared to a reference level).

In some embodiments, the subject has been diagnosed or identified as having a cancer. In some embodiments, the cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments, the cancer is selected from the group consisting of: renal clear cell carcinoma, uveal melanoma, tongue squamous cell carcinoma, breast cancer, and skin cancer. In some embodiments, the methods further comprise administering the selected treatment to the subject.

In some embodiments of any of the methods described herein, the STING antagonist or cGAS inhibitor is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the STING antagonists or cGAS inhibitors described herein, or a pharmaceutically acceptable salt, solvate, or co-crystal thereof. In some embodiments including a gain of function mutation in STING, a cGAS inhibitor is not employed in a method of the present disclosure.

Some embodiments of any of the methods described herein can further include recording the selected treatment in the subject's clinical record (e.g., a computer readable medium). Some embodiments of any of the methods described herein can further include administering one or more doses (e.g., at least two, at least four, at least six, at least eight, at least ten doses) of the selected treatment to the identified subject.

Additional exemplary aspects that can be used or incorporated in these methods are described herein.

### Methods of Selecting a Subject for Treatment

Also provided herein are methods of selecting a subject for treatment that include: (a) identifying a subject (e.g., any of the subjects described herein) having a cell (e.g., a cancer cell) having decreased ATM level and/or activity (e.g, a decrease of about 1% to about 99%, or any subranges of this range described herein) (e.g., as compared to a reference level) in a tumor sample; and (b) selecting an identified subject for treatment with a therapeutically effective amount of a STING antagonist or cGAS inhibitor (e.g., any of the exemplary STING antagonists or cGAS inhibitors described herein or known in the art) or a pharmaceutically acceptable salt, solvate, or co-crystal thereof.

Also provided herein are methods of selecting a subject for treatment that include selecting a subject (e.g., any of the subjects described herein) identified as having a cell (e.g., a cancer cell) having decreased ATM level and/or activity (e.g., a decrease to about 1% to about 99%, or any subranges of this range described herein) (e.g., as compared to a reference level) in a tumor sample, for treatment with a therapeutically effective amount of a STING antagonist or cGAS inhibitor (e.g., any of the exemplary STING antagonists or cGAS inhibitor described herein or known in the art) or a pharmaceutically acceptable salt, solvate, or co-crystal thereof.

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

In some embodiments, the subject is further identified as having an elevated level of cGAMP in a serum or tumor sample obtained from the subject (e.g., an increase of between 1% and 1000%, or any of the subranges of this range described herein) (e.g., as compared to a reference level). In some embodiments, the subject is further identified as having a cancer cell having increased cGAS/STING signaling pathway activity (e.g., an increase of between 1% and 1000%, or any of the subranges of this range described herein) (e.g., as compared to a reference level).

In some embodiments, the subject has been diagnosed or identified as having a cancer. In some embodiments, the cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments, the cancer is selected from the group consisting of: renal clear cell carcinoma, uveal melanoma, tongue squamous cell carcinoma, breast cancer, and skin cancer.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

### Methods of Selecting a Subject for Participation in a Clinical Trial

Provided herein are methods of selecting a subject (e.g., any of the exemplary subjects described herein) for participation in a clinical trial that include: (a) identifying a subject having a cancer cell having decreased ATM level and/or activity (e.g., a decrease of about 1% to about 99%, or any subranges of this range described herein) (e.g., as compared to a reference level) in a tumor sample; and (b) selecting the identified subject for participation in a clinical trial that comprises administration of a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof.

Also provided herein are methods of selecting a subject (e.g., any of the exemplary subjects described herein) for participation in a clinical trial that include: selecting a subject identified as having a cell (e.g., a cancer cell) having decreased ATM level and/or activity (e.g., a decrease of about 1% to about 99%, or any subranges of this range described herein) (e.g., as compared to a reference level) in a tumor sample for participation in a clinical trial that comprises administration of a treatment comprising a therapeutically effective amount of a STING antagonist or cGAS inhibitor (e.g., any of the exemplary STING antagonists or cGAS inhibitors described herein) or a pharmaceutically acceptable salt, solvate, or co-crystal thereof.

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

In some embodiments, the subject is further identified as having an elevated level of cGAMP in a serum or tumor sample from the subject (e.g., an increase of between 1% and 1000%, or any of the subranges of this range described herein) (e.g., as compared to a reference level). In some embodiments, the subject is further identified as having a cancer cell having increased cGAS/STING signaling pathway activity (e.g., an increase of between 1% and 1000%, or any of the subranges of this range described herein) (e.g., as compared to a reference level).

In some embodiments, the subject has been diagnosed or identified as having a cancer. In some embodiments, the cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments, the cancer is selected from the group consisting of: renal clear cell carcinoma, uveal melanoma, tongue squamous cell carcinoma, breast cancer, and skin cancer.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

Methods of Predicting a Subject's Responsiveness to a STING Antagonist or cGAS Inhibitor

Provided herein are methods of predicting a subject's (e.g., any of the exemplary subjects described herein) responsiveness to a compound of any one of Formulas I-XXIV or Formulas M1-M6 that include: (a) determining that a subject has a cancer cell having decreased ATM level and/or activity (e.g., a decrease of about 1% to about 99%, or any subranges of this range described herein) (e.g., as compared to a reference level) in a tumor sample; and (b) identifying that the subject determined to have decreased ATM level and/or activity (e.g., a decrease of about 1% to about 99%, or any subranges of this range described herein) (e.g., as compared to a reference level) in a tumor sample, in step (a) has an increased likelihood of being responsive to treatment with a compound of any one of Formulas I-XXIV or Formulas M1-M6.

Provided herein are methods of predicting a subject's (e.g., any of the exemplary subjects described herein) responsiveness to a STING antagonist or cGAS inhibitor that include: (a) determining that a subject has a cancer cell having decreased ATM level and/or activity (e.g., a decrease of about 1% to about 99%, or any subranges of this range described herein) (e.g., as compared to a reference level) in a tumor sample; and (b) identifying that the subject determined to have decreased ATM level and/or activity (e.g., a decrease of about 1% to about 99%, or any subranges of this range described herein) (e.g., as compared to a reference level) in a tumor sample, in step (a) has an increased likelihood of being responsive to treatment with a STING antagonist or a cGAS inhibitor.

Also provided herein are methods of predicting a subject's (e.g., any of the exemplary subjects described herein) responsiveness to a compound of any one of Formulas I-XXIV or Formulas M1-M6 that include: identifying a subject determined to have a cell (e.g., a cancer cell) having decreased ATM level and/or activity (e.g., a decrease of about 1% to about 99%, or any subranges of this range described herein) (e.g., as compared to a reference level) in a tumor sample, as having an increased likelihood of being responsive to treatment with a compound of any one of Formulas I-XXIV or Formulas M1-M6.

Also provided herein are methods of predicting a subject's (e.g., any of the exemplary subjects described herein) responsiveness to a STING antagonist or a cGAS inhibitor that include: identifying a subject determined to have a cell (e.g., a cancer cell) having decreased ATM level and/or activity (e.g., a decrease of about 1% to about 99%, or any subranges of this range described herein) (e.g., as compared to a reference level) in a tumor sample, as having an increased likelihood of being responsive to treatment with a STING antagonist or a cGAS inhibitor.

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

In some embodiments, the subject is further identified as having an elevated level of cGAMP in a serum or tumor sample from the subject (e.g., an increase of between 1% and 1000%, or any of the subranges of this range described herein) (e.g., as compared to a reference level). In some embodiments, the subject is further identified as having a cancer cell having increased cGAS/STING signaling pathway activity (e.g., an increase of between 1% and 1000%, or any of the subranges of this range described herein) (e.g., as compared to a reference level).

In some embodiments, the subject has been diagnosed or identified as having a cancer. In some embodiments, the cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer. In some embodiments, the cancer is selected from the group consisting of: renal clear cell carcinoma, uveal melanoma, tongue squamous cell carcinoma, breast cancer, and skin cancer.

In some embodiments, the methods further comprise administering a therapeutically effective amount of a STING antagonist or cGAS inhibitor to a subject identified as having an increased likelihood of being responsive to treatment with a STING antagonist or cGAS inhibitor.

In some embodiments of any of the methods described herein, the STING antagonist is an inhibitory nucleic acid (e.g., a short interfering RNA, an antisense nucleic acid, a cyclic dinucleotide, or a ribozyme). In some embodiments of any of the methods described herein, the STING antagonist or cGAS inhibitor is any of the compounds described herein, or a pharmaceutically acceptable salt, solvate, or co-crystal thereof.

Additional exemplary aspects that can be used or incorporated in these methods are described herein.

### Indications

In some embodiments, methods for treating a subject having condition, disease or disorder in which an increase in cGAS/STING signaling activity and/or a decrease in ATM level and/or activity contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same). In some embodiments of any of the methods described herein, the subject can have, or be identified or diagnosed as having, any of the conditions, diseases, or disorders in which an increase in cGAS/STING signaling activity and/or a decrease in ATM level and/or activity contributes to the pathology and/or symptoms and/or progression of the condition, disease, or disorder. In some embodiments of any of the methods described herein, the subject can be suspected of having or present with one or more symptoms of any of the conditions, diseases, or disorders described herein.

In some embodiments, the condition, disease or disorder is a cancer (e.g., renal clear cell carcinoma, kidney renal papillary cell carcinoma, chromophobe renal cell carcinoma, uveal melanoma, tongue squamous cell carcinoma, breast cancer, osteosarcoma, and skin cancer). In some embodiments, the condition, disease or disorder is a cancer (e.g., renal clear cell carcinoma, uveal melanoma, tongue squamous cell carcinoma, breast cancer, and skin cancer).

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the STING antagonist or cGAS inhibitor (e.g., any of the STING antagonists or cGAS inhibitors described herein or known in the art).

In certain embodiments, the second therapeutic agent or regimen is administered to the subject prior to contacting with or administering the STING antagonist or cGAS inhibitor (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the STING antagonist or cGAS inhibitor. By way of example, the second therapeutic agent or regimen and the STING antagonist or cGAS inhibitor are provided to the subject simultaneously in the same dosage form. As another example, the second therapeutic agent or regimen and the STING antagonist or cGAS inhibitor are provided to the subject concurrently in separate dosage forms.

In still other embodiments, the second therapeutic agent or regimen is administered to the subject after contacting with or administering the STING antagonist or cGAS inhibitor (e.g., about one hour after, or about 6 hours after, or about 12 hours after, or about 24 hours after, or about 48 hours after, or about 1 week after, or about 1 month after).

### Patient Selection

In some embodiments, the methods described herein include the step of identifying a subject (e.g., a patient) in need of treatment as having a cell (e.g., a cancer cell) having decreased ATM level and/or activity.

In some embodiments, the methods described herein further include the step of further identifying a subject (e.g., a patient) in need of treatment as having a cell (e.g., a cancer cell) having increased cGAS/STING signaling pathway activity (e.g., an increase of between 1% and 1000%, or any of the subranges of this range described herein) (e.g., as compared to a reference level). In some embodiments, the methods described herein further include identifying a subject (e.g., a patient) in need of treatment as having an elevated level of cGAMP in a serum or a tumor sample (e.g., an increase of between 1% and 1000%, or any of the subranges of this range described herein) (e.g., as compared to a reference level).

In some embodiments, the subject is identified as having a cancer cell having decreased ATM level. In some embodiments, the ATM level is a level of ATM protein in the cancer cell. In some embodiments, the identification of the subject as having a cancer cell having a decreased ATM level includes detecting a decreased level of ATM protein in the cancer cell. In some embodiments, the ATM level is a level of ATM mRNA in the cancer cell. In some embodiments, the identification of the subject as having a cancer cell having a decreased ATM level comprises detecting a decreased level of ATM mRNA in the cancer cell.

In some embodiments, the decreased ATM level and/or activity is a result of ATM gene loss in the cancer cell. In some embodiments, the ATM gene loss is loss of one allele of the ATM gene. In some embodiments, the ATM gene loss is loss of both alleles of the ATM gene. In some embodiments, the identification of the subject as having a cancer cell having decreased ATM level and/or activity comprises detecting ATM gene loss in the cancer cell.

In some embodiments, the decreased ATM level and/or activity is a result of one or more amino acid deletions, insertions, or post-translational modifications of a protein encoded by an ATM gene in the cancer cell. In some embodiments, the identification of the subject as having a cancer cell having decreased ATM level and/or activity comprises detecting one or more amino acid deletions, insertions, or post-translational modifications in a protein encoded by an ATM gene in the cancer cell. In some embodiments, the decreased ATM level and/or activity is a result of one or more inactivating amino acid substitutions in a protein encoded by an ATM gene in the cancer cell. In some embodiments, the identification of the subject as having a cancer cell having decreased ATM expression and/or activity comprises detecting one or more inactivating amino acid substitutions in a protein encoded by an ATM gene in the cancer cell.

Methods of Detecting the Level of ATM Activity and/or Expression

In some embodiments of any of the methods described herein, a mammalian cell having decreased level and/or activity of ATM can be identified by, e.g., detecting the presence of a mutation in an ATM gene (e.g., any of the exemplary mutations in an ATM gene described herein, such as an ATM gene loss (e.g., loss of one or both alleles of ATM), an amino acid deletion in the protein encoded by an ATM gene, an amino acid insertion in the protein encoded by an ATM gene, or an inactivating amino acid substitution in a protein encoded by an ATM gene). Non-limiting examples of assays that can be used to determine the level of the presence of any of these mutations (e.g., any of the mutations described herein) include Southern blot analysis, Northern blot analysis, mass spectrometry, UV absorbance, lab-on-a-chip, microfluidics, gene chip, intercalating dyes (e.g., ethidium bromide), gel electrophoresis, restriction digestion and electrophoresis, and sequencing (e.g., using any of the wide variety of sequencing methods described herein or known in the art), including polymerase chain reaction (PCR)-based methods, e.g., next generation sequencing, reverse transcription polymerase chain reaction (RT-PCR), TaqMan™, and microarray analysis.

For example, the detection of genomic DNA can include detection of the presence of one or more unique sequences found in genomic DNA (e.g., human genomic DNA) (e.g., satellite DNA sequences present in centromeres or heterochromatin, minisatellite sequences, microsatellite sequences, the sequence of a transposable element, a telomere sequence, a specific sequence (e.g., 250 base pairs to about 300 base pairs) containing one or more SNPs, or a specific sequence encoding a gene). Detection can be performed using labeled probes (e.g., fluorophore-, radioisotope-, enzyme-, quencher-, and enzyme-labeled probes), e.g., by hybridizing labeled probes to the genomic DNA present in the isolated genomic DNA sample or the control sample (e.g., in an electrophoretic gel) or hybridizing the labeled probes to the products of a PCR assay (e.g., a real-time PCR assay) or an assay that includes a PCR assay that utilized genomic DNA in the isolated genomic DNA test sample or the control sample as the template. Non-limiting examples of methods that can be used to generate probes include nick translation, random oligo primed synthesis, and end labeling.

A variety of assays for determining the genotype of a gene are known in the art. Non-limiting examples of such assays (which can be used in any of the methods described herein) include: dynamic allele-specific hybridization (see, e.g., Howell et al., Nature Biotechnol. 17:87-88, 1999), molecular beacon assays (see, e.g., Marras et al., “Genotyping Single Nucleotide Polymorphisms with Molecular Beacons,” In Kwok (Ed.), Single Nucleotide Polymorphisms: Methods and Protocols, Humana Press, Inc., Totowa, NJ, Vol. 212, pp. 111-128, 2003), microarrays (see, e.g., Affymetrix Human SNP 5.0 GeneChip), restriction fragment length polymorphism (RFLP) (see, e.g., Ota et al., Nature Protocols 2:2857-2864, 2007), PCR-based assays (e.g., tetraprimer ARMS-PCR (see, e.g., Zhang et al., Plos One 8:e62126, 2013), real-time PCR, allele-specific PCR (see, e.g., Gaudet et al., Methods Mol. Biol. 578:415-424, 2009), and TaqMan Assay SNP Genotyping (see, e.g., Woodward, Methods Mol. Biol. 1145:67-74, 2014, and TaqMan®OpenArray® Genotyping Plates from Life Technologies)), Flap endonuclease assays (also called Invader assays) (see, e.g., Olivier et al., Mutat. Res. 573:103-110, 2005), oligonucleotide ligation assays (see, e.g., Bruse et al., Biotechniques 45:559-571, 2008), single strand conformational polymorphism assays (see, e.g., Tahira et al., Human Mutat. 26:69-77, 2005), temperature gradient gel electrophoresis (see, e.g., Jones et al., “Temporal Temperature Gradient Electrophoresis for Detection of Single Nucleotide Polymorphisms,” in Single Nucleotide Polymophisms: Methods and Protocols, Volume 578, pp. 153-165, 2008) or temperature gradient capillary electrophoresis, denaturing high performance liquid chromatography (see, e.g., Yu et al., J. Clin. Pathol. 58:479-485, 2005), high-resolution melting of an amplified sequence containing the SNP (see, e.g., Wittwer et al., Clinical Chemistry 49:853-860, 2003), or sequencing (e.g., Maxam-Gilbert sequencing, chain-termination methods, shotgun sequencing, bridge PCR, and next-generation sequencing methods (e.g., massively parallel signature sequencing, polony sequencing, 454 pyrosequencing, Illumina (Solexa) sequencing, SOLiD sequencing, Ion Torrent semiconductor sequence, DNA nanoball sequencing, heliscope single molecule sequencing, and single molecule real-time sequencing). Additional details and a summary of various next-generation sequencing methods are described in Koboldt et al., Cell 155:27-38, 2013.

In some embodiments of any of the methods described herein, the genotyping of a gene includes a PCR assay (e.g., a real-time PCR-assay) (with or without a prior pre-amplification step (e.g., any of the pre-amplification methods described herein)). In some embodiments of any of the methods described herein the genotyping can be performed using TaqMan®-based sequencing (e.g., TaqMan®-based OpenArray® sequencing, e.g., high throughput TaqMan®-based Open Array® sequencing) (with or without a prior pre-amplification step (e.g., any of the pre-amplification methods described herein)).

In some embodiments of any of the methods described herein, the level of the protein or mRNA can be detected in a biological sample including blood, serum, exosomes, plasma, tissue, urine, feces, sputum, and cerebrospinal fluid.

Determination of a level of an ATM protein can be performed using commercially available assays (e.g., RayBiotch, Novus Bio, LSBio, Rocky Mountain Diagnostics). Additional methods for determining a level of an ATM protein can be performed using immunoblotting and proteomics techniques.

Non-limiting assays for ATM kinase activity are described in, e.g., Adams et al., Methods Mol. Biol. 1599:43-56, 2017; and Nyati et al., Methods Mol. Biol. 1596:131-145, 2017. Additional methods for determining ATM kinase activity are known in the art.

Methods of Detecting the Level of cGAS/STING Signaling Pathway Activity and/or Expression

In some embodiments of any of the methods described herein, the cGAS/STING signaling pathway activity is the secretion of a type I IFN or a type III IFN. In some embodiments of any of the methods described herein, the cGAS/STING signaling pathway activity is the secretion of IFN-α. In some embodiments of any of the methods described herein, the cGAS/STING signaling pathway activity is the secretion of IFN-β. Non-limiting examples of methods that can be used to detect the secretion of IFN-α and IFN-0 include immunohistochemistry, immunoassays, e.g., enzyme-linked immunosorbent assay (ELISA), sandwich ELISA, immunoprecipitation, and immunofluorescent assay.

Non-limiting methods of detecting cGAMP in serum or tissue include immunoassays, e.g., enzyme-linked immunosorbent assay (ELISA), sandwich ELISA, immunoprecipitation, and immunofluorescent assay) a mass spectrometry.

In some embodiments of any of the methods described herein, the cGAS/STING signaling pathway activity can be the level and/or activity of an upstream activator in the cGAS/STING signaling pathway (e.g., the level of one or more (e.g., two, three, four, five, or six) of MUS81 mRNA, MUS81 protein, IFI16 mRNA, IFI16 protein, cGAS mRNA, cGAS protein, DDX41 mRNA, DDX41 protein, EXO1 mRNA, EXO1 protein, DNA2 mRNA, DNA2 protein, RBBP8 mRNA, RBBP8 protein, MRE11 mRNA, or MRE11 protein in a mammalian cell (e.g., a mammalian cell obtained from a subject). In some embodiments of any of the methods described herein, the cGAS/STING signaling pathway activity can be determined by detecting the level and/or activity of an upstream suppressor of the cGAS/STING signaling pathway (e.g., the level of one or more (e.g., two, three, four, five, or six) of BRCA1 mRNA, BRCA1 protein, BRCA2 mRNA, BRCA2 protein, SAMHD1 mRNA, SAMHD1 protein, DNASE2 mRNA, DNASE2 protein, BLM mRNA, BLM protein, PARP1 mRNA, PARP1 protein, RPA1 mRNA, RPA1 protein, RAD51 mRNA, or RAD51 protein in a mammalian cell (e.g., a mammalian cell obtained from a subject).

Non-limiting assays that can be used to determine the level and/or activity of an upstream activator or upstream suppressor of the STING pathway include: Southern blot analysis, Northern blot analysis, polymerase chain reaction (PCR)-based methods, e.g., next generation sequencing, reverse transcription polymerase chain reaction (RT-PCR), TaqMan™, microarray analysis, immunohistochemistry, immunoassays, e.g., enzyme-linked immunosorbent assay (ELISA), sandwich ELISA, immunoprecipitation, immunofluorescent assay, mass spectrometry, immunoblot (Western blot), RIA, and flow cytometry.

In some embodiments of any of the methods described herein, a mammalian cell having an increased level of cGAS/STING signaling pathway activity can be identified by detecting the presence of one of more of the following the mammalian cell: a gain-of-function mutation in a cGAS/STING signaling pathway gene (e.g., a BRCA1 protein having a E111Gfs*3 frameshift insertion, numbered according to SEQ ID NO: 15, a BRCA1 protein having a N1784Kfs*3 frameshift insertion numbered according to SEQ ID NO: 25, a SAMH-IDI protein having a V133I amino acid substitution numbered according to SEQ ID NO: 27, a DNASE2 protein having R314W amino acid substitution numbered according to SEQ ID NO: 33, a BLM protein having a N515Mfs*16 frameshift deletion numbered according to SEQ ID NO: 37, a PARP1 protein having a S507Afs*17 frameshift deletion numbered according to SEQ ID NO: 43, a RPA1 mRNA splicing having a X12 splice mutation, or a RAD51 protein having R254* amino acid substitution numbered according to SEQ ID NO: 51).

Non-limiting examples of assays that can be used to determine the level of the presence of any of these mutations (e.g., any of the mutations described herein) include Southern blot analysis, Northern blot analysis, mass spectrometry, UV absorbance, lab-on-a-chip, microfluidics, gene chip, intercalating dyes (e.g., ethidium bromide), gel electrophoresis, restriction digestion and electrophoresis, and sequencing (e.g., using any of the wide variety of sequencing methods described herein or known in the art), including polymerase chain reaction (PCR)-based methods, e.g., next generation sequencing, reverse transcription polymerase chain reaction (RT-PCR), TaqMan™, and microarray analysis.

For example, the detection of genomic DNA can include detection of the presence of one or more unique sequences found in genomic DNA (e.g., human genomic DNA) (e.g., satellite DNA sequences present in centromeres or heterochromatin, minisatellite sequences, microsatellite sequences, the sequence of a transposable element, a telomere sequence, a specific sequence (e.g., 250 base pairs to about 300 base pairs) containing one or more SNPs, or a specific sequence encoding a gene). Detection can be performed using labeled probes (e.g., fluorophore-, radioisotope-, enzyme-, quencher-, and enzyme-labeled probes), e.g., by hybridizing labeled probes to the genomic DNA present in the isolated genomic DNA sample or the control sample (e.g., in an electrophoretic gel) or hybridizing the labeled probes to the products of a PCR assay (e.g., a real-time PCR assay) or an assay that includes a PCR assay that utilized genomic DNA in the isolated genomic DNA test sample or the control sample as the template. Non-limiting examples of methods that can be used to generate probes include nick translation, random oligo primed synthesis, and end labeling.

A variety of assays for determining the genotype of a gene are known in the art. Non-limiting examples of such assays (which can be used in any of the methods described herein) include: dynamic allele-specific hybridization (see, e.g., Howell et al., Nature Biotechnol. 17:87-88, 1999), molecular beacon assays (see, e.g., Marras et al., “Genotyping Single Nucleotide Polymorphisms with Molecular Beacons,” In Kwok (Ed.), Single Nucleotide Polymorphisms: Methods and Protocols, Humana Press, Inc., Totowa, NJ, Vol. 212, pp. 111-128, 2003), microarrays (see, e.g., Affymetrix Human SNP 5.0 GeneChip), restriction fragment length polymorphism (RFLP) (see, e.g., Ota et al., Nature Protocols 2:2857-2864, 2007), PCR-based assays (e.g., tetraprimer ARMS-PCR (see, e.g., Zhang et al., Plos One 8:e62126, 2013), real-time PCR, allele-specific PCR (see, e.g., Gaudet et al., Methods Mol. Biol. 578:415-424, 2009), and TaqMan Assay SNP Genotyping (see, e.g., Woodward, Methods Mol. Biol. 1145:67-74, 2014, and TaqMan® OpenArray® Genotyping Plates from Life Technologies)), Flap endonuclease assays (also called Invader assays) (see, e.g., Olivier et al., Mutat. Res. 573:103-110, 2005), oligonucleotide ligation assays (see, e.g., Bruse et al., Biotechniques 45:559-571, 2008), single strand conformational polymorphism assays (see, e.g., Tahira et al., Human Mutat. 26:69-77, 2005), temperature gradient gel electrophoresis (see, e.g., Jones et al., “Temporal Temperature Gradient Electrophoresis for Detection of Single Nucleotide Polymorphisms,” in Single Nucleotide Polymophisms: Methods and Protocols, Volume 578, pp. 153-165, 2008) or temperature gradient capillary electrophoresis, denaturing high performance liquid chromatography (see, e.g., Yu et al., J. Clin. Pathol. 58:479-485, 2005), high-resolution melting of an amplified sequence containing the SNP (see, e.g., Wittwer et al., Clinical Chemistry 49:853-860, 2003), or sequencing (e.g., Maxam-Gilbert sequencing, chain-termination methods, shotgun sequencing, bridge PCR, and next-generation sequencing methods (e.g., massively parallel signature sequencing, polony sequencing, 454 pyrosequencing, Illumina (Solexa) sequencing, SOLiD sequencing, Ion Torrent semiconductor sequence, DNA nanoball sequencing, heliscope single molecule sequencing, and single molecule real-time sequencing). Additional details and a summary of various next-generation sequencing methods are described in Koboldt et al., Cell 155:27-38, 2013.

In some embodiments of any of the methods described herein, the genotyping of a gene includes a PCR assay (e.g., a real-time PCR-assay) (with or without a prior pre-amplification step (e.g., any of the pre-amplification methods described herein)). In some embodiments of any of the methods described herein the genotyping can be performed using TaqMan®-based sequencing (e.g., TaqMan®-based OpenArray® sequencing, e.g., high throughput TaqMan®-based Open Array® sequencing) (with or without a prior pre-amplification step (e.g., any of the pre-amplification methods described herein)).

In some embodiments of any of the methods described herein, the level of the protein or mRNA can be detected in a biological sample including blood, serum, exosomes, plasma, tissue, urine, feces, sputum, and cerebrospinal fluid.

In some embodiments of any of the methods described herein, the level of at least one (e.g., 2, 3, 4, 5, 6, 7 or 8) parameters related to cGAS/STING signaling pathway activity and/or expression can be determined, e.g., in any combination.

In one aspect, the cell can be a cell isolated from a subject who has been screened for the presence of a cancer or an indication that is associated with an increase in a cGAS/STING signaling pathway activity and/or a decrease in ATM level or activity.

### Reference Levels

In some embodiments of any of the methods described herein, the reference level can be a corresponding level detected in a similar cell or sample obtained from a healthy subject (e.g., a subject that has not been diagnosed or identified as having a cancer, or any disorder associated with increased cGAS/STING signaling pathway activity and/or decreased ATM level and/or activity) (e.g., a subject who is not suspected or is not at increased risk of developing a cancer, or any disorder associated with increased cGAS/STING signaling pathway and/or decreased ATM level and/or activity activity and/or expression) (e.g., a subject that does not present with any symptom of a cancer, or any disorder associated with increased cGAS/STING signaling pathway activity and/or decreased ATM level and/or activity).

In some embodiments, a reference level can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the corresponding levels detected in similar samples in a population of healthy subjects (e.g., a population of subjects that have not been diagnosed or identified as having a cancer, or any disorder associated with increased cGAS/STING signaling pathway and/or decreased ATM level and/or activity) (e.g., a population of subjects who are not suspected or are not at increased risk of developing a cancer, or any disorder associated with increased cGAS/STING signaling pathway and/or decreased ATM level and/or activity) (e.g., a population of subjects that do not present with any symptom of a cancer, or any disorder associated with increased cGAS/STING signaling pathway and/or decreased ATM level and/or activity).

In some embodiments, a reference can be a corresponding level detected in a similar sample obtained from the subject at an earlier time point.

### STING Antagonists

In any of the methods described herein, the STING antagonist can be any of the STING antagonists described herein (e.g., any of the compounds described in this section). In any of the methods described herein, the STING antagonist has an IC₅₀ of between about 1 nM and about 10 μM for STING.

In some embodiments, the STING antagonist is a compound of Formula (I):

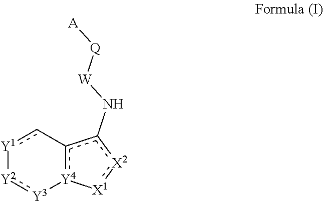

or a pharmaceutically acceptable salt thereof, or an N-oxide thereof, wherein:
Z, Y¹, Y², Y³, Y⁴, X¹, X², W, Q, and A can be as defined anywhere in WO 2020/010092, filed as PCT/US2019/040317 on Jul. 2, 2019; U.S. Provisional 62/693,768, filed on Jul. 3, 2018; and U.S. Provisional 62/861,825, filed on Jun. 14, 2019, each of which is incorporated herein by reference in its entirety.

In certain of these embodiments, Z, Y¹, Y², Y³, Y⁴, X¹, X², W, Q, and A are as defined in any one of claims 1 to 255 in WO 2020/010092, filed as PCT/US2019/040317 on Jul. 2, 2019, each of which is incorporated herein by reference in its entirety.

In certain embodiments, the STING antagonist is a compound as described in the table spanning pages 93 to 158 in WO 2020/010092, filed as PCT/US2019/040317 on Jul. 2, 2019, which is incorporated herein by reference in its entirety.

In some embodiments, the STING antagonist is a compound of Formula (II):

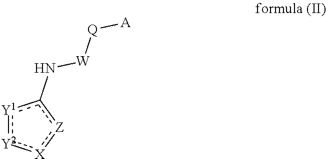

or a pharmaceutically acceptable salt thereof, wherein:
Y¹, Y², X, Z, W, Q, and A can be as defined anywhere in WO 2020/010155, filed as PCT/US2019/040418 on Jul. 2, 2019; U.S. Provisional 62/693,878, filed on Jul. 3, 2018; and U.S. Provisional 62/861,078, filed on Jun. 13, 2019, each of which is incorporated herein by reference in its entirety.

In certain of these embodiments, Y¹, Y², X, Z, W, Q, and A are as defined in any one of claims 1 to 115 in WO 2020/010155, filed as PCT/US2019/040418 on Jul. 2, 2019, each of which is incorporated herein by reference in its entirety.

In certain embodiments, the STING antagonist is a compound as described in the table spanning pages 34 to 44 in WO 2020/010155, filed as PCT/US2019/040418 on Jul. 2, 2019, which is incorporated herein by reference in its entirety.

In some embodiments, the STING antagonist is a compound of Formula (III):

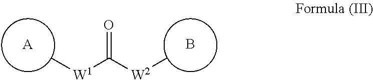

or a pharmaceutically acceptable salt thereof, or a tautomer thereof, wherein:
A, W¹, W², and B can be as defined anywhere in WO 2020/150417, filed as PCT/US2020/013786 on Jan. 16, 2020; U.S. Provisional 62/793,795, filed on Jan. 17, 2019; U.S. Provisional 62/861,865, filed on Jun. 14, 2019; U.S. Provisional 62/869,914, filed on Jul. 2, 2019; and U.S. Provisional 62/955,891, filed on Dec. 31, 2019, each of which is incorporated herein by reference in its entirety.

In certain of these embodiments, A, W¹, W², and B are as defined in any one of claims 1 to 116 in WO 2020/150417, filed as PCT/US2020/013786 on Jan. 16, 2020, each of which is incorporated herein by reference in its entirety.

In some embodiments, the STING antagonist is a compound of Formula (IV):

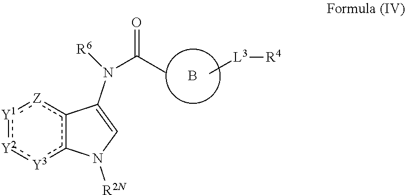

or a pharmaceutically acceptable salt thereof, or a tautomer thereof, wherein:
Z, Y¹, Y², Y³, R⁶, B, R^{2N}, L³, and R⁴ can be as defined anywhere in WO 2020/150417, filed as PCT/US2020/013786 on Jan. 16, 2020; U.S. Provisional 62/793,795, filed on Jan. 17, 2019; U.S. Provisional 62/861,865, filed on Jun. 14, 2019; U.S. Provisional 62/869,914, filed on Jul. 2, 2019; and U.S. Provisional 62/955,891, filed on Dec. 31, 2019, each of which is incorporated herein by reference in its entirety.

In certain of these embodiments, Z, Y¹, Y², Y³, R⁶, B, R^{2N}, L³, and R⁴ are as defined in any one of claims 117 to 223 in WO 2020/150417, filed as PCT/US2020/013786 on Jan. 16, 2020, each of which is incorporated herein by reference in its entirety.

In certain embodiments, the STING antagonist is a compound as described in Table C1 of WO 2020/150417, filed as PCT/US2020/013786 on Jan. 16, 2020, which is incorporated herein by reference in its entirety.

In some embodiments, the STING antagonist is a compound of Formula (V):

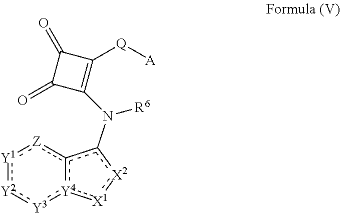

or a pharmaceutically acceptable salt thereof, or a tautomer thereof, wherein:
X¹, X², Y¹, Y², Y³, Y⁴, Z, Q, A, and R⁶ can be as defined anywhere in WO 2020/236586, filed as PCT/US2020/033127 on May 15, 2020; U.S. Provisional 62/849,811, filed on May 17, 2019; and U.S. Provisional 62/861,880, filed on Jun. 14, 2019; each of which is incorporated herein by reference in its entirety.

In certain of these embodiments, X¹, X², Y¹, Y², Y³, Y⁴, Z, Q, A, and R⁶ are as defined in any one of claims 1 to 18 and any one of the numbered clauses 1 to 271 in WO 2020/236586, filed as PCT/US2020/033127 on May 15, 2020, each of which is incorporated herein by reference in its entirety.

In certain embodiments, the STING antagonist is a compound as described in Table C1 of WO 2020/236586, filed as PCT/US2020/033127 on May 15, 2020, which is incorporated herein by reference in its entirety.

In some embodiments, the STING antagonist is a compound of Formula (VI):

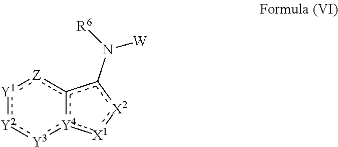

or a pharmaceutically acceptable salt thereof, or a tautomer thereof, wherein:
X¹, X², Y¹, Y², Y³, Y⁴, Z, W, and R⁶ can be as defined anywhere in WO 2020/243519 filed as PCT/US2020/035249 on May 29, 2020; U.S. Provisional 62/854,288, filed on May 29, 2019, which is incorporated herein by reference in its entirety.

In certain of these embodiments, X¹, X², Y¹, Y², Y³, Y⁴, Z, W, and R⁶ are as defined in any one of claims 1 to 16 and any one of numbered clauses 1-223 and 279-287 in WO 2020/243519 filed as PCT/US2020/035249 on May 29, 2020, each of which is incorporated herein by reference in its entirety.

In certain embodiments, the STING antagonist is a compound as described in the Table C1 of WO 2020/243519 filed as PCT/US2020/035249 on May 29, 2020, which is incorporated herein by reference in its entirety.

In some embodiments, the STING antagonist is a compound of Formula (VII):

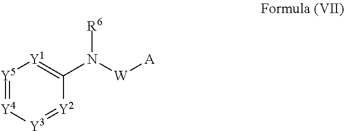

or a pharmaceutically acceptable salt thereof, or a tautomer thereof, wherein:
Y¹, Y², Y³, Y⁴, Y⁵, R⁶, W, and A can be as defined anywhere in WO 2020/252240 filed as PCT/US2020/037403 on Jun. 12, 2020; U.S. Provisional 62/861,714, filed on Jun. 14, 2019; and U.S. Provisional 62/955,924, filed on Dec. 31, 2019; each of which is incorporated herein by reference in its entirety.

In certain of these embodiments, Y¹, Y², Y³, Y⁴, Y⁵, R⁶, W, and A are as defined in any one of claims 1 to 16 and any one of numbered clauses 1 to 328 in PCT/US2020/037403 filed on Jun. 12, 2020, each of which is incorporated herein by reference in its entirety.

In certain embodiments, the STING antagonist is a compound as described in Table C1 of PCT/US2020/037403 filed on Jun. 12, 2020, which is incorporated herein by reference in its entirety.

In some embodiments, the STING antagonist is a compound of Formula (VIII):

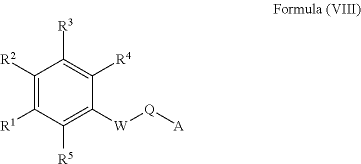

or a pharmaceutically acceptable salt thereof, wherein:
R¹, R², R³, R⁴, R⁵, W, Q, and A can be as defined anywhere in WO 2020/106741 filed as PCT/US2019/062245 on Nov. 19, 2019; U.S. Provisional 62/769,500, filed on Nov. 19, 2018; and U.S. Provisional 62/861,108, filed on Jun. 13, 2019; each of which is incorporated herein by reference in its entirety.

In certain of these embodiments, R¹, R², R³, R⁴, R⁵, W, Q, and A are as defined in any one of claims 1 to 118 in WO 2020/106741 filed as PCT/US2019/062245 on Nov. 19, 2019, each of which is incorporated herein by reference in its entirety.

In certain embodiments, the STING antagonist is a compound as described in table spanning pages 56-69 in WO 2020/106741 filed as PCT/US2019/062245 on Nov. 19, 2019, each of which is incorporated herein by reference in its entirety.

In some embodiments, the STING antagonist is a compound of Formula (IX):

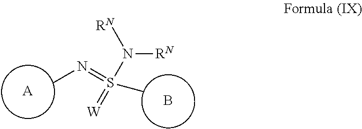

or a pharmaceutically acceptable salt thereof, or a tautomer thereof, wherein:
A, B, W, and R^{N} can be as defined anywhere in WO 2020/106736 filed as PCT/US2019/062238 on Nov. 19, 2019; U.S. Provisional 62/769,327, filed on Nov. 19, 2018 and U.S. Provisional 62/861,781, filed on Jun. 14, 2019, each of which is incorporated herein by reference in its entirety.

In certain of these embodiments, A, B, W, and R^{N} are as defined in any one of claims 1 to 298 in WO 2020/106736 filed as PCT/US2019/062238 on Nov. 19, 2019, each of which is incorporated herein by reference in its entirety.

In certain embodiments, the STING antagonist is a compound as described in Table 1A and Table 1B of WO 2020/106736 filed as PCT/US2019/062238 on Nov. 19, 2019, each of which is incorporated herein by reference in its entirety.

In some embodiments, the STING antagonist is a compound of Formula (X):

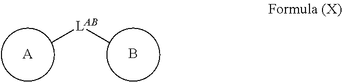

or a pharmaceutically acceptable salt thereof, or a tautomer thereof, wherein:
A, B, and L^{AB} can be as defined anywhere in WO 2020/150439 filed as PCT/US2020/013824 on Jan. 16, 2020; U.S. Provisional 62/793,623, filed on Jan. 17, 2019; and U.S. Provisional 62/861,702, filed on Jun. 14, 2019; each of which is incorporated herein by reference in its entirety.

In certain of these embodiments, A, B, and L^{AB} are as defined in any one of claims 1 to 116 and 172-249 in WO 2020/150439 filed as PCT/US2020/013824 on Jan. 16, 2020, each of which is incorporated herein by reference in its entirety.

In certain embodiments, the STING antagonist is a compound as described in Table C1 of WO 2020/150439 filed as PCT/US2020/013824 on Jan. 16, 2020, each of which is incorporated herein by reference in its entirety.

In some embodiments, the STING antagonist is a compound of Formula (XI):

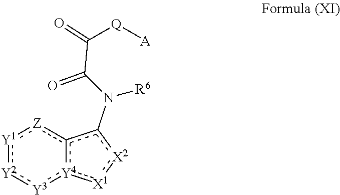

or a pharmaceutically acceptable salt thereof, or a tautomer therefore, wherein:
X¹, X², Y¹, Y², Y³, Y⁴, Z, Q, A, and R⁶ can be as defined anywhere in WO 2021/067791, filed as PCT/US2020/054054 on Oct. 2, 2020; U.S. Provisional 62/910,162, filed on Oct. 3, 2019; and U.S. Provisional 62/955,921, filed on Dec. 31, 2019; each of which is incorporated herein by reference in its entirety.

In certain of these embodiments, X¹, X², Y¹, Y², Y³, Y⁴, Z, Q, A, and R⁶ are as defined in any one of claims 1 to 16 and any one of the numbered clauses 1 to 179 in PCT/US2020/054054 filed on Oct. 2, 2020, each of which is incorporated herein by reference in its entirety.

In certain embodiments, the STING antagonist is a compound as described in Table C1 of PCT/US2020/054054 filed on Oct. 2, 2020, each of which is incorporated herein by reference in its entirety.

In some embodiments, the STING antagonist is a compound of Formula (XII):

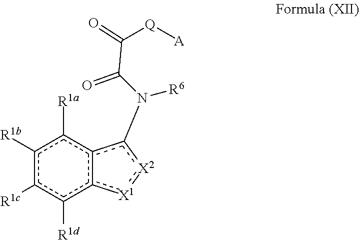

or a pharmaceutically acceptable salt thereof, or a tautomer thereof, wherein:
R^{1a}, R^{1b}, R^{1c}, R^{1d}, X¹, X², Q, A, and R⁶ can be as defined anywhere in WO 2021/067805 filed as PCT/US2020/054069 filed on Oct. 2, 2020; U.S. Provisional 62/910,160, filed on Oct. 3, 2019; and U.S. Provisional 62/955,867, filed on Dec. 31, 2019; each of which is incorporated herein by reference in its entirety.

In certain of these embodiments, R^{1a}, R^{1b}, R^{1c}, R^{1d}, X¹, X², Q, A, and R⁶ are as defined in any one of claims 1 to 16 and any one of the numbered clauses 1 to 296 in PCT/US2020/054069 filed on Oct. 2, 2020, each of which is incorporated herein by reference in its entirety.

In certain embodiments, the STING antagonist is a compound as described in Table C1 of in PCT/US2020/054069 filed on Oct. 2, 2020, each of which is incorporated herein by reference in its entirety.

In some embodiments, the STING antagonist is a compound of Formula (XIII):

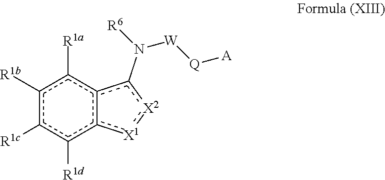

or a pharmaceutically acceptable salt, or a tautomer thereof, wherein:
R^{1a}, R^{1b}, R^{1c}, R^{1d}, X¹, X², W, Q, A, and R⁶ can be as defined anywhere in WO 2021/067801 filed as PCT/US2020/054064 on Oct. 2, 2020; U.S. Provisional 62/910,230, filed on Oct. 3, 2019; and U.S. Provisional 62/955,899, filed on Dec. 31, 2019; each of which is incorporated herein by reference in its entirety.

In certain of these embodiments, R^{1a}, R^{1b}, R^{1c}, R^{1d}, X¹, X², W, Q, A, and R⁶ are as defined in any one of claims 1 to 16 and any one of the numbered clauses 1 to 181 in PCT/US2020/054064 filed on Oct. 2, 2020, each of which is incorporated herein by reference in its entirety.

In certain embodiments, the STING antagonist is a compound as described in Table C1 of PCT/US2020/054064 filed on Oct. 2, 2020, each of which is incorporated herein by reference in its entirety.

In some embodiments, the STING antagonist is a compound of Formula (XIV):

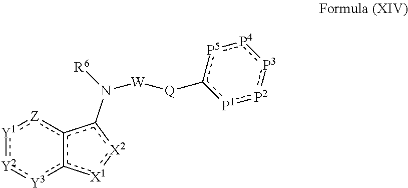

or a pharmaceutically acceptable salt thereof or a tautomer thereof, wherein:
Z, Y¹, Y², Y³, X¹, X², R⁶, W, Q, P¹, P², P³, P⁴, and P⁵ can be as defined anywhere in WO 2021/138419 filed as PCT/US2020/067463 on Dec. 30, 2020; U.S. Provisional 63/090,547 filed on Oct. 12, 2020; and U.S. Provisional 62/955,853 filed on Dec. 31, 2019, each of which is incorporated herein by reference in its entirety.

In certain of these embodiments, Z, Y¹, Y², Y³, X¹, X², R⁶, W, Q, P¹, P², P³, P⁴, and P⁵ are as defined in any one of claims 1 to 16 and any one of the numbered clauses 1 to 220 in U.S. Provisional 63/090,547 filed on Oct. 12, 2020, each of which is incorporated herein by reference in its entirety.

In certain embodiments, the STING antagonist is a compound as described in Table C1 of U.S. Provisional Application Ser. No. 63/090,547 filed on Oct. 12, 2020, each of which is incorporated herein by reference in its entirety.

In some embodiments, the STING antagonist is a compound of Formula (XV):

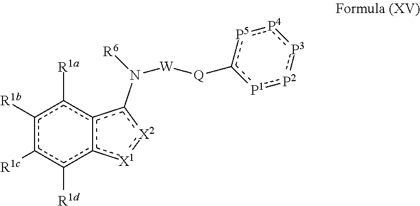

or a pharmaceutically acceptable salt thereof or a tautomer thereof, wherein:
R^{1a}, R^{1b}, R^{1c}, R^{1d}, X¹, X², R⁶, W, Q, P¹, P², P³, P⁴, and P⁵ can be as defined anywhere in WO 2021/138434 filed as PCT/US2020/067483 on Dec. 30, 2020; U.S. Provisional 63/090,538 filed on Oct. 12, 2020; and U.S. Provisional 62/955,839 filed on Dec. 31, 2019, each of which is incorporated herein by reference in its entirety.

In certain of these embodiments, R^{1a}, R^{1b}, R^{1c}, R^{1d}, X¹, X², R⁶, W, Q, P¹, P², P³, P⁴, and P⁵ are as defined in any one of claims 1 to 16 and any one of the numbered clauses 1 to 240 in U.S. Provisional 63/090,538 filed on Oct. 12, 2020, each of which is incorporated herein by reference in its entirety.

In certain embodiments, the STING antagonist is a compound as described in Table C1 of U.S. Provisional 63/090,538 filed on Oct. 12, 2020, each of which is incorporated herein by reference in its entirety.

In some embodiments, the STING antagonist is a compound of Formula (XVI):

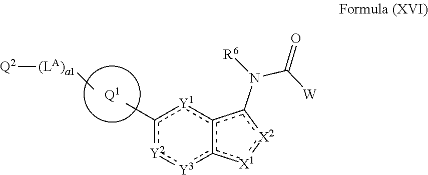

or a pharmaceutically acceptable salt thereof or a tautomer thereof, wherein
Q², L^{A}, at, Ring Q¹, Y¹, Y², Y³, X¹, X², R⁶ and W can be defined anywhere in PCT/US2021/041823, filed on Jul. 15, 2021; and U.S. Provisional 63/052,084 filed on Jul. 15, 2020, each of which is incorporated herein by reference in its entirety.

In certain of these embodiments, Q², L^{A}, at, Ring Q¹, Y¹, Y², Y³, X¹, X², R⁶ and W are as defined in any one of claims 1 to 20 and any one of the numbered clauses 1 to 176 in PCT/US2021/041823 filed on Jul. 15, 2021, each of which is incorporated herein by reference in its entirety.

In certain embodiments, the STING antagonist is a compound as described in Table C1 of PCT/US2021/041823 filed on Jul. 15, 2021, each of which is incorporated herein by reference in its entirety.

In some embodiments, the STING antagonist is a compound of Formula (XVII):

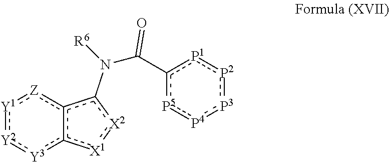

or a pharmaceutically acceptable salt thereof or a tautomer thereof, wherein
Z, Y¹, Y², Y³, X¹, X², R⁶, P¹, P², P³, P⁴, and P⁵ can be defined anywhere in PCT/US2021/041820, filed on Jul. 15, 2021; and U.S. Provisional 63/052,086 filed on Jul. 15, 2020, each of which is incorporated herein by reference in its entirety.

In certain of these embodiments, Z, Y¹, Y², Y³, X¹, X², R⁶, P¹, P², P³, P⁴, and P⁵ are as defined in any one of claims 1 to 19 and any one of the numbered clauses 1 to 193 in PCT/US2021/041820 filed on Jul. 15, 2021, each of which is incorporated herein by reference in its entirety.

In certain embodiments, the STING antagonist is a compound as described in Table C1 of PCT/US2021/041820 filed on Jul. 15, 2021, each of which is incorporated herein by reference in its entirety.

In some embodiments, the STING antagonist is a compound of Formula (XVIII):

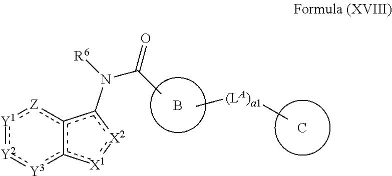

or a pharmaceutically acceptable salt thereof or a tautomer thereof, wherein
Z, Y¹, Y², Y³, X¹, X², R⁶, Ring B, L^{A}, al, and Ring C can be defined anywhere in PCT/US2021/041817, filed on Jul. 15, 2021; and U.S. Provisional 63/052,080 filed on Jul. 15, 2020, each of which is incorporated herein by reference in its entirety.

In certain of these embodiments, Z, Y¹, Y², Y³, X¹, X², R⁶, Ring B, L^{A}, al, and Ring C are as defined in any one of claims 1 to 20 and any one of the numbered clauses 1 to 196 in PCT/US2021/041817 filed on Jul. 15, 2021, each of which is incorporated herein by reference in its entirety.

In certain embodiments, the STING antagonist is a compound as described in Table C1 of PCT/US2021/041817 filed on Jul. 15, 2021, each of which is incorporated herein by reference in its entirety.

In some embodiments, the STING antagonist is a compound of Formula (XIX):

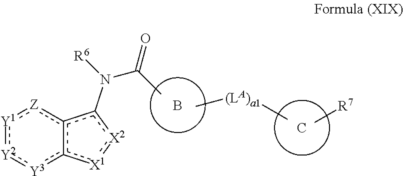

or a pharmaceutically acceptable salt thereof or a tautomer thereof, wherein
Z, Y¹, Y², Y³, X¹, X², R⁶, Ring B, L^{A}, a1, Ring C and R⁷ can be defined anywhere in PCT/US2021/041792, filed on Jul. 15, 2021; and U.S. Provisional 63/052,117 filed on Jul. 15, 2020, each of which is incorporated herein by reference in its entirety.

In certain of these embodiments, Z, Y¹, Y², Y³, X¹, X², R⁶, Ring B, L^{A}, al, Ring C and R⁷ are as defined in any one of claims 1 to 17 and any one of the numbered clauses 1 to 173 in PCT/US2021/041792, filed on Jul. 15, 2021, each of which is incorporated herein by reference in its entirety.

In certain embodiments, the STING antagonist is a compound as described in Table C1 of PCT/US2021/041792 filed on Jul. 15, 2021, each of which is incorporated herein by reference in its entirety.

In some embodiments, the STING antagonist is a compound of Formula (XX):

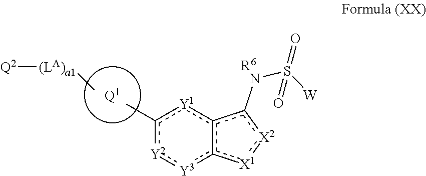

or a pharmaceutically acceptable salt thereof or a tautomer thereof, wherein
Q², L^{A}, a1, Q¹, Y¹, Y², Y³, X¹, X², R⁶ and W can be defined anywhere in U.S. utility application Ser. No. 17/376,823, filed on Jul. 15, 2021; and U.S. Provisional 63/052,076, filed on Jul. 15, 2020, each of which is incorporated herein by reference in its entirety.

In certain of these embodiments, Q², L^{A}, a1, Q¹, Y¹, Y², Y³, X¹, X², R⁶ and W and Ring C are as defined in any one of claims 1 to 19 and any one of the numbered clauses 1 to 186 in U.S. utility application Ser. No. 17/376,823 filed on Jul. 15, 2021, each of which is incorporated herein by reference in its entirety.

In certain embodiments, the STING antagonist is a compound as described in Table C1 of U.S. utility application Ser. No. 17/376,823 filed on Jul. 15, 2021, each of which is incorporated herein by reference in its entirety.

In some embodiments, the STING antagonist is a compound of Formula (XXI):

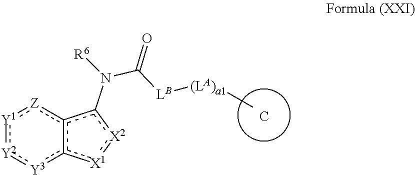

or a pharmaceutically acceptable salt thereof or a tautomer thereof, wherein
Z, Y¹, Y², Y³, X¹, X², R⁶, Ring B, L^{B}, L^{A}, at, and Ring C can be defined anywhere in U.S. utility application Ser. No. 17/376,829, filed on Jul. 15, 2021; and U.S. Provisional 63/052,052, filed on Jul. 15, 2020, each of which is incorporated herein by reference in its entirety.

In certain of these embodiments, Z, Y¹, Y², Y³, X¹, X², R⁶, Ring B, L^{B}, L^{A}, a1, and Ring C are as defined in any one of claims 1 to 17 and any one of the numbered clauses 1 to 181 in U.S. utility application Ser. No. 17/376,829 filed on Jul. 15, 2021, each of which is incorporated herein by reference in its entirety.

In certain embodiments, the STING antagonist is a compound as described in Table C1 of U.S. utility application Ser. No. 17/376,829 filed on Jul. 15, 2021, each of which is incorporated herein by reference in its entirety.

In some embodiments, the STING antagonist is a compound of Formula (XXII):

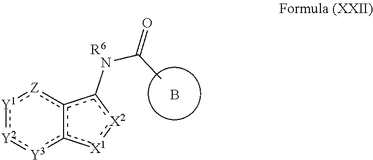

or a pharmaceutically acceptable salt thereof or a tautomer thereof, wherein
Z, Y¹, Y², Y³, X¹, X², R⁶, and Ring B can be defined anywhere in PCT/US2021/041758, filed on Jul. 15, 2021; and U.S. Provisional 63/052,083 filed on Jul. 15, 2020, each of which is incorporated herein by reference in its entirety.

In certain of these embodiments, Z, Y¹, Y², Y³, X¹, X², R⁶, and Ring B are as defined in any one of claims 1 to 18 and any one of the numbered clauses 1 to 157 in PCT/US2021/041758 filed on Jul. 15, 2021, each of which is incorporated herein by reference in its entirety.

In certain embodiments, the STING antagonist is a compound as described in Table C1 of PCT/US2021/041758 filed on Jul. 15, 2021, each of which is incorporated herein by reference in its entirety.

In some embodiments, the STING antagonist is a compound of Formula (XXIII):

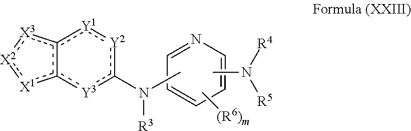

or a pharmaceutically acceptable salt thereof or a tautomer thereof, wherein
X¹, X², X³, Y¹, Y², Y³, R³, R⁴, R⁵, R⁶, and m can be defined anywhere in U.S. Provisional 63/126,332 filed on Dec. 16, 2020, each of which is incorporated herein by reference in its entirety.

In certain of these embodiments, X¹, X², X³, Y¹, Y², Y³, R³, R⁴, R⁵, R⁶, and m are as defined in any one of claims 1 to 20 and any one of the numbered clauses 1 to 174 in U.S. Provisional 63/126,332 filed on Dec. 16, 2020, each of which is incorporated herein by reference in its entirety.

In certain embodiments, the STING antagonist is a compound as described in Table C1 of U.S. Provisional 63/126,332 filed on Dec. 16, 2020, each of which is incorporated herein by reference in its entirety.

In some embodiments, the STING antagonist is a compound of Formula (XXIV):

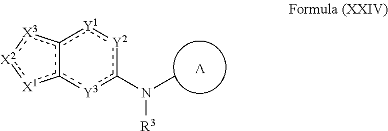

or a pharmaceutically acceptable salt thereof or a tautomer thereof, wherein
X¹, X², X³, Y¹, Y², Y³, R³, and Ring A can be defined anywhere in U.S. Provisional 63/126,286 filed on Dec. 16, 2020, each of which is incorporated herein by reference in its entirety.

In certain of these embodiments, X¹, X², X³, Y¹, Y², Y³, R³, and Ring A are as defined in any one of claims 1 to 23 and any one of the numbered clauses 1 to 183 in U.S. Provisional 63/126,286 filed on Dec. 16, 2020, each of which is incorporated herein by reference in its entirety.

In certain embodiments, the STING antagonist is a compound as described in Table C1 of U.S. Provisional 63/126,286 filed on Dec. 16, 2020, each of which is incorporated herein by reference in its entirety.

In some embodiments, the STING antagonist is a compound of Formula (M1):

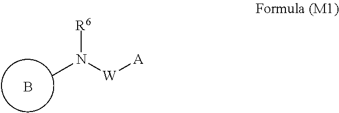

or a pharmaceutically acceptable salt thereof, wherein:
Ring B is selected from the group consisting of: (B-1), (B-2), and (B-3);

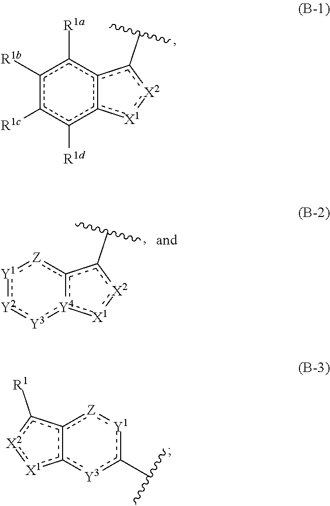

X¹ is selected from the group consisting of O, S, N, NR², and CR⁵;
X² is selected from the group consisting of O, S, N, NR⁴, and CR⁵;
each of Z, Y¹, Y², and Y³ is independently selected from the group consisting of: CR¹, N, and NR²;
Y⁴ is C or N;
each [symbol] is independently a single bond or a double bond;
provided that in (B-1), (B-2), and (B-3), the five-membered ring comprising X¹ and X² is heteroaryl;
provided that in (B-1), the 6-membered ring

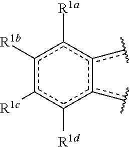

is aromatic;
provided that in (B-2), the 6-membered ring

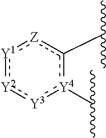

is aromatic, and one or more of Z, Y¹, Y², Y³, and Y⁴ in (B-2) is an independently selected heteroatom; and
provided that in (B-3), the 6-membered ring

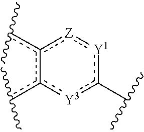

is aromatic;
W is selected from the group consisting of:
*C(═O)NR^{N}, *C(═S)NR^{N}, *C(═NR^{N})NR^{N}, *C(═NCN)NR^{N}, *C(═CNO₂)NR^{N}*S(O)₁₋₂NR^{N};

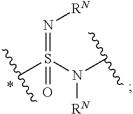

C(═O), *S(O)₂;

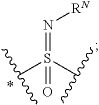

and

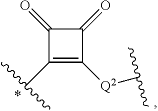

wherein Q² is selected from the group consisting of: a bond, NR^{N}, —S—, and —O—;
each R^{N} is independently selected from the group consisting of: H and R^{d}, and the asterisk represents point of attachment to NR⁶;
A is:
(i) —(Y^{A1})ₙ—Y^{A2}, wherein:
   n is 0 or1;
   Y^{A1} is C₁₋₆ alkylene, which is optionally substituted with 1-6 R^{a};
   Y^{A2} is:
      (a) C₃₋₂₀ cycloalkyl or C₃₋₂₀ cycloalkenyl, each of which is optionally substituted with 1-4 R^{b},
      (b) C₆₋₂₀ aryl which is optionally substituted with 1-4 R^{c},
      (c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or
      (d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},
or
(ii) C₁₋₂₀ alkyl, which is optionally substituted with 1-6 independently selected R^{a}; each of R¹, R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) C₁₋₆ alkyl, which is optionally substituted with 1-2 independently selected R^{a};
(ii) C₃₋₆ cycloalkyl, C₃₋₆ cycloalkenyl, or C₆₋₁₀ aryl;
(iii) heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂;
(iv) heteroaryl of 5-10 ring atoms, wherein 1-3 ring atoms are heteroatoms, each independently selected from the group consisting of N, N(H), N(R^{d}), O, and S(O)₀₋₂;
(v) —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and
(vi) H;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃-6 cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); and cyano;
each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; oxo; C₁₋₄ alkoxy optionally substituted with 1-2 independently selected R^{a}; C₁₋₄ haloalkoxy; —S(O)₁₋₂(C₁₋₄ alkyl) or —S(O)₁₋₂(C₁₋₄ haloalkyl); —NR^{e}R^{f}; —OH; —S(O)₁₋₂(NR′R″); —C₁₋₄ thioalkoxy or —C₁₋₄ thiohaloalkoxy; —NO₂; —SF₅; —C(═O)(C₁-10 alkyl); —C(═O)O(C₁₋₄ alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R^{f}), which are each independently selected from the group consisting of N(R^{d}), NH, O, and S;
-L¹ is a bond or C₁₋₃ alkylene;
-L² is —O—, —N(H)—, —N(C₁₋₃ alkyl)-, —S(O)₀₋₂—, or a bond;
-L³ is a bond or C₁₋₃ alkylene;
-L⁴ is —O—, —N(H)—, —N(C₁₋₃ alkyl)-, —S(O)₀₋₂—, or a bond;
each occurrence of R^{h} and R is independently selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In certain embodiments of Formula (M1), Ring B is (B-2)

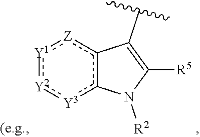

such as

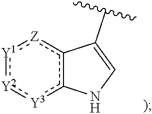

and W is selected from the group consisting of: *C(═O)NR^{N}*C(═S)NR^{N}, *C(═NR^{N})NR^{N}, *C(═NCN)NR^{N}, *C(═CNO₂)NR^{N}*S(O)₁₋₂NR^{N};

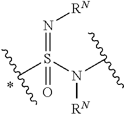

(e.g., C(═O)NR^{N}, such as C(═O)NH).

In certain embodiments of Formula (M1), Ring B is (B-1) (e.g.,

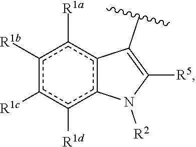

such as

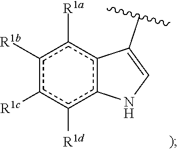

### or (B-2)

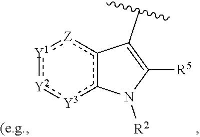

such as

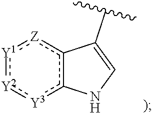

### and W is *C(═O).

In certain embodiments of Formula (M1), Ring B is (B-1)

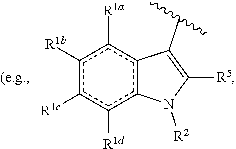

such as

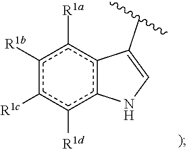

### or (B-2)

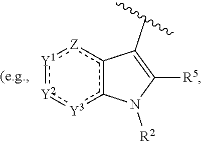

such as

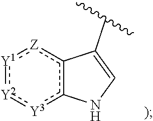

### and W is *S(O)₂ or

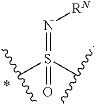

In certain embodiments of Formula (M1), Ring B is (B-1)

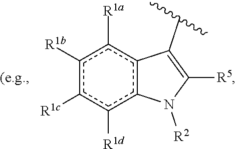

such as

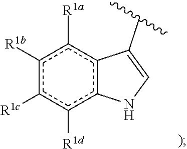

### or (B-2)

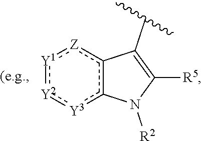

such as

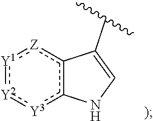

### and W is

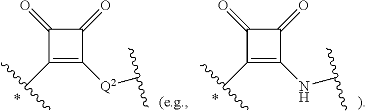

In certain embodiments of Formula (M1), Ring B is (B-3)

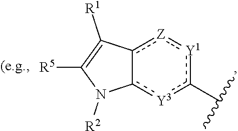

such as

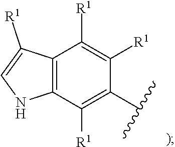

### and W is C(═O)NR^{N} (e.g., C(═O)NH).

In certain embodiments of Formula (M1), Ring B is (B-1)

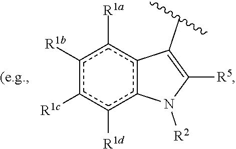

such as

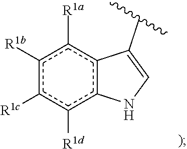

W is C(═O)NR^{N} (e.g., C(═O)NH); and one of R^{1a}, R^{1b}, R^{1c}, and R^{1d} (e.g., R^{1b}) is -L³-L⁴-Rⁱ (e.g., —Rⁱ).

In some embodiments, the STING antagonist is a compound of Formula (M2):

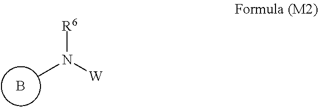

or a pharmaceutically acceptable salt thereof, wherein:
W is defined according to (AA) or (BB) below:
   (AA)
W is Q¹-Q²-A, wherein
Q¹ is selected from the group consisting of:
(a) phenyl optionally substituted with from 1-2 independently selected Rai; and
(b) heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected Rai;
Q² is selected from the group consisting of: a bond, —NH—, —N(C₁₋₃ alkyl)-, —O—, —C(═O), and —S(O)₀₋₂—;
A is as defined for Formula (M1) herein; or
   (BB)
W is selected from the group consisting of:
(a) C₇₋₂₀ bicyclic or polycyclic aryl, which is optionally substituted with from 1-4 R^{e}; and
(b) bicyclic or polycyclic heteroaryl including from 7-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};
each occurrence of R^{q1} is independently selected from the group consisting of:
(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (1) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo; and
Ring B, R⁶, R^{a}, R^{c}, R^{d}, R^{e}, R^{f}, R′, and R″ are each as defined for Formula (M1) herein.

In certain embodiments of Formula (M2), Ring B is (B-3)

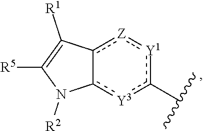

such as

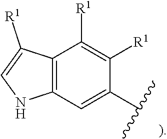

In certain embodiments of Formula (M2), Ring B is (B-1)

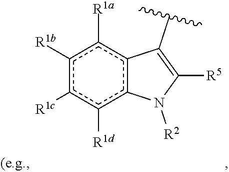

such as

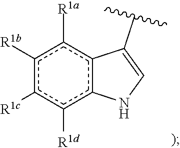

### or (B-2)

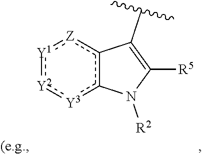

such as

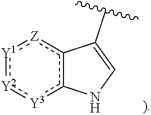

In some embodiments, the STING antagonist is a compound of Formula (M3):

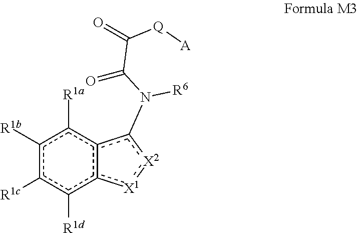

or a pharmaceutically acceptable salt thereof or a tautomer thereof,
   wherein:
X¹ is selected from the group consisting of O, S, N, NR², and CR⁵;
X² is selected from the group consisting of O, S, N, NR⁴, and CR⁵;
each [symbol] is independently a single bond or a double bond, provided that the five-membered ring comprising X¹ and X² is heteroaryl; and
the 6-membered ring

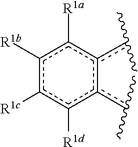

is aromatic;
Q-A is defined according to (A) or (B) below:
   (A)
Q is selected from the group consisting of: NH and N(C₁₋₆ alkyl) wherein the C₁₋₆ alkyl is optionally substituted with 1-2 independently selected R^{a}; and
A is:
(i) —(Y^{A1})ₙY^{A2}, wherein:
   n is 0 or 1;
   Y^{A1} is C₁₋₆ alkylene, which is optionally substituted with 1-6 substituents each independently selected from the group consisting of:
      oxo;
      R^{a};
      C₆₋₁₀ aryl optionally substituted with 1-4 independently selected C₁₋₄ alkyl; and
      heteroaryl of 5-10 ring atoms, wherein 1-4 ring atoms are heteroatoms, each independently selected from the group consisting of N, N(H), N(R^{d}), O, and S(O)₀₋₂, and wherein the heteroaryl ring is optionally substituted with 1-4 independently selected C₁₋₄ alkyl; or
   Y^{A1} is —Y^{A3}—Y^{A4}—Y^{A5} which is connected to Q via Y^{A3} wherein:
      Y^{A3} is a C₁₋₃ alkylene optionally substituted with 1-2 substituents each independently selected from the group consisting of oxo and R^{a};
   Y^{A4} is —O—, —NH—, —N(C₁₋₆ alkyl)-, or —S—; and
   Y^{A5} is a bond or C₁₋₃ alkylene which is optionally substituted with 1-2 independently selected R^{a}; and
Y^{A2} is:
   (a) C₃₋₂₀ cycloalkyl or C₃₋₂₀ cycloalkenyl, each of which is optionally substituted with 1-4 R^{b},
   (b) C₆₋₂₀ aryl which is optionally substituted with 1-4 R^{c};
   (c) heteroaryl of 5-20 ring atoms, wherein 1-3 ring atoms are heteroatoms, each independently selected from the group consisting of N, N(H), N(R^{d}), O, and S(O)₀₋₂, and wherein the heteroaryl ring is optionally substituted with 1-4 independently selected R^{c}; or
   (d) heterocyclyl or heterocycloalkenyl of 3-16 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl ring is optionally substituted with 1-4 independently selected R^{b},
or
(ii) —Z¹—Z²—Z³, wherein:
Z¹ is C₁₋₃ alkylene, which is optionally substituted with 1-4 R^{a};
Z² is —N(H)—, —N(R^{d})—, —O—, or —S—; and
Z³ is C₂₋₇ alkyl, which is optionally substituted with 1-4 R^{a}; or
(iii) C₁₋₂₀ alkyl, which is optionally substituted with 1-6 independently selected R^{a}, or
   (B)
Q and A, taken together, form:

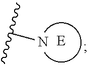

and
E is a ring of 3-16 ring atoms, wherein 0-3 ring atoms are heteroatoms (in addition to the nitrogen atom this is already present), each independently selected from the group consisting of N, N(H), N(R^{d}), O, and S(O)₀₋₂, and wherein the ring is optionally substituted with 1-4 independently selected R^{b},
each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-Rⁱ; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″); or
R^{1a} and R^{1b}, R^{1b} and R^{1c}, or R^{1c} and R^{1d}, taken together with the atoms connecting them, form a ring of 3-10 ring atoms, wherein 0-2 ring atoms are heteroatoms each independently selected from the group consisting of N, N(H), N(R^{d}), O, and S(O)₀₋₂; and wherein the ring is optionally substituted with 1-4 substituents each independently selected from the group consisting of C₁₋₆ alkyl, halo, C₁₋₆ haloalkyl, —OH, NR^{e}R^{f}, C₁₋₆ alkoxy, and C₁₋₆ haloalkoxy,
each occurrence of R² is independently selected from the group consisting of:
(i) C₁₋₆ alkyl, which is optionally substituted with 1-2 independently selected R^{a};
(ii) C₃₋₆ cycloalkyl, C₃₋₆ cycloalkenyl, or C₆₋₁₀ aryl;
(iii) heterocyclyl or heterocycloalkenyl of 3-10 ring atoms, wherein 1-3 ring atoms are heteroatoms, each independently selected from the group consisting of N, N(H), N(R^{d}), O, and S(O)₀₋₂;
(iv) heteroaryl of 5-10 ring atoms, wherein 1-3 ring atoms are heteroatoms, each independently selected from the group consisting of N, N(H), N(R^{d}), O, and S(O)₀₋₂;
(v) —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and
(vi) H;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; oxo; C₁₋₄ alkoxy optionally substituted with 1-2 independently selected R^{a}; C₁₋₄ haloalkoxy; —S(O)₁₋₂(C₁₋₄ alkyl) or —S(O)₁₋₂(C₁₋₄ haloalkyl); —NR^{e}R^{f}; —OH; —S(O)₁₋₂(NR′R″); —C₁₋₄ thioalkoxy or —C₁₋₄ thiohaloalkoxy; —NO₂; —SF₅; —C(═O)(C₁₋₁₀ alkyl); —C(═O)O(C₁₋₄ alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or R^{e} and R together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R^{e} and R), which are each independently selected from the group consisting of N(R^{d}), NH, O, and S;
-L¹ is a bond or C₁₋₃ alkylene;
-L² is —O—, —N(H)—, —N(C₁₋₃ alkyl)-, —S(O)₀₋₂—, or a bond;
R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
-L³ is a bond or C₁₋₃ alkylene;
-L⁴ is —O—, —N(H)—, —N(C₁₋₃ alkyl)-, —S(O)₀₋₂—, or a bond;
Rⁱ is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy; and
each occurrence of R′ and R″ is independently selected from the group consisting of: H, C₁₋₄ alkyl, C₆₋₁₀ aryl optionally substituted with 1-2 substituents selected from the group consisting of halo, C₁₋₄ alkyl, and C₁₋₄ haloalkyl, and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl; or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) 0-3 ring heteroatoms (in addition to the nitrogen atom attached to R′ and R″), which are each independently selected from the group consisting of N(H), N(C₁₋₆ alkyl), O, and S.

In certain embodiments of Formula (M3), the compound is a compound of Formula (M3A):

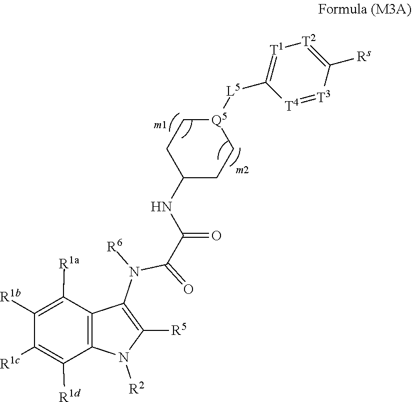

or a pharmaceutically acceptable salt thereof, wherein:
m1 and m2 are independently 0, 1, or 2;
Q⁵ is N or CH;
L⁵ is a bond, CH₂, —O—, —N(H)—, or —N(C₁₋₃ alkyl), provided that when Q⁵ is N, then L⁵ is a bond or CH₂;
T¹, T², T³, and T⁴ are each independently N, CH, or CR^{t}, provided that 1-4, such as 2, 3, or 4, of T¹-T⁴ is CH; and
each of R^{t} and R⁸ is independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy,
optionally wherein R² is H, and R⁵ is H; and
optionally wherein R^{1b} is halo, such as —F or —Cl; R^{1c} is H or halo, such as —H or —F; and R^{1a} and R^{1d} are H.

In some embodiments, the STING antagonist is a compound of Formula (M4):

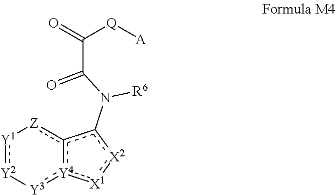

or a pharmaceutically acceptable salt thereof or a tautomer thereof,
wherein:
Z is selected from the group consisting of CR¹, N, and NR²;
each of Y¹, Y², and Y³ is independently selected from the group consisting of CR¹, N, and NR²;
Y⁴ is C or N, provided that one or more of Z, Y¹, Y², Y³, and Y⁴ is an independently selected heteroatom;
X¹ is selected from the group consisting of O, S, N, NR², and CR¹;
X² is selected from the group consisting of O, S, N, NR⁴, and CR⁵;
each [symbol] is independently a single bond or a double bond, provided that the five-membered ring comprising Y⁴, X¹, and X² is heteroaryl, and the 6-membered ring comprising Z, Y¹, Y², and Y³ is heteroaryl;
each occurrence of R¹ is independently selected from the group consisting of:
H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L³-L⁴-R; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each of R², R⁴, R⁵, R⁶, Q, A, R^{a}, R^{e}, R^{f}, L³, L⁴, Rⁱ, R′, and R″ are as defined for Formula M3 herein.

In some embodiments, the STING antagonist is a compound of Formula (M5):

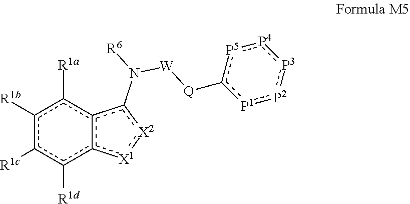

or a pharmaceutically acceptable salt thereof or a tautomer thereof, wherein:
X¹ is selected from the group consisting of O, S, N, NR², and CR¹;
X² is selected from the group consisting of O, S, N, NR⁴, and CR⁵;
each [symbol] is independently a single bond or a double bond, provided that:
the five-membered ring comprising X¹ and X² is heteroaryl;
the 6-membered ring

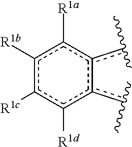

is aromatic; and
and the ring comprising P¹, P², P³, P⁴, and P⁵ is aromatic;
P¹, P², P³, P⁴, and P⁵ are defined according to (AA) or (BB):
   (AA)
each of P¹, P², P³, P⁴, and P⁵ is independently selected from the group consisting of: N, CH, CR⁷, and CR^{c}, provided that 1-2 of P¹, P², P³, P⁴, and P⁵ is an independently selected CR⁷; or
   (BB)
P¹ is absent, thereby providing a 5-membered ring,
each of P², P³, P⁴, and P⁵ is independently selected from the group consisting of O, S, N, NH, NR^{d}, NR⁷, CH, CR⁷, and CR^{c}, provided that 1-3 of P², P³, P⁴, and P⁵ is O, S, N, NH, NR^{d}, or NR⁷; and 1-2 of P², P³, P⁴, and P⁵ is an independently selected NR⁷ or CR⁷;
each R⁷ is independently selected from the group consisting of: —R⁸ and -L³-R⁹;
R⁸ and R⁹ are independently selected from the group consisting of
(a) C₃₋₁₂ cycloalkyl or C₃₋₁₂ cycloalkenyl, each of which is optionally substituted with 1-4 independently selected R^{7′};
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};
(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and
(d) C₆₋₁₀ aryl optionally substituted with 1-4 independently selected R^{7′};
-L³ is selected from the group consisting of —O—, —CH₂—, —S—, —NH—, S(O)₁₋₂, C(═O)NH, NHC(═O), C(═O)O, OC(═O), C(═O), NHS(O)₂, and S(O)₂NH;
each occurrence of R^{7′} is independently selected from the group consisting of: halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),
W is selected from the group consisting of:
(i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);
Q is selected from the group consisting of: NH, N(C₁₋₆ alkyl), *—NH—(C₁₋₃ alkylene)-, and *—N(C₁₋₆ alkyl)-(C₁₋₃ alkylene)-, wherein the C₁₋₆ alkyl is optionally substituted with 1-2 independently selected R^{a}, and the asterisk represents point of attachment to W;
each of R^{1a}, R^{1b}, R^{1c}, and R^{1d} is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); SF₅; —NR^{e}R^{f}; —OH; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) H;
(ii) C₁₋₆ alkyl, which is optionally substituted with 1-3 independently selected R^{a};
(iii) —C(O)(C₁₋₆ alkyl) optionally substituted with 1-3 independently selected R^{a};
(iv) —C(O)O(C₁₋₄ alkyl) optionally substituted with 1-3 independently R^{a};
(v) —CON(R′)(R″);
(vi) —S(O)₁₋₂(NR′R″);
(vii) —S(O)₁₋₂(C₁₋₄ alkyl) optionally substituted with 1-3 independently selected R^{a};
(viii) —OH;
(ix) C₁₋₄ alkoxy; and
(x) -L⁴-L⁵-R;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 independently selected C₁₋₄ alkyl;
each occurrence of R^{a} is independently selected from the group consisting of: —OH; —F; —Cl; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
each occurrence of R^{b} is independently selected from the group consisting of: C₁₋₁₀ alkyl optionally substituted with 1-6 independently selected R^{a}; C₁₋₄ haloalkyl; —OH; oxo; —F; —Cl; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of: halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —NR^{e}R^{f}; —OH; —S(O)₁₋₂(NR′R″); —C₁₋₄ thioalkoxy; —NO₂; —C(═O)(C₁₋₁₀ alkyl); —C(═O)O(C₁₋₄ alkyl); —C(═O)OH; —C(═O)N(R′)(R″); and -L¹-L²-R^{h};
R^{d} is selected from the group consisting of: C₁₋₆ alkyl optionally substituted with 1-3 substituents each independently selected from the group consisting of halo, C₁₋₃ alkoxy, C₁₋₃ haloalkoxy, and OH; C₃₋₆ cycloalkyl or C₃₋₆ cycloalkenyl, each optionally substituted with 1-3 substituents each independently selected from the group consisting of halo and OH; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy;
each occurrence of R^{e} and R^{f} is independently selected from the group consisting of: H; C₁₋₆ alkyl; C₁₋₆ haloalkyl; C₃₋₆ cycloalkyl or C₃₋₆ cycloalkenyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; and C₁₋₄ alkoxy; or
R^{e} and R^{f} together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) from 0-3 ring heteroatoms (in addition to the nitrogen atom attached to R^{e} and R), which are each independently selected from the group consisting of N(R^{d}), NH, O, and S;
-L¹ is a bond or C₁₋₃ alkylene; -L² is —O—, —N(H)—, —S(O)₀₋₂—, or a bond;
R^{h} is selected from the group consisting of:
C₃₋₈ cycloalkyl or C₃₋₈ cycloalkenyl, each optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄haloalkoxy; and
C₆₋₁₀ aryl, which is optionally substituted with 1-4 substituents independently selected from the group consisting of halo; C₁₋₄ alkyl optionally substituted with 1-2 independently selected R^{a}; C₁₋₄ haloalkyl; cyano; C₁₋₄ alkoxy; and C₁₋₄ haloalkoxy;
-L⁴- is selected from the group consisting of a bond, —C(O)—, —C(O)O—, —C(O)NH—, C(O)NR^{d}, S(O)₁₋₂, S(O)₁₋₂NH, and S(O)₁₋₂NR^{d};
-L⁵- is selected from the group consisting of a bond and C₁₋₄ alkylene;
Rⁱ is selected from the group consisting of:
C₃₋₈ cycloalkyl or C₃₋₈ cycloalkenyl, each optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heterocyclyl or heterocycloalkenyl, wherein the heterocyclyl or heterocycloalkenyl has 3-16 ring atoms, wherein 1-3 ring atoms are heteroatoms, each independently selected from the group consisting of N, N(H), N(R^{d}), O, and S(O)₀₋₂, wherein the heterocyclyl or heterocycloalkenyl is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy;
heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with 1-4 substituents independently selected from the group consisting of halo; OH; NR^{e}R^{f}; C₁₋₄ alkyl optionally substituted with 1-2 independently selected R^{a}; C₁₋₄ haloalkyl; cyano; C₁₋₄ alkoxy; and C₁₋₄haloalkoxy; and
each occurrence of R′ and R″ is independently selected from the group consisting of: H; —OH; C₁₋₄ alkyl; C₆₋₁₀ aryl optionally substituted with 1-2 substituents selected from the group consisting of halo, C₁₋₄ alkyl, and C₁₋₄ haloalkyl; and heteroaryl of 5-10 ring atoms, wherein 1-4 ring atoms are heteroatoms, each independently selected from the group consisting of N, N(H), N(R^{d}), O, and S(O)₀₋₂ and wherein the heteroaryl ring is optionally substituted with 1-4 substituents independently selected from the group consisting of halo, —OH, NH₂, NH(C₁₋₄ alkyl), N(C₁₋₄ alkyl)₂, C₁₋₄ alkyl, and C₁₋₄ haloalkyl;
or R′ and R″ together with the nitrogen atom to which each is attached forms a ring of 3-8 ring atoms, wherein the ring has: (a) 1-7 ring carbon atoms, each of which is substituted with 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₆ alkyl), O, and S.

In certain embodiments of Formula (M5), the compound is a compound of Formula (M5-1a), (M5-2a), or (M5-3a):

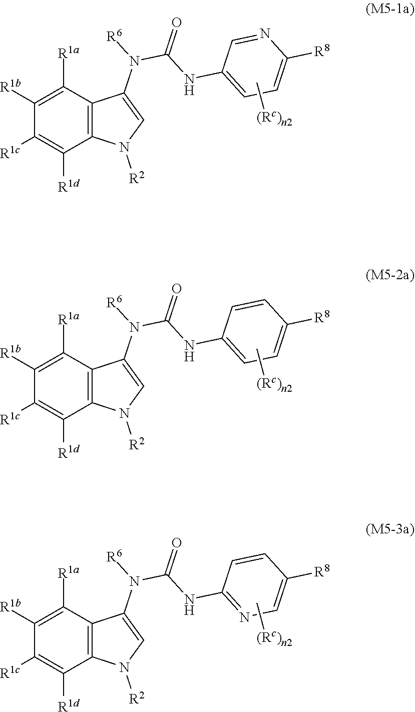

or a pharmaceutically acceptable salt thereof, wherein:
each of R^{1a}, R^{1b}, R^{1c}, R^{1d} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy;
n2 is 0, 1, or 2;
each R^{c} when present is independently selected from the group consisting of: halo, cyano, C₁₋₃ alkyl, and C₁₋₃ alkoxy;
R⁸ is selected from the group consisting of:

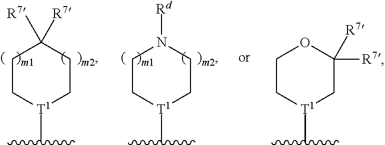

wherein m1 and m2 are independently 0, 1, or 2, and T¹ is CH or N; and
spirocyclic heterocyclyl of 6-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl ring is optionally substituted with 1-4 independently selected R^{7′}.

In some embodiments, the STING antagonist is a compound of Formula (M6):

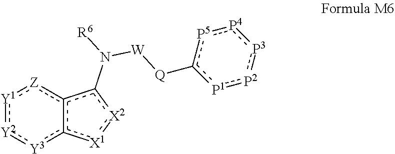

or a pharmaceutically acceptable salt thereof or a tautomer thereof, wherein:
each of Z, Y¹, Y², and Y³ is independently selected from the group consisting of CR¹, N, and NR², provided that 1-3 of Z, Y¹, Y², and Y³ is an independently selected N or NR²;
X¹ is selected from the group consisting of O, S, N, NR², and CR¹;
X² is selected from the group consisting of O, S, N, NR⁴, and CR⁵;
each [symbol] is independently a single bond or a double bond, provided that the five-membered ring comprising X¹ and X² is heteroaryl; the six-membered ring comprising Z, Y¹, Y², and Y³ is heteroaryl; and the ring comprising P¹, P², P³, P⁴, and P⁵ is aromatic;
W is selected from the group consisting of: (i) C(═O); (ii) C(═S); (iii) S(O)₁₋₂; (iv) C(═NR^{d}) or C(═N—CN); (v) C(═NH); (vi) C(═C—NO₂); (vii) S(═O)(═N(R^{d})); and (viii) S(═O)(═NH);
Q is selected from the group consisting of: NH, N(C₁₋₆ alkyl), *—NH—(C₁₋₃ alkylene)- and *—N(C₁₋₆ alkyl)-(C₁₋₃ alkylene)-, wherein the C₁₋₆ alkyl is optionally substituted with 1-2 independently selected R^{a}, and the asterisk represents the point of attachment to W;
P¹, P², P³, P⁴, and P⁵ are defined according to (AA) or (BB):
   (AA)
each of P¹, P², P³, P⁴, and P⁵ is independently selected from the group consisting of: N, CH, CR⁷, and CR^{e}, provided that: 1-2 of P¹, P², P³, P⁴, and P⁵ is an independently selected CR⁷; or
   (BB)
P¹ is absent, thereby providing a 5-membered ring,
each of P², P³, P⁴, and P⁵ is independently selected from the group consisting of O, S, N, NH, NR^{d}, NR⁷, CH, CR⁷, and CR^{c};
provided that 1-3 of P², P³, P⁴, and P⁵ is O, S, N, NH, NR^{d}, or NR⁷; and
1-2 of P², P³, P⁴, and P⁵ is an independently selected NR⁷ or CR⁷;
each R⁷ is independently selected from the group consisting of: —R⁸ and -L³-R⁹;
—R⁸ is selected from the group consisting of:
(a) C₃₋₁₂ cycloalkyl or C₃₋₁₂ cycloalkenyl, each of which is substituted with 1-4 independently selected R^{7′};
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is substituted with 1-4 independently selected R^{7′};
(c) C₃ cycloalkyl, C₃ cycloalkenyl, C₅ cycloalkyl, or C₅ cycloalkenyl, each of which is optionally substituted with 1-4 independently selected C₁₋₄ alkyl;
(d) C₇₋₁₂ cycloalkyl or C₇₋₁₂ cycloalkenyl, each of which is optionally substituted with 1-4 independently selected C₁₋₄ alkyl;
(e) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂ provided that the heterocyclyl is other than tetrahydropyranyl, and wherein one or more ring carbon atoms of the heterocyclyl or heterocycloalkenyl ring is optionally substituted with 1-4 independently selected C₁₋₄ alkyl;
(f) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the heteroaryl ring is optionally substituted with 1-4 independently selected R^{7′}; and
(g) C₆₋₁₀ aryl optionally substituted with 1-4 independently selected R^{7′};
-L³ is selected from the group consisting of —O—, —S—, —NH—, S(O)₁₋₂, —CH₂—, C(═O)NH, NHC(═O), C(═O)O, OC(═O), C(═O), NHS(O)₂, and S(O)₂NH;
—R⁹ is selected from the group consisting of:
(a) C₃₋₁₂ cycloalkyl or C₃₋₁₂ cycloalkenyl, each of which is optionally substituted with 1-4 independently selected R^{7′},
(b) heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein one or more ring carbon atoms of the heterocyclyl or heterocycloalkenyl ring is optionally substituted with 1-4 independently selected R^{7′};
(c) heteroaryl of 5-12 ring atoms, wherein 1-3 ring atoms are heteroatoms, each independently selected from the group consisting of N, N(H), N(R^{d}), O, and S(O)₀₋₂, and wherein one or more ring carbon atoms of the hetaroaryl ring is optionally substituted with 1-4 independently selected R^{7′}; and
(d) C₆₋₁₀ aryl optionally substituted with 1-4 independently selected R^{7′};
each occurrence of R^{7′} is independently selected from the group consisting of:
halo; —CN; —NO₂; —OH; —C₁₋₄ alkyl optionally substituted with 1-2 independently selected R^{a}; —C₂₋₄ alkenyl; —C₂₋₄ alkynyl; —C₁₋₄ haloalkyl; —C₁₋₆ alkoxy optionally substituted with 1-2 independently selected R^{a}; —C₁₋₆ haloalkoxy; S(O)₁₋₂(C₁₋₄ alkyl); —NR′R″; oxo; —S(O)₁₋₂(NR′R″); —C₁₋₄ thioalkoxy; —C(═O)(C₁₋₄ alkyl); —C(═O)O(C₁₋₄ alkyl); —C(═O)OH; and —C(═O)N(R′)(R″),
provided that when R⁷ is R⁸; and R⁸ is cycloalkyl, cycloalkenyl, heterocyclyl, or heterocycloalkenyl and substituted with 1-4 R^{7′}, then:
R⁸ cannot be monosubstituted with C₁₋₄ alkyl, and
when R⁸ is substituted with 2-4 R^{7′}, then at least one R^{7′} must be a substituent other than C₁₋₄ alkyl;
each occurrence of R¹ is independently selected from the group consisting of:
H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; -L¹-L²-R^{h}; —S(O)₁₋₂(C₁₋₄ alkyl); —S(O)(═NH)(C₁₋₄ alkyl); SF₅; —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; and —C(═O)N(R′)(R″);
each occurrence of R² is independently selected from the group consisting of:
(i) H;
(ii) C₁₋₆ alkyl optionally substituted with 1-3 independently selected R^{a};
(iii) —C(O)(C₁₋₆ alkyl) optionally substituted with 1-3 independently selected R^{a};
(iv) —C(O)O(C₁₋₄ alkyl) optionally substituted with 1-3 independently selected R^{a};
(v) —CON(R′)(R″);
(vi) —S(O)₁₋₂(NR′R″);
(vii) —S(O)₁₋₂(C₁₋₄ alkyl) optionally substituted with 1-3 independently selected R^{a};
(viii) —OH;
(ix) C₁₋₄ alkoxy; and
(x) -L⁴-L⁵-R;
R⁴ is selected from the group consisting of H and C₁₋₆ alkyl optionally substituted with 1-3 independently selected R^{a};
R⁵ is selected from the group consisting of H; halo; —OH; —C₁₋₄ alkyl; —C₁₋₄ haloalkyl; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano; and C₃₋₆ cycloalkyl or C₃₋₆ cycloalkenyl, each optionally substituted with 1-4 independently selected C₁₋₄ alkyl;
R⁶ is selected from the group consisting of H; C₁₋₆ alkyl optionally substituted with 1-3 independently selected R^{a}; —OH; C₁₋₄ alkoxy; C(═O)H; C(═O)(C₁₋₄ alkyl); C₆₋₁₀ aryl optionally substituted with 1-4 independently selected C₁₋₄ alkyl; and heteroaryl of 5-10 ring atoms, wherein 1-4 ring atoms are heteroatoms, each independently selected from the group consisting of N, N(H), N(R^{d}), O, and S(O)₀₋₂, wherein the heteroaryl ring is optionally substituted with 1-4 independently selected C₁₋₄ alkyl; and
each of R^{a}, R^{b}, R^{c}, R^{d}, R^{e}, R^{f}, -L¹, -L², R^{h}, -L⁴, L⁵, —Rⁱ, R′, and R″ is as defined in Formula (M5) herein.

In certain embodiments of Formula (M6), the compound is a compound of Formula (M6-3a) or (M6-3b):

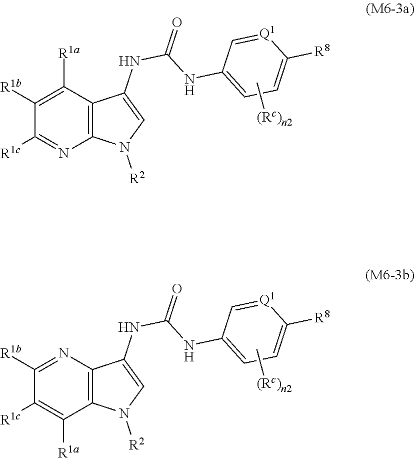

or a pharmaceutically acceptable salt thereof, wherein:
each of R^{a}, R^{1b}, and R^{1c} is independently selected from the group consisting of: H; halo; cyano; C₁₋₆ alkyl optionally substituted with 1-2 R^{a}; C₁₋₄ haloalkyl; C₁₋₄ alkoxy; and C₁₋₄ haloalkoxy; Q¹ is N or CH;
R⁸ is selected from the group consisting of:

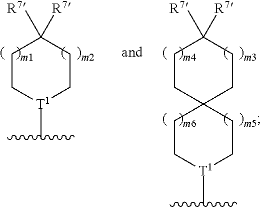

n2 is 0, 1, or 2;
each R^{c} when present is independently selected from the group consisting of: halo, cyano, C₁₋₃ alkyl, and C₁₋₃ alkoxy;
m1 and m2 are independently 0, 1, or 2; m3, m4, m5, and m6 are independently 0 or 1; and
T¹ is CH or N,
optionally wherein R² is H;
optionally wherein n2 is 1, and R^{c} is ortho to R⁸; and
optionally wherein each R^{7′} is independently halo, such as —F.

In some embodiments, the STING antagonist is selected from the group consisting of the compounds in Table C₁, or a pharmaceutically acceptable salt thereof.

TABLE C1

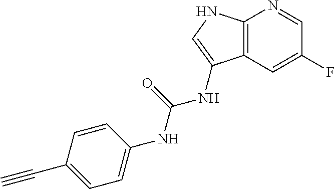 [SMILES: C#Cc1ccc(NC(=O)Nc2c[nH]c3ncc(F)cc23)cc1]

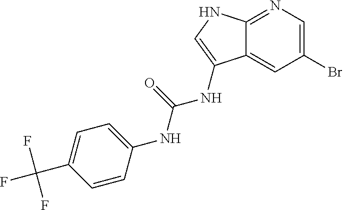 [SMILES: O=C(Nc1ccc(C(F)(F)F)cc1)Nc1c[nH]c2ncc(Br)cc12]

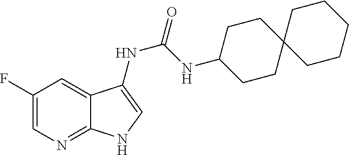 [SMILES: O=C(Nc1c[nH]c2ncc(F)cc12)NC1CCC2(CCCCC2)CC1]

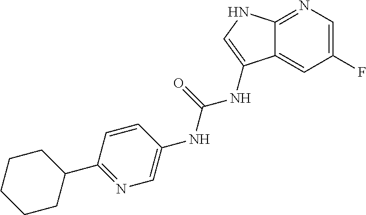 [SMILES: O=C(Nc1ccc(C2CCCCC2)nc1)Nc1c[nH]c2ncc(F)cc12]

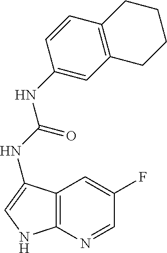 [SMILES: O=C(Nc1ccc2c(c1)CCCC2)Nc1c[nH]c2ncc(F)cc12]

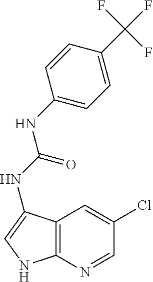 [SMILES: O=C(Nc1ccc(C(F)(F)F)cc1)Nc1c[nH]c2ncc(Cl)cc12]

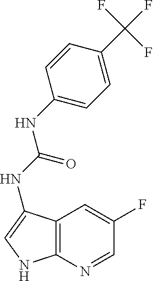 [SMILES: O=C(Nc1ccc(C(F)(F)F)cc1)Nc1c[nH]c2ncc(F)cc12]

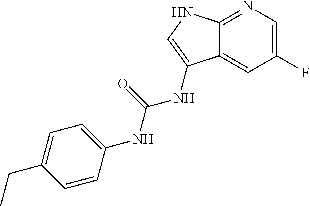 [SMILES: CCc1ccc(NC(=O)Nc2c[nH]c3ncc(F)cc23)cc1]

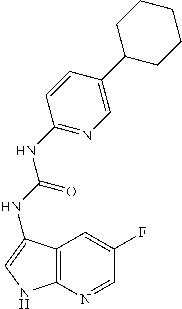 [SMILES: O=C(Nc1ccc(C2CCCCC2)cn1)Nc1c[nH]c2ncc(F)cc12]

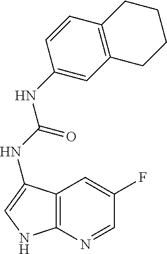 [SMILES: O=C(Nc1ccc2c(c1)CCCC2)Nc1c[nH]c2ncc(F)cc12]

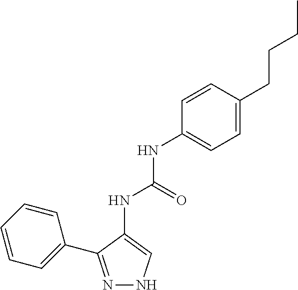 [SMILES: CCCCc1ccc(NC(=O)Nc2c[nH]nc2-c2ccccc2)cc1]

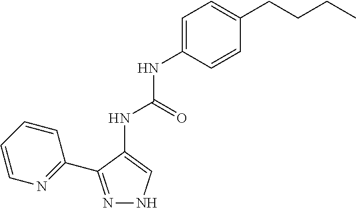 [SMILES: CCCCc1ccc(NC(=O)Nc2c[nH]nc2-c2ccccn2)cc1]

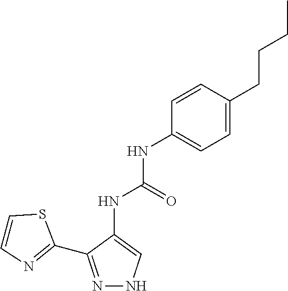 [SMILES: CCCCc1ccc(NC(=O)Nc2c[nH]nc2-c2nccs2)cc1]

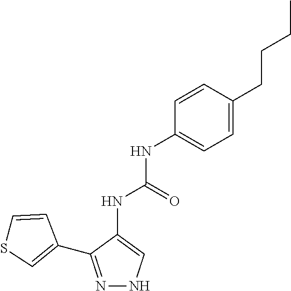 [SMILES: CCCCc1ccc(NC(=O)Nc2c[nH]nc2-c2ccsc2)cc1]

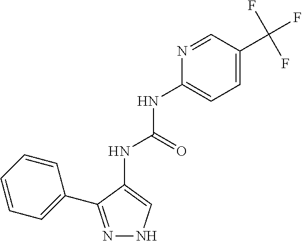 [SMILES: O=C(Nc1ccc(C(F)(F)F)cn1)Nc1c[nH]nc1-c1ccccc1]

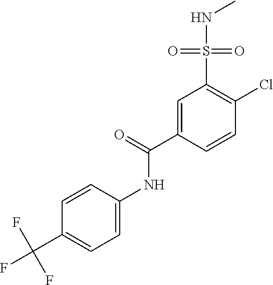 [SMILES: CNS(=O)(=O)c1cc(C(=O)Nc2ccc(C(F)(F)F)cc2)ccc1Cl]

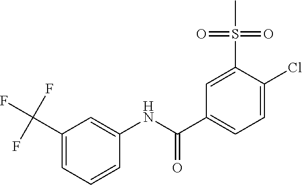 [SMILES: CS(=O)(=O)c1cc(C(=O)Nc2cccc(C(F)(F)F)c2)ccc1Cl]

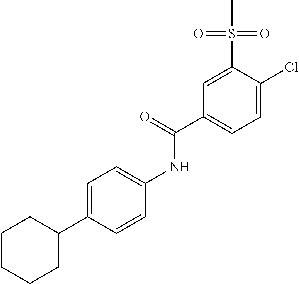 [SMILES: CS(=O)(=O)c1cc(C(=O)Nc2ccc(C3CCCCC3)cc2)ccc1Cl]

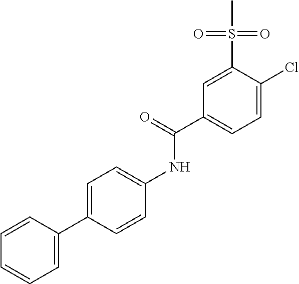 [SMILES: CS(=O)(=O)c1cc(C(=O)Nc2ccc(-c3ccccc3)cc2)ccc1Cl]

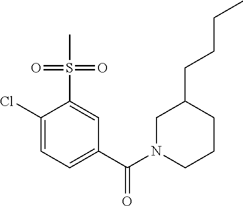 [SMILES: CCCCC1CCCN(C(=O)c2ccc(Cl)c(S(C)(=O)=O)c2)C1]

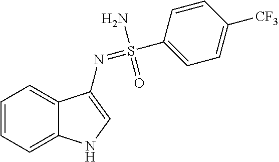 [SMILES: NS(=O)(=Nc1c[nH]c2ccccc12)c1ccc(C(F)(F)F)cc1]

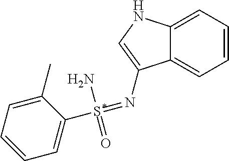

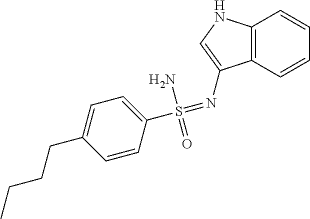 [SMILES: CCCCc1ccc(S(N)(=O)=Nc2c[nH]c3ccccc23)cc1]

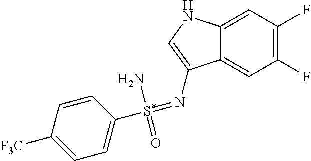

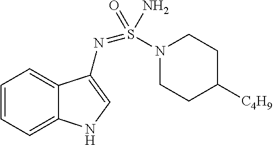

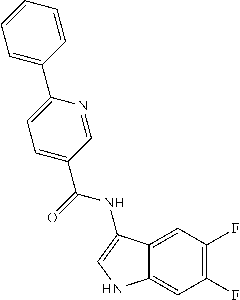 [SMILES: O=C(Nc1c[nH]c2cc(F)c(F)cc12)c1ccc(-c2ccccc2)nc1]

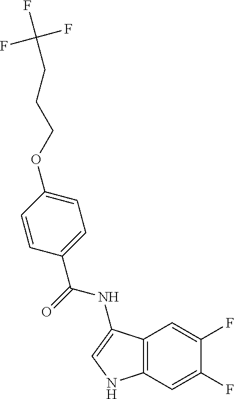 [SMILES: O=C(Nc1c[nH]c2cc(F)c(F)cc12)c1ccc(OCCCC(F)(F)F)cc1]

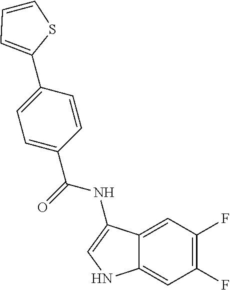 [SMILES: O=C(Nc1c[nH]c2cc(F)c(F)cc12)c1ccc(-c2cccs2)cc1]

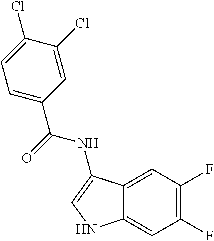 [SMILES: O=C(Nc1c[nH]c2cc(F)c(F)cc12)c1ccc(Cl)c(Cl)c1]

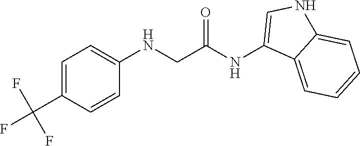 [SMILES: O=C(CNc1ccc(C(F)(F)F)cc1)Nc1c[nH]c2ccccc12]

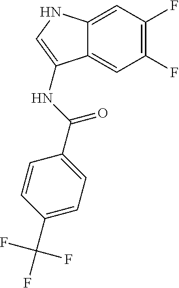 [SMILES: O=C(Nc1c[nH]c2cc(F)c(F)cc12)c1ccc(C(F)(F)F)cc1]

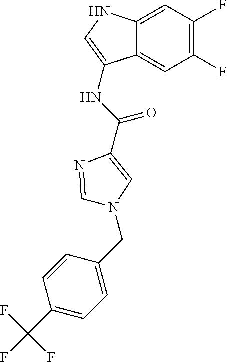 [SMILES: O=C(Nc1c[nH]c2cc(F)c(F)cc12)c1cn(Cc2ccc(C(F)(F)F)cc2)cn1]

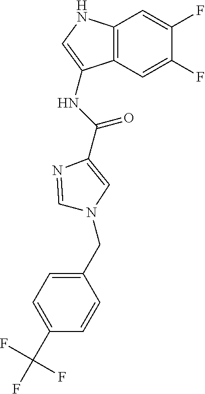 [SMILES: O=C(Nc1c[nH]c2cc(F)c(F)cc12)c1cn(Cc2ccc(C(F)(F)F)cc2)cn1]

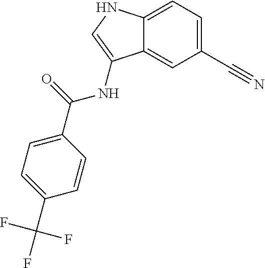 [SMILES: N#Cc1ccc2[nH]cc(NC(=O)c3ccc(C(F)(F)F)cc3)c2c1]

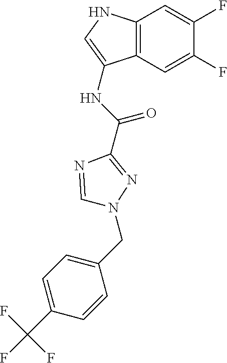 [SMILES: O=C(Nc1c[nH]c2cc(F)c(F)cc12)c1ncn(Cc2ccc(C(F)(F)F)cc2)n1]

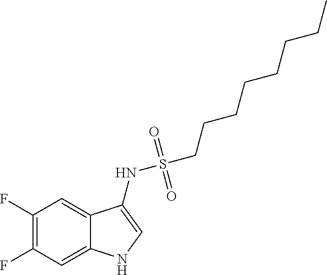 [SMILES: CCCCCCCCS(=O)(=O)Nc1c[nH]c2cc(F)c(F)cc12]

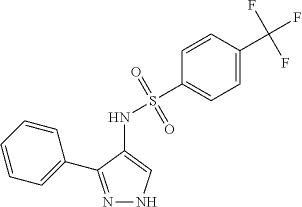 [SMILES: O=S(=O)(Nc1c[nH]nc1-c1ccccc1)c1ccc(C(F)(F)F)cc1]

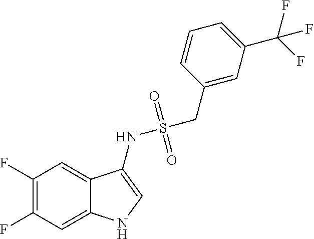 [SMILES: O=S(=O)(Cc1cccc(C(F)(F)F)c1)Nc1c[nH]c2cc(F)c(F)cc12]

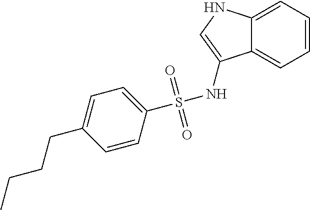 [SMILES: CCCCc1ccc(S(=O)(=O)Nc2c[nH]c3ccccc23)cc1]

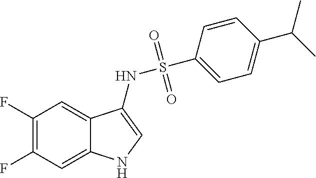 [SMILES: CC(C)c1ccc(S(=O)(=O)Nc2c[nH]c3cc(F)c(F)cc23)cc1]

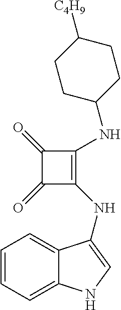

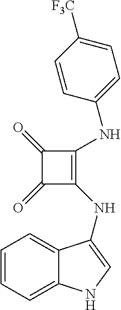 [SMILES: O=c1c(Nc2ccc(C(F)(F)F)cc2)c(Nc2c[nH]c3ccccc23)c1=O]

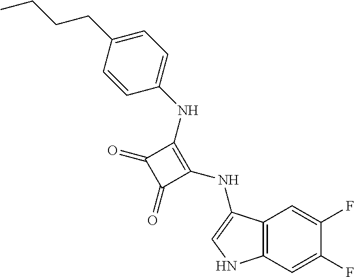 [SMILES: CCCCc1ccc(Nc2c(Nc3c[nH]c4cc(F)c(F)cc34)c(=O)c2=O)cc1]

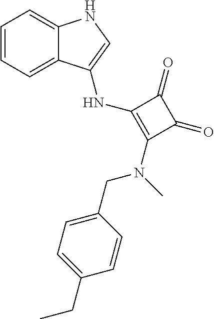 [SMILES: CCc1ccc(CN(C)c2c(Nc3c[nH]c4ccccc34)c(=O)c2=O)cc1]

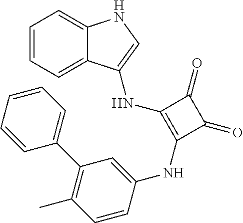 [SMILES: Cc1ccc(Nc2c(Nc3c[nH]c4ccccc34)c(=O)c2=O)cc1-c1ccccc1]

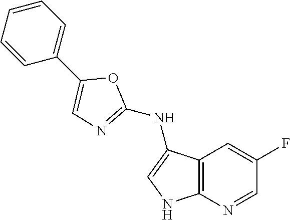 [SMILES: Fc1cnc2[nH]cc(Nc3ncc(-c4ccccc4)o3)c2c1]

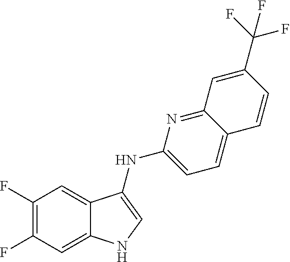 [SMILES: Fc1cc2[nH]cc(Nc3ccc4ccc(C(F)(F)F)cc4n3)c2cc1F]

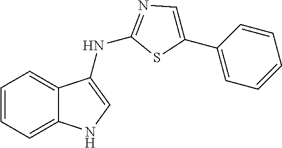 [SMILES: c1ccc(-c2cnc(Nc3c[nH]c4ccccc34)s2)cc1]

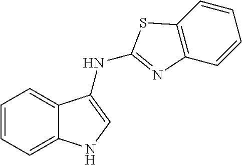 [SMILES: c1ccc2sc(Nc3c[nH]c4ccccc34)nc2c1]

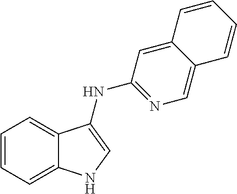 [SMILES: c1ccc2cc(Nc3c[nH]c4ccccc34)ncc2c1]

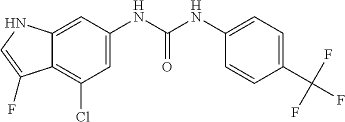 [SMILES: O=C(Nc1ccc(C(F)(F)F)cc1)Nc1cc(Cl)c2c(F)c[nH]c2c1]

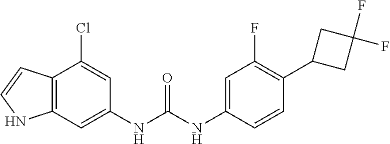 [SMILES: O=C(Nc1ccc(C2CC(F)(F)C2)c(F)c1)Nc1cc(Cl)c2cc[nH]c2c1]

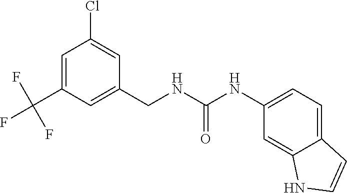 [SMILES: O=C(NCc1cc(Cl)cc(C(F)(F)F)c1)Nc1ccc2cc[nH]c2c1]

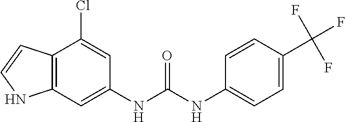 [SMILES: O=C(Nc1ccc(C(F)(F)F)cc1)Nc1cc(Cl)c2cc[nH]c2c1]

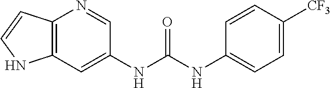 [SMILES: O=C(Nc1ccc(C(F)(F)F)cc1)Nc1cnc2cc[nH]c2c1]

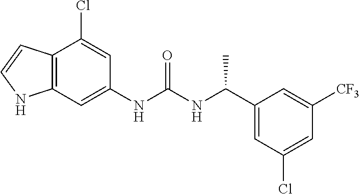 [SMILES: C[C@@H](NC(=O)Nc1cc(Cl)c2cc[nH]c2c1)c1cc(Cl)cc(C(F)(F)F)c1]

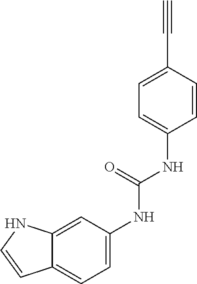 [SMILES: C#Cc1ccc(NC(=O)Nc2ccc3cc[nH]c3c2)cc1]

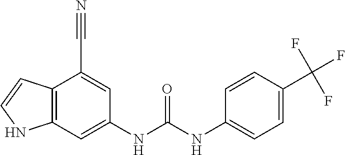 [SMILES: N#Cc1cc(NC(=O)Nc2ccc(C(F)(F)F)cc2)cc2[nH]ccc12]

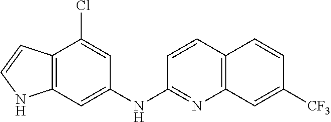 [SMILES: FC(F)(F)c1ccc2ccc(Nc3cc(Cl)c4cc[nH]c4c3)nc2c1]

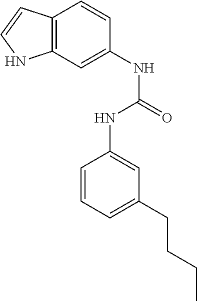 [SMILES: CCCCc1cccc(NC(=O)Nc2ccc3cc[nH]c3c2)c1]

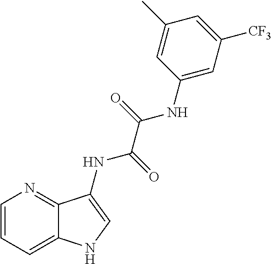 [SMILES: Cc1cc(NC(=O)C(=O)Nc2c[nH]c3cccnc23)cc(C(F)(F)F)c1]

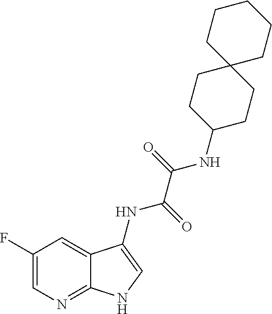 [SMILES: O=C(Nc1c[nH]c2ncc(F)cc12)C(=O)NC1CCC2(CCCCC2)CC1]

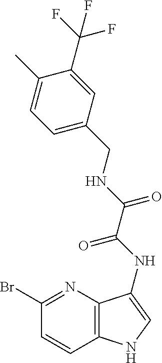 [SMILES: Cc1ccc(CNC(=O)C(=O)Nc2c[nH]c3ccc(Br)nc23)cc1C(F)(F)F]

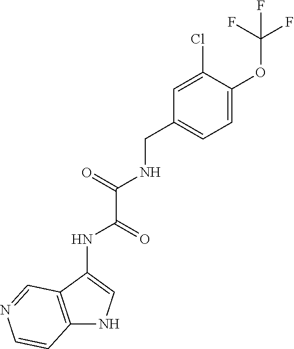 [SMILES: O=C(NCc1ccc(OC(F)(F)F)c(Cl)c1)C(=O)Nc1c[nH]c2ccncc12]

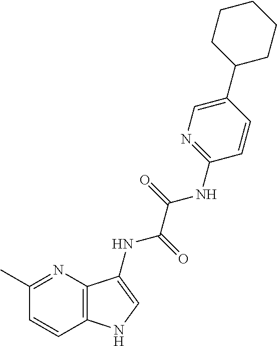 [SMILES: Cc1ccc2[nH]cc(NC(=O)C(=O)Nc3ccc(C4CCCCC4)cn3)c2n1]

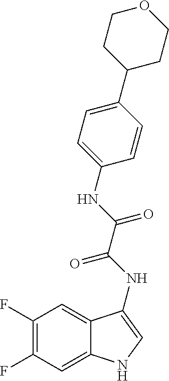 [SMILES: O=C(Nc1ccc(C2CCOCC2)cc1)C(=O)Nc1c[nH]c2cc(F)c(F)cc12]

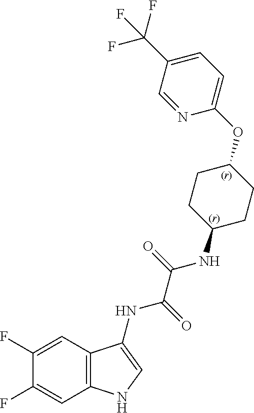 [SMILES: O=C(Nc1c[nH]c2cc(F)c(F)cc12)C(=O)N[C@H]1CC[C@H](Oc2ccc(C(F)(F)F)cn2)CC1]

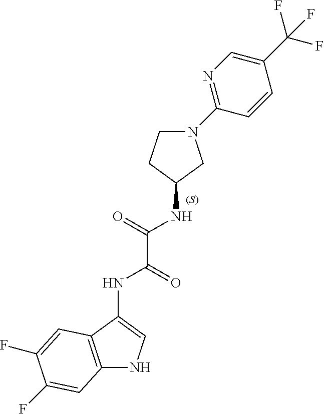

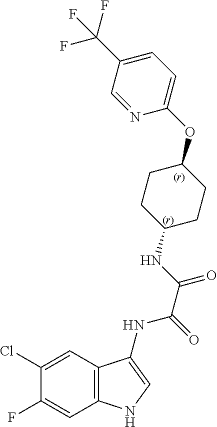 [SMILES: O=C(Nc1c[nH]c2cc(F)c(Cl)cc12)C(=O)N[C@H]1CC[C@H](Oc2ccc(C(F)(F)F)cn2)CC1]

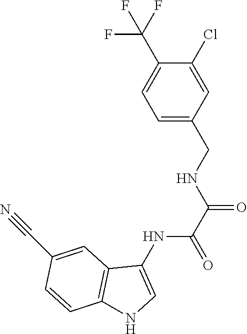 [SMILES: N#Cc1ccc2[nH]cc(NC(=O)C(=O)NCc3ccc(C(F)(F)F)c(Cl)c3)c2c1]

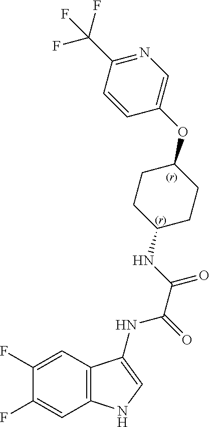 [SMILES: O=C(Nc1c[nH]c2cc(F)c(F)cc12)C(=O)N[C@H]1CC[C@H](Oc2ccc(C(F)(F)F)nc2)CC1]

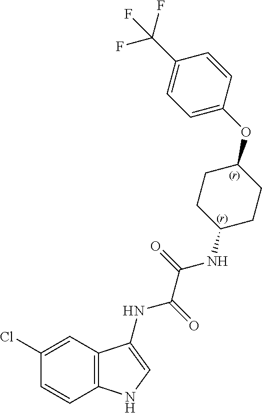 [SMILES: O=C(Nc1c[nH]c2ccc(Cl)cc12)C(=O)N[C@H]1CC[C@H](Oc2ccc(C(F)(F)F)cc2)CC1]

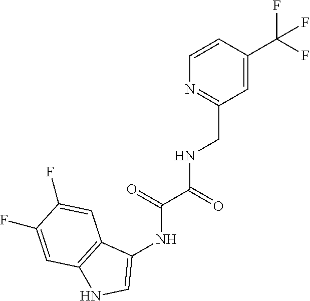 [SMILES: O=C(NCc1cc(C(F)(F)F)ccn1)C(=O)Nc1c[nH]c2cc(F)c(F)cc12]

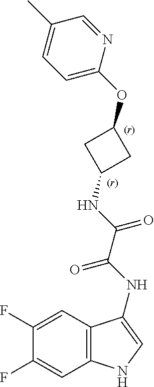 [SMILES: Cc1ccc(O[C@H]2C[C@H](NC(=O)C(=O)Nc3c[nH]c4cc(F)c(F)cc34)C2)nc1]

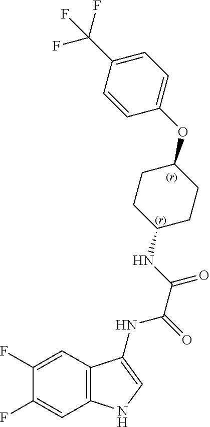 [SMILES: O=C(Nc1c[nH]c2cc(F)c(F)cc12)C(=O)N[C@H]1CC[C@H](Oc2ccc(C(F)(F)F)cc2)CC1]

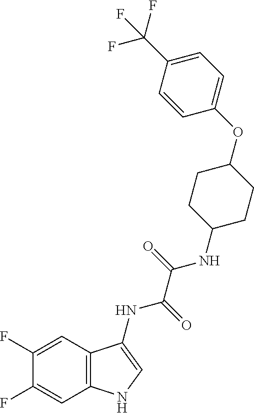 [SMILES: O=C(Nc1c[nH]c2cc(F)c(F)cc12)C(=O)NC1CCC(Oc2ccc(C(F)(F)F)cc2)CC1]

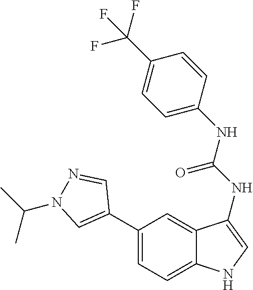 [SMILES: CC(C)n1cc(-c2ccc3[nH]cc(NC(=O)Nc4ccc(C(F)(F)F)cc4)c3c2)cn1]

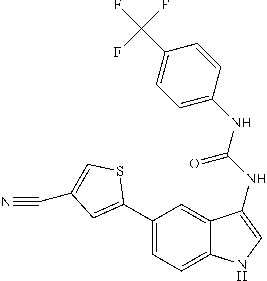 [SMILES: N#Cc1csc(-c2ccc3[nH]cc(NC(=O)Nc4ccc(C(F)(F)F)cc4)c3c2)c1]

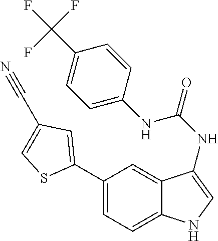 [SMILES: N#Cc1csc(-c2ccc3[nH]cc(NC(=O)Nc4ccc(C(F)(F)F)cc4)c3c2)c1]

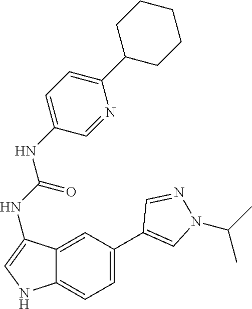 [SMILES: CC(C)n1cc(-c2ccc3[nH]cc(NC(=O)Nc4ccc(C5CCCCC5)nc4)c3c2)cn1]

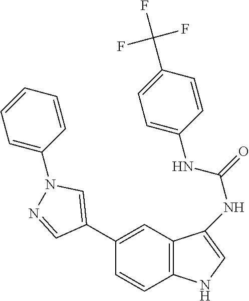 [SMILES: O=C(Nc1ccc(C(F)(F)F)cc1)Nc1c[nH]c2ccc(-c3cnn(-c4ccccc4)c3)cc12]

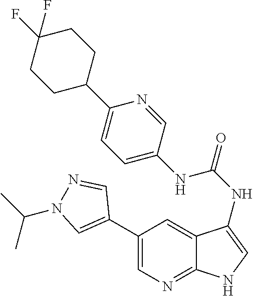 [SMILES: CC(C)n1cc(-c2cnc3[nH]cc(NC(=O)Nc4ccc(C5CCC(F)(F)CC5)nc4)c3c2)cn1]

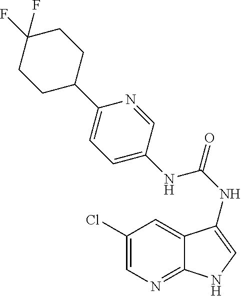 [SMILES: O=C(Nc1ccc(C2CCC(F)(F)CC2)nc1)Nc1c[nH]c2ncc(Cl)cc12]

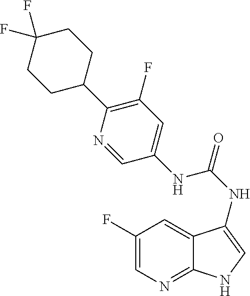 [SMILES: O=C(Nc1cnc(C2CCC(F)(F)CC2)c(F)c1)Nc1c[nH]c2ncc(F)cc12]

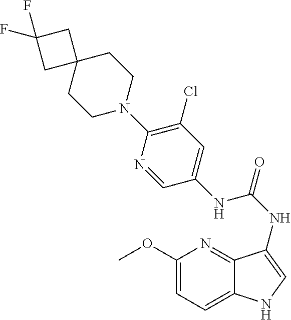 [SMILES: COc1ccc2[nH]cc(NC(=O)Nc3cnc(N4CCC5(CC4)CC(F)(F)C5)c(Cl)c3)c2n1]

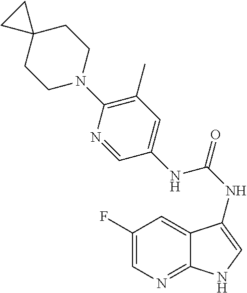 [SMILES: Cc1cc(NC(=O)Nc2c[nH]c3ncc(F)cc23)cnc1N1CCC2(CC1)CC2]

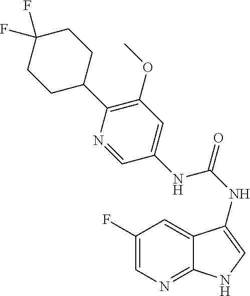 [SMILES: COc1cc(NC(=O)Nc2c[nH]c3ncc(F)cc23)cnc1C1CCC(F)(F)CC1]

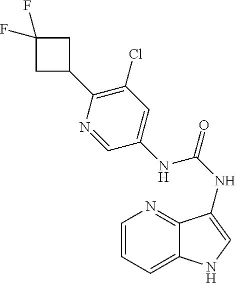 [SMILES: O=C(Nc1cnc(C2CC(F)(F)C2)c(Cl)c1)Nc1c[nH]c2cccnc12]

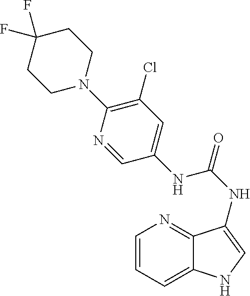 [SMILES: O=C(Nc1cnc(N2CCC(F)(F)CC2)c(Cl)c1)Nc1c[nH]c2cccnc12]

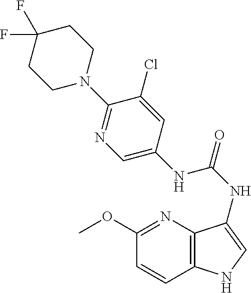 [SMILES: COc1ccc2[nH]cc(NC(=O)Nc3cnc(N4CCC(F)(F)CC4)c(Cl)c3)c2n1]

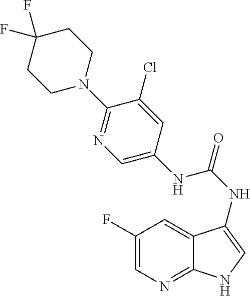 [SMILES: O=C(Nc1cnc(N2CCC(F)(F)CC2)c(Cl)c1)Nc1c[nH]c2ncc(F)cc12]

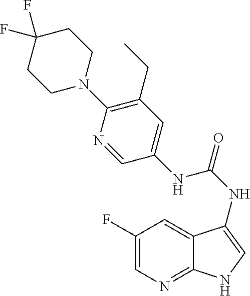 [SMILES: CCc1cc(NC(=O)Nc2c[nH]c3ncc(F)cc23)cnc1N1CCC(F)(F)CC1]

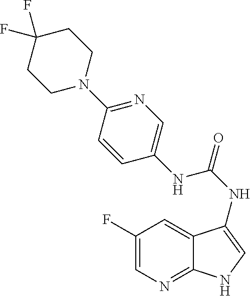 [SMILES: O=C(Nc1ccc(N2CCC(F)(F)CC2)nc1)Nc1c[nH]c2ncc(F)cc12]

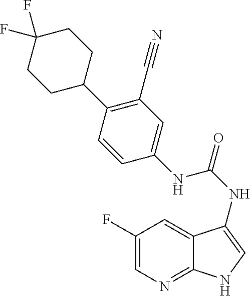 [SMILES: N#Cc1cc(NC(=O)Nc2c[nH]c3ncc(F)cc23)ccc1C1CCC(F)(F)CC1]

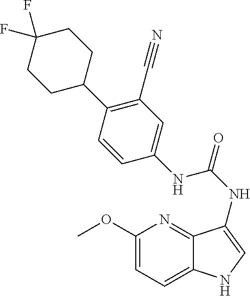 [SMILES: COc1ccc2[nH]cc(NC(=O)Nc3ccc(C4CCC(F)(F)CC4)c(C#N)c3)c2n1]

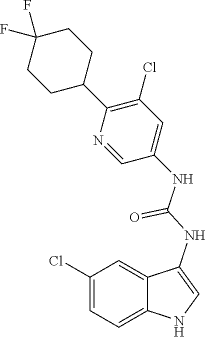 [SMILES: O=C(Nc1cnc(C2CCC(F)(F)CC2)c(Cl)c1)Nc1c[nH]c2ccc(Cl)cc12]

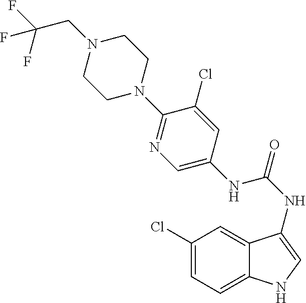 [SMILES: O=C(Nc1cnc(N2CCN(CC(F)(F)F)CC2)c(Cl)c1)Nc1c[nH]c2ccc(Cl)cc12]

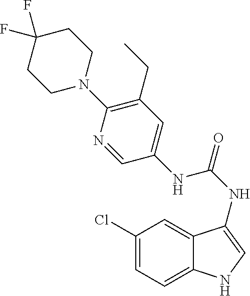 [SMILES: CCc1cc(NC(=O)Nc2c[nH]c3ccc(Cl)cc23)cnc1N1CCC(F)(F)CC1]

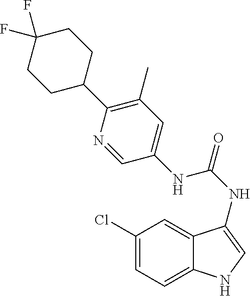 [SMILES: Cc1cc(NC(=O)Nc2c[nH]c3ccc(Cl)cc23)cnc1C1CCC(F)(F)CC1]

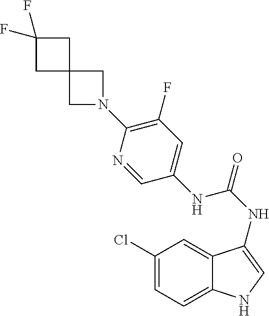 [SMILES: O=C(Nc1cnc(N2CC3(C2)CC(F)(F)C3)c(F)c1)Nc1c[nH]c2ccc(Cl)cc12]

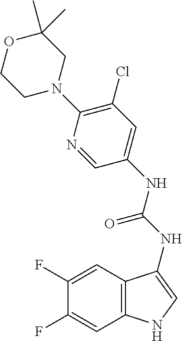 [SMILES: CC1(C)CN(c2ncc(NC(=O)Nc3c[nH]c4cc(F)c(F)cc34)cc2Cl)CCO1]

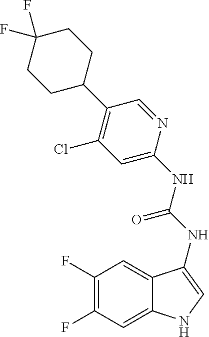 [SMILES: O=C(Nc1cc(Cl)c(C2CCC(F)(F)CC2)cn1)Nc1c[nH]c2cc(F)c(F)cc12]

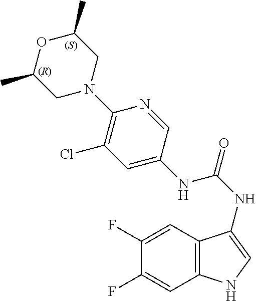

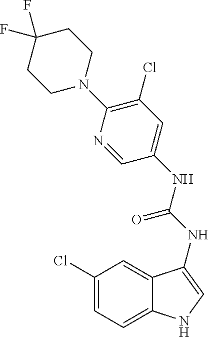 [SMILES: O=C(Nc1cnc(N2CCC(F)(F)CC2)c(Cl)c1)Nc1c[nH]c2ccc(Cl)cc12]

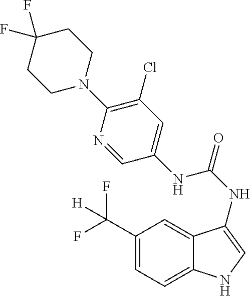 [SMILES: O=C(Nc1cnc(N2CCC(F)(F)CC2)c(Cl)c1)Nc1c[nH]c2ccc(C(F)F)cc12]

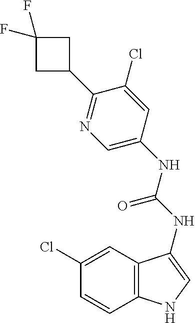 [SMILES: O=C(Nc1cnc(C2CC(F)(F)C2)c(Cl)c1)Nc1c[nH]c2ccc(Cl)cc12]

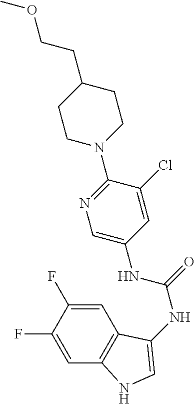 [SMILES: COCCC1CCN(c2ncc(NC(=O)Nc3c[nH]c4cc(F)c(F)cc34)cc2Cl)CC1]

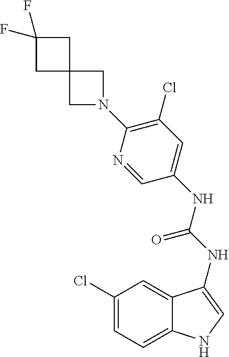 [SMILES: O=C(Nc1cnc(N2CC3(C2)CC(F)(F)C3)c(Cl)c1)Nc1c[nH]c2ccc(Cl)cc12]

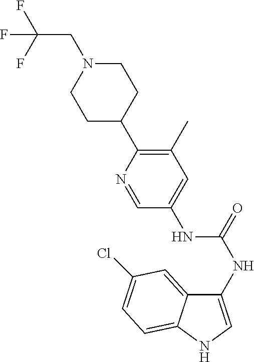 [SMILES: Cc1cc(NC(=O)Nc2c[nH]c3ccc(Cl)cc23)cnc1C1CCN(CC(F)(F)F)CC1]

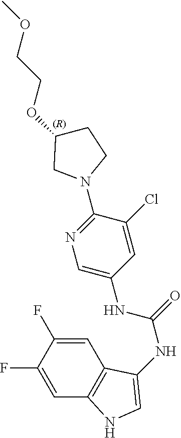

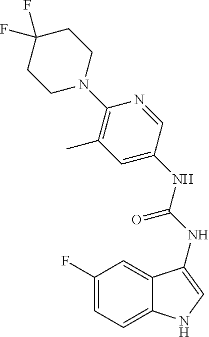 [SMILES: Cc1cc(NC(=O)Nc2c[nH]c3ccc(F)cc23)cnc1N1CCC(F)(F)CC1]

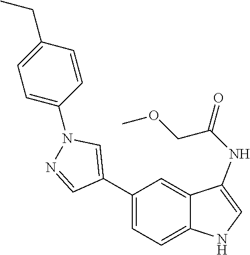 [SMILES: CCc1ccc(-n2cc(-c3ccc4[nH]cc(NC(=O)COC)c4c3)cn2)cc1]

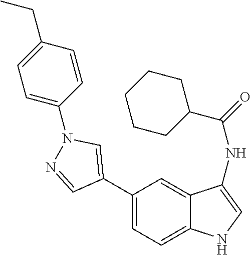 [SMILES: CCc1ccc(-n2cc(-c3ccc4[nH]cc(NC(=O)C5CCCCC5)c4c3)cn2)cc1]

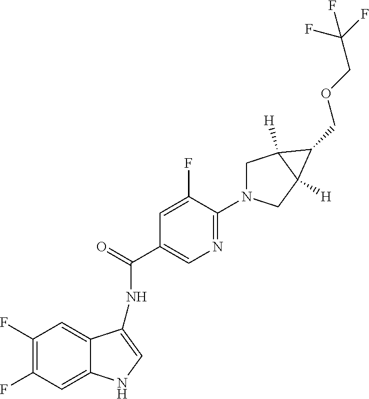 [SMILES: O=C(Nc1c[nH]c2cc(F)c(F)cc12)c1cnc(N2C[C@@H]3[C@@H](COCC(F)(F)F)[C@@H]3C2)c(F)c1]

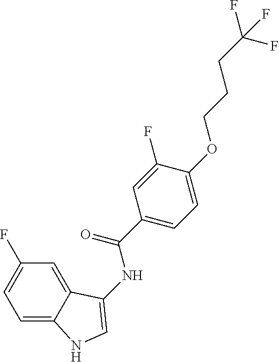 [SMILES: O=C(Nc1c[nH]c2ccc(F)cc12)c1ccc(OCCCC(F)(F)F)c(F)c1]

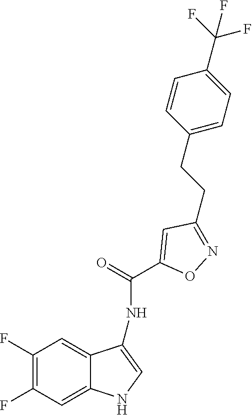 [SMILES: O=C(Nc1c[nH]c2cc(F)c(F)cc12)c1cc(CCc2ccc(C(F)(F)F)cc2)no1]

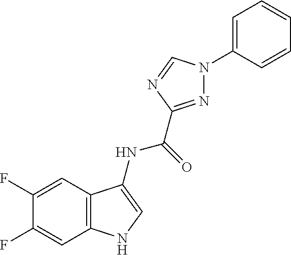 [SMILES: O=C(Nc1c[nH]c2cc(F)c(F)cc12)c1ncn(-c2ccccc2)n1]

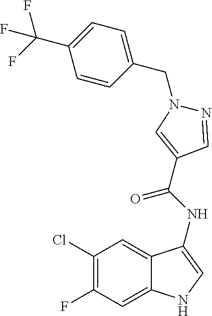 [SMILES: O=C(Nc1c[nH]c2cc(F)c(Cl)cc12)c1cnn(Cc2ccc(C(F)(F)F)cc2)c1]

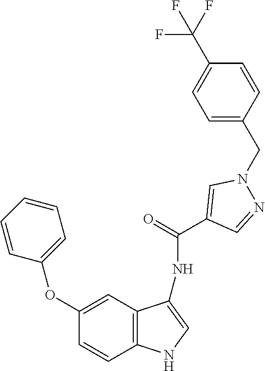 [SMILES: O=C(Nc1c[nH]c2ccc(Oc3ccccc3)cc12)c1cnn(Cc2ccc(C(F)(F)F)cc2)c1]

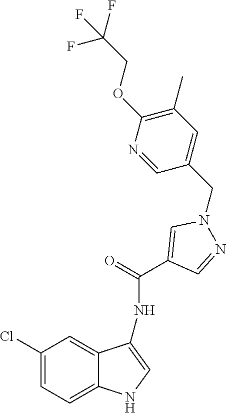 [SMILES: Cc1cc(Cn2cc(C(=O)Nc3c[nH]c4ccc(Cl)cc34)cn2)cnc1OCC(F)(F)F]

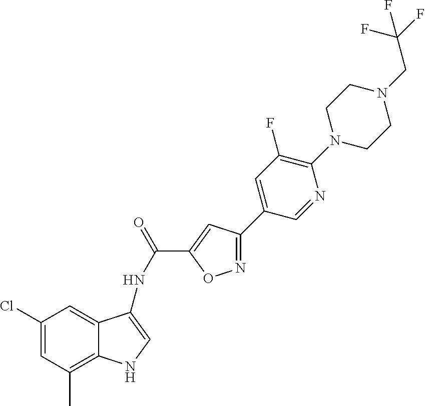 [SMILES: Cc1cc(Cl)cc2c(NC(=O)c3cc(-c4cnc(N5CCN(CC(F)(F)F)CC5)c(F)c4)no3)c[nH]c12]

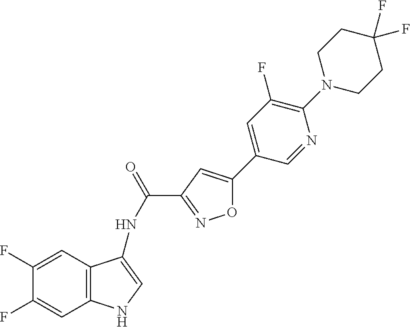 [SMILES: O=C(Nc1c[nH]c2cc(F)c(F)cc12)c1cc(-c2cnc(N3CCC(F)(F)CC3)c(F)c2)on1]

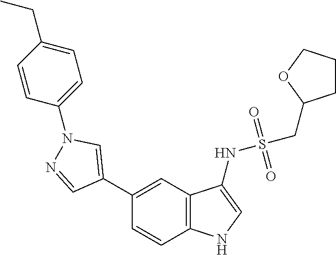 [SMILES: CCc1ccc(-n2cc(-c3ccc4[nH]cc(NS(=O)(=O)CC5CCCO5)c4c3)cn2)cc1]

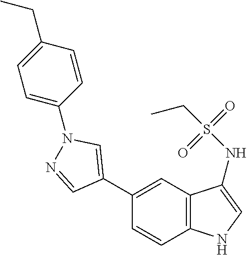 [SMILES: CCc1ccc(-n2cc(-c3ccc4[nH]cc(NS(=O)(=O)CC)c4c3)cn2)cc1]

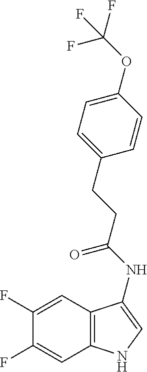 [SMILES: O=C(CCc1ccc(OC(F)(F)F)cc1)Nc1c[nH]c2cc(F)c(F)cc12]

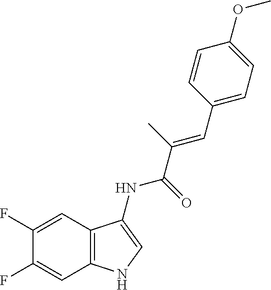 [SMILES: COc1ccc(/C=C(\C)C(=O)Nc2c[nH]c3cc(F)c(F)cc23)cc1]

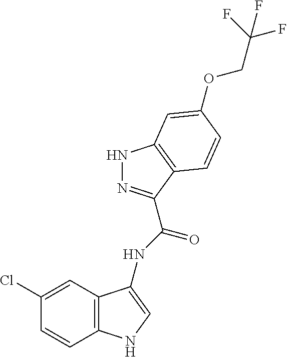 [SMILES: O=C(Nc1c[nH]c2ccc(Cl)cc12)c1n[nH]c2cc(OCC(F)(F)F)ccc12]

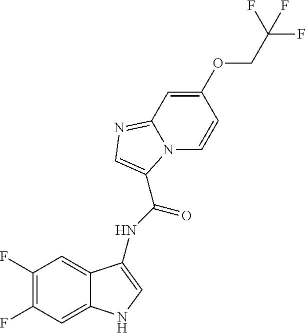 [SMILES: O=C(Nc1c[nH]c2cc(F)c(F)cc12)c1cnc2cc(OCC(F)(F)F)ccn12]

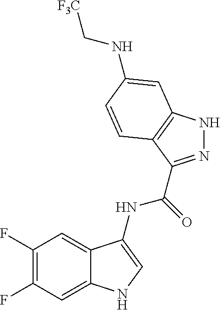 [SMILES: O=C(Nc1c[nH]c2cc(F)c(F)cc12)c1n[nH]c2cc(NCC(F)(F)F)ccc12]

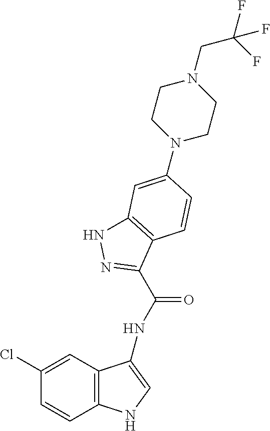 [SMILES: O=C(Nc1c[nH]c2ccc(Cl)cc12)c1n[nH]c2cc(N3CCN(CC(F)(F)F)CC3)ccc12]

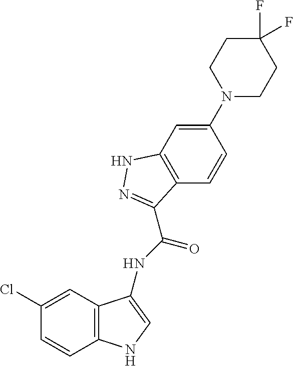 [SMILES: O=C(Nc1c[nH]c2ccc(Cl)cc12)c1n[nH]c2cc(N3CCC(F)(F)CC3)ccc12]

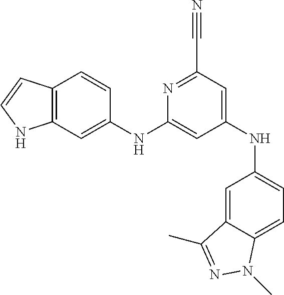 [SMILES: Cc1nn(C)c2ccc(Nc3cc(C#N)nc(Nc4ccc5cc[nH]c5c4)c3)cc12]

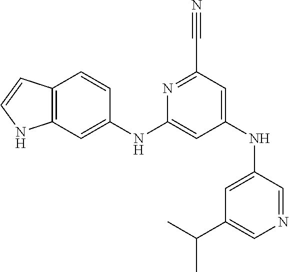 [SMILES: CC(C)c1cncc(Nc2cc(C#N)nc(Nc3ccc4cc[nH]c4c3)c2)c1]

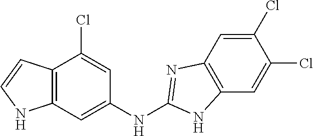 [SMILES: Clc1cc2nc(Nc3cc(Cl)c4cc[nH]c4c3)[nH]c2cc1Cl]

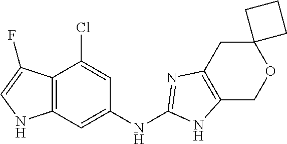 [SMILES: Fc1c[nH]c2cc(Nc3nc4c([nH]3)COC3(CCC3)C4)cc(Cl)c12]

### STING Inhibitory Nucleic Acids

In some embodiments of any of the methods described herein, the STING antagonist is an inhibitory nucleic acid. In some embodiments, the inhibitory nucleic acid is a short interfering RNA, an antisense nucleic acid, a cyclic dinucleotide, or a ribozyme.

Examples of aspects of these different oligonucleotides are described below. Any of the examples of inhibitory nucleic acids that are STING antagonists can decrease expression of STING mRNA in a mammalian cell (e.g., a human cell). Any of the inhibitory nucleic acids described herein can be synthesized in vitro.

Inhibitory nucleic acids that can decrease the expression of STING mRNA expression in a mammalian cell include antisense nucleic acid molecules, i.e., nucleic acid molecules whose nucleotide sequence is complementary to all or part of a STING mRNA (e.g., complementary to all or a part of any one of SEQ ID NOs: 1, 3, 5, or 7).

An antisense nucleic acid molecule can be complementary to all or part of a non-coding region of the coding strand of a nucleotide sequence encoding a STING protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding a STING protein described herein. Antisense nucleic acids targeting a nucleic acid encoding a STING protein can be designed using the software available at the Integrated DNA Technologies website.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-isopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding a STING protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An α-anomeric nucleic acid molecule forms specific double-stranded hybrids with complementary RNA in which, contrary to the usual, 0-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding a STING mRNA, e.g., specificity for any one of SEQ ID NOs: 1, 3, 5, or 7). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. STING mRNA can be used to select a catalytic RNA having a specific ribonuclease activity from a pool of RNA molecules. See, e.g., Bartel et al., Science 261:1411-1418, 1993.

Alternatively, a ribozyme having specificity for a STING mRNA sequence disclosed herein. For example, a derivative of a Tetrahymena L-19 IVS RNA can be constructed in which the nucleotide sequence of the active site is complementary to the nucleotide sequence to be cleaved in a STING mRNA (see, e.g., U.S. Pat. Nos. 4,987,071 and 5,116,742).

An inhibitory nucleic acid can also be a nucleic acid molecule that forms triple helical structures. For example, expression of a STING polypeptide can be inhibited by targeting nucleotide sequences complementary to the regulatory region of the gene encoding the STING polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

cGAS Inhibitors

In any of the methods described herein, the cGAS inhibitors can be any of the cGAS inhibitors described herein (e.g., any of the compounds described in this section). In any of the methods described herein, the cGAS inhibitor has an IC₅₀ of between about 1 nM and about 10 μM for cGAS.

In one aspect, the cGAS inhibitor is a compound selected from the group consisting of compounds in Table C₂ and pharmaceutically acceptable salts thereof.

TABLE C2
Structure

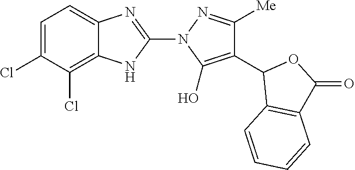 [SMILES: Cc1nn(-c2nc3ccc(Cl)c(Cl)c3[nH]2)c(O)c1C1OC(=O)c2ccccc21]

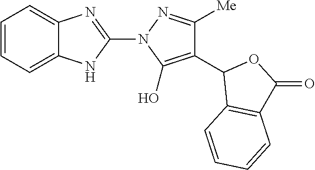 [SMILES: Cc1nn(-c2nc3ccccc3[nH]2)c(O)c1C1OC(=O)c2ccccc21]

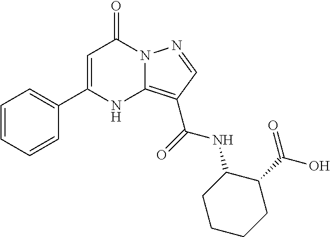

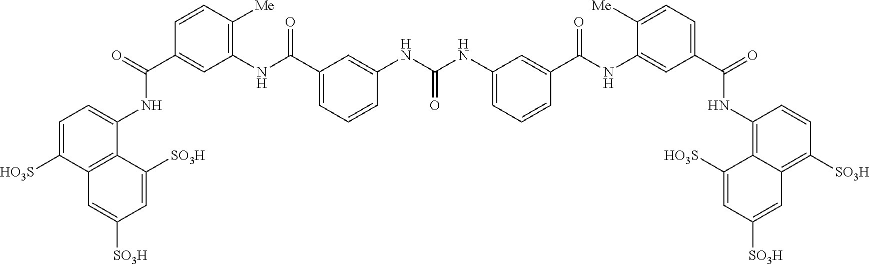 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

In some embodiments, the cGAS inhibitor is selected from the compounds disclosed in U.S. Provisional 62/355,403, filed on Jun. 28, 2016, which is incorporated herein by reference in its entirety.

In some embodiments, the cGAS inhibitor is selected from the compounds disclosed in U.S. Provisional 62/318,435, filed on Apr. 5, 2016, which is incorporated herein by reference in its entirety.

In some embodiments, the cGAS inhibitor is selected from the compounds disclosed in US Application 2018/0230115 A1, published Aug. 16, 2018, which is incorporated herein by reference in its entirety.

In some embodiments, the cGAS inhibitor is selected from the compounds disclosed in Vincent, J. et al. (2017) Nat. Commun. 8(1):750, which is incorporated herein by reference in its entirety.

In some embodiments, the cGAS inhibitor is selected from the compounds disclosed in Hall, J. et al. (2017) PLOS ONE 12(9):e184843, which is incorporated herein by reference in its entirety.

In some embodiments, the cGAS inhibitor is selected from the compounds disclosed in Wang, M. et al. (2018) Future Med. Chem. 10(11):1301-17, which is incorporated herein by reference in its entirety.

In some embodiments, the cGAS inhibitor is selected from the compounds disclosed in U.S. Provisional 62/559,482, filed on Sep. 15, 2017, which is incorporated herein by reference in its entirety.

In some embodiments, the cGAS inhibitor is selected from the compounds disclosed in U.S. Provisional 62/633,248, filed on Feb. 21, 2018, which is incorporated herein by reference in its entirety.

In some embodiments, the cGAS inhibitor is selected from the compounds disclosed in U.S. Provisional 62/687,769, filed on Jun. 20, 2018, which is incorporated herein by reference in its entirety.

### Pharmaceutical Compositions

In some embodiments, a STING antagonist or cGAS inhibitor (e.g., any of the STING antagonists or cGAS inhibitors described herein or known in the art) is administered as a pharmaceutical composition that includes the chemical entity and one or more pharmaceutically acceptable excipients, and optionally one or more additional therapeutic agents as described herein.

In some embodiments, the STING antagonist or cGAS inhibitor can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of the STING antagonists or cGAS inhibitors described herein. Dosage forms or compositions containing a STING antagonist or cGAS inhibitor as described herein in the range of 0.005% to 100% with the balance made up from non-toxic excipient may be prepared. The contemplated compositions may contain 0.001%-100% of a STING antagonist, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the STING antagonist or cGAS inhibitor (e.g., any of the exemplary STING antagonists or cGAS inhibitors described herein or known in the art) or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensionhs; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the STING antagonist or cGAS inhibitor in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the STING antagonist or cGAS inhibitor or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the STING antagonist or cGAS inhibitor with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the STING antagonist or cGAS inhibitor or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the STING antagonist or cGAS inhibitor is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a STING antagonist or cGAS inhibitor, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more STING antagonists or cGAS inhibitors or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the STING antagonist or cGAS inhibitor to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the STING antagonist or cGAS inhibitor are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Enema Formulations

In some embodiments, enema formulations containing a STING antagonist or cGAS inhibitor are provided in “ready-to-use” form.

In some embodiments, enema formulations containing a STING antagonist or cGAS inhibitor are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the STING antagonist or cGAS inhibitor (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhanceers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing a STING antagonist or cGAS inhibitor include water and one or more (e.g., all) of the following excipients:

One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);

One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;

One or more (e.g., one or two; e.g., two) buffers, such as phosphate buffer system (e.g., sodium dihydrogen phospahate dehydrate, disodium phosphate dodecahydrate);

One or more (e.g., one or two, e.g., two) glidants and/or lubricants, such as magnesium stearate and/or talc;

One or more (e.g., one or two; e.g., one) disintegrants, such as crospovidone; and

One or more (e.g., one or two; e.g., one) diluents, such as lactose (e.g., lactose monohydrate).

In certain of these embodiments, the STING antagonist is a compound of any one of Formulas I-XXIV or Formulas M1-M6 or a compound shown in any one of Table C1, or a pharmaceutically acceptable salt and/or hydrate and/or cocrystal thereof.

In certain embodiments, enema formulations containing a STING antagonist or cGAS inhibitor include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the STING antagonist is a compound of any one of Formulas I-XXIV or Formulas M1-M6 or a compound shown in any one of Table C1, or a pharmaceutically acceptable salt and/or hydrate and/or cocrystal thereof.

In certain embodiments, enema formulations containing a STING antagonist or cGAS inhibitor are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the STING antagonist or cGAS inhibitor (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the STING antagonist or cGAS inhibitor (e.g., a compound of any one of Formulas I-XXIV or Formulas M1-M6 or a compound shown in any one of Tables C₁-C₂, or a pharmaceutically acceptable salt and/or hydrate and/or cocrystal thereof) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the STING antagonist or cGAS inhibitor (e.g., a compound of any one of Formulas I-XXIV or Formulas M1-M6 or a compound shown in any one of Tables C1-C2, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of any one | 40 weight percent to about 80 weight percent |
| of Formulas I-XXIV or | (e.g., from about 50 weight percent to about 70 |
| Formulas M1-M6 or a | weight percent, from about 55 weight percent |
| compound shown in | to about 70 weight percent; from about 60 |
| Tables C1-C2 | weight percent to about 65 weight percent; e.g., |
| | about 62.1 weight percent) |
| Crospovidone (Kollidon | 0.5 weight percent to about 5 weight percent |
| CL) | (e.g., from about 0.5 weight percent to about 3 |
| | weight percent, from about 1 weight percent to |
| | about 3 weight percent; about 1.93 weight |
| | percent |
| lactose monohydrate | about 10 weight percent to about 50 weight |
| (Pharmatose 200M) | percent (e.g., from about 20 weight percent to |
| | about 40 weight percent, from about 25 weight |
| | percent to about 35 weight percent; e.g., about |
| | 31.03 weight percent |
| Povidone (Kollidon K30) | about 0.5 weight percent to about 5 weight |
| | percent (e.g., from about 1.5 weight percent to |
| | about 4.5 weight percent, from about 2 weight |
| | percent to about 3.5 weight percent; e.g., about |
| | 2.76 weight percent |
| Talc | 0.5 weight percent to about 5 weight percent |
| | (e.g., from about 0.5 weight percent to about 3 |
| | weight percent, from about 1 weight percent to |
| | about 3 weight percent; from about 1.5 weight |
| | percent to about 2.5 weight percent; from about |
| | 1.8 weight percent to about 2.2 weight percent; |
| | e.g., about 1.93 weight percent |
| Magnesium stearate | about 0.05 weight percent to about 1 weight |
| | percent (e.g., from about 0.05 weight percent |
| | to about 1 weight percent; from about 0.1 |
| | weight percent to about 1 weight percent; from |
| | about 0.1 weight percent to about 0.5 weight |
| | percent; e.g., about 0.27 weight percent |

In certain embodiments, component (i) includes the ingredients and amounts as shown in Table B.

TABLE B

| Ingredient | Weight Percent |
|---|---|
| A compound of any one of Formulas I-XXIV | About 62.1 weight percent) |
| or Formulas M1-M6 or a compound shown | |
| in any one of Tables C1-C2 | |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the STING antagonist or cGAS inhibitor, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′″) is present.

In certain embodiments, component (ii) includes water (up to 100%) and the ingredients and amounts as shown in Table C.

TABLE C

| Ingredient | Weight Percent |
|---|---|
| methyl cellulose (Methocel | 0.05 weight percent to about 5 weight |
| A15C premium) | percent (e.g., from about 0.05 weight |
| | percent to about 3 weight percent, from |
| | about 0.1 weight percent to about 3 |
| | weight percent; e.g., about 1.4 weight |
| | percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 |
| | weight percent; e.g., about 1.0 weight |
| | percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight |
| | percent; e.g., about 0.20 weight percent) |
| disodium phosphate | about 0.05 weight percent to about 1 |
| dodecahydrate | weight percent (e.g., from about 0.05 |
| | weight percent to about 0.5 weight |
| | percent; e.g., about 0.15 weight percent) |
| sodium dihydrogen phospahate | about 0.005 weight percent to about 0.5 |
| dihydrate | weight percent (e.g., from about 0.005 |
| | weight percent to about 0.3 weight |
| | percent; e.g., about 0.15 weight percent) |

In certain embodiments, component (ii) includes water (up to 1000%) and the ingredients and amounts as shown in Table D.

TABLE D

| Ingredient | Weight Percent |
|---|---|
| methyl cellulose (Methocel A15C | about 1.4 weight percent |
| premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

“Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield that is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device that includes a plastic bottle, a breakable capsule, and a rectal cannula and single flow pack.

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the STING antagonist or cGAS inhibitor is administered at a dosage of from about 0.001 mg/kg to about 500 mg/kg.

In some embodiments, enema formulations include from about 0.5 mg to about 2500 mg of the chemical entity in from about 1 mL to about 3000 mL of liquid carrier.

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a STING antagonist or cGAS inhibitor is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a STING antagonist or cGAS inhibitor is administered to an individual for a period of time followed by a separate period of time. In another embodiment, a STING antagonist or cGAS inhibitor is administered for a first period and a second period following the first period, with administration stopped during the second period, followed by a third period where administration of the STING antagonist or cGAS inhibitor is started and then a fourth period following the third period where administration is stopped. In an aspect of this embodiment, the period of administration of a STING antagonist or cGAS inhibitor followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Kits

Also provided herein are kits containing one or more (e.g., at least 1, 2, 3, 4, 5, 6, 7, 8, 9, 10, 11, 12, 14, 15, 16, 18, or 20) of any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. The kits described herein are not so limited; other variations will be apparent to one of ordinary skill in the art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Sequence Appendix

Human STING cDNA, Variant 1 (SEQ ID NO: 1)
ATGCCCCACTCCAGCCTGCATCCATCCATCCCGTGTCCCAGGGGTCACGGGG
CCCAGAAGGCAGCCTTGGTTCTGCTGAGTGCCTGCCTGGTGACCCTTTGGGGG
CTAGGAGAGCCACCAGAGCACACTCTCCGGTACCTGGTGCTCCACCTAGCCT
CCCTGCAGCTGGGACTGCTGTTAAACGGGGTCTGCAGCCTGGCTGAGGAGCT
GCGCCACATCCACTCCAGGTACCGGGGCAGCTACTGGAGGACTGTGCGGGCC
TGCCTGGGCTGCCCCCTCCGCCGTGGGGCCCTGTTGCTGCTGTCCATCTATTT
CTACTACTCCCTCCCAAATGCGGTCGGCCCGCCCTTCACTTGGATGCTTGCCC
TCCTGGGCCTCTCGCAGGCACTGAACATCCTCCTGGGCCTCAAGGGCCTGGC
CCCAGCTGAGATCTCTGCAGTGTGTGAAAAAGGGAATTTCAACGTGGCCCAT
GGGCTGGCATGGTCATATTACATCGGATATCTGCGGCTGATCCTGCCAGAGCT
CCAGGCCCGGATTCGAACTTACAATCAGCATTACAACAACCTGCTACGGGGT
GCAGTGAGCCAGCGGCTGTATATTCTCCTCCCATTGGACTGTGGGGTGCCTGA
TAACCTGAGTATGGCTGACCCCAACATTCGCTTCCTGGATAAACTGCCCCAGC
AGACCGGTGACCATGCTGGCATCAAGGATCGGGTTTACAGCAACAGCATCTA
TGAGCTTCTGGAGAACGGGCAGCGGGCGGGCACCTGTGTCCTGGAGTACGCC
ACCCCCTTGCAGACTTTGTTTGCCATGTCACAATACAGTCAAGCTGGCTTTAG
CCGGGAGGATAGGCTTGAGCAGGCCAAACTCTTCTGCCGGACACTTGAGGAC
ATCCTGGCAGATGCCCCTGAGTCTCAGAACAACTGCCGCCTCATTGCCTACCA
GGAACCTGCAGATGACAGCAGCTTCTCGCTGTCCCAGGAGGTTCTCCGGCAC
CTGCGGCAGGAGGAAAAGGAAGAGGTTACTGTGGGCAGCTTGAAGACCTCA
GCGGTGCCCAGTACCTCCACGATGTCCCAAGAGCCTGAGCTCCTCATCAGTG
GAATGGAAAAGCCCCTCCCTCTCCGCACGGATTTCTCTTGA
Human STING Protein, Variant 1 (SEQ ID NO: 2)
MPHSSLHPSIPCPRGHGAQKAALVLLSACLVTLWGLGEPPEHTLRYLVLHLASLQ
LGLLLNGVCSLAEELRHIHSRYRGSYWRTVRACLGCPLRRGALLLLSIYFYYSLP
NAVGPPFTWMLALLGLSQALNILLGLKGLAPAEISAVCEKGNFNVAHGLAWSYY
IGYLRLILPELQARIRTYNQHYNNLLRGAVSQRLYILLPLDCGVPDNLSMADPNIR
FLDKLPQQTGDHAGIKDRVYSNSIYELLENGQRAGTCVLEYATPLQTLFAMSQY
SQAGFSREDRLEQAKLFCRTLEDILADAPESQNNCRLIAYQEPADDSSFSLSQEVL
RHLRQEEKEEVTVGSLKTSAVPSTSTMSQEPELLISGMEKPLPLRTDFS
Human STING cDNA, Variant 2 (SEQ ID NO: 3)
ATGCCCCACTCCAGCCTGCATCCATCCATCCCGTGTCCCAGGGGTCACGGGG
CCCAGAAGGCAGCCTTGGTTCTGCTGAGTGCCTGCCTGGTGACCCTTTGGGGG
CTAGGAGAGCCACCAGAGCACACTCTCCGGTACCTGGTGCTCCACCTAGCCT
CCCTGCAGCTGGGACTGCTGTTAAACGGGGTCTGCAGCCTGGCTGAGGAGCT
GCGCCACATCCACTCCAGGTACCGGGGCAGCTACTGGAGGACTGTGCGGGCC
TGCCTGGGCTGCCCCCTCCGCCGTGGGGCCCTGTTGCTGCTGTCCATCTATTT
CTACTACTCCCTCCCAAATGCGGTCGGCCCGCCCTTCACTTGGATGCTTGCCC
TCCTGGGCCTCTCGCAGGCACTGAACATCCTCCTGGGCCTCAAGGGCCTGGC
CCCAGCTGAGATCTCTGCAGTGTGTGAAAAAGGGAATTTCAACGTGGCCCAT
GGGCTGGCATGGTCATATTACATCGGATATCTGCGGCTGATCCTGCCAGAGCT
CCAGGCCCGGATTCGAACTTACAATCAGCATTACAACAACCTGCTACGGGGT
GCAGTGAGCCAGCGGCTGTATATTCTCCTCCCATTGGACTGTGGGGTGCCTGA
TAACCTGAGTATGGCTGACCCCAACATTCGCTTCCTGGATAAACTGCCCCAGC
AGACCGGTGACCATGCTGGCATCAAGGATCGGGTTTACAGCAACAGCATCTA
TGAGCTTCTGGAGAACGGGCAGCGGAACCTGCAGATGACAGCAGCTTCTCGC
TGTCCCAGGAGGTTCTCCGGCACCTGCGGCAGGAGGAAAAGGAAGAGGTTAC
TGTGGGCAGCTTGA
Human STING Protein, Variant 2 (SEQ ID NO: 4)
MPHSSLHPSIPCPRGHGAQKAALVLLSACLVTLWGLGEPPEHTLRYLVLHLASLQ
LGLLLNGVCSLAEELRHIHSRYRGSYWRTVRACLGCPLRRGALLLLSIYFYYSLP
NAVGPPFTWMLALLGLSQALNILLGLKGLAPAEISAVCEKGNFNVAHGLAWSYY
IGYLRLILPELQARIRTYNQHYNNLLRGAVSQRLYILLPLDCGVPDNLSMADPNIR
FLDKLPQQTGDRAGIKDRVYSNSIYELLENGQRNLQMTAASRCPRRFSGTCGRR
KRKRLLWAA
Human STING cDNA, Variant 3 Precursor (SEQ ID NO: 5)
ATGCTTGCCCTCCTGGGCCTCTCGCAGGCACTGAACATCCTCCTGGGCCTCAA
GGGCCTGGCCCCAGCTGAGATCTCTGCAGTGTGTGAAAAAGGGAATTTCAAC
GTGGCCCATGGGCTGGCATGGTCATATTACATCGGATATCTGCGGCTGATCCT
GCCAGAGCTCCAGGCCCGGATTCGAACTTACAATCAGCATTACAACAACCTG
CTACGGGGTGCAGTGAGCCAGCGGCTGTATATTCTCCTCCCATTGGACTGTGG
GGTGCCTGATAACCTGAGTATGGCTGACCCCAACATTCGCTTCCTGGATAAAC
TGCCCCAGCAGACCGGTGACCATGCTGGCATCAAGGATCGGGTTTACAGCAA
CAGCATCTATGAGCTTCTGGAGAACGGGCAGCGGGCGGGCACCTGTGTCCTG
GAGTACGCCACCCCCTTGCAGACTTTGTTTGCCATGTCACAATACAGTCAAGC
TGGCTTTAGCCGGGAGGATAGGCTTGAGCAGGCCAAACTCTTCTGCCGGACA
CTTGAGGACATCCTGGCAGATGCCCCTGAGTCTCAGAACAACTGCCGCCTCA
TTGCCTACCAGGAACCTGCAGATGACAGCAGCTTCTCGCTGTCCCAGGAGGT
TCTCCGGCACCTGCGGCAGGAGGAAAAGGAAGAGGTTACTGTGGGCAGCTTG
AAGACCTCAGCGGTGCCCAGTACCTCCACGATGTCCCAAGAGCCTGAGCTCC
TCATCAGTGGAATGGAAAAGCCCCTCCCTCTCCGCACGGATTTCTCTTGA
Human STING Protein, Variant 3 Precursor (SEQ ID NO: 6)
MLALLGLSQALNILLGLKGLAPAEISAVCEKGNFNVAHGLAWSYYIGYLRLILPE
LQARIRTYNQHYNNLLRGAVSQRLYILLPLDCGVPDNLSMADPNIRFLDKLPQQT
GDHAGIKDRVYSNSIYELLENGQRAGTCVLEYATPLQTLFAMSQYSQAGFSRED
RLEQAKLFCRTLEDILADAPESQNNCRLIAYQEPADDSSFSLSQEVLRHLRQEEKE
EVTVGSLKTSAVPSTSTMSQEPELLISGMEKPLPLRTDFS
Human STING cDNA, Variant 3 Mature Sequence (SEQ ID NO: 7)
CTCAAGGGCCTGGCCCCAGCTGAGATCTCTGCAGTGTGTGAAAAAGGGAATT
TCAACGTGGCCCATGGGCTGGCATGGTCATATTACATCGGATATCTGCGGCTG
ATCCTGCCAGAGCTCCAGGCCCGGATTCGAACTTACAATCAGCATTACAACA
ACCTGCTACGGGGTGCAGTGAGCCAGCGGCTGTATATTCTCCTCCCATTGGAC
TGTGGGGTGCCTGATAACCTGAGTATGGCTGACCCCAACATTCGCTTCCTGGA
TAAACTGCCCCAGCAGACCGGTGACCATGCTGGCATCAAGGATCGGGTTTAC
AGCAACAGCATCTATGAGCTTCTGGAGAACGGGCAGCGGGCGGGCACCTGTG
TCCTGGAGTACGCCACCCCCTTGCAGACTTTGTTTGCCATGTCACAATACAGT
CAAGCTGGCTTTAGCCGGGAGGATAGGCTTGAGCAGGCCAAACTCTTCTGCC
GGACACTTGAGGACATCCTGGCAGATGCCCCTGAGTCTCAGAACAACTGCCG
CCTCATTGCCTACCAGGAACCTGCAGATGACAGCAGCTTCTCGCTGTCCCAG
GAGGTTCTCCGGCACCTGCGGCAGGAGGAAAAGGAAGAGGTTACTGTGGGC
AGCTTGAAGACCTCAGCGGTGCCCAGTACCTCCACGATGTCCCAAGAGCCTG
AGCTCCTCATCAGTGGAATGGAAAAGCCCCTCCCTCTCCGCACGGATTTCTCT
TGA
Human STING Protein, Variant 3 Mature Sequence (SEQ ID NO: 8)
LKGLAPAEISAVCEKGNFNVAHGLAWSYYIGYLRLILPELQARIRTYNQHYNNLL
RGAVSQRLYILLPLDCGVPDNLSMADPNIRFLDKLPQQTGDHAGIKDRVYSNSIY
ELLENGQRAGTCVLEYATPLQTLFAMSQYSQAGFSREDRLEQAKLFCRTLEDILA
DAPESQNNCRLIAYQEPADDSSFSLSQEVLRHLRQEEKEEVTVGSLKTSAVPSTST
MSQEPELLISGMEKPLPLRTDFS
Human TREX1 cDNA Sequence, Variant 1 (SEQ ID NO: 9)
ATGGGCTCGCAGGCCCTGCCCCCGGGGCCCATGCAGACCCTCATCTTTTTCGA
CATGGAGGCCACTGGCTTGCCCTTCTCCCAGCCCAAGGTCACGGAGCTGTGC
CTGCTGGCTGTCCACAGATGTGCCCTGGAGAGCCCCCCCACCTCTCAGGGGC
CACCTCCCACAGTTCCTCCACCACCGCGTGTGGTAGACAAGCTCTCCCTGTGT
GTGGCTCCGGGGAAGGCCTGCAGCCCTGCAGCCAGCGAGATCACAGGTCTGA
GCACAGCTGTGCTGGCAGCGCATGGGCGTCAATGTTTTGATGACAACCTGGC
CAACCTGCTCCTAGCCTTCCTGCGGCGCCAGCCACAGCCCTGGTGCCTGGTGG
CACACAATGGTGACCGCTACGACTTCCCCCTGCTCCAAGCAGAGCTGGCTAT
GCTGGGCCTCACCAGTGCTCTGGATGGTGCCTTCTGTGTGGATAGCATCACTG
CGCTGAAGGCCCTGGAGCGAGCAAGCAGCCCCTCAGAACACGGCCCAAGGA
AGAGCTATAGCCTAGGCAGCATCTACACTCGCCTGTATGGGCAGTCCCCTCC
AGACTCGCACACGGCTGAGGGTGATGTCCTGGCCCTGCTCAGCATCTGTCAG
TGGAGACCACAGGCCCTGCTGCGGTGGGTGGATGCTCACGCCAGGCCTTTCG
GCACCATCAGGCCCATGTATGGGGTCACAGCCTCTGCTAGGACCAAGCCAAG
ACCATCTGCTGTCACAACCACTGCACACCTGGCCACAACCAGGAACACTAGT
CCCAGCCTTGGAGAGAGCAGGGGTACCAAGGATCTTCCTCCAGTGAAGGACC
CTGGAGCCCTATCCAGGGAGGGGCTGCTGGCCCCACTGGGTCTGCTGGCCAT
CCTGACCTTGGCAGTAGCCACACTGTATGGACTATCCCTGGCCACACCTGGG
GAGTAG
Human TREX1 Protein Sequence, Variant 1 (SEQ ID NO: 10)
MGSQALPPGPMQTLIFFDMEATGLPFSQPKVTELCLLAVHRCALESPPTSQGPPPT
VPPPPRVVDKLSLCVAPGKACSPAASEITGLSTAVLAAHGRQCFDDNLANLLLAF
LRRQPQPWCLVAHNGDRYDFPLLQAELAMLGLTSALDGAFCVDSITALKALERA
SSPSEHGPRKSYSLGSIYTRLYGQSPPDSHTAEGDVLALLSICQWRPQALLRWVD
AHARPFGTIRPMYGVTASARTKPRPSAVTTTAHLATTRNTSPSLGESRGTKDLPP
VKDPGALSREGLLAPLGLLAILTLAVATLYGLSLATPGE
Human TREX1 cDNA Sequence, Variant 2 (SEQ ID NO: 11)
ATGCAGACCCTCATCTTTTTCGACATGGAGGCCACTGGCTTGCCCTTCTCCCA
GCCCAAGGTCACGGAGCTGTGCCTGCTGGCTGTCCACAGATGTGCCCTGGAG
AGCCCCCCCACCTCTCAGGGGCCACCTCCCACAGTTCCTCCACCACCGCGTGT
GGTAGACAAGCTCTCCCTGTGTGTGGCTCCGGGGAAGGCCTGCAGCCCTGCA
GCCAGCGAGATCACAGGTCTGAGCACAGCTGTGCTGGCAGCGCATGGGCGTC
AATGTTTTGATGACAACCTGGCCAACCTGCTCCTAGCCTTCCTGCGGCGCCAG
CCACAGCCCTGGTGCCTGGTGGCACACAATGGTGACCGCTACGACTTCCCCC
TGCTCCAAGCAGAGCTGGCTATGCTGGGCCTCACCAGTGCTCTGGATGGTGC
CTTCTGTGTGGATAGCATCACTGCGCTGAAGGCCCTGGAGCGAGCAAGCAGC
CCCTCAGAACACGGCCCAAGGAAGAGCTATAGCCTAGGCAGCATCTACACTC
GCCTGTATGGGCAGTCCCCTCCAGACTCGCACACGGCTGAGGGTGATGTCCT
GGCCCTGCTCAGCATCTGTCAGTGGAGACCACAGGCCCTGCTGCGGTGGGTG
GATGCTCACGCCAGGCCTTTCGGCACCATCAGGCCCATGTATGGGGTCACAG
CCTCTGCTAGGACCAAGCCAAGACCATCTGCTGTCACAACCACTGCACACCT
GGCCACAACCAGGAACACTAGTCCCAGCCTTGGAGAGAGCAGGGGTACCAA
GGATCTTCCTCCAGTGAAGGACCCTGGAGCCCTATCCAGGGAGGGGCTGCTG
GCCCCACTGGGTCTGCTGGCCATCCTGACCTTGGCAGTAGCCACACTGTATGG
ACTATCCCTGGCCACACCTGGGGAGTAG
Human TREX1 Protein Sequence, Variant 2 (SEQ ID NO: 12)
MQTLIFFDMEATGLPFSQPKVTELCLLAVHRCALESPPTSQGPPPTVPPPPRVVDK
LSLCVAPGKACSPAASEITGLSTAVLAAHGRQCFDDNLANLLLAFLRRQPQPWC
LVAHNGDRYDFPLLQAELAMLGLTSALDGAFCVDSITALKALERASSPSEHGPR
KSYSLGSIYTRLYGQSPPDSHTAEGDVLALLSICQWRPQALLRWVDAHARPFGTI
RPMYGVTASARTKPRPSAVTTTAHLATTRNTSPSLGESRGTKDLPPVKDPGALSR
EGLLAPLGLLAILTLAVATLYGLSLATPGE
Human TREX Protein Sequence, Variant 3 (SEQ ID NO: 13)
MGPGARRQGRIVQGRPEMCFCPPPTPLPPLRILTLGTHTPTPCSSPGSAAGTYPTM
GSQALPPGPMQTLIFFDMEATGLPFSQPKVTELCLLAVHRCALESPPTSQGPPPTV
PPPPRVVDKLSLCVAPGKACSPAASEITGLSTAVLAAHGRQCFDDNLANLLLAFL
RRQPQPWCLVAHNGDRYDFPLLQAELAMLGLTSALDGAFCVDSITALKALERAS
SPSEHGPRKSYSLGSIYTRLYGQSPPDSHTAEGDVLALLSICQWRPQALLRWVDA
HARPFGTIRPMYGVTASARTKPRPSAVTTTAHLATTRNTSPSLGESRGTKDLPPV
KDPGALSREGLLAPLGLLAILTLAVATLYGLSLATPGE
Human BRCA1 cDNA Sequence, Variant 1 (SEQ ID NO: 14)
ATGGATTTATCTGCTCTTCGCGTTGAAGAAGTACAAAATGTCATTAATGCTAT
GCAGAAAATCTTAGAGTGTCCCATCTGTCTGGAGTTGATCAAGGAACCTGTCT
CCACAAAGTGTGACCACATATTTTGCAAATTTTGCATGCTGAAACTTCTCAAC
CAGAAGAAAGGGCCTTCACAGTGTCCTTTATGTAAGAATGATATAACCAAAA
GGAGCCTACAAGAAAGTACGAGATTTAGTCAACTTGTTGAAGAGCTATTGAA
AATCATTTGTGCTTTTCAGCTTGACACAGGTTTGGAGTATGCAAACAGCTATA
ATTTTGCAAAAAAGGAAAATAACTCTCCTGAACATCTAAAAGATGAAGTTTC
TATCATCCAAAGTATGGGCTACAGAAACCGTGCCAAAAGACTTCTACAGAGT
GAACCCGAAAATCCTTCCTTGCAGGAAACCAGTCTCAGTGTCCAACTCTCTA
ACCTTGGAACTGTGAGAACTCTGAGGACAAAGCAGCGGATACAACCTCAAAA
GACGTCTGTCTACATTGAATTGGGATCTGATTCTTCTGAAGATACCGTTAATA
AGGCAACTTATTGCAGTGTGGGAGATCAAGAATTGTTACAAATCACCCCTCA
AGGAACCAGGGATGAAATCAGTTTGGATTCTGCAAAAAAGGCTGCTTGTGAA
TTTTCTGAGACGGATGTAACAAATACTGAACATCATCAACCCAGTAATAATG
ATTTGAACACCACTGAGAAGCGTGCAGCTGAGAGGCATCCAGAAAAGTATCA
GGGTAGTTCTGTTTCAAACTTGCATGTGGAGCCATGTGGCACAAATACTCATG
CCAGCTCATTACAGCATGAGAACAGCAGTTTATTACTCACTAAAGACAGAAT
GAATGTAGAAAAGGCTGAATTCTGTAATAAAAGCAAACAGCCTGGCTTAGCA
AGGAGCCAACATAACAGATGGGCTGGAAGTAAGGAAACATGTAATGATAGG
CGGACTCCCAGCACAGAAAAAAAGGTAGATCTGAATGCTGATCCCCTGTGTG
AGAGAAAAGAATGGAATAAGCAGAAACTGCCATGCTCAGAGAATCCTAGAG
ATACTGAAGATGTTCCTTGGATAACACTAAATAGCAGCATTCAGAAAGTTAA
TGAGTGGTTTTCCAGAAGTGATGAACTGTTAGGTTCTGATGACTCACATGATG
GGGAGTCTGAATCAAATGCCAAAGTAGCTGATGTATTGGACGTTCTAAATGA
GGTAGATGAATATTCTGGTTCTTCAGAGAAAATAGACTTACTGGCCAGTGAT
CCTCATGAGGCTTTAATATGTAAAAGTGAAAGAGTTCACTCCAAATCAGTAG
AGAGTAATATTGAAGACAAAATATTTGGGAAAACCTATCGGAAGAAGGCAA
GCCTCCCCAACTTAAGCCATGTAACTGAAAATCTAATTATAGGAGCATTTGTT
ACTGAGCCACAGATAATACAAGAGCGTCCCCTCACAAATAAATTAAAGCGTA
AAAGGAGACCTACATCAGGCCTTCATCCTGAGGATTTTATCAAGAAAGCAGA
TTTGGCAGTTCAAAAGACTCCTGAAATGATAAATCAGGGAACTAACCAAACG
GAGCAGAATGGTCAAGTGATGAATATTACTAATAGTGGTCATGAGAATAAAA
CAAAAGGTGATTCTATTCAGAATGAGAAAAATCCTAACCCAATAGAATCACT
CGAAAAAGAATCTGCTTTCAAAACGAAAGCTGAACCTATAAGCAGCAGTATA
AGCAATATGGAACTCGAATTAAATATCCACAATTCAAAAGCACCTAAAAAGA
ATAGGCTGAGGAGGAAGTCTTCTACCAGGCATATTCATGCGCTTGAACTAGT
AGTCAGTAGAAATCTAAGCCCACCTAATTGTACTGAATTGCAAATTGATAGTT
GTTCTAGCAGTGAAGAGATAAAGAAAAAAAAGTACAACCAAATGCCAGTCA
GGCACAGCAGAAACCTACAACTCATGGAAGGTAAAGAACCTGCAACTGGAG
CCAAGAAGAGTAACAAGCCAAATGAACAGACAAGTAAAAGACATGACAGCG
ATACTTTCCCAGAGCTGAAGTTAACAAATGCACCTGGTTCTTTTACTAAGTGT
TCAAATACCAGTGAACTTAAAGAATTTGTCAATCCTAGCCTTCCAAGAGAAG
AAAAAGAAGAGAAACTAGAAACAGTTAAAGTGTCTAATAATGCTGAAGACC
CCAAAGATCTCATGTTAAGTGGAGAAAGGGTTTTGCAAACTGAAAGATCTGT
AGAGAGTAGCAGTATTTCATTGGTACCTGGTACTGATTATGGCACTCAGGAA
AGTATCTCGTTACTGGAAGTTAGCACTCTAGGGAAGGCAAAAACAGAACCAA
ATAAATGTGTGAGTCAGTGTGCAGCATTTGAAAACCCCAAGGGACTAATTCA
TGGTTGTTCCAAAGATAATAGAAATGACACAGAAGGCTTTAAGTATCCATTG
GGACATGAAGTTAACCACAGTCGGGAAACAAGCATAGAAATGGAAGAAAGT
GAACTTGATGCTCAGTATTTGCAGAATACATTCAAGGTTTCAAAGCGCCAGTC
ATTTGCTCCGTTTTCAAATCCAGGAAATGCAGAAGAGGAATGTGCAACATTC
TCTGCCCACTCTGGGTCCTTAAAGAAACAAAGTCCAAAAGTCACTTTTGAATG
TGAACAAAAGGAAGAAAATCAAGGAAAGAATGAGTCTAATATCAAGCCTGT
ACAGACAGTTAATATCACTGCAGGCTTTCCTGTGGTTGGTCAGAAAGATAAG
CCAGTTGATAATGCCAAATGTAGTATCAAAGGAGGCTCTAGGTTTTGTCTATC
ATCTCAGTTCAGAGGCAACGAAACTGGACTCATTACTCCAAATAAACATGGA
CTTTTACAAAACCCATATCGTATACCACCACTTTTTCCCATCAAGTCATTTGTT
AAAACTAAATGTAAGAAAAATCTGCTAGAGGAAAACTTTGAGGAACATTCAA
TGTCACCTGAAAGAGAAATGGGAAATGAGAACATTCCAAGTACAGTGAGCA
CAATTAGCCGTAATAACATTAGAGAAAATGTTTTTAAAGAAGCCAGCTCAAG
CAATATTAATGAAGTAGGTTCCAGTACTAATGAAGTGGGCTCCAGTATTAAT
GAAATAGGTTCCAGTGATGAAAACATTCAAGCAGAACTAGGTAGAAACAGA
GGGCCAAAATTGAATGCTATGCTTAGATTAGGGGTTTTGCAACCTGAGGTCT
ATAAACAAAGTCTTCCTGGAAGTAATTGTAAGCATCCTGAAATAAAAAAGCA
AGAATATGAAGAAGTAGTTCAGACTGTTAATACAGATTTCTCTCCATATCTGA
TTTCAGATAACTTAGAACAGCCTATGGGAAGTAGTCATGCATCTCAGGTTTGT
TCTGAGACACCTGATGACCTGTTAGATGATGGTGAAATAAAGGAAGATACTA
GTTTTGCTGAAAATGACATTAAGGAAAGTTCTGCTGTTTTTAGCAAAAGCGTC
CAGAAAGGAGAGCTTAGCAGGAGTCCTAGCCCTTTCACCCATACACATTTGG
CTCAGGGTTACCGAAGAGGGGCCAAGAAATTAGAGTCCTCAGAAGAGAACTT
ATCTAGTGAGGATGAAGAGCTTCCCTGCTTCCAACACTTGTTATTTGGTAAAG
TAAACAATATACCTTCTCAGTCTACTAGGCATAGCACCGTTGCTACCGAGTGT
CTGTCTAAGAACACAGAGGAGAATTTATTATCATTGAAGAATAGCTTAAATG
ACTGCAGTAACCAGGTAATATTGGCAAAGGCATCTCAGGAACATCACCTTAG
TGAGGAAACAAAATGTTCTGCTAGCTTGTTTTCTTCACAGTGCAGTGAATTGG
AAGACTTGACTGCAAATACAAACACCCAGGATCCTTTCTTGATTGGTTCTTCC
AAACAAATGAGGCATCAGTCTGAAAGCCAGGGAGTTGGTCTGAGTGACAAG
GAATTGGTTTCAGATGATGAAGAAAGAGGAACGGGCTTGGAAGAAAATAAT
CAAGAAGAGCAAAGCATGGATTCAAACTTAGGTGAAGCAGCATCTGGGTGTG
AGAGTGAAACAAGCGTCTCTGAAGACTGCTCAGGGCTATCCTCTCAGAGTGA
CATTTTAACCACTCAGCAGAGGGATACCATGCAACATAACCTGATAAAGCTC
CAGCAGGAAATGGCTGAACTAGAAGCTGTGTTAGAACAGCATGGGAGCCAG
CCTTCTAACAGCTACCCTTCCATCATAAGTGACTCTTCTGCCCTTGAGGACCT
GCGAAATCCAGAACAAAGCACATCAGAAAAAGCAGTATTAACTTCACAGAA
AAGTAGTGAATACCCTATAAGCCAGAATCCAGAAGGCCTTTCTGCTGACAAG
TTTGAGGTGTCTGCAGATAGTTCTACCAGTAAAAATAAAGAACCAGGAGTGG
AAAGGTCATCCCCTTCTAAATGCCCATCATTAGATGATAGGTGGTACATGCAC
AGTTGCTCTGGGAGTCTTCAGAATAGAAACTACCCATCTCAAGAGGAGCTCA
TTAAGGTTGTTGATGTGGAGGAGCAACAGCTGGAAGAGTCTGGGCCACACGA
TTTGACGGAAACATCTTACTTGCCAAGGCAAGATCTAGAGGGAACCCCTTAC
CTGGAATCTGGAATCAGCCTCTTCTCTGATGACCCTGAATCTGATCCTTCTGA
AGACAGAGCCCCAGAGTCAGCTCGTGTTGGCAACATACCATCTTCAACCTCT
GCATTGAAAGTTCCCCAATTGAAAGTTGCAGAATCTGCCCAGAGTCCAGCTG
CTGCTCATACTACTGATACTGCTGGGTATAATGCAATGGAAGAAAGTGTGAG
CAGGGAGAAGCCAGAATTGACAGCTTCAACAGAAAGGGTCAACAAAAGAAT
GTCCATGGTGGTGTCTGGCCTGACCCCAGAAGAATTTATGCTCGTGTACAAGT
TTGCCAGAAAACACCACATCACTTTAACTAATCTAATTACTGAAGAGACTACT
CATGTTGTTATGAAAACAGATGCTGAGTTTGTGTGTGAACGGACACTGAAAT
ATTTTCTAGGAATTGCGGGAGGAAAATGGGTAGTTAGCTATTTCTGGGTGAC
CCAGTCTATTAAAGAAAGAAAAATGCTGAATGAGCATGATTTTGAAGTCAGA
GGAGATGTGGTCAATGGAAGAAACCACCAAGGTCCAAAGCGAGCAAGAGAA
TCCCAGGACAGAAAGATCTTCAGGGGGCTAGAAATCTGTTGCTATGGGCCCT
TCACCAACATGCCCACAGATCAACTGGAATGGATGGTACAGCTGTGTGGTGC
TTCTGTGGTGAAGGAGCTTTCATCATTCACCCTTGGCACAGGTGTCCACCCAA
TTGTGGTTGTGCAGCCAGATGCCTGGACAGAGGACAATGGCTTCCATGCAAT
TGGGCAGATGTGTGAGGCACCTGTGGTGACCCGAGAGTGGGTGTTGGACAGT
GTAGCACTCTACCAGTGCCAGGAGCTGGACACCTACCTGATACCCCAGATCC
CCCACAGCCACTACTGA
Human BRCA1 Protein Sequence, Variant 1 (SEQ ID NO: 15)
MDLSALRVEEVQNVINAMQKILECPICLELIKEPVSTKCDHIFCKFCMLKLLNQK
KGPSQCPLCKNDITKRSLQESTRFSQLVEELLKIICAFQLDTGLEYANSYNFAKKE
NNSPEHLKDEVSIIQSMGYRNRAKRLLQSEPENPSLQETSLSVQLSNLGTVRTLRT
KQRIQPQKTSVYIELGSDSSEDTVNKATYCSVGDQELLQITPQGTRDEISLDSAKK
AACEFSETDVTNTEHHQPSNNDLNTTEKRAAERHPEKYQGSSVSNLHVEPCGTN
THASSLQHENSSLLLTKDRMNVEKAEFCNKSKQPGLARSQHNRWAGSKETCND
RRTPSTEKKVDLNADPLCERKEWNKQKLPCSENPRDTEDVPWITLNSSIQKVNE
WFSRSDELLGSDDSHDGESESNAKVADVLDVLNEVDEYSGSSEKIDLLASDPHE
ALICKSERVHSKSVESNIEDKIFGKTYRKKASLPNLSHVTENLIIGAFVTEPQIIQER
PLTNKLKRKRRPTSGLHPEDFIKKADLAVQKTPEMINQGTNQTEQNGQVMNITN
SGHENKTKGDSIQNEKNPNPIESLEKESAFKTKAEPISSSISNMELELNIHNSKAPK
KNRLRRKSSTRHIHALELVVSRNLSPPNCTELQIDSCSSSEEIKKKKYNQMPVRHS
RNLQLMEGKEPATGAKKSNKPNEQTSKRHDSDTFPELKLTNAPGSFTKCSNTSEL
KEFVNPSLPREEKEEKLETVKVSNNAEDPKDLMLSGERVLQTERSVESSSISLVPG
TDYGTQESISLLEVSTLGKAKTEPNKCVSQCAAFENPKGLIHGCSKDNRNDTEGF
KYPLGHEVNHSRETSIEMEESELDAQYLQNTFKVSKRQSFAPFSNPGNAEEECAT
FSAHSGSLKKQSPKVTFECEQKEENQGKNESNIKPVQTVNITAGFPVVGQKDKPV
DNAKCSIKGGSRFCLSSQFRGNETGLITPNKHGLLQNPYRIPPLFPIKSFVKTKCKK
NLLEENFEEHSMSPEREMGNENIPSTVSTISRNNIRENVFKEASSSNINEVGSSTNE
VGSSINEIGSSDENIQAELGRNRGPKLNAMLRLGVLQPEVYKQSLPGSNCKHPEIK
KQEYEEVVQTVNTDFSPYLISDNLEQPMGSSHASQVCSETPDDLLDDGEIKEDTS
FAENDIKESSAVFSKSVQKGELSRSPSPFTHTHLAQGYRRGAKKLESSEENLSSED
EELPCFQHLLFGKVNNIPSQSTRHSTVATECLSKNTEENLLSLKNSLNDCSNQVIL
AKASQEHHLSEETKCSASLFSSQCSELEDLTANTNTQDPFLIGSSKQMRHQSESQ
GVGLSDKELVSDDEERGTGLEENNQEEQSMDSNLGEAASGCESETSVSEDCSGL
SSQSDILTTQQRDTMQHNLIKLQQEMAELEAVLEQHGSQPSNSYPSIISDSSALED
LRNPEQSTSEKAVLTSQKSSEYPISQNPEGLSADKFEVSADSSTSKNKEPGVERSS
PSKCPSLDDRWYMHSCSGSLQNRNYPSQEELIKVVDVEEQQLEESGPHDLTETSY
LPRQDLEGTPYLESGISLFSDDPESDPSEDRAPESARVGNIPSSTSALKVPQLKVAE
SAQSPAAAHTTDTAGYNAMEESVSREKPELTASTERVNKRMSMVVSGLTPEEF
MLVYKFARKHHITLTNLITEETTHVVMKTDAEFVCERTLKYFLGIAGGKWVVSY
FWVTQSIKERKMLNEHDFEVRGDVVNGRNHQGPKRARESQDRKIFRGLEICCYG
PFTNMPTDQLEWMVQLCGASVVKELSSFTLGTGVHPIVVVQPDAWTEDNGFHAI
GQMCEAPVVTREWVLDSVALYQCQELDTYLIPQIPHSHY
Human BRCA1 cDNA Sequence, Variant 2 (SEQ ID NO: 16)
ATGCTGAAACTTCTCAACCAGAAGAAAGGGCCTTCACAGTGTCCTTTATGTA
AGAATGATATAACCAAAAGGAGCCTACAAGAAAGTACGAGATTTAGTCAACT
TGTTGAAGAGCTATTGAAAATCATTTGTGCTTTTCAGCTTGACACAGGTTTGG
AGTATGCAAACAGCTATAATTTTGCAAAAAAGGAAAATAACTCTCCTGAACA
TCTAAAAGATGAAGTTTCTATCATCCAAAGTATGGGCTACAGAAACCGTGCC
AAAAGACTTCTACAGAGTGAACCCGAAAATCCTTCCTTGCAGGAAACCAGTC
TCAGTGTCCAACTCTCTAACCTTGGAACTGTGAGAACTCTGAGGACAAAGCA
GCGGATACAACCTCAAAAGACGTCTGTCTACATTGAATTGGGATCTGATTCTT
CTGAAGATACCGTTAATAAGGCAACTTATTGCAGTGTGGGAGATCAAGAATT
GTTACAAATCACCCCTCAAGGAACCAGGGATGAAATCAGTTTGGATTCTGCA
AAAAAGGCTGCTTGTGAATTTTCTGAGACGGATGTAACAAATACTGAACATC
ATCAACCCAGTAATAATGATTTGAACACCACTGAGAAGCGTGCAGCTGAGAG
GCATCCAGAAAAGTATCAGGGTAGTTCTGTTTCAAACTTGCATGTGGAGCCA
TGTGGCACAAATACTCATGCCAGCTCATTACAGCATGAGAACAGCAGTTTAT
TACTCACTAAAGACAGAATGAATGTAGAAAAGGCTGAATTCTGTAATAAAAG
CAAACAGCCTGGCTTAGCAAGGAGCCAACATAACAGATGGGCTGGAAGTAA
GGAAACATGTAATGATAGGCGGACTCCCAGCACAGAAAAAAAGGTAGATCT
GAATGCTGATCCCCTGTGTGAGAGAAAAGAATGGAATAAGCAGAAACTGCC
ATGCTCAGAGAATCCTAGAGATACTGAAGATGTTCCTTGGATAACACTAAAT
AGCAGCATTCAGAAAGTTAATGAGTGGTTTTCCAGAAGTGATGAACTGTTAG
GTTCTGATGACTCACATGATGGGGAGTCTGAATCAAATGCCAAAGTAGCTGA
TGTATTGGACGTTCTAAATGAGGTAGATGAATATTCTGGTTCTTCAGAGAAAA
TAGACTTACTGGCCAGTGATCCTCATGAGGCTTTAATATGTAAAAGTGAAAG
AGTTCACTCCAAATCAGTAGAGAGTAATATTGAAGACAAAATATTTGGGAAA
ACCTATCGGAAGAAGGCAAGCCTCCCCAACTTAAGCCATGTAACTGAAAATC
TAATTATAGGAGCATTTGTTACTGAGCCACAGATAATACAAGAGCGTCCCCT
CACAAATAAATTAAAGCGTAAAAGGAGACCTACATCAGGCCTTCATCCTGAG
GATTTTATCAAGAAAGCAGATTTGGCAGTTCAAAAGACTCCTGAAATGATAA
ATCAGGGAACTAACCAAACGGAGCAGAATGGTCAAGTGATGAATATTACTAA
TAGTGGTCATGAGAATAAAACAAAAGGTGATTCTATTCAGAATGAGAAAAAT
CCTAACCCAATAGAATCACTCGAAAAAGAATCTGCTTTCAAAACGAAAGCTG
AACCTATAAGCAGCAGTATAAGCAATATGGAACTCGAATTAAATATCCACAA
TTCAAAAGCACCTAAAAAGAATAGGCTGAGGAGGAAGTCTTCTACCAGGCAT
ATTCATGCGCTTGAACTAGTAGTCAGTAGAAATCTAAGCCCACCTAATTGTAC
TGAATTGCAAATTGATAGTTGTTCTAGCAGTGAAGAGATAAAGAAAAAAAAG
TACAACCAAATGCCAGTCAGGCACAGCAGAAACCTACAACTCATGGAAGGT
AAAGAACCTGCAACTGGAGCCAAGAAGAGTAACAAGCCAAATGAACAGACA
AGTAAAAGACATGACAGCGATACTTTCCCAGAGCTGAAGTTAACAAATGCAC
CTGGTTCTTTTACTAAGTGTTCAAATACCAGTGAACTTAAAGAATTTGTCAAT
CCTAGCCTTCCAAGAGAAGAAAAAGAAGAGAAACTAGAAACAGTTAAAGTG
TCTAATAATGCTGAAGACCCCAAAGATCTCATGTTAAGTGGAGAAAGGGTTT
TGCAAACTGAAAGATCTGTAGAGAGTAGCAGTATTTCATTGGTACCTGGTAC
TGATTATGGCACTCAGGAAAGTATCTCGTTACTGGAAGTTAGCACTCTAGGG
AAGGCAAAAACAGAACCAAATAAATGTGTGAGTCAGTGTGCAGCATTTGAA
AACCCCAAGGGACTAATTCATGGTTGTTCCAAAGATAATAGAAATGACACAG
AAGGCTTTAAGTATCCATTGGGACATGAAGTTAACCACAGTCGGGAAACAAG
CATAGAAATGGAAGAAAGTGAACTTGATGCTCAGTATTTGCAGAATACATTC
AAGGTTTCAAAGCGCCAGTCATTTGCTCCGTTTTCAAATCCAGGAAATGCAG
AAGAGGAATGTGCAACATTCTCTGCCCACTCTGGGTCCTTAAAGAAACAAAG
TCCAAAAGTCACTTTTGAATGTGAACAAAAGGAAGAAAATCAAGGAAAGAA
TGAGTCTAATATCAAGCCTGTACAGACAGTTAATATCACTGCAGGCTTTCCTG
TGGTTGGTCAGAAAGATAAGCCAGTTGATAATGCCAAATGTAGTATCAAAGG
AGGCTCTAGGTTTTGTCTATCATCTCAGTTCAGAGGCAACGAAACTGGACTCA
TTACTCCAAATAAACATGGACTTTTACAAAACCCATATCGTATACCACCACTT
TTTCCCATCAAGTCATTTGTTAAAACTAAATGTAAGAAAAATCTGCTAGAGG
AAAACTTTGAGGAACATTCAATGTCACCTGAAAGAGAAATGGGAAATGAGA
ACATTCCAAGTACAGTGAGCACAATTAGCCGTAATAACATTAGAGAAAATGT
TTTTAAAGAAGCCAGCTCAAGCAATATTAATGAAGTAGGTTCCAGTACTAAT
GAAGTGGGCTCCAGTATTAATGAAATAGGTTCCAGTGATGAAAACATTCAAG
CAGAACTAGGTAGAAACAGAGGGCCAAAATTGAATGCTATGCTTAGATTAGG
GGTTTTGCAACCTGAGGTCTATAAACAAAGTCTTCCTGGAAGTAATTGTAAGC
ATCCTGAAATAAAAAAGCAAGAATATGAAGAAGTAGTTCAGACTGTTAATAC
AGATTTCTCTCCATATCTGATTTCAGATAACTTAGAACAGCCTATGGGAAGTA
GTCATGCATCTCAGGTTTGTTCTGAGACACCTGATGACCTGTTAGATGATGGT
GAAATAAAGGAAGATACTAGTTTTGCTGAAAATGACATTAAGGAAAGTTCTG
CTGTTTTTAGCAAAAGCGTCCAGAAAGGAGAGCTTAGCAGGAGTCCTAGCCC
TTTCACCCATACACATTTGGCTCAGGGTTACCGAAGAGGGGCCAAGAAATTA
GAGTCCTCAGAAGAGAACTTATCTAGTGAGGATGAAGAGCTTCCCTGCTTCC
AACACTTGTTATTTGGTAAAGTAAACAATATACCTTCTCAGTCTACTAGGCAT
AGCACCGTTGCTACCGAGTGTCTGTCTAAGAACACAGAGGAGAATTTATTAT
CATTGAAGAATAGCTTAAATGACTGCAGTAACCAGGTAATATTGGCAAAGGC
ATCTCAGGAACATCACCTTAGTGAGGAAACAAAATGTTCTGCTAGCTTGTTTT
CTTCACAGTGCAGTGAATTGGAAGACTTGACTGCAAATACAAACACCCAGGA
TCCTTTCTTGATTGGTTCTTCCAAACAAATGAGGCATCAGTCTGAAAGCCAGG
GAGTTGGTCTGAGTGACAAGGAATTGGTTTCAGATGATGAAGAAAGAGGAAC
GGGCTTGGAAGAAAATAATCAAGAAGAGCAAAGCATGGATTCAAACTTAGG
TGAAGCAGCATCTGGGTGTGAGAGTGAAACAAGCGTCTCTGAAGACTGCTCA
GGGCTATCCTCTCAGAGTGACATTTTAACCACTCAGCAGAGGGATACCATGC
AACATAACCTGATAAAGCTCCAGCAGGAAATGGCTGAACTAGAAGCTGTGTT
AGAACAGCATGGGAGCCAGCCTTCTAACAGCTACCCTTCCATCATAAGTGAC
TCTTCTGCCCTTGAGGACCTGCGAAATCCAGAACAAAGCACATCAGAAAAAG
CAGTATTAACTTCACAGAAAAGTAGTGAATACCCTATAAGCCAGAATCCAGA
AGGCCTTTCTGCTGACAAGTTTGAGGTGTCTGCAGATAGTTCTACCAGTAAAA
ATAAAGAACCAGGAGTGGAAAGGTCATCCCCTTCTAAATGCCCATCATTAGA
TGATAGGTGGTACATGCACAGTTGCTCTGGGAGTCTTCAGAATAGAAACTAC
CCATCTCAAGAGGAGCTCATTAAGGTTGTTGATGTGGAGGAGCAACAGCTGG
AAGAGTCTGGGCCACACGATTTGACGGAAACATCTTACTTGCCAAGGCAAGA
TCTAGAGGGAACCCCTTACCTGGAATCTGGAATCAGCCTCTTCTCTGATGACC
CTGAATCTGATCCTTCTGAAGACAGAGCCCCAGAGTCAGCTCGTGTTGGCAA
CATACCATCTTCAACCTCTGCATTGAAAGTTCCCCAATTGAAAGTTGCAGAAT
CTGCCCAGAGTCCAGCTGCTGCTCATACTACTGATACTGCTGGGTATAATGCA
ATGGAAGAAAGTGTGAGCAGGGAGAAGCCAGAATTGACAGCTTCAACAGAA
AGGGTCAACAAAAGAATGTCCATGGTGGTGTCTGGCCTGACCCCAGAAGAAT
TTATGCTCGTGTACAAGTTTGCCAGAAAACACCACATCACTTTAACTAATCTA
ATTACTGAAGAGACTACTCATGTTGTTATGAAAACAGATGCTGAGTTTGTGTG
TGAACGGACACTGAAATATTTTCTAGGAATTGCGGGAGGAAAATGGGTAGTT
AGCTATTTCTGGGTGACCCAGTCTATTAAAGAAAGAAAAATGCTGAATGAGC
ATGATTTTGAAGTCAGAGGAGATGTGGTCAATGGAAGAAACCACCAAGGTCC
AAAGCGAGCAAGAGAATCCCAGGACAGAAAGATCTTCAGGGGGCTAGAAAT
CTGTTGCTATGGGCCCTTCACCAACATGCCCACAGATCAACTGGAATGGATG
GTACAGCTGTGTGGTGCTTCTGTGGTGAAGGAGCTTTCATCATTCACCCTTGG
CACAGGTGTCCACCCAATTGTGGTTGTGCAGCCAGATGCCTGGACAGAGGAC
AATGGCTTCCATGCAATTGGGCAGATGTGTGAGGCACCTGTGGTGACCCGAG
AGTGGGTGTTGGACAGTGTAGCACTCTACCAGTGCCAGGAGCTGGACACCTA
CCTGATACCCCAGATCCCCCACAGCCACTACTGA
Human BRCA1 Protein Sequence, Variant 2 (SEQ ID NO: 17)
MLKLLNQKKGPSQCPLCKNDITKRSLQESTRFSQLVEELLKIICAFQLDTGLEYAN
SYNFAKKENNSPEHLKDEVSIIQSMGYRNRAKRLLQSEPENPSLQETSLSVQLSNL
GTVRTLRTKQRIQPQKTSVYIELGSDSSEDTVNKATYCSVGDQELLQITPQGTRD
EISLDSAKKAACEFSETDVTNTEHHQPSNNDLNTTEKRAAERHPEKYQGSSVSNL
HVEPCGTNTHASSLQHENSSLLLTKDRMNVEKAEFCNKSKQPGLARSQHNRWA
GSKETCNDRRTPSTEKKVDLNADPLCERKEWNKQKLPCSENPRDTEDVPWITLN
SSIQKVNEWFSRSDELLGSDDSHDGESESNAKVADVLDVLNEVDEYSGSSEKIDL
LASDPHEALICKSERVHSKSVESNIEDKIFGKTYRKKASLPNLSHVTENLIIGAFVT
EPQIIQERPLTNKLKRKRRPTSGLHPEDFIKKADLAVQKTPEMINQGTNQTEQNG
QVMNITNSGHENKTKGDSIQNEKNPNPIESLEKESAFKTKAEPISSSISNMELELNI
HNSKAPKKNRLRRKSSTRHIHALELVVSRNLSPPNCTELQIDSCSSSEEIKKKKYN
QMPVRHSRNLQLMEGKEPATGAKKSNKPNEQTSKRHDSDTFPELKLTNAPGSFT
KCSNTSELKEFVNPSLPREEKEEKLETVKVSNNAEDPKDLMLSGERVLQTERSVE
SSSISLVPGTDYGTQESISLLEVSTLGKAKTEPNKCVSQCAAFENPKGLIHGCSKD
NRNDTEGFKYPLGHEVNHSRETSIEMEESELDAQYLQNTFKVSKRQSFAPFSNPG
NAEEECATFSAHSGSLKKQSPKVTFECEQKEENQGKNESNIKPVQTVNITAGFPV
VGQKDKPVDNAKCSIKGGSRFCLSSQFRGNETGLITPNKHGLLQNPYRIPPLFPIK
SFVKTKCKKNLLEENFEEHSMSPEREMGNENIPSTVSTISRNNIRENVFKEASSSNI
NEVGSSTNEVGSSINEIGSSDENIQAELGRNRGPKLNAMLRLGVLQPEVYKQSLP
GSNCKHPEIKKQEYEEVVQTVNTDFSPYLISDNLEQPMGSSHASQVCSETPDDLL
DDGEIKEDTSFAENDIKESSAVFSKSVQKGELSRSPSPFTHTHLAQGYRRGAKKL
ESSEENLSSEDEELPCFQHLLFGKVNNIPSQSTRHSTVATECLSKNTEENLLSLKNS
LNDCSNQVILAKASQEHHLSEETKCSASLFSSQCSELEDLTANTNTQDPFLIGSSK
QMRHQSESQGVGLSDKELVSDDEERGTGLEENNQEEQSMDSNLGEAASGCESET
SVSEDCSGLSSQSDILTTQQRDTMQHNLIKLQQEMAELEAVLEQHGSQPSNSYPSI
ISDSSALEDLRNPEQSTSEKAVLTSQKSSEYPISQNPEGLSADKFEVSADSSTSKNK
EPGVERSSPSKCPSLDDRWYMHSCSGSLQNRNYPSQEELIKVVDVEEQQLEESGP
HDLTETSYLPRQDLEGTPYLESGISLFSDDPESDPSEDRAPESARVGNIPSSTSALK
VPQLKVAESAQSPAAAHTTDTAGYNAMEESVSREKPELTASTERVNKRMSMVV
SGLTPEEFMLVYKFARKHHITLTNLITEETTHVVMKTDAEFVCERTLKYFLGIAG
GKWVVSYFWVTQSIKERKMLNEHDFEVRGDVVNGRNHQGPKRARESQDRKIFR
GLEICCYGPFTNMPTDQLEWMVQLCGASVVKELSSFTLGTGVHPIVVVQPDAWT
EDNGFHAIGQMCEAPVVTREWVLDSVALYQCQELDTYLIPQIPHSHY
Human BRCA1 cDNA Sequence, Variant 3 (SEQ ID NO: 18)
ATGGATTTATCTGCTCTTCGCGTTGAAGAAGTACAAAATGTCATTAATGCTAT
GCAGAAAATCTTAGAGTGTCCCATCTGTCTGGAGTTGATCAAGGAACCTGTCT
CCACAAAGTGTGACCACATATTTTGCAAATTTTGCATGCTGAAACTTCTCAAC
CAGAAGAAAGGGCCTTCACAGTGTCCTTTATGTAAGAATGATATAACCAAAA
GGAGCCTACAAGAAAGTACGAGATTTAGTCAACTTGTTGAAGAGCTATTGAA
AATCATTTGTGCTTTTCAGCTTGACACAGGTTTGGAGTATGCAAACAGCTATA
ATTTTGCAAAAAAGGAAAATAACTCTCCTGAACATCTAAAAGATGAAGTTTC
TATCATCCAAAGTATGGGCTACAGAAACCGTGCCAAAAGACTTCTACAGAGT
GAACCCGAAAATCCTTCCTTGCAGGAAACCAGTCTCAGTGTCCAACTCTCTA
ACCTTGGAACTGTGAGAACTCTGAGGACAAAGCAGCGGATACAACCTCAAAA
GACGTCTGTCTACATTGAATTGGGATCTGATTCTTCTGAAGATACCGTTAATA
AGGCAACTTATTGCAGTGTGGGAGATCAAGAATTGTTACAAATCACCCCTCA
AGGAACCAGGGATGAAATCAGTTTGGATTCTGCAAAAAAGGCTGCTTGTGAA
TTTTCTGAGACGGATGTAACAAATACTGAACATCATCAACCCAGTAATAATG
ATTTGAACACCACTGAGAAGCGTGCAGCTGAGAGGCATCCAGAAAAGTATCA
GGGTGAAGCAGCATCTGGGTGTGAGAGTGAAACAAGCGTCTCTGAAGACTGC
TCAGGGCTATCCTCTCAGAGTGACATTTTAACCACTCAGCAGAGGGATACCA
TGCAACATAACCTGATAAAGCTCCAGCAGGAAATGGCTGAACTAGAAGCTGT
GTTAGAACAGCATGGGAGCCAGCCTTCTAACAGCTACCCTTCCATCATAAGT
GACTCTTCTGCCCTTGAGGACCTGCGAAATCCAGAACAAAGCACATCAGAAA
AAGTATTAACTTCACAGAAAAGTAGTGAATACCCTATAAGCCAGAATCCAGA
AGGCCTTTCTGCTGACAAGTTTGAGGTGTCTGCAGATAGTTCTACCAGTAAAA
ATAAAGAACCAGGAGTGGAAAGGTCATCCCCTTCTAAATGCCCATCATTAGA
TGATAGGTGGTACATGCACAGTTGCTCTGGGAGTCTTCAGAATAGAAACTAC
CCATCTCAAGAGGAGCTCATTAAGGTTGTTGATGTGGAGGAGCAACAGCTGG
AAGAGTCTGGGCCACACGATTTGACGGAAACATCTTACTTGCCAAGGCAAGA
TCTAGAGGGAACCCCTTACCTGGAATCTGGAATCAGCCTCTTCTCTGATGACC
CTGAATCTGATCCTTCTGAAGACAGAGCCCCAGAGTCAGCTCGTGTTGGCAA
CATACCATCTTCAACCTCTGCATTGAAAGTTCCCCAATTGAAAGTTGCAGAAT
CTGCCCAGAGTCCAGCTGCTGCTCATACTACTGATACTGCTGGGTATAATGCA
ATGGAAGAAAGTGTGAGCAGGGAGAAGCCAGAATTGACAGCTTCAACAGAA
AGGGTCAACAAAAGAATGTCCATGGTGGTGTCTGGCCTGACCCCAGAAGAAT
TTATGCTCGTGTACAAGTTTGCCAGAAAACACCACATCACTTTAACTAATCTA
ATTACTGAAGAGACTACTCATGTTGTTATGAAAACAGATGCTGAGTTTGTGTG
TGAACGGACACTGAAATATTTTCTAGGAATTGCGGGAGGAAAATGGGTAGTT
AGCTATTTCTGGGTGACCCAGTCTATTAAAGAAAGAAAAATGCTGAATGAGC
ATGATTTTGAAGTCAGAGGAGATGTGGTCAATGGAAGAAACCACCAAGGTCC
AAAGCGAGCAAGAGAATCCCAGGACAGAAAGATCTTCAGGGGGCTAGAAAT
CTGTTGCTATGGGCCCTTCACCAACATGCCCACAGATCAACTGGAATGGATG
GTACAGCTGTGTGGTGCTTCTGTGGTGAAGGAGCTTTCATCATTCACCCTTGG
CACAGGTGTCCACCCAATTGTGGTTGTGCAGCCAGATGCCTGGACAGAGGAC
AATGGCTTCCATGCAATTGGGCAGATGTGTGAGGCACCTGTGGTGACCCGAG
AGTGGGTGTTGGACAGTGTAGCACTCTACCAGTGCCAGGAGCTGGACACCTA
CCTGATACCCCAGATCCCCCACAGCCACTACTGA
Human BRCA1 Protein Sequence, Variant 3 (SEQ ID NO: 19)
MDLSALRVEEVQNVINAMQKILECPICLELIKEPVSTKCDHIFCKFCMLKLLNQK
KGPSQCPLCKNDITKRSLQESTRFSQLVEELLKIICAFQLDTGLEYANSYNFAKKE
NNSPEHLKDEVSIIQSMGYRNRAKRLLQSEPENPSLQETSLSVQLSNLGTVRTLRT
KQRIQPQKTSVYIELGSDSSEDTVNKATYCSVGDQELLQITPQGTRDEISLDSAKK
AACEFSETDVTNTEHHQPSNNDLNTTEKRAAERHPEKYQGEAASGCESETSVSE
DCSGLSSQSDILTTQQRDTMQHNLIKLQQEMAELEAVLEQHGSQPSNSYPSIISDS
SALEDLRNPEQSTSEKVLTSQKSSEYPISQNPEGLSADKFEVSADSSTSKNKEPGV
ERSSPSKCPSLDDRWYMHSCSGSLQNRNYPSQEELIKVVDVEEQQLEESGPHDLT
ETSYLPRQDLEGTPYLESGISLFSDDPESDPSEDRAPESARVGNIPSSTSALKVPQL
KVAESAQSPAAAHTTDTAGYNAMEESVSREKPELTASTERVNKRMSMVVSGLT
PEEFMLVYKFARKHHITLTNLITEETTHVVMKTDAEFVCERTLKYFLGIAGGKW
VVSYFWVTQSIKERKMLNEHDFEVRGDVVNGRNHQGPKRARESQDRKIFRGLEI
CCYGPFTNMPTDQLEWMVQLCGASVVKELSSFTLGTGVHPIVVVQPDAWTEDN
GFHAIGQMCEAPVVTREWVLDSVALYQCQELDTYLIPQIPHSHY
Human BRCA1 cDNA Sequence, Variant 4 (SEQ ID NO: 20)
ATGGATTTATCTGCTCTTCGCGTTGAAGAAGTACAAAATGTCATTAATGCTAT
GCAGAAAATCTTAGAGTGTCCCATCTGTCTGGAGTTGATCAAGGAACCTGTCT
CCACAAAGTGTGACCACATATTTTGCAAATTTTGCATGCTGAAACTTCTCAAC
CAGAAGAAAGGGCCTTCACAGTGTCCTTTATGTAAGAATGATATAACCAAAA
GGAGCCTACAAGAAAGTACGAGATTTAGTCAACTTGTTGAAGAGCTATTGAA
AATCATTTGTGCTTTTCAGCTTGACACAGGTTTGGAGTATGCAAACAGCTATA
ATTTTGCAAAAAAGGAAAATAACTCTCCTGAACATCTAAAAGATGAAGTTTC
TATCATCCAAAGTATGGGCTACAGAAACCGTGCCAAAAGACTTCTACAGAGT
GAACCCGAAAATCCTTCCTTGCAGGAAACCAGTCTCAGTGTCCAACTCTCTA
ACCTTGGAACTGTGAGAACTCTGAGGACAAAGCAGCGGATACAACCTCAAAA
GACGTCTGTCTACATTGAATTGGGATCTGATTCTTCTGAAGATACCGTTAATA
AGGCAACTTATTGCAGTGTGGGAGATCAAGAATTGTTACAAATCACCCCTCA
AGGAACCAGGGATGAAATCAGTTTGGATTCTGCAAAAAAGGCTGCTTGTGAA
TTTTCTGAGACGGATGTAACAAATACTGAACATCATCAACCCAGTAATAATG
ATTTGAACACCACTGAGAAGCGTGCAGCTGAGAGGCATCCAGAAAAGTATCA
GGGTGAAGCAGCATCTGGGTGTGAGAGTGAAACAAGCGTCTCTGAAGACTGC
TCAGGGCTATCCTCTCAGAGTGACATTTTAACCACTCAGCAGAGGGATACCA
TGCAACATAACCTGATAAAGCTCCAGCAGGAAATGGCTGAACTAGAAGCTGT
GTTAGAACAGCATGGGAGCCAGCCTTCTAACAGCTACCCTTCCATCATAAGT
GACTCTTCTGCCCTTGAGGACCTGCGAAATCCAGAACAAAGCACATCAGAAA
AAGTATTAACTTCACAGAAAAGTAGTGAATACCCTATAAGCCAGAATCCAGA
AGGCCTTTCTGCTGACAAGTTTGAGGTGTCTGCAGATAGTTCTACCAGTAAAA
ATAAAGAACCAGGAGTGGAAAGGTCATCCCCTTCTAAATGCCCATCATTAGA
TGATAGGTGGTACATGCACAGTTGCTCTGGGAGTCTTCAGAATAGAAACTAC
CCATCTCAAGAGGAGCTCATTAAGGTTGTTGATGTGGAGGAGCAACAGCTGG
AAGAGTCTGGGCCACACGATTTGACGGAAACATCTTACTTGCCAAGGCAAGA
TCTAGAGGGAACCCCTTACCTGGAATCTGGAATCAGCCTCTTCTCTGATGACC
CTGAATCTGATCCTTCTGAAGACAGAGCCCCAGAGTCAGCTCGTGTTGGCAA
CATACCATCTTCAACCTCTGCATTGAAAGTTCCCCAATTGAAAGTTGCAGAAT
CTGCCCAGAGTCCAGCTGCTGCTCATACTACTGATACTGCTGGGTATAATGCA
ATGGAAGAAAGTGTGAGCAGGGAGAAGCCAGAATTGACAGCTTCAACAGAA
AGGGTCAACAAAAGAATGTCCATGGTGGTGTCTGGCCTGACCCCAGAAGAAT
TTATGCTCGTGTACAAGTTTGCCAGAAAACACCACATCACTTTAACTAATCTA
ATTACTGAAGAGACTACTCATGTTGTTATGAAAACAGATGCTGAGTTTGTGTG
TGAACGGACACTGAAATATTTTCTAGGAATTGCGGGAGGAAAATGGGTAGTT
AGCTATTTCTGGGTGACCCAGTCTATTAAAGAAAGAAAAATGCTGAATGAGC
ATGATTTTGAAGTCAGAGGAGATGTGGTCAATGGAAGAAACCACCAAGGTCC
AAAGCGAGCAAGAGAATCCCAGGACAGAAAGATCTTCAGGGGGCTAGAAAT
CTGTTGCTATGGGCCCTTCACCAACATGCCCACAGGGTGTCCACCCAATTGTG
GTTGTGCAGCCAGATGCCTGGACAGAGGACAATGGCTTCCATGCAATTGGGC
AGATGTGTGA
Human BRCA1 Protein Sequence, Variant 4 (SEQ ID NO: 21)
MDLSALRVEEVQNVINAMQKILECPICLELIKEPVSTKCDHIFCKFCMLKLLNQK
KGPSQCPLCKNDITKRSLQESTRFSQLVEELLKIICAFQLDTGLEYANSYNFAKKE
NNSPEHLKDEVSIIQSMGYRNRAKRLLQSEPENPSLQETSLSVQLSNLGTVRTLRT
KQRIQPQKTSVYIELGSDSSEDTVNKATYCSVGDQELLQITPQGTRDEISLDSAKK
AACEFSETDVTNTEHHQPSNNDLNTTEKRAAERHPEKYQGEAASGCESETSVSE
DCSGLSSQSDILTTQQRDTMQHNLIKLQQEMAELEAVLEQHGSQPSNSYPSIISDS
SALEDLRNPEQSTSEKVLTSQKSSEYPISQNPEGLSADKFEVSADSSTSKNKEPGV
ERSSPSKCPSLDDRWYMHSCSGSLQNRNYPSQEELIKVVDVEEQQLEESGPHDLT
ETSYLPRQDLEGTPYLESGISLFSDDPESDPSEDRAPESARVGNIPSSTSALKVPQL
KVAESAQSPAAAHTTDTAGYNAMEESVSREKPELTASTERVNKRMSMVVSGLT
PEEFMLVYKFARKHHITLTNLITEETTHVVMKTDAEFVCERTLKYFLGIAGGKW
VVSYFWVTQSIKERKMLNEHDFEVRGDVVNGRNHQGPKRARESQDRKIFRGLEI
CCYGPFTNMPTGCPPNCGCAARCLDRGQWLPCNWADV
Human BRCA1 cDNA Sequence, Variant 5 (SEQ ID NO: 22)
ATGGATTTATCTGCTCTTCGCGTTGAAGAAGTACAAAATGTCATTAATGCTAT
GCAGAAAATCTTAGAGTGTCCCATCTGTCTGGAGTTGATCAAGGAACCTGTCT
CCACAAAGTGTGACCACATATTTTGCAAATTTTGCATGCTGAAACTTCTCAAC
CAGAAGAAAGGGCCTTCACAGTGTCCTTTATGTAAGAATGATATAACCAAAA
GGAGCCTACAAGAAAGTACGAGATTTAGTCAACTTGTTGAAGAGCTATTGAA
AATCATTTGTGCTTTTCAGCTTGACACAGGTTTGGAGTATGCAAACAGCTATA
ATTTTGCAAAAAAGGAAAATAACTCTCCTGAACATCTAAAAGATGAAGTTTC
TATCATCCAAAGTATGGGCTACAGAAACCGTGCCAAAAGACTTCTACAGAGT
GAACCCGAAAATCCTTCCTTGCAGGAAACCAGTCTCAGTGTCCAACTCTCTA
ACCTTGGAACTGTGAGAACTCTGAGGACAAAGCAGCGGATACAACCTCAAAA
GACGTCTGTCTACATTGAATTGGGATCTGATTCTTCTGAAGATACCGTTAATA
AGGCAACTTATTGCAGTGTGGGAGATCAAGAATTGTTACAAATCACCCCTCA
AGGAACCAGGGATGAAATCAGTTTGGATTCTGCAAAAAAGGCTGCTTGTGAA
TTTTCTGAGACGGATGTAACAAATACTGAACATCATCAACCCAGTAATAATG
ATTTGAACACCACTGAGAAGCGTGCAGCTGAGAGGCATCCAGAAAAGTATCA
GGGTAGTTCTGTTTCAAACTTGCATGTGGAGCCATGTGGCACAAATACTCATG
CCAGCTCATTACAGCATGAGAACAGCAGTTTATTACTCACTAAAGACAGAAT
GAATGTAGAAAAGGCTGAATTCTGTAATAAAAGCAAACAGCCTGGCTTAGCA
AGGAGCCAACATAACAGATGGGCTGGAAGTAAGGAAACATGTAATGATAGG
CGGACTCCCAGCACAGAAAAAAAGGTAGATCTGAATGCTGATCCCCTGTGTG
AGAGAAAAGAATGGAATAAGCAGAAACTGCCATGCTCAGAGAATCCTAGAG
ATACTGAAGATGTTCCTTGGATAACACTAAATAGCAGCATTCAGAAAGTTAA
TGAGTGGTTTTCCAGAAGTGATGAACTGTTAGGTTCTGATGACTCACATGATG
GGGAGTCTGAATCAAATGCCAAAGTAGCTGATGTATTGGACGTTCTAAATGA
GGTAGATGAATATTCTGGTTCTTCAGAGAAAATAGACTTACTGGCCAGTGAT
CCTCATGAGGCTTTAATATGTAAAAGTGAAAGAGTTCACTCCAAATCAGTAG
AGAGTAATATTGAAGACAAAATATTTGGGAAAACCTATCGGAAGAAGGCAA
GCCTCCCCAACTTAAGCCATGTAACTGAAAATCTAATTATAGGAGCATTTGTT
ACTGAGCCACAGATAATACAAGAGCGTCCCCTCACAAATAAATTAAAGCGTA
AAAGGAGACCTACATCAGGCCTTCATCCTGAGGATTTTATCAAGAAAGCAGA
TTTGGCAGTTCAAAAGACTCCTGAAATGATAAATCAGGGAACTAACCAAACG
GAGCAGAATGGTCAAGTGATGAATATTACTAATAGTGGTCATGAGAATAAAA
CAAAAGGTGATTCTATTCAGAATGAGAAAAATCCTAACCCAATAGAATCACT
CGAAAAAGAATCTGCTTTCAAAACGAAAGCTGAACCTATAAGCAGCAGTATA
AGCAATATGGAACTCGAATTAAATATCCACAATTCAAAAGCACCTAAAAAGA
ATAGGCTGAGGAGGAAGTCTTCTACCAGGCATATTCATGCGCTTGAACTAGT
AGTCAGTAGAAATCTAAGCCCACCTAATTGTACTGAATTGCAAATTGATAGTT
GTTCTAGCAGTGAAGAGATAAAGAAAAAAAAGTACAACCAAATGCCAGTCA
GGCACAGCAGAAACCTACAACTCATGGAAGGTAAAGAACCTGCAACTGGAG
CCAAGAAGAGTAACAAGCCAAATGAACAGACAAGTAAAAGACATGACAGCG
ATACTTTCCCAGAGCTGAAGTTAACAAATGCACCTGGTTCTTTTACTAAGTGT
TCAAATACCAGTGAACTTAAAGAATTTGTCAATCCTAGCCTTCCAAGAGAAG
AAAAAGAAGAGAAACTAGAAACAGTTAAAGTGTCTAATAATGCTGAAGACC
CCAAAGATCTCATGTTAAGTGGAGAAAGGGTTTTGCAAACTGAAAGATCTGT
AGAGAGTAGCAGTATTTCATTGGTACCTGGTACTGATTATGGCACTCAGGAA
AGTATCTCGTTACTGGAAGTTAGCACTCTAGGGAAGGCAAAAACAGAACCAA
ATAAATGTGTGAGTCAGTGTGCAGCATTTGAAAACCCCAAGGGACTAATTCA
TGGTTGTTCCAAAGATAATAGAAATGACACAGAAGGCTTTAAGTATCCATTG
GGACATGAAGTTAACCACAGTCGGGAAACAAGCATAGAAATGGAAGAAAGT
GAACTTGATGCTCAGTATTTGCAGAATACATTCAAGGTTTCAAAGCGCCAGTC
ATTTGCTCCGTTTTCAAATCCAGGAAATGCAGAAGAGGAATGTGCAACATTC
TCTGCCCACTCTGGGTCCTTAAAGAAACAAAGTCCAAAAGTCACTTTTGAATG
TGAACAAAAGGAAGAAAATCAAGGAAAGAATGAGTCTAATATCAAGCCTGT
ACAGACAGTTAATATCACTGCAGGCTTTCCTGTGGTTGGTCAGAAAGATAAG
CCAGTTGATAATGCCAAATGTAGTATCAAAGGAGGCTCTAGGTTTTGTCTATC
ATCTCAGTTCAGAGGCAACGAAACTGGACTCATTACTCCAAATAAACATGGA
CTTTTACAAAACCCATATCGTATACCACCACTTTTTCCCATCAAGTCATTTGTT
AAAACTAAATGTAAGAAAAATCTGCTAGAGGAAAACTTTGAGGAACATTCAA
TGTCACCTGAAAGAGAAATGGGAAATGAGAACATTCCAAGTACAGTGAGCA
CAATTAGCCGTAATAACATTAGAGAAAATGTTTTTAAAGAAGCCAGCTCAAG
CAATATTAATGAAGTAGGTTCCAGTACTAATGAAGTGGGCTCCAGTATTAAT
GAAATAGGTTCCAGTGATGAAAACATTCAAGCAGAACTAGGTAGAAACAGA
GGGCCAAAATTGAATGCTATGCTTAGATTAGGGGTTTTGCAACCTGAGGTCT
ATAAACAAAGTCTTCCTGGAAGTAATTGTAAGCATCCTGAAATAAAAAAGCA
AGAATATGAAGAAGTAGTTCAGACTGTTAATACAGATTTCTCTCCATATCTGA
TTTCAGATAACTTAGAACAGCCTATGGGAAGTAGTCATGCATCTCAGGTTTGT
TCTGAGACACCTGATGACCTGTTAGATGATGGTGAAATAAAGGAAGATACTA
GTTTTGCTGAAAATGACATTAAGGAAAGTTCTGCTGTTTTTAGCAAAAGCGTC
CAGAAAGGAGAGCTTAGCAGGAGTCCTAGCCCTTTCACCCATACACATTTGG
CTCAGGGTTACCGAAGAGGGGCCAAGAAATTAGAGTCCTCAGAAGAGAACTT
ATCTAGTGAGGATGAAGAGCTTCCCTGCTTCCAACACTTGTTATTTGGTAAAG
TAAACAATATACCTTCTCAGTCTACTAGGCATAGCACCGTTGCTACCGAGTGT
CTGTCTAAGAACACAGAGGAGAATTTATTATCATTGAAGAATAGCTTAAATG
ACTGCAGTAACCAGGTAATATTGGCAAAGGCATCTCAGGAACATCACCTTAG
TGAGGAAACAAAATGTTCTGCTAGCTTGTTTTCTTCACAGTGCAGTGAATTGG
AAGACTTGACTGCAAATACAAACACCCAGGATCCTTTCTTGATTGGTTCTTCC
AAACAAATGAGGCATCAGTCTGAAAGCCAGGGAGTTGGTCTGAGTGACAAG
GAATTGGTTTCAGATGATGAAGAAAGAGGAACGGGCTTGGAAGAAAATAAT
CAAGAAGAGCAAAGCATGGATTCAAACTTAGGTGAAGCAGCATCTGGGTGTG
AGAGTGAAACAAGCGTCTCTGAAGACTGCTCAGGGCTATCCTCTCAGAGTGA
CATTTTAACCACTCAGCAGAGGGATACCATGCAACATAACCTGATAAAGCTC
CAGCAGGAAATGGCTGAACTAGAAGCTGTGTTAGAACAGCATGGGAGCCAG
CCTTCTAACAGCTACCCTTCCATCATAAGTGACTCTTCTGCCCTTGAGGACCT
GCGAAATCCAGAACAAAGCACATCAGAAAAAGATTCGCATATACATGGCCA
AAGGAACAACTCCATGTTTTCTAAAAGGCCTAGAGAACATATATCAGTATTA
ACTTCACAGAAAAGTAGTGAATACCCTATAAGCCAGAATCCAGAAGGCCTTT
CTGCTGACAAGTTTGAGGTGTCTGCAGATAGTTCTACCAGTAAAAATAAAGA
ACCAGGAGTGGAAAGGTCATCCCCTTCTAAATGCCCATCATTAGATGATAGG
TGGTACATGCACAGTTGCTCTGGGAGTCTTCAGAATAGAAACTACCCATCTCA
AGAGGAGCTCATTAAGGTTGTTGATGTGGAGGAGCAACAGCTGGAAGAGTCT
GGGCCACACGATTTGACGGAAACATCTTACTTGCCAAGGCAAGATCTAGAGG
GAACCCCTTACCTGGAATCTGGAATCAGCCTCTTCTCTGATGACCCTGAATCT
GATCCTTCTGAAGACAGAGCCCCAGAGTCAGCTCGTGTTGGCAACATACCAT
CTTCAACCTCTGCATTGAAAGTTCCCCAATTGAAAGTTGCAGAATCTGCCCAG
AGTCCAGCTGCTGCTCATACTACTGATACTGCTGGGTATAATGCAATGGAAG
AAAGTGTGAGCAGGGAGAAGCCAGAATTGACAGCTTCAACAGAAAGGGTCA
ACAAAAGAATGTCCATGGTGGTGTCTGGCCTGACCCCAGAAGAATTTATGCT
CGTGTACAAGTTTGCCAGAAAACACCACATCACTTTAACTAATCTAATTACTG
AAGAGACTACTCATGTTGTTATGAAAACAGATGCTGAGTTTGTGTGTGAACG
GACACTGAAATATTTTCTAGGAATTGCGGGAGGAAAATGGGTAGTTAGCTAT
TTCTGGGTGACCCAGTCTATTAAAGAAAGAAAAATGCTGAATGAGCATGATT
TTGAAGTCAGAGGAGATGTGGTCAATGGAAGAAACCACCAAGGTCCAAAGC
GAGCAAGAGAATCCCAGGACAGAAAGATCTTCAGGGGGCTAGAAATCTGTT
GCTATGGGCCCTTCACCAACATGCCCACAGATCAACTGGAATGGATGGTACA
GCTGTGTGGTGCTTCTGTGGTGAAGGAGCTTTCATCATTCACCCTTGGCACAG
GTGTCCACCCAATTGTGGTTGTGCAGCCAGATGCCTGGACAGAGGACAATGG
CTTCCATGCAATTGGGCAGATGTGTGAGGCACCTGTGGTGACCCGAGAGTGG
GTGTTGGACAGTGTAGCACTCTACCAGTGCCAGGAGCTGGACACCTACCTGA
TACCCCAGATCCCCCACAGCCACTACTGA
Human BRCA1 Protein Sequence, Variant 5 (SEQ ID NO: 23)
MDLSALRVEEVQNVINAMQKILECPICLELIKEPVSTKCDHIFCKFCMLKLLNQK
KGPSQCPLCKNDITKRSLQESTRFSQLVEELLKIICAFQLDTGLEYANSYNFAKKE
NNSPEHLKDEVSIIQSMGYRNRAKRLLQSEPENPSLQETSLSVQLSNLGTVRTLRT
KQRIQPQKTSVYIELGSDSSEDTVNKATYCSVGDQELLQITPQGTRDEISLDSAKK
AACEFSETDVTNTEHHQPSNNDLNTTEKRAAERHPEKYQGSSVSNLHVEPCGTN
THASSLQHENSSLLLTKDRMNVEKAEFCNKSKQPGLARSQHNRWAGSKETCND
RRTPSTEKKVDLNADPLCERKEWNKQKLPCSENPRDTEDVPWITLNSSIQKVNE
WFSRSDELLGSDDSHDGESESNAKVADVLDVLNEVDEYSGSSEKIDLLASDPHE
ALICKSERVHSKSVESNIEDKIFGKTYRKKASLPNLSHVTENLIIGAFVTEPQIIQER
PLTNKLKRKRRPTSGLHPEDFIKKADLAVQKTPEMINQGTNQTEQNGQVMNITN
SCHENKTKGDSIQNEKNPNPIESLEKESAFKTKAEPISSSISNMELELNIHNSKAPK
KNRLRRKSSTRHIHALELVVSRNLSPPNCTELQIDSCSSSEEIKKKKYNQMPVRHS
RNLQLMEGKEPATGAKKSNKPNEQTSKRHDSDTFPELKLTNAPGSFTKCSNTSEL
KEFVNPSLPREEKEEKLETVKVSNNAEDPKDLMLSGERVLQTERSVESSSISLVPG
TDYGTQESISLLEVSTLGKAKTEPNKCVSQCAAFENPKGLIHGCSKDNRNDTEGF
KYPLGHEVNHSRETSIEMEESELDAQYLQNTFKVSKRQSFAPFSNPGNAEEECAT
FSAHSGSLKKQSPKVTFECEQKEENQGKNESNIKPVQTVNITAGFPVVGQKDKPV
DNAKCSIKGGSRFCLSSQFRGNETGLITPNKHGLLQNPYRIPPLFPIKSFVKTKCKK
NLLEENFEEHSMSPEREMGNENIPSTVSTISRNNIRENVFKEASSSNINEVGSSTNE
VGSSINEIGSSDENIQAELGRNRGPKLNAMLRLGVLQPEVYKQSLPGSNCKHPEIK
KQEYEEVVQTVNTDFSPYLISDNLEQPMGSSHASQVCSETPDDLLDDGEIKEDTS
FAENDIKESSAVFSKSVQKGELSRSPSPFTHTHLAQGYRRGAKKLESSEENLSSED
EELPCFQHLLFGKVNNIPSQSTRHSTVATECLSKNTEENLLSLKNSLNDCSNQVIL
AKASQEHHLSEETKCSASLFSSQCSELEDLTANTNTQDPFLIGSSKQMRHQSESQ
GVGLSDKELVSDDEERGTGLEENNQEEQSMDSNLGEAASGCESETSVSEDCSGL
SSQSDILTTQQRDTMQHNLIKLQQEMAELEAVLEQHGSQPSNSYPSIISDSSALED
LRNPEQSTSEKDSHIHGQRNNSMFSKRPREHISVLTSQKSSEYPISQNPEGLSADK
FEVSADSSTSKNKEPGVERSSPSKCPSLDDRWYMHSCSGSLQNRNYPSQEELIKV
VDVEEQQLEESGPHDLTETSYLPRQDLEGTPYLESGISLFSDDPESDPSEDRAPES
ARVGNIPSSTSALKVPQLKVAESAQSPAAAHTTDTAGYNAMEESVSREKPELTAS
TERVNKRMSMVVSGLTPEEFMLVYKFARKHHITLTNLITEETTHVVMKTDAEFV
CERTLKYFLGIAGGKWVVSYFWVTQSIKERKMLNEHDFEVRGDVVNGRNHQGP
KRARESQDRKIFRGLEICCYGPFTNMPTDQLEWMVQLCGASVVKELSSFTLGTG
VHPIVVVQPDAWTEDNGFHAIGQMCEAPVVTREWVLDSVALYQCQELDTYLIP
QIPHSHY
Human BRCA2 cDNA Sequence (SEQ ID NO: 24)
ATGCCTATTGGATCCAAAGAGAGGCCAACATTTTTTGAAATTTTTAAGACACG
CTGCAACAAAGCAGATTTAGGACCAATAAGTCTTAATTGGTTTGAAGAACTT
TCTTCAGAAGCTCCACCCTATAATTCTGAACCTGCAGAAGAATCTGAACATA
AAAACAACAATTACGAACCAAACCTATTTAAAACTCCACAAAGGAAACCATC
TTATAATCAGCTGGCTTCAACTCCAATAATATTCAAAGAGCAAGGGCTGACT
CTGCCGCTGTACCAATCTCCTGTAAAAGAATTAGATAAATTCAAATTAGACTT
AGGAAGGAATGTTCCCAATAGTAGACATAAAAGTCTTCGCACAGTGAAAACT
AAAATGGATCAAGCAGATGATGTTTCCTGTCCACTTCTAAATTCTTGTCTTAG
TGAAAGTCCTGTTGTTCTACAATGTACACATGTAACACCACAAAGAGATAAG
TCAGTGGTATGTGGGAGTTTGTTTCATACACCAAAGTTTGTGAAGGGTCGTCA
GACACCAAAACATATTTCTGAAAGTCTAGGAGCTGAGGTGGATCCTGATATG
TCTTGGTCAAGTTCTTTAGCTACACCACCCACCCTTAGTTCTACTGTGCTCATA
GTCAGAAATGAAGAAGCATCTGAAACTGTATTTCCTCATGATACTACTGCTA
ATGTGAAAAGCTATTTTTCCAATCATGATGAAAGTCTGAAGAAAAATGATAG
ATTTATCGCTTCTGTGACAGACAGTGAAAACACAAATCAAAGAGAAGCTGCA
AGTCATGGATTTGGAAAAACATCAGGGAATTCATTTAAAGTAAATAGCTGCA
AAGACCACATTGGAAAGTCAATGCCAAATGTCCTAGAAGATGAAGTATATGA
AACAGTTGTAGATACCTCTGAAGAAGATAGTTTTTCATTATGTTTTTCTAAAT
GTAGAACAAAAAATCTACAAAAAGTAAGAACTAGCAAGACTAGGAAAAAAA
TTTTCCATGAAGCAAACGCTGATGAATGTGAAAAATCTAAAAACCAAGTGAA
AGAAAAATACTCATTTGTATCTGAAGTGGAACCAAATGATACTGATCCATTA
GATTCAAATGTAGCAAATCAGAAGCCCTTTGAGAGTGGAAGTGACAAAATCT
CCAAGGAAGTTGTACCGTCTTTGGCCTGTGAATGGTCTCAACTAACCCTTTCA
GGTCTAAATGGAGCCCAGATGGAGAAAATACCCCTATTGCATATTTCTTCATG
TGACCAAAATATTTCAGAAAAAGACCTATTAGACACAGAGAACAAAAGAAA
GAAAGATTTTCTTACTTCAGAGAATTCTTTGCCACGTATTTCTAGCCTACCAA
AATCAGAGAAGCCATTAAATGAGGAAACAGTGGTAAATAAGAGAGATGAAG
AGCAGCATCTTGAATCTCATACAGACTGCATTCTTGCAGTAAAGCAGGCAAT
ATCTGGAACTTCTCCAGTGGCTTCTTCATTTCAGGGTATCAAAAAGTCTATAT
TCAGAATAAGAGAATCACCTAAAGAGACTTTCAATGCAAGTTTTTCAGGTCA
TATGACTGATCCAAACTTTAAAAAAGAAACTGAAGCCTCTGAAAGTGGACTG
GAAATACATACTGTTTGCTCACAGAAGGAGGACTCCTTATGTCCAAATTTAAT
TGATAATGGAAGCTGGCCAGCCACCACCACACAGAATTCTGTAGCTTTGAAG
AATGCAGGTTTAATATCCACTTTGAAAAAGAAAACAAATAAGTTTATTTATG
CTATACATGATGAAACATCTTATAAAGGAAAAAAAATACCGAAAGACCAAA
AATCAGAACTAATTAACTGTTCAGCCCAGTTTGAAGCAAATGCTTTTGAAGC
ACCACTTACATTTGCAAATGCTGATTCAGGTTTATTGCATTCTTCTGTGAAAA
GAAGCTGTTCACAGAATGATTCTGAAGAACCAACTTTGTCCTTAACTAGCTCT
TTTGGGACAATTCTGAGGAAATGTTCTAGAAATGAAACATGTTCTAATAATA
CAGTAATCTCTCAGGATCTTGATTATAAAGAAGCAAAATGTAATAAGGAAAA
ACTACAGTTATTTATTACCCCAGAAGCTGATTCTCTGTCATGCCTGCAGGAAG
GACAGTGTGAAAATGATCCAAAAAGCAAAAAAGTTTCAGATATAAAAGAAG
AGGTCTTGGCTGCAGCATGTCACCCAGTACAACATTCAAAAGTGGAATACAG
TGATACTGACTTTCAATCCCAGAAAAGTCTTTTATATGATCATGAAAATGCCA
GCACTCTTATTTTAACTCCTACTTCCAAGGATGTTCTGTCAAACCTAGTCATG
ATTTCTAGAGGCAAAGAATCATACAAAATGTCAGACAAGCTCAAAGGTAACA
ATTATGAATCTGATGTTGAATTAACCAAAAATATTCCCATGGAAAAGAATCA
AGATGTATGTGCTTTAAATGAAAATTATAAAAACGTTGAGCTGTTGCCACCTG
AAAAATACATGAGAGTAGCATCACCTTCAAGAAAGGTACAATTCAACCAAAA
CACAAATCTAAGAGTAATCCAAAAAAATCAAGAAGAAACTACTTCAATTTCA
AAAATAACTGTCAATCCAGACTCTGAAGAACTTTTCTCAGACAATGAGAATA
ATTTTGTCTTCCAAGTAGCTAATGAAAGGAATAATCTTGCTTTAGGAAATACT
AAGGAACTTCATGAAACAGACTTGACTTGTGTAAACGAACCCATTTTCAAGA
ACTCTACCATGGTTTTATATGGAGACACAGGTGATAAACAAGCAACCCAAGT
GTCAATTAAAAAAGATTTGGTTTATGTTCTTGCAGAGGAGAACAAAAATAGT
GTAAAGCAGCATATAAAAATGACTCTAGGTCAAGATTTAAAATCGGACATCT
CCTTGAATATAGATAAAATACCAGAAAAAAATAATGATTACATGAACAAATG
GGCAGGACTCTTAGGTCCAATTTCAAATCACAGTTTTGGAGGTAGCTTCAGA
ACAGCTTCAAATAAGGAAATCAAGCTCTCTGAACATAACATTAAGAAGAGCA
AAATGTTCTTCAAAGATATTGAAGAACAATATCCTACTAGTTTAGCTTGTGTT
GAAATTGTAAATACCTTGGCATTAGATAATCAAAAGAAACTGAGCAAGCCTC
AGTCAATTAATACTGTATCTGCACATTTACAGAGTAGTGTAGTTGTTTCTGAT
TGTAAAAATAGTCATATAACCCCTCAGATGTTATTTTCCAAGCAGGATTTTAA
TTCAAACCATAATTTAACACCTAGCCAAAAGGCAGAAATTACAGAACTTTCT
ACTATATTAGAAGAATCAGGAAGTCAGTTTGAATTTACTCAGTTTAGAAAAC
CAAGCTACATATTGCAGAAGAGTACATTTGAAGTGCCTGAAAACCAGATGAC
TATCTTAAAGACCACTTCTGAGGAATGCAGAGATGCTGATCTTCATGTCATAA
TGAATGCCCCATCGATTGGTCAGGTAGACAGCAGCAAGCAATTTGAAGGTAC
AGTTGAAATTAAACGGAAGTTTGCTGGCCTGTTGAAAAATGACTGTAACAAA
AGTGCTTCTGGTTATTTAACAGATGAAAATGAAGTGGGGTTTAGGGGCTTTTA
TTCTGCTCATGGCACAAAACTGAATGTTTCTACTGAAGCTCTGCAAAAAGCTG
TGAAACTGTTTAGTGATATTGAGAATATTAGTGAGGAAACTTCTGCAGAGGT
ACATCCAATAAGTTTATCTTCAAGTAAATGTCATGATTCTGTTGTTTCAATGTT
TAAGATAGAAAATCATAATGATAAAACTGTAAGTGAAAAAAATAATAAATG
CCAACTGATATTACAAAATAATATTGAAATGACTACTGGCACTTTTGTTGAAG
AAATTACTGAAAATTACAAGAGAAATACTGAAAATGAAGATAACAAATATA
CTGCTGCCAGTAGAAATTCTCATAACTTAGAATTTGATGGCAGTGATTCAAGT
AAAAATGATACTGTTTGTATTCATAAAGATGAAACGGACTTGCTATTTACTGA
TCAGCACAACATATGTCTTAAATTATCTGGCCAGTTTATGAAGGAGGGAAAC
ACTCAGATTAAAGAAGATTTGTCAGATTTAACTTTTTTGGAAGTTGCGAAAGC
TCAAGAAGCATGTCATGGTAATACTTCAAATAAAGAACAGTTAACTGCTACT
AAAACGGAGCAAAATATAAAAGATTTTGAGACTTCTGATACATTTTTTCAGA
CTGCAAGTGGGAAAAATATTAGTGTCGCCAAAGAGTCATTTAATAAAATTGT
AAATTTCTTTGATCAGAAACCAGAAGAATTGCATAACTTTTCCTTAAATTCTG
AATTACATTCTGACATAAGAAAGAACAAAATGGACATTCTAAGTTATGAGGA
AACAGACATAGTTAAACACAAAATACTGAAAGAAAGTGTCCCAGTTGGTACT
GGAAATCAACTAGTGACCTTCCAGGGACAACCCGAACGTGATGAAAAGATCA
AAGAACCTACTCTATTGGGTTTTCATACAGCTAGCGGGAAAAAAGTTAAAAT
TGCAAAGGAATCTTTGGACAAAGTGAAAAACCTTTTTGATGAAAAAGAGCAA
GGTACTAGTGAAATCACCAGTTTTAGCCATCAATGGGCAAAGACCCTAAAGT
ACAGAGAGGCCTGTAAAGACCTTGAATTAGCATGTGAGACCATTGAGATCAC
AGCTGCCCCAAAGTGTAAAGAAATGCAGAATTCTCTCAATAATGATAAAAAC
CTTGTTTCTATTGAGACTGTGGTGCCACCTAAGCTCTTAAGTGATAATTTATG
TAGACAAACTGAAAATCTCAAAACATCAAAAAGTATCTTTTTGAAAGTTAAA
GTACATGAAAATGTAGAAAAAGAAACAGCAAAAAGTCCTGCAACTTGTTACA
CAAATCAGTCCCCTTATTCAGTCATTGAAAATTCAGCCTTAGCTTTTTACACA
AGTTGTAGTAGAAAAACTTCTGTGAGTCAGACTTCATTACTTGAAGCAAAAA
AATGGCTTAGAGAAGGAATATTTGATGGTCAACCAGAAAGAATAAATACTGC
AGATTATGTAGGAAATTATTTGTATGAAAATAATTCAAACAGTACTATAGCT
GAAAATGACAAAAATCATCTCTCCGAAAAACAAGATACTTATTTAAGTAACA
GTAGCATGTCTAACAGCTATTCCTACCATTCTGATGAGGTATATAATGATTCA
GGATATCTCTCAAAAAATAAACTTGATTCTGGTATTGAGCCAGTATTGAAGA
ATGTTGAAGATCAAAAAAACACTAGTTTTTCCAAAGTAATATCCAATGTAAA
AGATGCAAATGCATACCCACAAACTGTAAATGAAGATATTTGCGTTGAGGAA
CTTGTGACTAGCTCTTCACCCTGCAAAAATAAAAATGCAGCCATTAAATTGTC
CATATCTAATAGTAATAATTTTGAGGTAGGGCCACCTGCATTTAGGATAGCCA
GTGGTAAAATCGTTTGTGTTTCACATGAAACAATTAAAAAAGTGAAAGACAT
ATTTACAGACAGTTTCAGTAAAGTAATTAAGGAAAACAACGAGAATAAATCA
AAAATTTGCCAAACGAAAATTATGGCAGGTTGTTACGAGGCATTGGATGATT
CAGAGGATATTCTTCATAACTCTCTAGATAATGATGAATGTAGCACGCATTCA
CATAAGGTTTTTGCTGACATTCAGAGTGAAGAAATTTTACAACATAACCAAA
ATATGTCTGGATTGGAGAAAGTTTCTAAAATATCACCTTGTGATGTTAGTTTG
GAAACTTCAGATATATGTAAATGTAGTATAGGGAAGCTTCATAAGTCAGTCT
CATCTGCAAATACTTGTGGGATTTTTAGCACAGCAAGTGGAAAATCTGTCCA
GGTATCAGATGCTTCATTACAAAACGCAAGACAAGTGTTTTCTGAAATAGAA
GATAGTACCAAGCAAGTCTTTTCCAAAGTATTGTTTAAAAGTAACGAACATTC
AGACCAGCTCACAAGAGAAGAAAATACTGCTATACGTACTCCAGAACATTTA
ATATCCCAAAAAGGCTTTTCATATAATGTGGTAAATTCATCTGCTTTCTCTGG
ATTTAGTACAGCAAGTGGAAAGCAAGTTTCCATTTTAGAAAGTTCCTTACACA
AAGTTAAGGGAGTGTTAGAGGAATTTGATTTAATCAGAACTGAGCATAGTCT
TCACTATTCACCTACGTCTAGACAAAATGTATCAAAAATACTTCCTCGTGTTG
ATAAGAGAAACCCAGAGCACTGTGTAAACTCAGAAATGGAAAAAACCTGCA
GTAAAGAATTTAAATTATCAAATAACTTAAATGTTGAAGGTGGTTCTTCAGA
AAATAATCACTCTATTAAAGTTTCTCCATATCTCTCTCAATTTCAACAAGACA
AACAACAGTTGGTATTAGGAACCAAAGTGTCACTTGTTGAGAACATTCATGT
TTTGGGAAAAGAACAGGCTTCACCTAAAAACGTAAAAATGGAAATTGGTAAA
ACTGAAACTTTTTCTGATGTTCCTGTGAAAACAAATATAGAAGTTTGTTCTAC
TTACTCCAAAGATTCAGAAAACTACTTTGAAACAGAAGCAGTAGAAATTGCT
AAAGCTTTTATGGAAGATGATGAACTGACAGATTCTAAACTGCCAAGTCATG
CCACACATTCTCTTTTTACATGTCCCGAAAATGAGGAAATGGTTTTGTCAAAT
TCAAGAATTGGAAAAAGAAGAGGAGAGCCCCTTATCTTAGTGGGAGAACCCT
CAATCAAAAGAAACTTATTAAATGAATTTGACAGGATAATAGAAAATCAAGA
AAAATCCTTAAAGGCTTCAAAAAGCACTCCAGATGGCACAATAAAAGATCGA
AGATTGTTTATGCATCATGTTTCTTTAGAGCCGATTACCTGTGTACCCTTTCGC
ACAACTAAGGAACGTCAAGAGATACAGAATCCAAATTTTACCGCACCTGGTC
AAGAATTTCTGTCTAAATCTCATTTGTATGAACATCTGACTTTGGAAAAATCT
TCAAGCAATTTAGCAGTTTCAGGACATCCATTTTATCAAGTTTCTGCTACAAG
AAATGAAAAAATGAGACACTTGATTACTACAGGCAGACCAACCAAAGTCTTT
GTTCCACCTTTTAAAACTAAATCACATTTTCACAGAGTTGAACAGTGTGTTAG
GAATATTAACTTGGAGGAAAACAGACAAAAGCAAAACATTGATGGACATGG
CTCTGATGATAGTAAAAATAAGATTAATGACAATGAGATTCATCAGTTTAAC
AAAAACAACTCCAATCAAGCAGTAGCTGTAACTTTCACAAAGTGTGAAGAAG
AACCTTTAGATTTAATTACAAGTCTTCAGAATGCCAGAGATATACAGGATAT
GCGAATTAAGAAGAAACAAAGGCAACGCGTCTTTCCACAGCCAGGCAGTCTG
TATCTTGCAAAAACATCCACTCTGCCTCGAATCTCTCTGAAAGCAGCAGTAGG
AGGCCAAGTTCCCTCTGCGTGTTCTCATAAACAGCTGTATACGTATGGCGTTT
CTAAACATTGCATAAAAATTAACAGCAAAAATGCAGAGTCTTTTCAGTTTCA
CACTGAAGATTATTTTGGTAAGGAAAGTTTATGGACTGGAAAAGGAATACAG
TTGGCTGATGGTGGATGGCTCATACCCTCCAATGATGGAAAGGCTGGAAAAG
AAGAATTTTATAGGGCTCTGTGTGACACTCCAGGTGTGGATCCAAAGCTTATT
TCTAGAATTTGGGTTTATAATCACTATAGATGGATCATATGGAAACTGGCAGC
TATGGAATGTGCCTTTCCTAAGGAATTTGCTAATAGATGCCTAAGCCCAGAA
AGGGTGCTTCTTCAACTAAAATACAGATATGATACGGAAATTGATAGAAGCA
GAAGATCGGCTATAAAAAAGATAATGGAAAGGGATGACACAGCTGCAAAAA
CACTTGTTCTCTGTGTTTCTGACATAATTTCATTGAGCGCAAATATATCTGAA
ACTTCTAGCAATAAAACTAGTAGTGCAGATACCCAAAAAGTGGCCATTATTG
AACTTACAGATGGGTGGTATGCTGTTAAGGCCCAGTTAGATCCTCCCCTCTTA
GCTGTCTTAAAGAATGGCAGACTGACAGTTGGTCAGAAGATTATTCTTCATG
GAGCAGAACTGGTGGGCTCTCCTGATGCCTGTACACCTCTTGAAGCCCCAGA
ATCTCTTATGTTAAAGATTTCTGCTAACAGTACTCGGCCTGCTCGCTGGTATA
CCAAACTTGGATTCTTTCCTGACCCTAGACCTTTTCCTCTGCCCTTATCATCGC
TTTTCAGTGATGGAGGAAATGTTGGTTGTGTTGATGTAATTATTCAAAGAGCA
TACCCTATACAGTGGATGGAGAAGACATCATCTGGATTATACATATTTCGCA
ATGAAAGAGAGGAAGAAAAGGAAGCAGCAAAATATGTGGAGGCCCAACAA
AAGAGACTAGAAGCCTTATTCACTAAAATTCAGGAGGAATTTGAAGAACATG
AAGAAAACACAACAAAACCATATTTACCATCACGTGCACTAACAAGACAGC
AAGTTCGTGCTTTGCAAGATGGTGCAGAGCTTTATGAAGCAGTGAAGAATGC
AGCAGACCCAGCTTACCTTGAGGGTTATTTCAGTGAAGAGCAGTTAAGAGCC
TTGAATAATCACAGGCAAATGTTGAATGATAAGAAACAAGCTCAGATCCAGT
TGGAAATTAGGAAGGCCATGGAATCTGCTGAACAAAAGGAACAAGGTTTATC
AAGGGATGTCACAACCGTGTGGAAGTTGCGTATTGTAAGCTATTCAAAAAAA
GAAAAAGATTCAGTTATACTGAGTATTTGGCGTCCATCATCAGATTTATATTC
TCTGTTAACAGAAGGAAAGAGATACAGAATTTATCATCTTGCAACTTCAAAA
TCTAAAAGTAAATCTGAAAGAGCTAACATACAGTTAGCAGCGACAAAAAAA
ACTCAGTATCAACAACTACCGGTTTCAGATGAAATTTTATTTCAGATTTACCA
GCCACGGGAGCCCCTTCACTTCAGCAAATTTTTAGATCCAGACTTTCAGCCAT
CTTGTTCTGAGGTGGACCTAATAGGATTTGTCGTTTCTGTTGTGAAAAAAACA
GGACTTGCCCCTTTCGTCTATTTGTCAGACGAATGTTACAATTTACTGGCAAT
AAAGTTTTGGATAGACCTTAATGAGGACATTATTAAGCCTCATATGTTAATTG
CTGCAAGCAACCTCCAGTGGCGACCAGAATCCAAATCAGGCCTTCTTACTTT
ATTTGCTGGAGATTTTTCTGTGTTTTCTGCTAGTCCAAAAGAGGGCCACTTTC
AAGAGACATTCAACAAAATGAAAAATACTGTTGAGAATATTGACATACTTTG
CAATGAAGCAGAAAACAAGCTTATGCATATACTGCATGCAAATGATCCCAAG
TGGTCCACCCCAACTAAAGACTGTACTTCAGGGCCGTACACTGCTCAAATCA
TTCCTGGTACAGGAAACAAGCTTCTGATGTCTTCTCCTAATTGTGAGATATAT
TATCAAAGTCCTTTATCACTTTGTATGGCCAAAAGGAAGTCTGTTTCCACACC
TGTCTCAGCCCAGATGACTTCAAAGTCTTGTAAAGGGGAGAAAGAGATTGAT
GACCAAAAGAACTGCAAAAAGAGAAGAGCCTTGGATTTCTTGAGTAGACTGC
CTTTACCTCCACCTGTTAGTCCCATTTGTACATTTGTTTCTCCGGCTGCACAGA
AGGCATTTCAGCCACCAAGGAGTTGTGGCACCAAATACGAAACACCCATAAA
GAAAAAAGAACTGAATTCTCCTCAGATGACTCCATTTAAAAAATTCAATGAA
ATTTCTCTTTTGGAAAGTAATTCAATAGCTGACGAAGAACTTGCATTGATAAA
TACCCAAGCTCTTTTGTCTGGTTCAACAGGAGAAAAACAATTTATATCTGTCA
GTGAATCCACTAGGACTGCTCCCACCAGTTCAGAAGATTATCTCAGACTGAA
ACGACGTTGTACTACATCTCTGATCAAAGAACAGGAGAGTTCCCAGGCCAGT
ACGGAAGAATGTGAGAAAAATAAGCAGGACACAATTACAACTAAAAAATAT
ATCTAA
Human BRCA2 Protein Sequence (SEQ ID NO: 25)
MPIGSKERPTFFEIFKTRCNKADLGPISLNWFEELSSEAPPYNSEPAEESEHKNNN
YEPNLFKTPQRKPSYNQLASTPIIFKEQGLTLPLYQSPVKELDKFKLDLGRNVPNS
RHKSLRTVKTKMDQADDVSCPLLNSCLSESPVVLQCTHVTPQRDKSVVCGSLFH
TPKFVKGRQTPKHISESLGAEVDPDMSWSSSLATPPTLSSTVLIVRNEEASETVFP
HDTTANVKSYFSNHDESLKKNDRFIASVTDSENTNQREAASHGFGKTSGNSFKV
NSCKDHIGKSMPNVLEDEVYETVVDTSEEDSFSLCFSKCRTKNLQKVRTSKTRK
KIFHEANADECEKSKNQVKEKYSFVSEVEPNDTDPLDSNVANQKPFESGSDKISK
EVVPSLACEWSQLTLSGLNGAQMEKIPLLHISSCDQNISEKDLLDTENKRKKDFL
TSENSLPRISSLPKSEKPLNEETVVNKRDEEQHLESHTDCILAVKQAISGTSPVASS
FQGIKKSIFRIRESPKETFNASFSGHMTDPNFKKETEASESGLEIHTVCSQKEDSLC
PNLIDNGSWPATTTQNSVALKNAGLISTLKKKTNKFIYAIHDETSYKGKKIPKDQ
KSELINCSAQFEANAFEAPLTFANADSGLLHSSVKRSCSQNDSEEPTLSLTSSFGTI
LRKCSRNETCSNNTVISQDLDYKEAKCNKEKLQLFITPEADSLSCLQEGQCENDP
KSKKVSDIKEEVLAAACHPVQHSKVEYSDTDFQSQKSLLYDHENASTLILTPTSK
DVLSNLVMISRGKESYKMSDKLKGNNYESDVELTKNIPMEKNQDVCALNENYK
NVELLPPEKYMRVASPSRKVQFNQNTNLRVIQKNQEETTSISKITVNPDSEELFSD
NENNFVFQVANERNNLALGNTKELHETDLTCVNEPIFKNSTMVLYGDTGDKQA
TQVSIKKDLVYVLAEENKNSVKQHIKMTLGQDLKSDISLNIDKIPEKNNDYMNK
WAGLLGPISNHSFGGSFRTASNKEIKLSEHNIKKSKMFFKDIEEQYPTSLACVEIV
NTLALDNQKKLSKPQSINTVSAHLQSSVVVSDCKNSHITPQMLFSKQDFNSNHNL
TPSQKAEITELSTILEESGSQFEFTQFRKPSYILQKSTFEVPENQMTILKTTSEECRD
ADLHVIMNAPSIGQVDSSKQFEGTVEIKRKFAGLLKNDCNKSASGYLTDENEVG
FRGFYSAHGTKLNVSTEALQKAVKLFSDIENISEETSAEVHPISLSSSKCHDSVVS
MFKIENHNDKTVSEKNNKCQLILQNNIEMTTGTFVEEITENYKRNTENEDNKYTA
ASRNSHNLEFDGSDSSKNDTVCIHKDETDLLFTDQHNICLKLSGQFMKEGNTQIK
EDLSDLTFLEVAKAQEACHGNTSNKEQLTATKTEQNIKDFETSDTFFQTASGKNI
SVAKESFNKIVNFFDQKPEELHNFSLNSELHSDIRKNKMDILSYEETDIVKHKILK
ESVPVGTGNQLVTFQGQPERDEKIKEPTLLGFHTASGKKVKIAKESLDKVKNLFD
EKEQGTSEITSFSHQWAKTLKYREACKDLELACETIEITAAPKCKEMQNSLNNDK
NLVSIETVVPPKLLSDNLCRQTENLKTSKSIFLKVKVHENVEKETAKSPATCYTN
QSPYSVIENSALAFYTSCSRKTSVSQTSLLEAKKWLREGIFDGQPERINTADYVGN
YLYENNSNSTIAENDKNHLSEKQDTYLSNSSMSNSYSYHSDEVYNDSGYLSKNK
LDSGIEPVLKNVEDQKNTSFSKVISNVKDANAYPQTVNEDICVEELVTSSSPCKN
KNAAIKLSISNSNNFEVGPPAFRIASGKIVCVSHETIKKVKDIFTDSFSKVIKENNE
NKSKICQTKIMAGCYEALDDSEDILHNSLDNDECSTHSHKVFADIQSEEILQHNQ
NMSGLEKVSKISPCDVSLETSDICKCSIGKLHKSVSSANTCGIFSTASGKSVQVSD
ASLQNARQVFSEIEDSTKQVFSKVLFKSNEHSDQLTREENTAIRTPEHLISQKGFS
YNVVNSSAFSGFSTASGKQVSILESSLHKVKGVLEEFDLIRTEHSLHYSPTSRQNV
SKILPRVDKRNPEHCVNSEMEKTCSKEFKLSNNLNVEGGSSENNHSIKVSPYLSQ
FQQDKQQLVLGTKVSLVENIHVLGKEQASPKNVKMEIGKTETFSDVPVKTNIEV
CSTYSKDSENYFETEAVEIAKAFMEDDELTDSKLPSHATHSLFTCPENEEMVLSN
SRIGKRRGEPLILVGEPSIKRNLLNEFDRIIENQEKSLKASKSTPDGTIKDRRLFMH
HVSLEPITCVPFRTTKERQEIQNPNFTAPGQEFLSKSHLYEHLTLEKSSSNLAVSG
HPFYQVSATRNEKMRHLITTGRPTKVFVPPFKTKSHFHRVEQCVRNINLEENRQK
QNIDGHGSDDSKNKINDNEIHQFNKNNSNQAVAVTFTKCEEEPLDLITSLQNARD
IQDMRIKKKQRQRVFPQPGSLYLAKTSTLPRISLKAAVGGQVPSACSHKQLYTYG
VSKHCIKINSKNAESFQFHTEDYFGKESLWTGKGIQLADGGWLIPSNDGKAGKEE
FYRALCDTPGVDPKLISRIWVYNHYRWIIWKLAAMECAFPKEFANRCLSPERVLL
QLKYRYDTEIDRSRRSAIKKIMERDDTAAKTLVLCVSDIISLSANISETSSNKTSSA
DTQKVAIIELTDGWYAVKAQLDPPLLAVLKNGRLTVGQKIILHGAELVGSPDAC
TPLEAPESLMLKISANSTRPARWYTKLGFFPDPRPFPLPLSSLFSDGGNVGCVDVII
QRAYPIQWMEKTSSGLYIFRNEREEEKEAAKYVEAQQKRLEALFTKIQEEFEEHE
ENTTKPYLPSRALTRQQVRALQDGAELYEAVKNAADPAYLEGYFSEEQLRALNN
HRQMLNDKKQAQIQLEIRKAMESAEQKEQGLSRDVTTVWKLRIVSYSKKEKDS
VILSIWRPSSDLYSLLTEGKRYRIYHLATSKSKSKSERANIQLAATKKTQYQQLPV
SDEILFQIYQPREPLHFSKFLDPDFQPSCSEVDLIGFVVSVVKKTGLAPFVYLSDEC
YNLLAIKFWIDLNEDIIKPHMLIAASNLQWRPESKSGLLTLFAGDFSVFSASPKEG
HFQETFNKMKNTVENIDILCNEAENKLMHILHANDPKWSTPTKDCTSGPYTAQII
PGTGNKLLMSSPNCEIYYQSPLSLCMAKRKSVSTPVSAQMTSKSCKGEKEIDDQK
NCKKRRALDFLSRLPLPPPVSPICTFVSPAAQKAFQPPRSCGTKYETPIKKKELNSP
QMTPFKKFNEISLLESNSIADEELALINTQALLSGSTGEKQFISVSESTRTAPTSSED
YLRLKRRCTTSLIKEQESSQASTEECEKNKQDTITTKKYI
Human SAMHD1 cDNA Sequence, Variant 1 (SEQ ID NO: 26)
ATGCAGCGAGCCGATTCCGAGCAGCCCTCCAAGCGTCCCCGTTGCGATGACA
GCCCGAGAACCCCCTCAAACACCCCTTCCGCAGAGGCAGACTGGTCCCCGGG
CCTGGAACTCCATCCCGACTACAAGACATGGGGTCCGGAGCAGGTGTGCTCC
TTCCTCAGGCGCGGTGGCTTTGAAGAGCCGGTGCTGCTGAAGAACATCCGAG
AAAATGAAATCACAGGCGCATTACTGCCTTGTCTTGATGAGTCTCGTTTTGAA
AATCTTGGAGTAAGTTCCTTGGGGGAGAGGAAGAAGCTGCTTAGTTATATCC
AGCGATTGGTTCAAATCCACGTTGATACAATGAAGGTAATTAATGATCCTATC
CATGGCCACATTGAGCTCCACCCTCTCCTCGTCCGAATCATTGATACACCTCA
ATTTCAACGTCTTCGATACATCAAACAGCTGGGAGGTGGTTACTATGTTTTTC
CAGGAGCTTCACACAATCGATTTGAGCATAGTCTAGGGGTGGGGTATCTAGC
AGGATGTCTAGTTCACGCACTGGGTGAAAAACAACCAGAGCTGCAGATAAGT
GAACGAGATGTTCTCTGTGTTCAGATTGCTGGACTTTGTCATGATCTCGGTCA
TGGGCCATTTTCTCACATGTTTGATGGACGATTTATTCCACTTGCTCGCCCGG
AGGTGAAATGGACGCATGAACAAGGCTCAGTTATGATGTTTGAGCACCTTAT
TAATTCTAATGGAATTAAGCCTGTCATGGAACAATATGGTCTCATCCCTGAAG
AAGATATTTGCTTTATAAAGGAACAAATTGTAGGACCACTTGAATCACCTGTC
GAAGATTCATTGTGGCCATATAAAGGGCGTCCTGAAAACAAAAGCTTCCTTT
ATGAGATAGTATCTAATAAAAGAAATGGCATTGATGTGGACAAATGGGATTA
TTTTGCCAGGGACTGCCATCATCTTGGAATCCAAAATAATTTTGATTACAAGC
GCTTTATTAAGTTTGCCCGTGTCTGTGAAGTAGACAATGAGTTGCGTATTTGT
GCTAGAGATAAGGAAGTTGGAAATCTGTATGACATGTTCCACACTCGCAACT
CTTTACACCGTAGAGCTTATCAACACAAAGTTGGCAACATTATTGATACAATG
ATTACAGATGCTTTCCTCAAAGCAGATGACTACATAGAGATTACAGGTGCTG
GAGGAAAAAAGTATCGCATTTCTACAGCAATTGACGACATGGAAGCCTATAC
TAAGCTGACAGATAACATTTTTCTGGAGATTTTATACTCTACTGATCCCAAAT
TGAAAGACGCACGAGAGATTTTAAAACAAATTGAATACCGTAATCTATTCAA
GTATGTGGGTGAGACGCAGCCAACAGGACAAATAAAGATTAAAAGGGAGGA
CTATGAATCTCTTCCAAAAGAGGTTGCCAGTGCTAAACCCAAAGTATTGCTA
GACGTGAAACTGAAGGCTGAAGATTTTATAGTGGATGTTATCAACATGGATT
ATGGAATGCAAGAAAAGAATCCAATTGATCATGTTAGCTTCTATTGTAAGAC
TGCCCCCAACAGAGCAATCAGGATTACTAAAAACCAGGTTTCACAACTTCTG
CCAGAGAAATTTGCAGAGCAGCTGATTCGAGTATATTGTAAGAAGGTGGACA
GAAAGAGTTTGTATGCCGCAAGACAATATTTTGTTCAGTGGTGTGCAGACAG
AAATTTCACCAAGCCGCAGGATGGCGATGTTATAGCCCCACTCATAACACCT
CAAAAAAAGGAATGGAACGACAGTACTTCAGTCCAAAATCCAACTCGCCTCC
GAGAAGCATCCAAAAGCAGAGTCCAGCTTTTTAAAGATGACCCAATGTGA
Human SAMHD1 Protein Sequence, Variant 1 (SEQ ID NO: 27)
MQRADSEQPSKRPRCDDSPRTPSNTPSAEADWSPGLELHPDYKTWGPEQVCSFL
RRGGFEEPVLLKNIRENEITGALLPCLDESRFENLGVSSLGERKKLLSYIQRLVQIH
VDTMKVINDPIHGHIELHPLLVRIIDTPQFQRLRYIKQLGGGYYVFPGASHNRFEH
SLGVGYLAGCLVHALGEKQPELQISERDVLCVQIAGLCHDLGHGPFSHMFDGRFI
PLARPEVKWTHEQGSVMMFEHLINSNGIKPVMEQYGLIPEEDICFIKEQIVGPLES
PVEDSLWPYKGRPENKSFLYEIVSNKRNGIDVDKWDYFARDCHHLGIQNNFDYK
RFIKFARVCEVDNELRICARDKEVGNLYDMFHTRNSLHRRAYQHKVGNIIDTMIT
DAFLKADDYIEITGAGGKKYRISTAIDDMEAYTKLTDNIFLEILYSTDPKLKDARE
ILKQIEYRNLFKYVGETQPTGQIKIKREDYESLPKEVASAKPKVLLDVKLKAEDFI
VDVINMDYGMQEKNPIDHVSFYCKTAPNRAIRITKNQVSQLLPEKFAEQLIRVYC
KKVDRKSLYAARQYFVQWCADRNFTKPQDGDVIAPLITPQKKEWNDSTSVQNP
TRLREASKSRVQLFKDDPM
Human SAMHD1 cDNA Sequence, Variant 2 (SEQ ID NO: 28)
ATGCAGCGAGCCGATTCCGAGCAGCCCTCCAAGCGTCCCCGTTGCGATGACA
GCCCGAGAACCCCCTCAAACACCCCTTCCGCAGAGGCAGACTGGTCCCCGGG
CCTGGAACTCCATCCCGACTACAAGACATGGGGTCCGGAGCAGGTGTGCTCC
TTCCTCAGGCGCGGTGGCTTTGAAGAGCCGGTGCTGCTGAAGAACATCCGAG
AAAATGAAATCACAGGCGCATTACTGCCTTGTCTTGATGAGTCTCGTTTTGAA
AATCTTGGAGTAAGTTCCTTGGGGGAGAGGAAGAAGCTGCTTAGTTATATCC
AGCGATTGGTTCAAATCCACGTTGATACAATGAAGGTAATTAATGATCCTATC
CATGGCCACATTGAGCTCCACCCTCTCCTCGTCCGAATCATTGATACACCTCA
ATTTCAACGTCTTCGATACATCAAACAGCTGGGAGGTGGTTACTATGTTTTTC
CAGGAGCTTCACACAATCGATTTGAGCATAGTCTAGGGGTGGGGTATCTAGC
AGGATGTCTAGTTCACGCACTGGGTGAAAAACAACCAGAGCTGCAGATAAGT
GAACGAGATGTTCTCTGTGTTCAGATTGCTGGACTTTGTCATGATCTCGGTCA
TGGGCCATTTTCTCACATGTTTGATGGACGATTTATTCCACTTGCTCGCCCGG
AGGTGAAATGGACGCATGAACAAGGCTCAGTTATGATGTTTGAGCACCTTAT
TAATTCTAATGGAATTAAGCCTGTCATGGAACAATATGGTCTCATCCCTGAAG
AAGATATTTGCTTTATAAAGGAACAAATTGTAGGACCACTTGAATCACCTGTC
GAAGATTCATTGTGGCCATATAAAGGGCGTCCTGAAAACAAAAGCTTCCTTT
ATGAGATAGTATCTAATAAAAGAAATGGCATTGATGTGGACAAATGGGATTA
TTTTGCCAGGGACTGCCATCATCTTGGAATCCAAAATAATTTTGATTACAAGC
GCTTTATTAAGTTTGCCCGTGTCTGTGAAGTAGACAATGAGTTGCGTATTTGT
GCTAGAGATAAGGAAGTTGGAAATCTGTATGACATGTTCCACACTCGCAACT
CTTTACACCGTAGAGCTTATCAACACAAAGTTGGCAACATTATTGATACAATG
ATTACAGATGCTTTCCTCAAAGCAGATGACTACATAGAGATTACAGGTGCTG
GAGGAAAAAAGTATCGCATTTCTACAGCAATTGACGACATGGAAGCCTATAC
TAAGCTGACAGATAACATTTTTCTGGAGATTTTATACTCTACTGATCCCAAAT
TGAAAGACGCACGAGAGATTTTAAAACAAATTGAATACCGTAATCTATTCAA
GTATGTGGGTGAGACGCAGCCAACAGGACAAATAAAGATTAAAAGGGAGGA
CTATGAATCTCTTCCAAAAGAGGTTGCCAGTGCTAAACCCAAAGTATTGCTA
GACGTGAAACTGAAGGCTGAAGATTTTATAGTGGATGTTTCACAACTTCTGCC
AGAGAAATTTGCAGAGCAGCTGATTCGAGTATATTGTAAGAAGGTGGACAGA
AAGAGTTTGTATGCCGCAAGACAATATTTTGTTCAGTGGTGTGCAGACAGAA
ATTTCACCAAGCCGCAGGATGGCGATGTTATAGCCCCACTCATAACACCTCA
AAAAAAGGAATGGAACGACAGTACTTCAGTCCAAAATCCAACTCGCCTCCGA
GAAGCATCCAAAAGCAGAGTCCAGCTTTTTAAAGATGACCCAATGTGA
Human SAMHD1 Protein Sequence, Variant 2 (SEQ ID NO: 29)
MQRADSEQPSKRPRCDDSPRTPSNTPSAEADWSPGLELHPDYKTWGPEQVCSFL
RRGGFEEPVLLKNIRENEITGALLPCLDESRFENLGVSSLGERKKLLSYIQRLVQIH
VDTMKVINDPIHGHIELHPLLVRIIDTPQFQRLRYIKQLGGGYYVFPGASHNRFEH
SLGVGYLAGCLVHALGEKQPELQISERDVLCVQIAGLCHDLGHGPFSHMFDGRFI
PLARPEVKWTHEQGSVMMFEHLINSNGIKPVMEQYGLIPEEDICFIKEQIVGPLES
PVEDSLWPYKGRPENKSFLYEIVSNKRNGIDVDKWDYFARDCHHLGIQNNFDYK
RFIKFARVCEVDNELRICARDKEVGNLYDMFHTRNSLHRRAYQHKVGNIIDTMIT
DAFLKADDYIEITGAGGKKYRISTAIDDMEAYTKLTDNIFLEILYSTDPKLKDARE
ILKQIEYRNLFKYVGETQPTGQIKIKREDYESLPKEVASAKPKVLLDVKLKAEDFI
VDVSQLLPEKFAEQLIRVYCKKVDRKSLYAARQYFVQWCADRNFTKPQDGDVI
APLITPQKKEWNDSTSVQNPTRLREASKSRVQLFKDDPM
Human SAMHD1 cDNA Sequence, Variant 3 (SEQ ID NO: 30)
ATGCAGCGAGCCGATTCCGAGCAGCCCTCCAAGCGTCCCCGTTGCGATGACA
GCCCGAGAACCCCCTCAAACACCCCTTCCGCAGAGGCAGACTGGTCCCCGGG
CCTGGAACTCCATCCCGACTACAAGACATGGGGTCCGGAGCAGGTGTGCTCC
TTCCTCAGGCGCGGTGGCTTTGAAGAGCCGGTGCTGCTGAAGAACATCCGAG
AAAATGAAATCACAGGCGCATTACTGCCTTGTCTTGATGAGTCTCGTTTTGAA
AATCTTGGAGTAAGTTCCTTGGGGGAGAGGAAGAAGCTGCTTAGTTATATCC
AGCGATTGGTTCAAATCCACGTTGATACAATGAAGGTAATTAATGATCCTATC
CATGGCCACATTGAGCTCCACCCTCTCCTCGTCCGAATCATTGATACACCTCA
ATTTCAACGTCTTCGATACATCAAACAGCTGGGAGGTGGTTACTATGTTTTTC
CAGGAGCTTCACACAATCGATTTGAGCATAGTCTAGGGGTGGGGTATCTAGC
AGGATGTCTAGTTCACGCACTGGGTGAAAAACAACCAGAGCTGCAGATAAGT
GAACGAGATGTTCTCTGTGTTCAGATTGCTGGACTTTGTCATGATCTCGGTCA
TGGGCCATTTTCTCACATGTTTGATGGACGATTTATTCCACTTGCTCGCCCGG
AGGTGAAATGGACGCATGAACAAGGCTCAGTTATGATGTTTGAGCACCTTAT
TAATTCTAATGGAATTAAGCCTGTCATGGAACAATATGGTCTCATCCCTGAAG
AAGATATTTGCTTTATAAAGGAACAAATTGTAGGACCACTTGAATCACCTGTC
GAAGATTCATTGTGGCCATATAAAGGGCGTCCTGAAAACAAAAGCTTCCTTT
ATGAGATAGTATCTAATAAAAGAAATGGCATTGATGTGGACAAATGGGATTA
TTTTGCCAGGGACTGCCATCATCTTGGAATCCAAAATAATTTTGATTACAAGC
GCTTTATTAAGTTTGCCCGTGTCTGTGAAGTAGACAATGAGTTGCGTATTTGT
GCTAGAGATAAGGAAGTTGGAAATCTGTATGACATGTTCCACACTCGCAACT
CTTTACACCGTAGAGCTTATCAACACAAAGTTGGCAACATTATTGATACAATG
ATTACAGATGCTTTCCTCAAAGCAGATGACTACATAGAGATTACAGGTGCTG
GAGGAAAAAAGTATCGCATTTCTACAGCAATTGACGACATGGAAGCCTATAC
TAAGCTGACAGATAACATTTTTCTGGAGATTTTATACTCTACTGATCCCAAAT
TGAAAGACGCACGAGAGATTTTAAAACAAATTGAATACCGTAATCTATTCAA
GTATGTGGGTGAGACGCAGCCAACAGGACAAATAAAGATTAAAAGGGAGGA
CTATGAATCTCTTCCAAAAGAGGTTGCCAGTGCTAAACCCAAAGTATTGCTA
GACGTGAAACTGAAGGCTGAAGATTTTATAGTGGATGTTATCAACATGGATT
ATGGAATGCAAGAAAAGAATCCAATTGATCATGTTAGCTTCTATTGTAAGAC
TGCCCCCAACAGAGCAATCAGGATTACTAAAAACCAGGTTTCACAACTTCTG
CCAGAGAAATTTGCAGAGCAGCTGATTCGAGTATATTGTAAGAAGGTGGACA
GAAAGAGTTTGTATGCCGCAAGACAATATTTTGTTCAGTGGTGTGCAGACAG
AAATTTCACCAAGCCGCAGTCTCCCACCAGAGCCTCCCACTGA
Human SAMHD1 Protein Sequence, Variant 3 (SEQ ID NO: 31)
MQRADSEQPSKRPRCDDSPRTPSNTPSAEADWSPGLELHPDYKTWGPEQVCSFL
RRGGFEEPVLLKNIRENEITGALLPCLDESRFENLGVSSLGERKKLLSYIQRLVQIH
VDTMKVINDPIHGHIELHPLLVRIIDTPQFQRLRYIKQLGGGYYVFPGASHNRFEH
SLGVGYLAGCLVHALGEKQPELQISERDVLCVQIAGLCHDLGHGPFSHMFDGRFI
PLARPEVKWTHEQGSVMMFEHLINSNGIKPVMEQYGLIPEEDICFIKEQIVGPLES
PVEDSLWPYKGRPENKSFLYEIVSNKRNGIDVDKWDYFARDCHHLGIQNNFDYK
RFIKFARVCEVDNELRICARDKEVGNLYDMFHTRNSLHRRAYQHKVGNIIDTMIT
DAFLKADDYIEITGAGGKKYRISTAIDDMEAYTKLTDNIFLEILYSTDPKLKDARE
ILKQIEYRNLFKYVGETQPTGQIKIKREDYESLPKEVASAKPKVLLDVKLKAEDFI
VDVINMDYGMQEKNPIDHVSFYCKTAPNRAIRITKNQVSQLLPEKFAEQLIRVYC
KKVDRKSLYAARQYFVQWCADRNFTKPQSPTRASH
Human DNASE2 Precursor cDNA Sequence (SEQ ID NO: 32)
ATGATCCCGCTGCTGCTGGCAGCGCTGCTGTGCGTCCCCGCCGGGGCCCTGA
CCTGCTACGGGGACTCCGGGCAGCCTGTAGACTGGTTCGTGGTCTACAAGCT
GCCAGCTCTTAGAGGGTCCGGGGAGGCGGCGCAGAGAGGGCTGCAGTACAA
GTATCTGGACGAGAGCTCCGGAGGCTGGCGGGACGGCAGGGCACTCATCAA
CAGCCCGGAGGGGGCCGTGGGCCGAAGCCTGCAGCCGCTGTACCGGAGCAA
CACCAGCCAGCTCGCCTTCCTGCTCTACAATGACCAACCGCCTCAACCCAGC
AAGGCTCAGGACTCTTCCATGCGTGGGCACACGAAGGGTGTCCTGCTCCTTG
ACCACGATGGGGGCTTCTGGCTGGTCCACAGTGTACCTAACTTCCCTCCACCG
GCCTCCTCTGCTGCATACAGCTGGCCTCATAGCGCCTGTACCTACGGGCAGAC
CCTGCTCTGTGTGTCTTTTCCCTTCGCTCAGTTCTCGAAGATGGGCAAGCAGC
TGACCTACACCTACCCCTGGGTCTATAACTACCAGCTGGAAGGGATCTTTGCC
CAGGAATTCCCCGACTTGGAGAATGTGGTCAAGGGCCACCACGTTAGCCAAG
AACCCTGGAACAGCAGCATCACACTCACATCCCAGGCCGGGGCTGTTTTCCA
GAGCTTTGCCAAGTTCAGCAAATTTGGAGATGACCTGTACTCCGGCTGGTTGG
CAGCAGCCCTTGGTACCAACCTGCAGGTCCAGTTCTGGCACAAAACTGTAGG
CATCCTGCCCTCTAACTGCTCGGATATCTGGCAGGTTCTGAATGTGAACCAGA
TAGCTTTCCCTGGACCAGCCGGCCCAAGCTTCAACAGCACAGAGGACCACTC
CAAATGGTGCGTGTCCCCAAAAGGGCCCTGGACCTGCGTGGGTGACATGAAT
CGGAACCAGGGAGAGGAGCAACGGGGTGGGGGCACACTGTGTGCCCAGCTG
CCAGCCCTCTGGAAAGCCTTCCAGCCGCTGGTGAAGAACTACCAGCCCTGTA
ATGGCATGGCCAGGAAGCCCAGCAGAGCTTATAAGATCTAA
Human DNASE2 Precursor Protein Sequence (SEQ ID NO: 33)
MIPLLLAALLCVPAGALTCYGDSGQPVDWFVVYKLPALRGSGEAAQRGLQYKY
LDESSGGWRDGRALINSPEGAVGRSLQPLYRSNTSQLAFLLYNDQPPQPSKAQDS
SMRGHTKGVLLLDHDGGFWLVHSVPNFPPPASSAAYSWPHSACTYGQTLLCVSF
PFAQFSKMGKQLTYTYPWVYNYQLEGIFAQEFPDLENVVKGHHVSQEPWNSSIT
LTSQAGAVFQSFAKFSKFGDDLYSGWLAAALGTNLQVQFWHKTVGILPSNCSDI
WQVLNVNQIAFPGPAGPSFNSTEDHSKWCVSPKGPWTCVGDMNRNQGEEQRG
GGTLCAQLPALWKAFQPLVKNYQPCNGMARKPSRAYKI
Human DNASE2 Mature cDNA Sequence (SEQ ID NO: 34)
TGCTACGGGGACTCCGGGCAGCCTGTAGACTGGTTCGTGGTCTACAAGCTGC
CAGCTCTTAGAGGGTCCGGGGAGGCGGCGCAGAGAGGGCTGCAGTACAAGT
ATCTGGACGAGAGCTCCGGAGGCTGGCGGGACGGCAGGGCACTCATCAACA
GCCCGGAGGGGGCCGTGGGCCGAAGCCTGCAGCCGCTGTACCGGAGCAACA
CCAGCCAGCTCGCCTTCCTGCTCTACAATGACCAACCGCCTCAACCCAGCAA
GGCTCAGGACTCTTCCATGCGTGGGCACACGAAGGGTGTCCTGCTCCTTGAC
CACGATGGGGGCTTCTGGCTGGTCCACAGTGTACCTAACTTCCCTCCACCGGC
CTCCTCTGCTGCATACAGCTGGCCTCATAGCGCCTGTACCTACGGGCAGACCC
TGCTCTGTGTGTCTTTTCCCTTCGCTCAGTTCTCGAAGATGGGCAAGCAGCTG
ACCTACACCTACCCCTGGGTCTATAACTACCAGCTGGAAGGGATCTTTGCCCA
GGAATTCCCCGACTTGGAGAATGTGGTCAAGGGCCACCACGTTAGCCAAGAA
CCCTGGAACAGCAGCATCACACTCACATCCCAGGCCGGGGCTGTTTTCCAGA
GCTTTGCCAAGTTCAGCAAATTTGGAGATGACCTGTACTCCGGCTGGTTGGCA
GCAGCCCTTGGTACCAACCTGCAGGTCCAGTTCTGGCACAAAACTGTAGGCA
TCCTGCCCTCTAACTGCTCGGATATCTGGCAGGTTCTGAATGTGAACCAGATA
GCTTTCCCTGGACCAGCCGGCCCAAGCTTCAACAGCACAGAGGACCACTCCA
AATGGTGCGTGTCCCCAAAAGGGCCCTGGACCTGCGTGGGTGACATGAATCG
GAACCAGGGAGAGGAGCAACGGGGGGGGGCACACTGTGTGCCCAGCTGCC
AGCCCTCTGGAAAGCCTTCCAGCCGCTGGTGAAGAACTACCAGCCCTGTAAT
GGCATGGCCAGGAAGCCCAGCAGAGCTTATAAGATCTAA
Human DNASE2 Mature Protein Sequence (SEQ ID NO: 35)
CYGDSGQPVDWFVVYKLPALRGSGEAAQRGLQYKYLDESSGGWRDGRALINSP
EGAVGRSLQPLYRSNTSQLAFLLYNDQPPQPSKAQDSSMRGHTKGVLLLDHDGG
FWLVHSVPNFPPPASSAAYSWPHSACTYGQTLLCVSFPFAQFSKMGKQLTYTYP
WVYNYQLEGIFAQEFPDLENVVKGHHVSQEPWNSSITLTSQAGAVFQSFAKFSK
FGDDLYSGWLAAALGTNLQVQFWHKTVGILPSNCSDIWQVLNVNQIAFPGPAGP
SFNSTEDHSKWCVSPKGPWTCVGDMNRNQGEEQRGGGTLCAQLPALWKAFQP
LVKNYQPCNGMARKPSRAYKI
Human BLM cDNA Sequence, Variant 1 (SEQ ID NO: 36)
ATGGCTGCTGTTCCTCAAAATAATCTACAGGAGCAACTAGAACGTCACTCAG
CCAGAACACTTAATAATAAATTAAGTCTTTCAAAACCAAAATTTTCAGGTTTC
ACTTTTAAAAAGAAAACATCTTCAGATAACAATGTATCTGTAACTAATGTGTC
AGTAGCAAAAACACCTGTATTAAGAAATAAAGATGTTAATGTTACCGAAGAC
TTTTCCTTCAGTGAACCTCTACCCAACACCACAAATCAGCAAAGGGTCAAGG
ACTTCTTTAAAAATGCTCCAGCAGGACAGGAAACACAGAGAGGTGGATCAAA
ATCATTATTGCCAGATTTCTTGCAGACTCCGAAGGAAGTTGTATGCACTACCC
AAAACACACCAACTGTAAAGAAATCCCGGGATACTGCTCTCAAGAAATTAGA
ATTTAGTTCTTCACCAGATTCTTTAAGTACCATCAATGATTGGGATGATATGG
ATGACTTTGATACTTCTGAGACTTCAAAATCATTTGTTACACCACCCCAAAGT
CACTTTGTAAGAGTAAGCACTGCTCAGAAATCAAAAAAGGGTAAGAGAAACT
TTTTTAAAGCACAGCTTTATACAACAAACACAGTAAAGACTGATTTGCCTCCA
CCCTCCTCTGAAAGCGAGCAAATAGATTTGACTGAGGAACAGAAGGATGACT
CAGAATGGTTAAGCAGCGATGTGATTTGCATCGATGATGGCCCCATTGCTGA
AGTGCATATAAATGAAGATGCTCAGGAAAGTGACTCTCTGAAAACTCATTTG
GAAGATGAAAGAGATAATAGCGAAAAGAAGAAGAATTTGGAAGAAGCTGAA
TTACATTCAACTGAGAAAGTTCCATGTATTGAATTTGATGATGATGATTATGA
TACGGATTTTGTTCCACCTTCTCCAGAAGAAATTATTTCTGCTTCTTCTTCCTC
TTCAAAATGCCTTAGTACGTTAAAGGACCTTGACACCTCTGACAGAAAAGAG
GATGTTCTTAGCACATCAAAAGATCTTTTGTCAAAACCTGAGAAAATGAGTA
TGCAGGAGCTGAATCCAGAAACCAGCACAGACTGTGACGCTAGACAGATAA
GTTTACAGCAGCAGCTTATTCATGTGATGGAGCACATCTGTAAATTAATTGAT
ACTATTCCTGATGATAAACTGAAACTTTTGGATTGTGGGAACGAACTGCTTCA
GCAGCGGAACATAAGAAGGAAACTTCTAACGGAAGTAGATTTTAATAAAAGT
GATGCCAGTCTTCTTGGCTCATTGTGGAGATACAGGCCTGATTCACTTGATGG
CCCTATGGAGGGTGATTCCTGCCCTACAGGGAATTCTATGAAGGAGTTAAAT
TTTTCACACCTTCCCTCAAATTCTGTTTCTCCTGGGGACTGTTTACTGACTACC
ACCCTAGGAAAGACAGGATTCTCTGCCACCAGGAAGAATCTTTTTGAAAGGC
CTTTATTCAATACCCATTTACAGAAGTCCTTTGTAAGTAGCAACTGGGCTGAA
ACACCAAGACTAGGAAAAAAAAATGAAAGCTCTTATTTCCCAGGAAATGTTC
TCACAAGCACTGCTGTGAAAGATCAGAATAAACATACTGCTTCAATAAATGA
CTTAGAAAGAGAAACCCAACCTTCCTATGATATTGATAATTTTGACATAGATG
ACTTTGATGATGATGATGACTGGGAAGACATAATGCATAATTTAGCAGCCAG
CAAATCTTCCACAGCTGCCTATCAACCCATCAAGGAAGGTCGGCCAATTAAA
TCAGTATCAGAAAGACTTTCCTCAGCCAAGACAGACTGTCTTCCAGTGTCATC
TACTGCTCAAAATATAAACTTCTCAGAGTCAATTCAGAATTATACTGACAAGT
CAGCACAAAATTTAGCATCCAGAAATCTGAAACATGAGCGTTTCCAAAGTCT
TAGTTTTCCTCATACAAAGGAAATGATGAAGATTTTTCATAAAAAATTTGGCC
TGCATAATTTTAGAACTAATCAGCTAGAGGCGATCAATGCTGCACTGCTTGGT
GAAGACTGTTTTATCCTGATGCCGACTGGAGGTGGTAAGAGTTTGTGTTACCA
GCTCCCTGCCTGTGTTTCTCCTGGGGTCACTGTTGTCATTTCTCCCTTGAGATC
ACTTATCGTAGATCAAGTCCAAAAGCTGACTTCCTTGGATATTCCAGCTACAT
ATCTGACAGGTGATAAGACTGACTCAGAAGCTACAAATATTTACCTCCAGTT
ATCAAAAAAAGACCCAATCATAAAACTTCTATATGTCACTCCAGAAAAGATC
TGTGCAAGTAACAGACTCATTTCTACTCTGGAGAATCTCTATGAGAGGAAGC
TCTTGGCACGTTTTGTTATTGATGAAGCACATTGTGTCAGTCAGTGGGGACAT
GATTTTCGTCAAGATTACAAAAGAATGAATATGCTTCGCCAGAAGTTTCCTTC
TGTTCCGGTGATGGCTCTTACGGCCACAGCTAATCCCAGGGTACAGAAGGAC
ATCCTGACTCAGCTGAAGATTCTCAGACCTCAGGTGTTTAGCATGAGCTTTAA
CAGACATAATCTGAAATACTATGTATTACCGAAAAAGCCTAAAAAGGTGGCA
TTTGATTGCCTAGAATGGATCAGAAAGCACCACCCATATGATTCAGGGATAA
TTTACTGCCTCTCCAGGCGAGAATGTGACACCATGGCTGACACGTTACAGAG
AGATGGGCTCGCTGCTCTTGCTTACCATGCTGGCCTCAGTGATTCTGCCAGAG
ATGAAGTGCAGCAGAAGTGGATTAATCAGGATGGCTGTCAGGTTATCTGTGC
TACAATTGCATTTGGAATGGGGATTGACAAACCGGACGTGCGATTTGTGATT
CATGCATCTCTCCCTAAATCTGTGGAGGGTTACTACCAAGAATCTGGCAGAG
CTGGAAGAGATGGGGAAATATCTCACTGCCTGCTTTTCTATACCTATCATGAT
GTGACCAGACTGAAAAGACTTATAATGATGGAAAAAGATGGAAACCATCAT
ACAAGAGAAACTCACTTCAATAATTTGTATAGCATGGTACATTACTGTGAAA
ATATAACGGAATGCAGGAGAATACAGCTTTTGGCCTACTTTGGTGAAAATGG
ATTTAATCCTGATTTTTGTAAGAAACACCCAGATGTTTCTTGTGATAATTGCT
GTAAAACAAAGGATTATAAAACAAGAGATGTGACTGACGATGTGAAAAGTA
TTGTAAGATTTGTTCAAGAACATAGTTCATCACAAGGAATGAGAAATATAAA
ACATGTAGGTCCTTCTGGAAGATTTACTATGAATATGCTGGTCGACATTTTCT
TGGGGAGTAAGAGTGCAAAAATCCAGTCAGGTATATTTGGAAAAGGATCTGC
TTATTCACGACACAATGCCGAAAGACTTTTTAAAAAGCTGATACTTGACAAG
ATTTTGGATGAAGACTTATATATCAATGCCAATGACCAGGCGATCGCTTATGT
GATGCTCGGAAATAAAGCCCAAACTGTACTAAATGGCAATTTAAAGGTAGAC
TTTATGGAAACAGAAAATTCCAGCAGTGTGAAAAAACAAAAAGCGTTAGTAG
CAAAAGTGTCTCAGAGGGAAGAGATGGTTAAAAAATGTCTTGGAGAACTTAC
AGAAGTCTGCAAATCTCTGGGGAAAGTTTTTGGTGTCCATTACTTCAATATTT
TTAATACCGTCACTCTCAAGAAGCTTGCAGAATCTTTATCTTCTGATCCTGAG
GTTTTGCTTCAAATTGATGGTGTTACTGAAGACAAACTGGAAAAATATGGTG
CGGAAGTGATTTCAGTATTACAGAAATACTCTGAATGGACATCGCCAGCTGA
AGACAGTTCCCCAGGGATAAGCCTGTCCAGCAGCAGAGGCCCCGGAAGAAG
TGCCGCTGAGGAGCTCGACGAGGAAATACCCGTATCTTCCCACTACTTTGCA
AGTAAAACCAGAAATGAAAGGAAGAGGAAAAAGATGCCAGCCTCCCAAAGG
TCTAAGAGGAGAAAAACTGCTTCCAGTGGTTCCAAGGCAAAGGGGGGGTCTG
CCACATGTAGAAAGATATCTTCCAAAACGAAATCCTCCAGCATCATTGGATC
CAGTTCAGCCTCACATACTTCTCAAGCGACATCAGGAGCCAATAGCAAATTG
GGGATTATGGCTCCACCGAAGCCTATAAATAGACCGTTTCTTAAGCCTTCATA
TGCATTCTCATAA
Human BLM Protein Sequence, Variant 1 (SEQ ID NO: 37)
MAAVPQNNLQEQLERHSARTLNNKLSLSKPKFSGFTFKKKTSSDNNVSVTNVSV
AKTPVLRNKDVNVTEDFSFSEPLPNTTNQQRVKDFFKNAPAGQETQRGGSKSLL
PDFLQTPKEVVCTTQNTPTVKKSRDTALKKLEFSSSPDSLSTINDWDDMDDFDTS
ETSKSFVTPPQSHFVRVSTAQKSKKGKRNFFKAQLYTTNTVKTDLPPPSSESEQID
LTEEQKDDSEWLSSDVICIDDGPIAEVHINEDAQESDSLKTHLEDERDNSEKKKN
LEEAELHSTEKVPCIEFDDDDYDTDFVPPSPEEIISASSSSSKCLSTLKDLDTSDRK
EDVLSTSKDLLSKPEKMSMQELNPETSTDCDARQISLQQQLIHVMEHICKLIDTIP
DDKLKLLDCGNELLQQRNIRRKLLTEVDFNKSDASLLGSLWRYRPDSLDGPMEG
DSCPTGNSMKELNFSHLPSNSVSPGDCLLTTTLGKTGFSATRKNLFERPLFNTHL
QKSFVSSNWAETPRLGKKNESSYFPGNVLTSTAVKDQNKHTASINDLERETQPSY
DIDNFDIDDFDDDDDWEDIMHNLAASKSSTAAYQPIKEGRPIKSVSERLSSAKTD
CLPVSSTAQNINFSESIQNYTDKSAQNLASRNLKHERFQSLSFPHTKEMMKIFHK
KFGLHNFRTNQLEAINAALLGEDCFILMPTGGGKSLCYQLPACVSPGVTVVISPL
RSLIVDQVQKLTSLDIPATYLTGDKTDSEATNIYLQLSKKDPIIKLLYVTPEKICAS
NRLISTLENLYERKLLARFVIDEAHCVSQWGHDFRQDYKRMNMLRQKFPSVPV
MALTATANPRVQKDILTQLKILRPQVFSMSFNRHNLKYYVLPKKPKKVAFDCLE
WIRKHHPYDSGIIYCLSRRECDTMADTLQRDGLAALAYHAGLSDSARDEVQQK
WINQDGCQVICATIAFGMGIDKPDVRFVIHASLPKSVEGYYQESGRAGRDGEISH
CLLFYTYHDVTRLKRLIMMEKDGNHHTRETHFNNLYSMVHYCENITECRRIQLL
AYFGENGFNPDFCKKHPDVSCDNCCKTKDYKTRDVTDDVKSIVRFVQEHSSSQG
MRNIKHVGPSGRFTMNMLVDIFLGSKSAKIQSGIFGKGSAYSRHNAERLFKKLIL
DKILDEDLYINANDQAIAYVMLGNKAQTVLNGNLKVDFMETENSSSVKKQKAL
VAKVSQREEMVKKCLGELTEVCKSLGKVFGVHYFNIFNTVTLKKLAESLSSDPE
VLLQIDGVTEDKLEKYGAEVISVLQKYSEWTSPAEDSSPGISLSSSRGPGRSAAEE
LDEEIPVSSHYFASKTRNERKRKKMPASQRSKRRKTASSGSKAKGGSATCRKISS
KTKSSSIIGSSSASHTSQATSGANSKLGIMAPPKPINRPFLKPSYAFS
Human BLM cDNA Sequence, Variant 2 (SEQ ID NO: 38)
ATGGCTGCTGTTCCTCAAAATAATCTACAGGAGCAACTAGAACGTCACTCAG
CCAGAACACTTAATAATAAATTAAGTCTTTCAAAACCAAAATTTTCAGGTTTC
ACTTTTAAAAAGAAAACATCTTCAGATAACAATGTATCTGTAACTAATGTGTC
AGTAGCAAAAACACCTGTATTAAGAAATAAAGATGTTAATGTTACCGAAGAC
TTTTCCTTCAGTGAACCTCTACCCAACACCACAAATCAGCAAAGGGTCAAGG
ACTTCTTTAAAAATGCTCCAGCAGGACAGGAAACACAGAGAGGTGGATCAAA
ATCATTATTGCCAGATTTCTTGCAGACTCCGAAGGAAGTTGTATGCACTACCC
AAAACACACCAACTGTAAAGAAATCCCGGGATACTGCTCTCAAGAAATTAGA
ATTTAGTTCTTCACCAGATTCTTTAAGTACCATCAATGATTGGGATGATATGG
ATGACTTTGATACTTCTGAGACTTCAAAATCATTTGTTACACCACCCCAAAGT
CACTTTGTAAGAGTAAGCACTGCTCAGAAATCAAAAAAGGGTAAGAGAAACT
TTTTTAAAGCACAGCTTTATACAACAAACACAGTAAAGACTGATTTGCCTCCA
CCCTCCTCTGAAAGCGAGCAAATAGATTTGACTGAGGAACAGAAGGATGACT
CAGAATGGTTAAGCAGCGATGTGATTTGCATCGATGATGGCCCCATTGCTGA
AGTGCATATAAATGAAGATGCTCAGGAAAGTGACTCTCTGAAAACTCATTTG
GAAGATGAAAGAGATAATAGCGAAAAGAAGAAGAATTTGGAAGAAGCTGAA
TTACATTCAACTGAGAAAGTTCCATGTATTGAATTTGATGATGATGATTATGA
TACGGATTTTGTTCCACCTTCTCCAGAAGAAATTATTTCTGCTTCTTCTTCCTC
TTCAAAATGCCTTAGTACGTTAAAGGACCTTGACACCTCTGACAGAAAAGAG
GATGTTCTTAGCACATCAAAAGATCTTTTGTCAAAACCTGAGAAAATGAGTA
TGCAGGAGCTGAATCCAGAAACCAGCACAGACTGTGACGCTAGACAGATAA
GTTTACAGCAGCAGCTTATTCATGTGATGGAGCACATCTGTAAATTAATTGAT
ACTATTCCTGATGATAAACTGAAACTTTTGGATTGTGGGAACGAACTGCTTCA
GCAGCGGAACATAAGAAGGAAACTTCTAACGGAAGTAGATTTTAATAAAAGT
GATGCCAGTCTTCTTGGCTCATTGTGGAGATACAGGCCTGATTCACTTGATGG
CCCTATGGAGGGTGATTCCTGCCCTACAGGGAATTCTATGAAGGAGTTAAAT
TTTTCACACCTTCCCTCAAATTCTGTTTCTCCTGGGGACTGTTTACTGACTACC
ACCCTAGGAAAGACAGGATTCTCTGCCACCAGGAAGAATCTTTTTGAAAGGC
CTTTATTCAATACCCATTTACAGAAGTCCTTTGTAAGTAGCAACTGGGCTGAA
ACACCAAGACTAGGAAAAAAAAATGAAAGCTCTTATTTCCCAGGAAATGTTC
TCACAAGCACTGCTGTGAAAGATCAGAATAAACATACTGCTTCAATAAATGA
CTTAGAAAGAGAAACCCAACCTTCCTATGATATTGATAATTTTGACATAGATG
ACTTTGATGATGATGATGACTGGGAAGACATAATGCATAATTTAGCAGCCAG
CAAATCTTCCACAGCTGCCTATCAACCCATCAAGGAAGGTCGGCCAATTAAA
TCAGTATCAGAAAGACTTTCCTCAGCCAAGACAGACTGTCTTCCAGTGTCATC
TACTGCTCAAAATATAAACTTCTCAGAGTCAATTCAGAATTATACTGACAAGT
CAGCACAAAATTTAGCATCCAGAAATCTGAAACATGAGCGTTTCCAAAGTCT
TAGTTTTCCTCATACAAAGGAAATGATGAAGATTTTTCATAAAAAATTTGGCC
TGCATAATTTTAGAACTAATCAGCTAGAGGCGATCAATGCTGCACTGCTTGGT
GAAGACTGTTTTATCCTGATGCCGACTGGAGGTGGTAAGAGTTTGTGTTACCA
GCTCCCTGCCTGTGTTTCTCCTGGGGTCACTGTTGTCATTTCTCCCTTGAGATC
ACTTATCGTAGATCAAGTCCAAAAGCTGACTTCCTTGGATATTCCAGCTACAT
ATCTGACAGGTGATAAGACTGACTCAGAAGCTACAAATATTTACCTCCAGTT
ATCAAAAAAAGACCCAATCATAAAACTTCTATATGTCACTCCAGAAAAGATC
TGTGCAAGTAACAGACTCATTTCTACTCTGGAGAATCTCTATGAGAGGAAGC
TCTTGGCACGTTTTGTTATTGATGAAGCACATTGTGTCAGTCAGTGGGGACAT
GATTTTCGTCAAGATTACAAAAGAATGAATATGCTTCGCCAGAAGTTTCCTTC
TGTTCCGGTGATGGCTCTTACGGCCACAGCTAATCCCAGGGTACAGAAGGAC
ATCCTGACTCAGCTGAAGATTCTCAGACCTCAGGTGTTTAGCATGAGCTTTAA
CAGACATAATCTGAAATACTATGTATTACCGAAAAAGCCTAAAAAGGTGGCA
TTTGATTGCCTAGAATGGATCAGAAAGCACCACCCATATGATTCAGGGATAA
TTTACTGCCTCTCCAGGCGAGAATGTGACACCATGGCTGACACGTTACAGAG
AGATGGGCTCGCTGCTCTTGCTTACCATGCTGGCCTCAGTGATTCTGCCAGAG
ATGAAGTGCAGCAGAAGTGGATTAATCAGGATGGCTGTCAGGTTATCTGTGC
TACAATTGCATTTGGAATGGGGATTGACAAACCGGACGTGCGATTTGTGATT
CATGCATCTCTCCCTAAATCTGTGGAGGGTTACTACCAAGAATCTGGCAGAG
CTGGAAGAGATGGGGAAATATCTCACTGCCTGCTTTTCTATACCTATCATGAT
GTGACCAGACTGAAAAGACTTATAATGATGGAAAAAGATGGAAACCATCAT
ACAAGAGAAACTCACTTCAATAATTTGTATAGCATGGTACATTACTGTGAAA
ATATAACGGAATGCAGGAGAATACAGCTTTTGGCCTACTTTGGTGAAAATGG
ATTTAATCCTGATTTTTGTAAGAAACACCCAGATGTTTCTTGTGATAATTGCT
GTAAAACAAAGGATTATAAAACAAGAGATGTGACTGACGATGTGAAAAGTA
TTGTAAGATTTGTTCAAGAACATAGTTCATCACAAGGAATGAGAAATATAAA
ACATGTAGGTCCTTCTGGAAGATTTACTATGAATATGCTGGTCGACATTTTCT
TGGAATCTTTATCTTCTGATCCTGAGGTTTTGCTTCAAATTGATGGTGTTACTG
AAGACAAACTGGAAAAATATGGTGCGGAAGTGATTTCAGTATTACAGAAATA
CTCTGAATGGACATCGCCAGCTGAAGACAGTTCCCCAGGGATAAGCCTGTCC
AGCAGCAGAGGCCCCGGAAGAAGTGCCGCTGAGGAGCTCGACGAGGAAATA
CCCGTATCTTCCCACTACTTTGCAAGTAAAACCAGAAATGAAAGGAAGAGGA
AAAAGATGCCAGCCTCCCAAAGGTCTAAGAGGAGAAAAACTGCTTCCAGTGG
TTCCAAGGCAAAGGGGGGGTCTGCCACATGTAGAAAGATATCTTCCAAAACG
AAATCCTCCAGCATCATTGGATCCAGTTCAGCCTCACATACTTCTCAAGCGAC
ATCAGGAGCCAATAGCAAATTGGGGATTATGGCTCCACCGAAGCCTATAAAT
AGACCGTTTCTTAAGCCTTCATATGCATTCTCATAA
Human BLM Protein Sequence, Variant 2 (SEQ ID NO: 39)
MAAVPQNNLQEQLERHSARTLNNKLSLSKPKFSGFTFKKKTSSDNNVSVTNVSV
AKTPVLRNKDVNVTEDFSFSEPLPNTTNQQRVKDFFKNAPAGQETQRGGSKSLL
PDFLQTPKEVVCTTQNTPTVKKSRDTALKKLEFSSSPDSLSTINDWDDMDDFDTS
ETSKSFVTPPQSHFVRVSTAQKSKKGKRNFFKAQLYTTNTVKTDLPPPSSESEQID
LTEEQKDDSEWLSSDVICIDDGPIAEVHINEDAQESDSLKTHLEDERDNSEKKKN
LEEAELHSTEKVPCIEFDDDDYDTDFVPPSPEEIISASSSSSKCLSTLKDLDTSDRK
EDVLSTSKDLLSKPEKMSMQELNPETSTDCDARQISLQQQLIHVMEHICKLIDTIP
DDKLKLLDCGNELLQQRNIRRKLLTEVDFNKSDASLLGSLWRYRPDSLDGPMEG
DSCPTGNSMKELNFSHLPSNSVSPGDCLLTTTLGKTGFSATRKNLFERPLFNTHL
QKSFVSSNWAETPRLGKKNESSYFPGNVLTSTAVKDQNKHTASINDLERETQPSY
DIDNFDIDDFDDDDDWEDIMHNLAASKSSTAAYQPIKEGRPIKSVSERLSSAKTD
CLPVSSTAQNINFSESIQNYTDKSAQNLASRNLKHERFQSLSFPHTKEMMKIFHK
KFGLHNFRTNQLEAINAALLGEDCFILMPTGGGKSLCYQLPACVSPGVTVVISPL
RSLIVDQVQKLTSLDIPATYLTGDKTDSEATNIYLQLSKKDPIIKLLYVTPEKICAS
NRLISTLENLYERKLLARFVIDEAHCVSQWGHDFRQDYKRMNMLRQKFPSVPV
MALTATANPRVQKDILTQLKILRPQVFSMSFNRHNLKYYVLPKKPKKVAFDCLE
WIRKHHPYDSGIIYCLSRRECDTMADTLQRDGLAALAYHAGLSDSARDEVQQK
WINQDGCQVICATIAFGMGIDKPDVRFVIHASLPKSVEGYYQESGRAGRDGEISH
CLLFYTYHDVTRLKRLIMMEKDGNHHTRETHFNNLYSMVHYCENITECRRIQLL
AYFGENGFNPDFCKKHPDVSCDNCCKTKDYKTRDVTDDVKSIVRFVQEHSSSQG
MRNIKHVGPSGRFTMNMLVDIFLESLSSDPEVLLQIDGVTEDKLEKYGAEVISVL
QKYSEWTSPAEDSSPGISLSSSRGPGRSAAEELDEEIPVSSHYFASKTRNERKRKK
MPASQRSKRRKTASSGSKAKGGSATCRKISSKTKSSSIIGSSSASHTSQATSGANS
KLGIMAPPKPINRPFLKPSYAFS
Human BLM cDNA Sequence, Variant 3 (SEQ ID NO: 40)
ATGGAGCACATCTGTAAATTAATTGATACTATTCCTGATGATAAACTGAAACT
TTTGGATTGTGGGAACGAACTGCTTCAGCAGCGGAACATAAGAAGGAAACTT
CTAACGGAAGTAGATTTTAATAAAAGTGATGCCAGTCTTCTTGGCTCATTGTG
GAGATACAGGCCTGATTCACTTGATGGCCCTATGGAGGGTGATTCCTGCCCTA
CAGGGAATTCTATGAAGGAGTTAAATTTTTCACACCTTCCCTCAAATTCTGTT
TCTCCTGGGGACTGTTTACTGACTACCACCCTAGGAAAGACAGGATTCTCTGC
CACCAGGAAGAATCTTTTTGAAAGGCCTTTATTCAATACCCATTTACAGAAGT
CCTTTGTAAGTAGCAACTGGGCTGAAACACCAAGACTAGGAAAAAAAAATG
AAAGCTCTTATTTCCCAGGAAATGTTCTCACAAGCACTGCTGTGAAAGATCA
GAATAAACATACTGCTTCAATAAATGACTTAGAAAGAGAAACCCAACCTTCC
TATGATATTGATAATTTTGACATAGATGACTTTGATGATGATGATGACTGGGA
AGACATAATGCATAATTTAGCAGCCAGCAAATCTTCCACAGCTGCCTATCAA
CCCATCAAGGAAGGTCGGCCAATTAAATCAGTATCAGAAAGACTTTCCTCAG
CCAAGACAGACTGTCTTCCAGTGTCATCTACTGCTCAAAATATAAACTTCTCA
GAGTCAATTCAGAATTATACTGACAAGTCAGCACAAAATTTAGCATCCAGAA
ATCTGAAACATGAGCGTTTCCAAAGTCTTAGTTTTCCTCATACAAAGGAAATG
ATGAAGATTTTTCATAAAAAATTTGGCCTGCATAATTTTAGAACTAATCAGCT
AGAGGCGATCAATGCTGCACTGCTTGGTGAAGACTGTTTTATCCTGATGCCGA
CTGGAGGTGGTAAGAGTTTGTGTTACCAGCTCCCTGCCTGTGTTTCTCCTGGG
GTCACTGTTGTCATTTCTCCCTTGAGATCACTTATCGTAGATCAAGTCCAAAA
GCTGACTTCCTTGGATATTCCAGCTACATATCTGACAGGTGATAAGACTGACT
CAGAAGCTACAAATATTTACCTCCAGTTATCAAAAAAAGACCCAATCATAAA
ACTTCTATATGTCACTCCAGAAAAGATCTGTGCAAGTAACAGACTCATTTCTA
CTCTGGAGAATCTCTATGAGAGGAAGCTCTTGGCACGTTTTGTTATTGATGAA
GCACATTGTGTCAGTCAGTGGGGACATGATTTTCGTCAAGATTACAAAAGAA
TGAATATGCTTCGCCAGAAGTTTCCTTCTGTTCCGGTGATGGCTCTTACGGCC
ACAGCTAATCCCAGGGTACAGAAGGACATCCTGACTCAGCTGAAGATTCTCA
GACCTCAGGTGTTTAGCATGAGCTTTAACAGACATAATCTGAAATACTATGTA
TTACCGAAAAAGCCTAAAAAGGTGGCATTTGATTGCCTAGAATGGATCAGAA
AGCACCACCCATATGATTCAGGGATAATTTACTGCCTCTCCAGGCGAGAATG
TGACACCATGGCTGACACGTTACAGAGAGATGGGCTCGCTGCTCTTGCTTACC
ATGCTGGCCTCAGTGATTCTGCCAGAGATGAAGTGCAGCAGAAGTGGATTAA
TCAGGATGGCTGTCAGGTTATCTGTGCTACAATTGCATTTGGAATGGGGATTG
ACAAACCGGACGTGCGATTTGTGATTCATGCATCTCTCCCTAAATCTGTGGAG
GGTTACTACCAAGAATCTGGCAGAGCTGGAAGAGATGGGGAAATATCTCACT
GCCTGCTTTTCTATACCTATCATGATGTGACCAGACTGAAAAGACTTATAATG
ATGGAAAAAGATGGAAACCATCATACAAGAGAAACTCACTTCAATAATTTGT
ATAGCATGGTACATTACTGTGAAAATATAACGGAATGCAGGAGAATACAGCT
TTTGGCCTACTTTGGTGAAAATGGATTTAATCCTGATTTTTGTAAGAAACACC
CAGATGTTTCTTGTGATAATTGCTGTAAAACAAAGGATTATAAAACAAGAGA
TGTGACTGACGATGTGAAAAGTATTGTAAGATTTGTTCAAGAACATAGTTCAT
CACAAGGAATGAGAAATATAAAACATGTAGGTCCTTCTGGAAGATTTACTAT
GAATATGCTGGTCGACATTTTCTTGGGGAGTAAGAGTGCAAAAATCCAGTCA
GGTATATTTGGAAAAGGATCTGCTTATTCACGACACAATGCCGAAAGACTTTT
TAAAAAGCTGATACTTGACAAGATTTTGGATGAAGACTTATATATCAATGCC
AATGACCAGGCGATCGCTTATGTGATGCTCGGAAATAAAGCCCAAACTGTAC
TAAATGGCAATTTAAAGGTAGACTTTATGGAAACAGAAAATTCCAGCAGTGT
GAAAAAACAAAAAGCGTTAGTAGCAAAAGTGTCTCAGAGGGAAGAGATGGT
TAAAAAATGTCTTGGAGAACTTACAGAAGTCTGCAAATCTCTGGGGAAAGT
TTTTGGTGTCCATTACTTCAATATTTTTAATACCGTCACTCTCAAGAAGCTTGC
AGAATCTTTATCTTCTGATCCTGAGGTTTTGCTTCAAATTGATGGTGTTACTGA
AGACAAACTGGAAAAATATGGTGCGGAAGTGATTTCAGTATTACAGAAATAC
TCTGAATGGACATCGCCAGCTGAAGACAGTTCCCCAGGGATAAGCCTGTCCA
GCAGCAGAGGCCCCGGAAGAAGTGCCGCTGAGGAGCTCGACGAGGAAATAC
CCGTATCTTCCCACTACTTTGCAAGTAAAACCAGAAATGAAAGGAAGAGGAA
AAAGATGCCAGCCTCCCAAAGGTCTAAGAGGAGAAAAACTGCTTCCAGTGGT
TCCAAGGCAAAGGGGGGGTCTGCCACATGTAGAAAGATATCTTCCAAAACGA
AATCCTCCAGCATCATTGGATCCAGTTCAGCCTCACATACTTCTCAAGCGACA
TCAGGAGCCAATAGCAAATTGGGGATTATGGCTCCACCGAAGCCTATAAATA
GACCGTTTCTTAAGCCTTCATATGCATTCTCATAA
Human BLM Protein Sequence, Variant 3 (SEQ ID NO: 41)
MEHICKLIDTIPDDKLKLLDCGNELLQQRNIRRKLLTEVDFNKSDASLLGSLWRY
RPDSLDGPMEGDSCPTGNSMKELNFSHLPSNSVSPGDCLLTTTLGKTGFSATRKN
LFERPLFNTHLQKSFVSSNWAETPRLGKKNESSYFPGNVLTSTAVKDQNKHTASI
NDLERETQPSYDIDNFDIDDFDDDDDWEDIMHNLAASKSSTAAYQPIKEGRPIKS
VSERLSSAKTDCLPVSSTAQNINFSESIQNYTDKSAQNLASRNLKHERFQSLSFPH
TKEMMKIFHKKFGLHNFRTNQLEAINAALLGEDCFILMPTGGGKSLCYQLPACV
SPGVTVVISPLRSLIVDQVQKLTSLDIPATYLTGDKTDSEATNIYLQLSKKDPIIKL
LYVTPEKICASNRLISTLENLYERKLLARFVIDEAHCVSQWGHDFRQDYKRMNM
LRQKFPSVPVMALTATANPRVQKDILTQLKILRPQVFSMSFNRHNLKYYVLPKKP
KKVAFDCLEWIRKHHPYDSGIIYCLSRRECDTMADTLQRDGLAALAYHAGLSDS
ARDEVQQKWINQDGCQVICATIAFGMGIDKPDVRFVIHASLPKSVEGYYQESGR
AGRDGEISHCLLFYTYHDVTRLKRLIMMEKDGNHHTRETHFNNLYSMVHYCENI
TECRRIQLLAYFGENGFNPDFCKKHPDVSCDNCCKTKDYKTRDVTDDVKSIVRF
VQEHSSSQGMRNIKHVGPSGRFTMNMLVDIFLGSKSAKIQSGIFGKGSAYSRHNA
ERLFKKLILDKILDEDLYINANDQAIAYVMLGNKAQTVLNGNLKVDFMETENSS
SVKKQKALVAKVSQREEMVKKCLGELTEVCKSLGKVFGVHYFNIFNTVTLKKL
AESLSSDPEVLLQIDGVTEDKLEKYGAEVISVLQKYSEWTSPAEDSSPGISLSSSR
GPGRSAAEELDEEIPVSSHYFASKTRNERKRKKMPASQRSKRRKTASSGSKAKG
GSATCRKISSKTKSSSIIGSSSASHTSQATSGANSKLGIMAPPKPINRPFLKPSYAFS
Human PARP1 cDNA sequence (SEQ ID NO: 42)
ATGGCGGAGTCTTCGGATAAGCTCTATCGAGTCGAGTACGCCAAGAGCGGGC
GCGCCTCTTGCAAGAAATGCAGCGAGAGCATCCCCAAGGACTCGCTCCGGAT
GGCCATCATGGTGCAGTCGCCCATGTTTGATGGAAAAGTCCCACACTGGTAC
CACTTCTCCTGCTTCTGGAAGGTGGGCCACTCCATCCGGCACCCTGACGTTGA
GGTGGATGGGTTCTCTGAGCTTCGGTGGGATGACCAGCAGAAAGTCAAGAAG
ACAGCGGAAGCTGGAGGAGTGACAGGCAAAGGCCAGGATGGAATTGGTAGC
AAGGCAGAGAAGACTCTGGGTGACTTTGCAGCAGAGTATGCCAAGTCCAACA
GAAGTACGTGCAAGGGGTGTATGGAGAAGATAGAAAAGGGCCAGGTGCGCC
TGTCCAAGAAGATGGTGGACCCGGAGAAGCCACAGCTAGGCATGATTGACCG
CTGGTACCATCCAGGCTGCTTTGTCAAGAACAGGGAGGAGCTGGGTTTCCGG
CCCGAGTACAGTGCGAGTCAGCTCAAGGGCTTCAGCCTCCTTGCTACAGAGG
ATAAAGAAGCCCTGAAGAAGCAGCTCCCAGGAGTCAAGAGTGAAGGAAAGA
GAAAAGGCGATGAGGTGGATGGAGTGGATGAAGTGGCGAAGAAGAAATCTA
AAAAAGAAAAAGACAAGGATAGTAAGCTTGAAAAAGCCCTAAAGGCTCAGA
ACGACCTGATCTGGAACATCAAGGACGAGCTAAAGAAAGTGTGTTCAACTAA
TGACCTGAAGGAGCTACTCATCTTCAACAAGCAGCAAGTGCCTTCTGGGGAG
TCGGCGATCTTGGACCGAGTAGCTGATGGCATGGTGTTCGGTGCCCTCCTTCC
CTGCGAGGAATGCTCGGGTCAGCTGGTCTTCAAGAGCGATGCCTATTACTGC
ACTGGGGACGTCACTGCCTGGACCAAGTGTATGGTCAAGACACAGACACCCA
ACCGGAAGGAGTGGGTAACCCCAAAGGAATTCCGAGAAATCTCTTACCTCAA
GAAATTGAAGGTTAAAAAACAGGACCGTATATTCCCCCCAGAAACCAGCGCC
TCCGTGGCGGCCACGCCTCCGCCCTCCACAGCCTCGGCTCCTGCTGCTGTGAA
CTCCTCTGCTTCAGCAGATAAGCCATTATCCAACATGAAGATCCTGACTCTCG
GGAAGCTGTCCCGGAACAAGGATGAAGTGAAGGCCATGATTGAGAAACTCG
GGGGGAAGTTGACGGGGACGGCCAACAAGGCTTCCCTGTGCATCAGCACCA
AAAAGGAGGTGGAAAAGATGAATAAGAAGATGGAGGAAGTAAAGGAAGCC
AACATCCGAGTTGTGTCTGAGGACTTCCTCCAGGACGTCTCCGCCTCCACCAA
GAGCCTTCAGGAGTTGTTCTTAGCGCACATCTTGTCCCCTTGGGGGGCAGAGG
TGAAGGCAGAGCCTGTTGAAGTTGTGGCCCCAAGAGGGAAGTCAGGGGCTGC
GCTCTCCAAAAAAAGCAAGGGCCAGGTCAAGGAGGAAGGTATCAACAAATC
TGAAAAGAGAATGAAATTAACTCTTAAAGGAGGAGCAGCTGTGGATCCTGAT
TCTGGACTGGAACACTCTGCGCATGTCCTGGAGAAAGGTGGGAAGGTCTTCA
GTGCCACCCTTGGCCTGGTGGACATCGTTAAAGGAACCAACTCCTACTACAA
GCTGCAGCTTCTGGAGGACGACAAGGAAAACAGGTATTGGATATTCAGGTCC
TGGGGCCGTGTGGGTACGGTGATCGGTAGCAACAAACTGGAACAGATGCCGT
CCAAGGAGGATGCCATTGAGCACTTCATGAAATTATATGAAGAAAAAACCGG
GAACGCTTGGCACTCCAAAAATTTCACGAAGTATCCCAAAAAGTTCTACCCC
CTGGAGATTGACTATGGCCAGGATGAAGAGGCAGTGAAGAAGCTGACAGTA
AATCCTGGCACCAAGTCCAAGCTCCCCAAGCCAGTTCAGGACCTCATCAAGA
TGATCTTTGATGTGGAAAGTATGAAGAAAGCCATGGTGGAGTATGAGATCGA
CCTTCAGAAGATGCCCTTGGGGAAGCTGAGCAAAAGGCAGATCCAGGCCGC
ATACTCCATCCTCAGTGAGGTCCAGCAGGCGGTGTCTCAGGGCAGCAGCGAC
TCTCAGATCCTGGATCTCTCAAATCGCTTTTACACCCTGATCCCCCACGACTT
TGGGATGAAGAAGCCTCCGCTCCTGAACAATGCAGACAGTGTGCAGGCCAAG
GTGGAAATGCTTGACAACCTGCTGGACATCGAGGTGGCCTACAGTCTGCTCA
GGGGAGGGTCTGATGATAGCAGCAAGGATCCCATCGATGTCAACTATGAGAA
GCTCAAAACTGACATTAAGGTGGTTGACAGAGATTCTGAAGAAGCCGAGATC
ATCAGGAAGTATGTTAAGAACACTCATGCAACCACACACAATGCGTATGACT
TGGAAGTCATCGATATCTTTAAGATAGAGCGTGAAGGCGAATGCCAGCGTTA
CAAGCCCTTTAAGCAGCTTCATAACCGAAGATTGCTGTGGCACGGGTCCAGG
ACCACCAACTTTGCTGGGATCCTGTCCCAGGGTCTTCGGATAGCCCCGCCTGA
AGCGCCCGTGACAGGCTACATGTTTGGTAAAGGGATCTATTTCGCTGACATG
GTCTCCAAGAGTGCCAACTACTGCCATACGTCTCAGGGAGACCCAATAGGCT
TAATCCTGTTGGGAGAAGTTGCCCTTGGAAACATGTATGAACTGAAGCACGC
TTCACATATCAGCAAGTTACCCAAGGGCAAGCACAGTGTCAAAGGTTTGGGC
AAAACTACCCCTGATCCTTCAGCTAACATTAGTCTGGATGGTGTAGACGTTCC
TCTTGGGACCGGGATTTCATCTGGTGTGAATGACACCTCTCTACTATATAACG
AGTACATTGTCTATGATATTGCTCAGGTAAATCTGAAGTATCTGCTGAAACTG
AAATTCAATTTTAAGACCTCCCTGTGGTAA
Human PARP protein sequence (SEQ ID NO: 43)
MAESSDKLYRVEYAKSGRASCKKCSESIPKDSLRMAIMVQSPMFDGKVPHWYH
FSCFWKVGHSIRHPDVEVDGFSELRWDDQQKVKKTAEAGGVTGKGQDGIGSKA
EKTLGDFAAEYAKSNRSTCKGCMEKIEKGQVRLSKKMVDPEKPQLGMIDRWYH
PGCFVKNREELGFRPEYSASQLKGFSLLATEDKEALKKQLPGVKSEGKRKGDEV
DGVDEVAKKKSKKEKDKDSKLEKALKAQNDLIWNIKDELKKVCSTNDLKELLIF
NKQQVPSGESAILDRVADGMVFGALLPCEECSGQLVFKSDAYYCTGDVTAWTK
CMVKTQTPNRKEWVTPKEFREISYLKKLKVKKQDRIFPPETSASVAATPPPSTAS
APAAVNSSASADKPLSNMKILTLGKLSRNKDEVKAMIEKLGGKLTGTANKASLC
ISTKKEVEKMNKKMEEVKEANIRVVSEDFLQDVSASTKSLQELFLAHILSPWGAE
VKAEPVEVVAPRGKSGAALSKKSKGQVKEEGINKSEKRMKLTLKGGAAVDPDS
GLEHSAHVLEKGGKVFSATLGLVDIVKGTNSYYKLQLLEDDKENRYWIFRSWG
RVGTVIGSNKLEQMPSKEDAIEHFMKLYEEKTGNAWHSKNFTKYPKKFYPLEID
YGQDEEAVKKLTVNPGTKSKLPKPVQDLIKMIFDVESMKKAMVEYEIDLQKMPL
GKLSKRQIQAAYSILSEVQQAVSQGSSDSQILDLSNRFYTLIPHDFGMKKPPLLNN
ADSVQAKVEMLDNLLDIEVAYSLLRGGSDDSSKDPIDVNYEKLKTDIKVVDRDS
EEAEIIRKYVKNTHATTHNAYDLEVIDIFKIEREGECQRYKPFKQLHNRRLLWHG
SRTTNFAGILSQGLRIAPPEAPVTGYMFGKGIYFADMVSKSANYCHTSQGDPIGLI
LLGEVALGNMYELKHASHISKLPKGKHSVKGLGKTTPDPSANISLDGVDVPLGT
GISSGVNDTSLLYNEYIVYDIAQVNLKYLLKLKFNFKTSLW
Human RPA1 cDNA Sequence, Variant 1 (SEQ ID NO: 44)
ATGGTCGGCCAACTGAGCGAGGGGGCCATTGCGGCCATCATGCAGAAGGGG
GATACAAACATAAAGCCCATCCTCCAAGTCATCAACATCCGTCCCATTACTA
CGGGGAATAGTCCGCCGCGTTATCGACTGCTCATGAGTGATGGATTGAACAC
TCTATCCTCTTTCATGTTGGCGACACAGTTGAACCCTCTCGTGGAGGAAGAAC
AATTGTCCAGCAACTGTGTATGCCAGATTCACAGATTTATTGTGAACACTCTG
AAAGACGGAAGGAGAGTAGTTATCTTGATGGAATTAGAAGTTTTGAAGTCAG
CTGAAGCAGTTGGAGTGAAGATTGGCAATCCAGTGCCCTATAATGAAGGACT
CGGGCAGCCGCAAGTAGCTCCTCCAGCGCCAGCAGCCAGCCCAGCAGCAAG
CAGCAGGCCCCAGCCGCAGAATGGAAGCTCGGGAATGGGTTCTACTGTTTCT
AAGGCTTATGGTGCTTCAAAGACATTTGGAAAAGCTGCAGGTCCCAGCCTGT
CACACACTTCTGGGGGAACACAGTCCAAAGTGGTGCCCATTGCCAGCCTCAC
TCCTTACCAGTCCAAGTGGACCATTTGTGCTCGTGTTACCAACAAAAGTCAGA
TCCGTACCTGGAGCAACTCCCGAGGGGAAGGGAAGCTTTTCTCCCTAGAACT
GGTTGACGAAAGTGGTGAAATCCGAGCTACAGCTTTCAATGAGCAAGTGGAC
AAGTTCTTTCCTCTTATTGAAGTGAACAAGGTGTATTATTTCTCGAAAGGCAC
CCTGAAGATTGCTAACAAGCAGTTCACAGCTGTTAAAAATGACTACGAGATG
ACCTTCAATAACGAGACTTCCGTCATGCCCTGTGAGGACGACCATCATTTACC
TACGGTTCAGTTTGATTTCACGGGGATTGATGACCTCGAGAACAAGTCGAAA
GACTCACTTGTAGACATCATCGGGATCTGCAAGAGCTATGAAGACGCCACTA
AAATCACAGTGAGGTCTAACAACAGAGAAGTTGCCAAGAGGAATATCTACTT
GATGGACACATCCGGGAAGGTGGTGACTGCTACACTGTGGGGGGAAGATGCT
GATAAATTTGATGGTTCTAGACAGCCCGTGTTGGCTATCAAAGGAGCCCGAG
TCTCTGATTTCGGTGGACGGAGCCTCTCCGTGCTGTCTTCAAGCACTATCATT
GCGAATCCTGACATCCCAGAGGCCTATAAGCTTCGTGGATGGTTTGACGCAG
AAGGACAAGCCTTAGATGGTGTTTCCATCTCTGATCTAAAGAGCGGCGGAGT
CGGAGGGAGTAACACCAACTGGAAAACCTTGTATGAGGTCAAATCCGAGAA
CCTGGGCCAAGGCGACAAGCCGGACTACTTTAGTTCTGTGGCCACAGTGGTG
TATCTTCGCAAAGAGAACTGCATGTACCAAGCCTGCCCGACTCAGGACTGCA
ATAAGAAAGTGATTGATCAACAGAATGGATTGTACCGCTGTGAGAAGTGCGA
CACCGAATTTCCCAATTTCAAGTACCGCATGATCCTGTCAGTAAATATTGCAG
ATTTTCAAGAGAATCAGTGGGTGACTTGTTTCCAGGAGTCTGCTGAAGCTATC
CTTGGACAAAATGCTGCTTATCTTGGGGAATTAAAAGACAAGAATGAACAGG
CATTTGAAGAAGTTTTCCAGAATGCCAACTTCCGATCTTTCATATTCAGAGTC
AGGGTCAAAGTGGAGACCTACAACGACGAGTCTCGAATTAAGGCCACTGTGA
TGGACGTGAAGCCCGTGGACTACAGAGAGTATGGCCGAAGGCTGGTCATGAG
CATCAGGAGAAGTGCATTGATGTGA
Human RPA1 Protein Sequence, Variant 1 (SEQ ID NO: 45)
MVGQLSEGAIAAIMQKGDTNIKPILQVINIRPITTGNSPPRYRLLMSDGLNTLSSF
MLATQLNPLVEEEQLSSNCVCQIHRFIVNTLKDGRRVVILMELEVLKSAEAVGVK
IGNPVPYNEGLGQPQVAPPAPAASPAASSRPQPQNGSSGMGSTVSKAYGASKTF
GKAAGPSLSHTSGGTQSKVVPIASLTPYQSKWTICARVTNKSQIRTWSNSRGEGK
LFSLELVDESGEIRATAFNEQVDKFFPLIEVNKVYYFSKGTLKIANKQFTAVKND
YEMTFNNETSVMPCEDDHHLPTVQFDFTGIDDLENKSKDSLVDIIGICKSYEDAT
KITVRSNNREVAKRNIYLMDTSGKVVTATLWGEDADKFDGSRQPVLAIKGARVS
DFGGRSLSVLSSSTIIANPDIPEAYKLRGWFDAEGQALDGVSISDLKSGGVGGSNT
NWKTLYEVKSENLGQGDKPDYFSSVATVVYLRKENCMYQACPTQDCNKKVID
QQNGLYRCEKCDTEFPNFKYRMILSVNIADFQENQWVTCFQESAEAILGQNAAY
LGELKDKNEQAFEEVFQNANFRSFIFRVRVKVETYNDESRIKATVMDVKPVDYR
EYGRRLVMSIRRSALM
Human RPA1 cDNA Sequence, Variant 2 (SEQ ID NO: 46)
ATGCAGAAGGGGGATACAAACATAAAGCCCATCCTCCAAGTCATCAACATCC
GTCCCATTACTACGGGGAATAGTCCGCCGCGTTATCGACTGCTCATGAGTGAT
GGATTGAACACTCTATCCTCTTTCATGTTGGCGACACAGTTGAACCCTCTCGT
GGAGGAAGAACAATTGTCCAGCAACTGTGTATGCCAGATTCACAGATTTATT
GTGAACACTCTGAAAGACGGAAGGAGAGTAGTTATCTTGATGGAATTAGAAG
TTTTGAAGTCAGCTGAAGCAGTTGGAGTGAAGATTGGCAATCCAGTGCCCTA
TAATGAAGGACTCGGGCAGCCGCAAGTAGCTCCTCCAGCGCCAGCAGCCAGC
CCAGCAGCAAGCAGCAGGCCCCAGCCGCAGAATGGAAGCTCGGGAATGGGT
TCTACTGTTTCTAAGGCTTATGGTGCTTCAAAGACATTTGGAAAAGCTGCAGG
TCCCAGCCTGTCACACACTTCTGGGGGAACACAGTCCAAAGTGGTGCCCATT
GCCAGCCTCACTCCTTACCAGTCCAAGTGGACCATTTGTGCTCGTGTTACCAA
CAAAAGTCAGATCCGTACCTGGAGCAACTCCCGAGGGGAAGGGAAGCTTTTC
TCCCTAGAACTGGTTGACGAAAGTGGTGAAATCCGAGCTACAGCTTTCAATG
AGCAAGTGGACAAGTTCTTTCCTCTTATTGAAGTGAACAAGGTGTATTATTTC
TCGAAAGGCACCCTGAAGATTGCTAACAAGCAGTTCACAGCTGTTAAAAATG
ACTACGAGATGACCTTCAATAACGAGACTTCCGTCATGCCCTGTGAGGACGA
CCATCATTTACCTACGGTTCAGTTTGATTTCACGGGGATTGATGACCTCGAGA
ACAAGTCGAAAGACTCACTTGTAGACATCATCGGGATCTGCAAGAGCTATGA
AGACGCCACTAAAATCACAGTGAGGTCTAACAACAGAGAAGTTGCCAAGAG
GAATATCTACTTGATGGACACATCCGGGAAGGTGGTGACTGCTACACTGTGG
GGGGAAGATGCTGATAAATTTGATGGTTCTAGACAGCCCGTGTTGGCTATCA
AAGGAGCCCGAGTCTCTGATTTCGGTGGACGGAGCCTCTCCGTGCTGTCTTCA
AGCACTATCATTGCGAATCCTGACATCCCAGAGGCCTATAAGCTTCGTGGAT
GGTTTGACGCAGAAGGACAAGCCTTAGATGGTGTTTCCATCTCTGATCTAAA
GAGCGGCGGAGTCGGAGGGAGTAACACCAACTGGAAAACCTTGTATGAGGT
CAAATCCGAGAACCTGGGCCAAGGCGACAAGCCGGACTACTTTAGTTCTGTG
GCCACAGTGGTGTATCTTCGCAAAGAGAACTGCATGTACCAAGCCTGCCCGA
CTCAGGACTGCAATAAGAAAGTGATTGATCAACAGAATGGATTGTACCGCTG
TGAGAAGTGCGACACCGAATTTCCCAATTTCAAGTACCGCATGATCCTGTCA
GTAAATATTGCAGATTTTCAAGAGAATCAGTGGGTGACTTGTTTCCAGGAGTC
TGCTGAAGCTATCCTTGGACAAAATGCTGCTTATCTTGGGGAATTAAAAGAC
AAGAATGAACAGGCATTTGAAGAAGTTTTCCAGAATGCCAACTTCCGATCTT
TCATATTCAGAGTCAGGGTCAAAGTGGAGACCTACAACGACGAGTCTCGAAT
TAAGGCCACTGTGATGGACGTGAAGCCCGTGGACTACAGAGAGTATGGCCGA
AGGCTGGTCATGAGCATCAGGAGAAGTGCATTGATGTGA
Human RPA1 Protein Sequence, Variant 2 (SEQ ID NO: 47)
MQKGDTNIKPILQVINIRPITTGNSPPRYRLLMSDGLNTLSSFMLATQLNPLVEEE
QLSSNCVCQIHRFIVNTLKDGRRVVILMELEVLKSAEAVGVKIGNPVPYNEGLGQ
PQVAPPAPAASPAASSRPQPQNGSSGMGSTVSKAYGASKTFGKAAGPSLSHTSG
GTQSKVVPIASLTPYQSKWTICARVTNKSQIRTWSNSRGEGKLFSLELVDESGEIR
ATAFNEQVDKFFPLIEVNKVYYFSKGTLKIANKQFTAVKNDYEMTFNNETSVMP
CEDDHHLPTVQFDFTGIDDLENKSKDSLVDIIGICKSYEDATKITVRSNNREVAKR
NIYLMDTSGKVVTATLWGEDADKFDGSRQPVLAIKGARVSDFGGRSLSVLSSSTI
IANPDIPEAYKLRGWFDAEGQALDGVSISDLKSGGVGGSNTNWKTLYEVKSENL
GQGDKPDYFSSVATVVYLRKENCMYQACPTQDCNKKVIDQQNGLYRCEKCDTE
FPNFKYRMILSVNIADFQENQWVTCFQESAEAILGQNAAYLGELKDKNEQAFEE
VFQNANFRSFIFRVRVKVETYNDESRIKATVMDVKPVDYREYGRRLVMSIRRSA
LM
Human RPA1 cDNA Sequence, Variant 3 (SEQ ID NO: 48)
ATGGTCGGCCAACTGAGCGAGGGGGCCATTGCGGCCATCATGCAGAAGGGG
GATACAAACATAAAGCCCATCCTCCAAGTCATCAACATCCGTCCCATTACTA
CGGGGAATAGTCCGCCGCGTTATCGACTGCTCATGAGTGATGGATTGAACAC
TCTATCCTCTTTCATGTTGGCGACACAGTTGAACCCTCTCGTGGAGGAAGAAC
AATTGTCCAGCAACTGTGTATGCCAGATTCACAGATTTATTGTGAACACTCTG
AAAGACGGAAGGAGAGTAGTTATCTTGATGGAATTAGAAGTTTTGAAGTCAG
CTGAAGCAGTTGGAGTGAAGATTGGCAATCCAGTGCCCTATAATGAAGGACT
CGGGCAGCCGCAAGTAGCTCCTCCAGCGCCAGCAGCCAGCCCAGCAGCAAG
CAGCAGGCCCCAGCCGCAGAATGGAAGCTCGGGAATGGGTTCTACTGTTTCT
AAGGCTTATGGTGCTTCAAAGACATTTGGAAAAGCTGCAGGTCCCAGCCTGT
CACACACTTCTGGGGGAACACAGTCCAAAGTGGTGCCCATTGCCAGCCTCAC
TCCTTACCAGTCCAAGTGGACCATTTGTGCTCGTGTTACCAACAAAAGTCAGA
TCCGTACCTGGAGCAACTCCCGAGGGGAAGGGAAGCTTTTCTCCCTAGAACT
GGTTGACGAAAGTGGTGAAATCCGAGCTACAGCTTTCAATGAGCAAGTGGAC
AAGTTCTTTCCTCTTATTGAAGTGAACAAGGTGTATTATTTCTCGAAAGGCAC
CCTGAAGATTGCTAACAAGCAGTTCACAGCTGTTAAAAATGACTACGAGATG
ACCTTCAATAACGAGACTTCCGTCATGCCCTGTGAGGACGACCATCATTTACC
TACGGTTCAGTTTGATTTCACGGGGATTGATGACCTCGAGAACAAGTCGAAA
GACTCACTTGTAGACATCATCGGGATCTGCAAGAGCTATGAAGACGCCACTA
AAATCACAGTGAGGTCTAACAACAGAGAAGTTGCCAAGAGGAATATCTACTT
GATGGACACATCCGGGAAGGTGGTGACTGCTACACTGTGGGGGGAAGATGCT
GATAAATTTGATGGTTCTAGACAGCCCGTGTTGGCTATCAAAGGAGCCCGAG
TCTCTGATTTCGGTGGACGGAGCCTCTCCGTGCTGTCTTCAAGCACTATCATT
GCGAATCCTGACATCCCAGAGGCCTATAAGCTTCGTGGATGGTTTGACGCAG
AAGGACAAGCCTTAGATGGTGTTTCCATCTCTGATCTAAAGAGCGGCGGAGT
CGGAGGGAGTAACACCAACTGGAAAACCTTGTATGAGGTCAAATCCGAGAA
CCTGGGCCAAGGCGACAAGGTAAATATTGCAGATTTTCAAGAGAATCAGTGG
GTGACTTGTTTCCAGGAGTCTGCTGAAGCTATCCTTGGACAAAATGCTGCTTA
TCTTGGGGAATTAAAAGACAAGAATGAACAGGCATTTGAAGAAGTTTTCCAG
AATGCCAACTTCCGATCTTTCATATTCAGAGTCAGGGTCAAAGTGGAGACCT
ACAACGACGAGTCTCGAATTAAGGCCACTGTGATGGACGTGAAGCCCGTGGA
CTACAGAGAGTATGGCCGAAGGCTGGTCATGAGCATCAGGAGAAGTGCATTG
ATGTGA
Human RPA1 Protein Sequence, Variant 3 (SEQ ID NO: 49)
MVGQLSEGAIAAIMQKGDTNIKPILQVINIRPITTGNSPPRYRLLMSDGLNTLSSF
MLATQLNPLVEEEQLSSNCVCQIHRFIVNTLKDGRRVVILMELEVLKSAEAVGVK
IGNPVPYNEGLGQPQVAPPAPAASPAASSRPQPQNGSSGMGSTVSKAYGASKTF
GKAAGPSLSHTSGGTQSKVVPIASLTPYQSKWTICARVTNKSQIRTWSNSRGEGK
LFSLELVDESGEIRATAFNEQVDKFFPLIEVNKVYYFSKGTLKIANKQFTAVKND
YEMTFNNETSVMPCEDDHHLPTVQFDFTGIDDLENKSKDSLVDIIGICKSYEDAT
KITVRSNNREVAKRNIYLMDTSGKVVTATLWGEDADKFDGSRQPVLAIKGARVS
DFGGRSLSVLSSSTIIANPDIPEAYKLRGWFDAEGQALDGVSISDLKSGGVGGSNT
NWKTLYEVKSENLGQGDKVNIADFQENQWVTCFQESAEAILGQNAAYLGELKD
KNEQAFEEVFQNANFRSFIFRVRVKVETYNDESRIKATVMDVKPVDYREYGRRL
VMSIRRSALM
Human RAD51 cDNA Sequence, Variant 1 (SEQ ID NO: 50)
ATGGCAATGCAGATGCAGCTTGAAGCAAATGCAGATACTTCAGTGGAAGAAG
AAAGCTTTGGCCCACAACCCATTTCACGGTTAGAGCAGTGTGGCATAAATGC
CAACGATGTGAAGAAATTGGAAGAAGCTGGATTCCATACTGTGGAGGCTGTT
GCCTATGCGCCAAAGAAGGAGCTAATAAATATTAAGGGAATTAGTGAAGCCA
AAGCTGATAAAATTCTGGCTGAGGCAGCTAAATTAGTTCCAATGGGTTTCAC
CACTGCAACTGAATTCCACCAAAGGCGGTCAGAGATCATACAGATTACTACT
GGCTCCAAAGAGCTTGACAAACTACTTCAAGGTGGAATTGAGACTGGATCTA
TCACAGAAATGTTTGGAGAATTCCGAACTGGGAAGACCCAGATCTGTCATAC
GCTAGCTGTCACCTGCCAGCTTCCCATTGACCGGGGTGGAGGTGAAGGAAAG
GCCATGTACATTGACACTGAGGGTACCTTTAGGCCAGAACGGCTGCTGGCAG
TGGCTGAGAGGTATGGTCTCTCTGGCAGTGATGTCCTGGATAATGTAGCATAT
GCTCGAGCGTTCAACACAGACCACCAGACCCAGCTCCTTTATCAAGCATCAG
CCATGATGGTAGAATCTAGGTATGCACTGCTTATTGTAGACAGTGCCACCGCC
CTTTACAGAACAGACTACTCGGGTCGAGGTGAGCTTTCAGCCAGGCAGATGC
ACTTGGCCAGGTTTCTGCGGATGCTTCTGCGACTCGCTGATGAGTTTGGTGTA
GCAGTGGTAATCACTAATCAGGTGGTAGCTCAAGTGGATGGAGCAGCGATGT
TTGCTGCTGATCCCAAAAAACCTATTGGAGGAAATATCATCGCCCATGCATC
AACAACCAGATTGTATCTGAGGAAAGGAAGAGGGGAAACCAGAATCTGCAA
AATCTACGACTCTCCCTGTCTTCCTGAAGCTGAAGCTATGTTCGCCATTAATG
CAGATGGAGTGGGAGATGCCAAAGACTGA
Human RAD51 Protein Sequence, Variant 1 (SEQ ID NO: 51)
MAMQMQLEANADTSVEEESFGPQPISRLEQCGINANDVKKLEEAGFHTVEAVAY
APKKELINIKGISEAKADKILAEAAKLVPMGFTTATEFHQRRSEIIQITTGSKELDK
LLQGGIETGSITEMFGEFRTGKTQICHTLAVTCQLPIDRGGGEGKAMYIDTEGTFR
PERLLAVAERYGLSGSDVLDNVAYARAFNTDHQTQLLYQASAMMVESRYALLI
VDSATALYRTDYSGRGELSARQMHLARFLRMLLRLADEFGVAVVITNQVVAQV
DGAAMFAADPKKPIGGNIIAHASTTRLYLRKGRGETRICKIYDSPCLPEAEAMFAI
NADGVGDAKD
Human RAD51 cDNA Sequence, Variant 2 (SEQ ID NO: 52)
ATGGCAATGCAGATGCAGCTTGAAGCAAATGCAGATACTTCAGTGGAAGAAG
AAAGCTTTGGCCCACAACCCATTTCACGGTTAGAGCAGTGTGGCATAAATGC
CAACGATGTGAAGAAATTGGAAGAAGCTGGATTCCATACTGTGGAGGCTGTT
GCCTATGCGCCAAAGAAGGAGCTAATAAATATTAAGGGAATTAGTGAAGCCA
AAGCTGATAAAATTCTGACGGAGTCTCGCTCTGTTGCCAGGCTGGAGTGCAA
TAGCGTGATCTTGGTCTACTGCACCCTCCGCCTCTCAGGTTCAAGTGATTCTC
CTGCCTCAGCCTCCCGAGTAGTTGGGACTACAGGTGGAATTGAGACTGGATC
TATCACAGAAATGTTTGGAGAATTCCGAACTGGGAAGACCCAGATCTGTCAT
ACGCTAGCTGTCACCTGCCAGCTTCCCATTGACCGGGGTGGAGGTGAAGGAA
AGGCCATGTACATTGACACTGAGGGTACCTTTAGGCCAGAACGGCTGCTGGC
AGTGGCTGAGAGGTATGGTCTCTCTGGCAGTGATGTCCTGGATAATGTAGCA
TATGCTCGAGCGTTCAACACAGACCACCAGACCCAGCTCCTTTATCAAGCAT
CAGCCATGATGGTAGAATCTAGGTATGCACTGCTTATTGTAGACAGTGCCAC
CGCCCTTTACAGAACAGACTACTCGGGTCGAGGTGAGCTTTCAGCCAGGCAG
ATGCACTTGGCCAGGTTTCTGCGGATGCTTCTGCGACTCGCTGATGAGTTTGG
TGTAGCAGTGGTAATCACTAATCAGGTGGTAGCTCAAGTGGATGGAGCAGCG
ATGTTTGCTGCTGATCCCAAAAAACCTATTGGAGGAAATATCATCGCCCATGC
ATCAACAACCAGATTGTATCTGAGGAAAGGAAGAGGGGAAACCAGAATCTG
CAAAATCTACGACTCTCCCTGTCTTCCTGAAGCTGAAGCTATGTTCGCCATTA
ATGCAGATGGAGTGGGAGATGCCAAAGACTGA
Human RAD51 Protein Sequence, Variant 2 (SEQ ID NO: 53)
MAMQMQLEANADTSVEEESFGPQPISRLEQCGINANDVKKLEEAGFHTVEAVAY
APKKELINIKGISEAKADKILTESRSVARLECNSVILVYCTLRLSGSSDSPASASRV
VGTTGGIETGSITEMFGEFRTGKTQICHTLAVTCQLPIDRGGGEGKAMYIDTEGTF
RPERLLAVAERYGLSGSDVLDNVAYARAFNTDHQTQLLYQASAMMVESRYALL
IVDSATALYRTDYSGRGELSARQMHLARFLRMLLRLADEFGVAVVITNQVVAQ
VDGAAMFAADPKKPIGGNIIAHASTTRLYLRKGRGETRICKIYDSPCLPEAEAMF
AINADGVGDAKD
Human RAD51 cDNA Sequence, Variant 3 (SEQ ID NO: 54)
ATGGCAATGCAGATGCAGCTTGAAGCAAATGCAGATACTTCAGTGGAAGAAG
AAAGCTTTGGCCCACAACCCATTTCACGGTTAGAGCAGTGTGGCATAAATGC
CAACGATGTGAAGAAATTGGAAGAAGCTGGATTCCATACTGTGGAGGCTGTT
GCCTATGCGCCAAAGAAGGAGCTAATAAATATTAAGGGAATTAGTGAAGCCA
AAGCTGATAAAATTCTGGCTGAGGCAGCTAAATTAGTTCCAATGGGTTTCAC
CACTGCAACTGAATTCCACCAAAGGCGGTCAGAGATCATACAGATTACTACT
GGCTCCAAAGAGCTTGACAAACTACTTCAAGGTGGAATTGAGACTGGATCTA
TCACAGAAATGTTTGGAGAATTCCGAACTGGGAAGACCCAGATCTGTCATAC
GCTAGCTGTCACCTGCCAGCTTCCCATTGACCGGGGTGGAGGTGAAGGAAAG
GCCATGTACATTGACACTGAGGGTACCTTTAGGCCAGAACGGCTGCTGGCAG
TGGCTGAGAGGTATGGTCTCTCTGGCAGTGATGTCCTGGATAATGTAGCATAT
GCTCGAGCGTTCAACACAGACCACCAGACCCAGCTCCTTTATCAAGCATCAG
CCATGATGGTAGAATCTAGGTATGCACTGCTTATTGTAGACAGTGCCACCGCC
CTTTACAGAACAGACTACTCGGGTCGAGGTGAGCTTTCAGCCAGGCAGATGC
ACTTGGCCAGGTTTCTGCGGATGCTTCTGCGACTCGCTGATGAGATTGTATCT
GAGGAAAGGAAGAGGGGAAACCAGAATCTGCAAAATCTACGACTCTCCCTG
TCTTCCTGA
Human RAD51 Protein Sequence, Variant 3 (SEQ ID NO: 55)
MAMQMQLEANADTSVEEESFGPQPISRLEQCGINANDVKKLEEAGFHTVEAVAY
APKKELINIKGISEAKADKILAEAAKLVPMGFTTATEFHQRRSEIIQITTGSKELDK
LLQGGIETGSITEMFGEFRTGKTQICHTLAVTCQLPIDRGGGEGKAMYIDTEGTFR
PERLLAVAERYGLSGSDVLDNVAYARAFNTDHQTQLLYQASAMMVESRYALLI
VDSATALYRTDYSGRGELSARQMHLARFLRMLLRLADEIVSEERKRGNQNLQN
LRLSLSS
Human MUS81 cDNA Sequence, Variant 1 (SEQ ID NO: 56)
ATGGCGGCCCCGGTCCGCCTGGGCCGGAAGCGCCCGCTGCCTGCCTGTCCCA
ACCCGCTCTTCGTTCGCTGGCTGACCGAGTGGCGGGACGAGGCGACCCGCAG
CAGGCGCCGCACGCGCTTCGTATTTCAGAAGGCGCTGCGTTCCCTCCGACGG
TACCCACTGCCGCTGCGCAGCGGGAAGGAAGCTAAGATCCTACAGCACTTCG
GAGACGGGCTCTGCCGGATGCTGGACGAGCGGCTGCAGCGGCACCGAACAT
CGGGCGGTGACCATGCCCCGGACTCACCATCTGGAGAGAACAGTCCAGCCCC
GCAGGGGCGACTTGCGGAAGTCCAGGACTCTTCCATGCCAGTTCCTGCCCAG
CCCAAAGCGGGAGGCTCTGGCAGCTACTGGCCAGCTCGGCACTCAGGAGCCC
GAGTGATACTGCTGGTGCTCTACCGGGAGCACCTGAATCCTAATGGTCACCA
CTTCTTAACCAAGGAGGAGCTGCTGCAGAGGTGTGCTCAGAAGTCCCCCAGG
GTAGCCCCTGGGAGTGCTCGACCCTGGCCAGCCCTCCGCTCCCTCCTTCACAG
GAACCTGGTCCTCAGGACACACCAGCCAGCCAGGTACTCATTGACCCCAGAG
GGCCTGGAGCTGGCCCAGAAGTTGGCCGAGTCAGAAGGCCTGAGCTTGCTGA
ATGTGGGCATCGGGCCCAAGGAGCCCCCTGGGGAGGAGACAGCAGTGCCAG
GAGCAGCTTCAGCAGAGCTTGCCAGTGAAGCAGGGGTCCAGCAGCAGCCACT
GGAGCTGAGGCCTGGAGAGTACAGGGTGCTGTTGTGTGTGGACATTGGCGAG
ACCCGGGGGGGCGGGCACAGGCCGGAGCTGCTCCGAGAGCTACAGCGGCTG
CACGTGACCCACACGGTGCGCAAGCTGCACGTTGGAGATTTTGTGTGGGTGG
CCCAGGAGACCAATCCTAGAGACCCAGCAGCAAACCCTGGGGAGTTGGTACT
GGATCACATTGTGGAGCGCAAGCGACTGGATGACCTTTGCAGCAGCATCATC
GACGGCCGCTTCCGGGAGCAGAAGTTCCGGCTGAAGCGCTGTGGTCTGGAGC
GCCGGGTATACCTGGTGGAAGAGCATGGTTCCGTCCACAACCTCAGCCTTCC
TGAGAGCACACTGCTGCAGGCTGTCACCAACACTCAGGTCATTGATGGCTTTT
TTGTGAAGCGCACAGCAGACATTAAGGAGTCAGCCGCCTACCTGGCCCTCTT
GACGCGGGGCCTGCAGAGACTCTACCAGGGCCACACCCTACGCAGCCGCCCC
TGGGGAACCCCTGGGAACCCTGAATCAGGGGCCATGACCTCTCCAAACCCTC
TCTGCTCACTCCTCACCTTCAGTGACTTCAACGCAGGAGCCATCAAGAATAA
GGCCCAGTCGGTGCGAGAAGTGTTTGCCCGGCAGCTGATGCAGGTGCGCGGA
GTGAGTGGGGAGAAGGCAGCAGCCCTGGTGGATCGATACAGCACCCCTGCC
AGCCTCCTGGCCGCCTATGATGCCTGTGCCACCCCCAAGGAACAAGAGACAC
TGCTGAGCACCATTAAGTGTGGGCGTCTACAGAGGAATCTGGGGCCTGCTCT
GAGCAGGACCTTATCCCAGCTCTACTGCAGCTACGGCCCCTTGACCTGA
Human MUS81 Protein Sequence, Variant 1 (SEQ ID NO: 57)
MAAPVRLGRKRPLPACPNPLFVRWLTEWRDEATRSRRRTRFVFQKALRSLRRYP
LPLRSGKEAKILQHFGDGLCRMLDERLQRHRTSGGDHAPDSPSGENSPAPQGRL
AEVQDSSMPVPAQPKAGGSGSYWPARHSGARVILLVLYREHLNPNGHHFLTKEE
LLQRCAQKSPRVAPGSARPWPALRSLLHRNLVLRTHQPARYSLTPEGLELAQKL
AESEGLSLLNVGIGPKEPPGEETAVPGAASAELASEAGVQQQPLELRPGEYRVLL
CVDIGETRGGGHRPELLRELQRLHVTHTVRKLHVGDFVWVAQETNPRDPAANP
GELVLDHIVERKRLDDLCSSIIDGRFREQKFRLKRCGLERRVYLVEEHGSVHNLS
LPESTLLQAVTNTQVIDGFFVKRTADIKESAAYLALLTRGLQRLYQGHTLRSRPW
GTPGNPESGAMTSPNPLCSLLTFSDFNAGAIKNKAQSVREVFARQLMQVRGVSG
EKAAALVDRYSTPASLLAAYDACATPKEQETLLSTIKCGRLQRNLGPALSRTLSQ
LYCSYGPLT
Human MUS81 cDNA Sequence, Variant 2 (SEQ ID NO: 58)
ATGGCGGCCCCGGTCCGCCTGGGCCGGAAGCGCCCGCTGCCTGCCTGTCCCA
ACCCGCTCTTCGTTCGCTGGCTGACCGAGTGGCGGGACGAGGCGACCCGCAG
CAGGCGCCGCACGCGCTTCGTATTTCAGAAGGCGCTGCGTTCCCTCCGACGG
TACCCACTGCCGCTGCGCAGCGGGAAGGAAGCTAAGATCCTACAGCACTTCG
GAGACGGGCTCTGCCGGATGCTGGACGAGCGGCTGCAGCGGCACCGAACAT
CGGGCGGTGACCATGCCCCGGACTCACCATCTGGAGAGAACAGTCCAGCCCC
GCAGGGGCGACTTGCGGAAGTCCAGGACTCTTCCATGCCAGTTCCTGCCCAG
CCCAAAGCGGGAGGCTCTGGCAGCTACTGGCCAGCTCGGCACTCAGGAGCCC
GAGTGATACTGCTGGTGCTCTACCGGGAGCACCTGAATCCTAATGGTCACCA
CTTCTTAACCAAGGAGGAGCTGCTGCAGAGGTGTGCTCAGAAGTCCCCCAGG
GTAGCCCCTGGGAGTGCTCGACCCTGGCCAGCCCTCCGCTCCCTCCTTCACAG
GAACCTGGTCCTCAGGACACACCAGCCAGCCAGGTACTCATTGACCCCAGAG
GGCCTGGAGCTGGCCCAGAAGTTGGCCGAGTCAGAAGGCCTGAGCTTGCTGA
ATGTGGGCATCGGGCCCAAGGAGCCCCCTGGGGAGGAGACAGCAGTGCCAG
GAGCAGCTTCAGCAGAGCTTGCCAGTGAAGCAGGGGTCCAGCAGCAGCCACT
GGAGCTGAGGCCTGGAGAGTACAGGGTGCTGTTGTGTGTGGACATTGGCGAG
ACCCGGGGGGGCGGGCACAGGCCGGAGCTGCTCCGAGAGCTACAGCGGCTG
CACGTGACCCACACGGTGCGCAAGCTGCACGTTGGAGATTTTGTGTGGGTGG
CCCAGGAGACCAATCCTAGAGACCCAGCAAACCCTGGGGAGTTGGTACTGGA
TCACATTGTGGAGCGCAAGCGACTGGATGACCTTTGCAGCAGCATCATCGAC
GGCCGCTTCCGGGAGCAGAAGTTCCGGCTGAAGCGCTGTGGTCTGGAGCGCC
GGGTATACCTGGTGGAAGAGCATGGTTCCGTCCACAACCTCAGCCTTCCTGA
GAGCACACTGCTGCAGGCTGTCACCAACACTCAGGTCATTGATGGCTTTTTTG
TGAAGCGCACAGCAGACATTAAGGAGTCAGCCGCCTACCTGGCCCTCTTGAC
GCGGGGCCTGCAGAGACTCTACCAGGGCCACACCCTACGCAGCCGCCCCTGG
GGAACCCCTGGGAACCCTGAATCAGGGGCCATGACCTCTCCAAACCCTCTCT
GCTCACTCCTCACCTTCAGTGACTTCAACGCAGGAGCCATCAAGAATAAGGC
CCAGTCGGTGCGAGAAGTGTTTGCCCGGCAGCTGATGCAGGTGCGCGGAGTG
AGTGGGGAGAAGGCAGCAGCCCTGGTGGATCGATACAGCACCCCTGCCAGC
CTCCTGGCCGCCTATGATGCCTGTGCCACCCCCAAGGAACAAGAGACACTGC
TGAGCACCATTAAGTGTGGGCGTCTACAGAGGAATCTGGGGCCTGCTCTGAG
CAGGACCTTATCCCAGCTCTACTGCAGCTACGGCCCCTTGACCTGA
Human MUS81 Protein Sequence, Variant 2 (SEQ ID NO: 59)
MAAPVRLGRKRPLPACPNPLFVRWLTEWRDEATRSRRRTRFVFQKALRSLRRYP
LPLRSGKEAKILQHFGDGLCRMLDERLQRHRTSGGDHAPDSPSGENSPAPQGRL
AEVQDSSMPVPAQPKAGGSGSYWPARHSGARVILLVLYREHLNPNGHHFLTKEE
LLQRCAQKSPRVAPGSARPWPALRSLLHRNLVLRTHQPARYSLTPEGLELAQKL
AESEGLSLLNVGIGPKEPPGEETAVPGAASAELASEAGVQQQPLELRPGEYRVLL
CVDIGETRGGGHRPELLRELQRLHVTHTVRKLHVGDFVWVAQETNPRDPANPG
ELVLDHIVERKRLDDLCSSIIDGRFREQKFRLKRCGLERRVYLVEEHGSVHNLSLP
ESTLLQAVTNTQVIDGFFVKRTADIKESAAYLALLTRGLQRLYQGHTLRSRPWGT
PGNPESGAMTSPNPLCSLLTFSDFNAGAIKNKAQSVREVFARQLMQVRGVSGEK
AAALVDRYSTPASLLAAYDACATPKEQETLLSTIKCGRLQRNLGPALSRTLSQLY
CSYGPLT
Human IFI16 cDNA Sequence, Variant 1 (SEQ ID NO: 60)
ATGGGAAAAAAATACAAGAACATTGTTCTACTAAAAGGATTAGAGGTCATCA
ATGATTATCATTTTAGAATGGTTAAGTCCTTACTGAGCAACGATTTAAAACTT
AATTTAAAAATGAGAGAAGAGTATGACAAAATTCAGATTGCTGACTTGATGG
AAGAAAAGTTCCGAGGTGATGCTGGTTTGGGCAAACTAATAAAAATTTTCGA
AGATATACCAACGCTTGAAGACCTGGCTGAAACTCTTAAAAAAGAAAAGTTA
AAAGTAAAAGGACCAGCCCTATCAAGAAAGAGGAAGAAGGAAGTGGATGCT
ACTTCACCTGCACCCTCCACAAGCAGCACTGTCAAAACTGAAGGAGCAGAGG
CAACTCCTGGAGCTCAGAAAAGAAAAAAATCAACCAAAGAAAAGGCTGGAC
CCAAAGGGAGTAAGGTGTCCGAGGAACAGACTCAGCCTCCCTCTCCTGCAGG
AGCCGGCATGTCCACAGCCATGGGCCGTTCCCCATCTCCCAAGACCTCATTGT
CAGCTCCACCCAACAGTTCTTCAACTGAGAACCCGAAAACAGTGGCCAAATG
TCAGGTAACTCCCAGAAGAAATGTTCTCCAAAAACGCCCAGTGATAGTGAAG
GTACTGAGTACAACAAAGCCATTTGAATATGAGACCCCAGAAATGGAGAAA
AAAATAATGTTTCATGCTACAGTGGCTACACAGACACAGTTCTTCCATGTGAA
GGTTTTAAACACCAGCTTGAAGGAGAAATTCAATGGAAAGAAAATCATCATC
ATATCAGATTATTTGGAATATGATAGTCTCCTAGAGGTCAATGAAGAATCTAC
TGTATCTGAAGCTGGTCCTAACCAAACGTTTGAGGTTCCAAATAAAATCATCA
ACAGAGCAAAGGAAACTCTGAAGATTGATATTCTTCACAAACAAGCTTCAGG
AAATATTGTATATGGGGTATTTATGCTACATAAGAAAACAGTAAATCAGAAG
ACCACAATCTACGAAATTCAGGATGATAGAGGAAAAATGGATGTAGTGGGG
ACAGGACAATGTCACAATATCCCCTGTGAAGAAGGAGATAAGCTCCAACTTT
TCTGCTTTCGACTTAGAAAAAAGAACCAGATGTCAAAACTGATTTCAGAAAT
GCATAGTTTTATCCAGATAAAGAAAAAAACAAACCCGAGAAACAATGACCCC
AAGAGCATGAAGCTACCCCAGGAACAGCGTCAGCTTCCATATCCTTCAGAGG
CCAGCACAACCTTCCCTGAGAGCCATCTTCGGACTCCTCAGATGCCACCAAC
AACTCCATCCAGCAGTTTCTTCACCAAGAAAAGTGAAGACACAATCTCCAAA
ATGAATGACTTCATGAGGATGCAGATACTGAAGGAAGGGAGTCATTTTCCAG
GACCGTTCATGACCAGCATAGGCCCAGCTGAGAGCCATCCCCACACTCCTCA
GATGCCTCCATCAACACCAAGCAGCAGTTTCTTAACCACGAAAAGTGAAGAC
ACAATCTCCAAAATGAATGACTTCATGAGGATGCAGATACTGAAGGAAGGGA
GTCATTTTCCAGGACCGTTCATGACCAGCATAGGCCCAGCTGAGAGCCATCC
CCACACTCCTCAGATGCCTCCATCAACACCAAGCAGCAGTTTCTTAACCACGT
TGAAACCAAGACTGAAGACTGAACCTGAAGAAGTTTCCATAGAAGACAGTGC
CCAGAGTGACCTCAAAGAAGTGATGGTGCTGAACGCAACAGAATCATTTGTA
TATGAGCCCAAAGAGCAGAAGAAAATGTTTCATGCCACAGTGGCAACTGAGA
ATGAAGTCTTCCGAGTGAAGGTTTTTAATATTGACCTAAAGGAGAAGTTCAC
CCCAAAGAAGATCATTGCCATAGCAAATTATGTTTGCCGCAATGGGTTCCTG
GAGGTATATCCTTTCACACTTGTGGCTGATGTGAATGCTGACCGAAACATGG
AGATCCCAAAAGGATTGATTAGAAGTGCCAGCGTAACTCCTAAAATCAATCA
GCTTTGCTCACAAACTAAAGGAAGTTTTGTGAATGGGGTGTTTGAGGTACAT
AAGAAAAATGTAAGGGGTGAATTCACTTATTATGAAATACAAGATAATACAG
GGAAGATGGAAGTGGTGGTGCATGGACGACTGACCACAATCAACTGTGAGG
AAGGAGATAAACTGAAACTCACCTGCTTTGAATTGGCACCGAAAAGTGGGAA
TACCGGGGAGTTGAGATCTGTAATTCATAGTCACATCAAGGTCATCAAGACC
AGGAAAAACAAGAAAGACATACTCAATCCTGATTCAAGTATGGAAACTTCAC
CAGACTTTTTCTTCTAA
Human IFI16 Protein Sequence, Variant 1 (SEQ ID NO: 61)
MGKKYKNIVLLKGLEVINDYHFRMVKSLLSNDLKLNLKMREEYDKIQIADLMEE
KFRGDAGLGKLIKIFEDIPTLEDLAETLKKEKLKVKGPALSRKRKKEVDATSPAPS
TSSTVKTEGAEATPGAQKRKKSTKEKAGPKGSKVSEEQTQPPSPAGAGMSTAMG
RSPSPKTSLSAPPNSSSTENPKTVAKCQVTPRRNVLQKRPVIVKVLSTTKPFEYET
PEMEKKIMFHATVATQTQFFHVKVLNTSLKEKFNGKKIIIISDYLEYDSLLEVNEE
STVSEAGPNQTFEVPNKIINRAKETLKIDILHKQASGNIVYGVFMLHKKTVNQKT
TIYEIQDDRGKMDVVGTGQCHNIPCEEGDKLQLFCFRLRKKNQMSKLISEMHSFI
QIKKKTNPRNNDPKSMKLPQEQRQLPYPSEASTTFPESHLRTPQMPPTTPSSSFFT
KKSEDTISKMNDFMRMQILKEGSHFPGPFMTSIGPAESHPHTPQMPPSTPSSSFLT
TKSEDTISKMNDFMRMQILKEGSHFPGPFMTSIGPAESHPHTPQMPPSTPSSSFLTT
LKPRLKTEPEEVSIEDSAQSDLKEVMVLNATESFVYEPKEQKKMFHATVATENE
VFRVKVFNIDLKEKFTPKKIIAIANYVCRNGFLEVYPFTLVADVNADRNMEIPKG
LIRSASVTPKINQLCSQTKGSFVNGVFEVHKKNVRGEFTYYEIQDNTGKMEVVV
HGRLTTINCEEGDKLKLTCFELAPKSGNTGELRSVIHSHIKVIKTRKNKKDILNPD
SSMETSPDFFF
Human IFI16 cDNA Sequence, Variant 2 (SEQ ID NO: 62)
ATGGGAAAAAAATACAAGAACATTGTTCTACTAAAAGGATTAGAGGTCATCA
ATGATTATCATTTTAGAATGGTTAAGTCCTTACTGAGCAACGATTTAAAACTT
AATTTAAAAATGAGAGAAGAGTATGACAAAATTCAGATTGCTGACTTGATGG
AAGAAAAGTTCCGAGGTGATGCTGGTTTGGGCAAACTAATAAAAATTTTCGA
AGATATACCAACGCTTGAAGACCTGGCTGAAACTCTTAAAAAAGAAAAGTTA
AAAGTAAAAGGACCAGCCCTATCAAGAAAGAGGAAGAAGGAAGTGGATGCT
ACTTCACCTGCACCCTCCACAAGCAGCACTGTCAAAACTGAAGGAGCAGAGG
CAACTCCTGGAGCTCAGAAAAGAAAAAAATCAACCAAAGAAAAGGCTGGAC
CCAAAGGGAGTAAGGTGTCCGAGGAACAGACTCAGCCTCCCTCTCCTGCAGG
AGCCGGCATGTCCACAGCCATGGGCCGTTCCCCATCTCCCAAGACCTCATTGT
CAGCTCCACCCAACAGTTCTTCAACTGAGAACCCGAAAACAGTGGCCAAATG
TCAGGTAACTCCCAGAAGAAATGTTCTCCAAAAACGCCCAGTGATAGTGAAG
GTACTGAGTACAACAAAGCCATTTGAATATGAGACCCCAGAAATGGAGAAA
AAAATAATGTTTCATGCTACAGTGGCTACACAGACACAGTTCTTCCATGTGAA
GGTTTTAAACACCAGCTTGAAGGAGAAATTCAATGGAAAGAAAATCATCATC
ATATCAGATTATTTGGAATATGATAGTCTCCTAGAGGTCAATGAAGAATCTAC
TGTATCTGAAGCTGGTCCTAACCAAACGTTTGAGGTTCCAAATAAAATCATCA
ACAGAGCAAAGGAAACTCTGAAGATTGATATTCTTCACAAACAAGCTTCAGG
AAATATTGTATATGGGGTATTTATGCTACATAAGAAAACAGTAAATCAGAAG
ACCACAATCTACGAAATTCAGGATGATAGAGGAAAAATGGATGTAGTGGGG
ACAGGACAATGTCACAATATCCCCTGTGAAGAAGGAGATAAGCTCCAACTTT
TCTGCTTTCGACTTAGAAAAAAGAACCAGATGTCAAAACTGATTTCAGAAAT
GCATAGTTTTATCCAGATAAAGAAAAAAACAAACCCGAGAAACAATGACCCC
AAGAGCATGAAGCTACCCCAGGAACAGCGTCAGCTTCCATATCCTTCAGAGG
CCAGCACAACCTTCCCTGAGAGCCATCTTCGGACTCCTCAGATGCCACCAAC
AACTCCATCCAGCAGTTTCTTCACCAAGAAAAGTGAAGACACAATCTCCAAA
ATGAATGACTTCATGAGGATGCAGATACTGAAGGAAGGGAGTCATTTTCCAG
GACCGTTCATGACCAGCATAGGCCCAGCTGAGAGCCATCCCCACACTCCTCA
GATGCCTCCATCAACACCAAGCAGCAGTTTCTTAACCACGTTGAAACCAAGA
CTGAAGACTGAACCTGAAGAAGTTTCCATAGAAGACAGTGCCCAGAGTGACC
TCAAAGAAGTGATGGTGCTGAACGCAACAGAATCATTTGTATATGAGCCCAA
AGAGCAGAAGAAAATGTTTCATGCCACAGTGGCAACTGAGAATGAAGTCTTC
CGAGTGAAGGTTTTTAATATTGACCTAAAGGAGAAGTTCACCCCAAAGAAGA
TCATTGCCATAGCAAATTATGTTTGCCGCAATGGGTTCCTGGAGGTATATCCT
TTCACACTTGTGGCTGATGTGAATGCTGACCGAAACATGGAGATCCCAAAAG
GATTGATTAGAAGTGCCAGCGTAACTCCTAAAATCAATCAGCTTTGCTCACA
AACTAAAGGAAGTTTTGTGAATGGGGTGTTTGAGGTACATAAGAAAAATGTA
AGGGGTGAATTCACTTATTATGAAATACAAGATAATACAGGGAAGATGGAAG
TGGTGGTGCATGGACGACTGACCACAATCAACTGTGAGGAAGGAGATAAACT
GAAACTCACCTGCTTTGAATTGGCACCGAAAAGTGGGAATACCGGGGAGTTG
AGATCTGTAATTCATAGTCACATCAAGGTCATCAAGACCAGGAAAAACAAGA
AAGACATACTCAATCCTGATTCAAGTATGGAAACTTCACCAGACTTTTTCTTC
TAA
Human IFI16 Protein Sequence, Variant 2 (SEQ ID NO: 63)
MGKKYKNIVLLKGLEVINDYHFRMVKSLLSNDLKLNLKMREEYDKIQIADLMEE
KFRGDAGLGKLIKIFEDIPTLEDLAETLKKEKLKVKGPALSRKRKKEVDATSPAPS
TSSTVKTEGAEATPGAQKRKKSTKEKAGPKGSKVSEEQTQPPSPAGAGMSTAMG
RSPSPKTSLSAPPNSSSTENPKTVAKCQVTPRRNVLQKRPVIVKVLSTTKPFEYET
PEMEKKIMFHATVATQTQFFHVKVLNTSLKEKFNGKKIIIISDYLEYDSLLEVNEE
STVSEAGPNQTFEVPNKIINRAKETLKIDILHKQASGNIVYGVFMLHKKTVNQKT
TIYEIQDDRGKMDVVGTGQCHNIPCEEGDKLQLFCFRLRKKNQMSKLISEMHSFI
QIKKKTNPRNNDPKSMKLPQEQRQLPYPSEASTTFPESHLRTPQMPPTTPSSSFFT
KKSEDTISKMNDFMRMQILKEGSHFPGPFMTSIGPAESHPHTPQMPPSTPSSSFLT
TLKPRLKTEPEEVSIEDSAQSDLKEVMVLNATESFVYEPKEQKKMFHATVATENE
VFRVKVFNIDLKEKFTPKKIIAIANYVCRNGFLEVYPFTLVADVNADRNMEIPKG
LIRSASVTPKINQLCSQTKGSFVNGVFEVHKKNVRGEFTYYEIQDNTGKMEVVV
HGRLTTINCEEGDKLKLTCFELAPKSGNTGELRSVIHSHIKVIKTRKNKKDILNPD
SSMETSPDFFF
Human IFI16 cDNA Sequence, Variant 3 (SEQ ID NO: 64)
ATGGGAAAAAAATACAAGAACATTGTTCTACTAAAAGGATTAGAGGTCATCA
ATGATTATCATTTTAGAATGGTTAAGTCCTTACTGAGCAACGATTTAAAACTT
AATTTAAAAATGAGAGAAGAGTATGACAAAATTCAGATTGCTGACTTGATGG
AAGAAAAGTTCCGAGGTGATGCTGGTTTGGGCAAACTAATAAAAATTTTCGA
AGATATACCAACGCTTGAAGACCTGGCTGAAACTCTTAAAAAAGAAAAGTTA
AAAGTAAAAGGACCAGCCCTATCAAGAAAGAGGAAGAAGGAAGTGGATGCT
ACTTCACCTGCACCCTCCACAAGCAGCACTGTCAAAACTGAAGGAGCAGAGG
CAACTCCTGGAGCTCAGAACCCGAAAACAGTGGCCAAATGTCAGGTAACTCC
CAGAAGAAATGTTCTCCAAAAACGCCCAGTGATAGTGAAGGTACTGAGTACA
ACAAAGCCATTTGAATATGAGACCCCAGAAATGGAGAAAAAAATAATGTTTC
ATGCTACAGTGGCTACACAGACACAGTTCTTCCATGTGAAGGTTTTAAACACC
AGCTTGAAGGAGAAATTCAATGGAAAGAAAATCATCATCATATCAGATTATT
TGGAATATGATAGTCTCCTAGAGGTCAATGAAGAATCTACTGTATCTGAAGC
TGGTCCTAACCAAACGTTTGAGGTTCCAAATAAAATCATCAACAGAGCAAAG
GAAACTCTGAAGATTGATATTCTTCACAAACAAGCTTCAGGAAATATTGTAT
ATGGGGTATTTATGCTACATAAGAAAACAGTAAATCAGAAGACCACAATCTA
CGAAATTCAGGATGATAGAGGAAAAATGGATGTAGTGGGGACAGGACAATG
TCACAATATCCCCTGTGAAGAAGGAGATAAGCTCCAACTTTTCTGCTTTCGAC
TTAGAAAAAAGAACCAGATGTCAAAACTGATTTCAGAAATGCATAGTTTTAT
CCAGATAAAGAAAAAAACAAACCCGAGAAACAATGACCCCAAGAGCATGAA
GCTACCCCAGGAACAGCGTCAGCTTCCATATCCTTCAGAGGCCAGCACAACC
TTCCCTGAGAGCCATCTTCGGACTCCTCAGATGCCACCAACAACTCCATCCAG
CAGTTTCTTCACCAAGAAAAGTGAAGACACAATCTCCAAAATGAATGACTTC
ATGAGGATGCAGATACTGAAGGAAGGGAGTCATTTTCCAGGACCGTTCATGA
CCAGCATAGGCCCAGCTGAGAGCCATCCCCACACTCCTCAGATGCCTCCATC
AACACCAAGCAGCAGTTTCTTAACCACGAAAAGTGAAGACACAATCTCCAAA
ATGAATGACTTCATGAGGATGCAGATACTGAAGGAAGGGAGTCATTTTCCAG
GACCGTTCATGACCAGCATAGGCCCAGCTGAGAGCCATCCCCACACTCCTCA
GATGCCTCCATCAACACCAAGCAGCAGTTTCTTAACCACGTTGAAACCAAGA
CTGAAGACTGAACCTGAAGAAGTTTCCATAGAAGACAGTGCCCAGAGTGACC
TCAAAGAAGTGATGGTGCTGAACGCAACAGAATCATTTGTATATGAGCCCAA
AGAGCAGAAGAAAATGTTTCATGCCACAGTGGCAACTGAGAATGAAGTCTTC
CGAGTGAAGGTTTTTAATATTGACCTAAAGGAGAAGTTCACCCCAAAGAAGA
TCATTGCCATAGCAAATTATGTTTGCCGCAATGGGTTCCTGGAGGTATATCCT
TTCACACTTGTGGCTGATGTGAATGCTGACCGAAACATGGAGATCCCAAAAG
GATTGATTAGAAGTGCCAGCGTAACTCCTAAAATCAATCAGCTTTGCTCACA
AACTAAAGGAAGTTTTGTGAATGGGGTGTTTGAGGTACATAAGAAAAATGTA
AGGGGTGAATTCACTTATTATGAAATACAAGATAATACAGGGAAGATGGAAG
TGGTGGTGCATGGACGACTGACCACAATCAACTGTGAGGAAGGAGATAAACT
GAAACTCACCTGCTTTGAATTGGCACCGAAAAGTGGGAATACCGGGGAGTTG
AGATCTGTAATTCATAGTCACATCAAGGTCATCAAGACCAGGAAAAACAAGA
AAGACATACTCAATCCTGATTCAAGTATGGAAACTTCACCAGACTTTTTCTTC
TAA
Human IFI16 Protein Sequence, Variant 3 (SEQ ID NO: 65)
MGKKYKNIVLLKGLEVINDYHFRMVKSLLSNDLKLNLKMREEYDKIQIADLMEE
KFRGDAGLGKLIKIFEDIPTLEDLAETLKKEKLKVKGPALSRKRKKEVDATSPAPS
TSSTVKTEGAEATPGAQNPKTVAKCQVTPRRNVLQKRPVIVKVLSTTKPFEYETP
EMEKKIMFHATVATQTQFFHVKVLNTSLKEKFNGKKIIIISDYLEYDSLLEVNEES
TVSEAGPNQTFEVPNKIINRAKETLKIDILHKQASGNIVYGVFMLHKKTVNQKTTI
YEIQDDRGKMDVVGTGQCHNIPCEEGDKLQLFCFRLRKKNQMSKLISEMHSFIQI
KKKTNPRNNDPKSMKLPQEQRQLPYPSEASTTFPESHLRTPQMPPTTPSSSFFTKK
SEDTISKMNDFMRMQILKEGSHFPGPFMTSIGPAESHPHTPQMPPSTPSSSFLTTKS
EDTISKMNDFMRMQILKEGSHFPGPFMTSIGPAESHPHTPQMPPSTPSSSFLTTLKP
RLKTEPEEVSIEDSAQSDLKEVMVLNATESFVYEPKEQKKMFHATVATENEVFR
VKVFNIDLKEKFTPKKIIAIANYVCRNGFLEVYPFTLVADVNADRNMEIPKGLIRS
ASVTPKINQLCSQTKGSFVNGVFEVHKKNVRGEFTYYEIQDNTGKMEVVVHGRL
TTINCEEGDKLKLTCFELAPKSGNTGELRSVIHSHIKVIKTRKNKKDILNPDSSME
TSPDFFF
Human cGAS cDNA Sequence (SEQ ID NO: 66)
ATGCAGCCTTGGCACGGAAAGGCCATGCAGAGAGCTTCCGAGGCCGGAGCC
ACTGCCCCCAAGGCTTCCGCACGGAATGCCAGGGGCGCCCCGATGGATCCCA
CCGAGTCTCCGGCTGCCCCCGAGGCCGCCCTGCCTAAGGCGGGAAAGTTCGG
CCCCGCCAGGAAGTCGGGATCCCGGCAGAAAAAGAGCGCCCCGGACACCCA
GGAGAGGCCGCCCGTCCGCGCAACTGGGGCCCGCGCCAAAAAGGCCCCTCA
GCGCGCCCAGGACACGCAGCCGTCTGACGCCACCAGCGCCCCTGGGGCAGA
GGGGCTGGAGCCTCCTGCGGCTCGGGAGCCGGCTCTTTCCAGGGCTGGTTCTT
GCCGCCAGAGGGGCGCGCGCTGCTCCACGAAGCCAAGACCTCCGCCCGGGCC
CTGGGACGTGCCCAGCCCCGGCCTGCCGGTCTCGGCCCCCATTCTCGTACGG
AGGGATGCGGCGCCTGGGGCCTCGAAGCTCCGGGCGGTTTTGGAGAAGTTGA
AGCTCAGCCGCGATGATATCTCCACGGCGGCGGGGATGGTGAAAGGGGTTGT
GGACCACCTGCTGCTCAGACTGAAGTGCGACTCCGCGTTCAGAGGCGTCGGG
CTGCTGAACACCGGGAGCTACTATGAGCACGTGAAGATTTCTGCACCTAATG
AATTTGATGTCATGTTTAAACTGGAAGTCCCCAGAATTCAACTAGAAGAATA
TTCCAACACTCGTGCATATTACTTTGTGAAATTTAAAAGAAATCCGAAAGAA
AATCCTCTGAGTCAGTTTTTAGAAGGTGAAATATTATCAGCTTCTAAGATGCT
GTCAAAGTTTAGGAAAATCATTAAGGAAGAAATTAACGACATTAAAGATACA
GATGTCATCATGAAGAGGAAAAGAGGAGGGAGCCCTGCTGTAACACTTCTTA
TTAGTGAAAAAATATCTGTGGATATAACCCTGGCTTTGGAATCAAAAAGTAG
CTGGCCTGCTAGCACCCAAGAAGGCCTGCGCATTCAAAACTGGCTTTCAGCA
AAAGTTAGGAAGCAACTACGACTAAAGCCATTTTACCTTGTACCCAAGCATG
CAAAGGAAGGAAATGGTTTCCAAGAAGAAACATGGCGGCTATCCTTCTCTCA
CATCGAAAAGGAAATTTTGAACAATCATGGAAAATCTAAAACGTGCTGTGAA
AACAAAGAAGAGAAATGTTGCAGGAAAGATTGTTTAAAACTAATGAAATAC
CTTTTAGAACAGCTGAAAGAAAGGTTTAAAGACAAAAAACATCTGGATAAAT
TCTCTTCTTATCATGTGAAAACTGCCTTCTTTCACGTATGTACCCAGAACCCTC
AAGACAGTCAGTGGGACCGCAAAGACCTGGGCCTCTGCTTTGATAACTGCGT
GACATACTTTCTTCAGTGCCTCAGGACAGAAAAACTTGAGAATTATTTTATTC
CTGAATTCAATCTATTCTCTAGCAACTTAATTGACAAAAGAAGTAAGGAATTT
CTGACAAAGCAAATTGAATATGAAAGAAACAATGAGTTTCCAGTTTTTGATG
AATTTTGA
Human cGAS Protein Sequence (SEQ ID NO: 67)
MQPWHGKAMQRASEAGATAPKASARNARGAPMDPTESPAAPEAALPKAGKFG
PARKSGSRQKKSAPDTQERPPVRATGARAKKAPQRAQDTQPSDATSAPGAEGLE
PPAAREPALSRAGSCRQRGARCSTKPRPPPGPWDVPSPGLPVSAPILVRRDAAPG
ASKLRAVLEKLKLSRDDISTAAGMVKGVVDHLLLRLKCDSAFRGVGLLNTGSY
YEHVKISAPNEFDVMFKLEVPRIQLEEYSNTRAYYFVKFKRNPKENPLSQFLEGEI
LSASKMLSKFRKIIKEEINDIKDTDVIMKRKRGGSPAVTLLISEKISVDITLALESKS
SWPASTQEGLRIQNWLSAKVRKQLRLKPFYLVPKHAKEGNGFQEETWRLSFSHI
EKEILNNHGKSKTCCENKEEKCCRKDCLKLMKYLLEQLKERFKDKKHLDKFSSY
HVKTAFFHVCTQNPQDSQWDRKDLGLCFDNCVTYFLQCLRTEKLENYFIPEFNL
FSSNLIDKRSKEFLTKQIEYERNNEFPVFDEF
Human DDX41 cDNA Sequence, Variant 1 (SEQ ID NO: 68)
ATGGAGGAGTCGGAACCCGAACGGAAGCGGGCTCGCACCGACGAGGTGCCT
GCCGGAGGAAGCCGCTCCGAGGCGGAAGATGAGGACGACGAGGACTACGTG
CCCTATGTGCCGTTACGGCAGCGCCGGCAGCTACTGCTCCAGAAGCTGCTGC
AGCGAAGACGCAAGGGAGCTGCGGAGGAAGAGCAGCAGGACAGCGGTAGTG
AACCCCGGGGAGATGAGGACGACATCCCGCTAGGCCCTCAGTCCAACGTCAG
CCTCCTGGATCAGCACCAGCACCTTAAAGAGAAGGCTGAAGCGCGCAAAGA
GTCTGCCAAGGAGAAGCAGCTGAAGGAAGAAGAGAAGATCCTGGAGAGTGT
TGCCGAGGGCCGAGCATTGATGTCAGTGAAGGAGATGGCTAAGGGCATTACG
TATGATGACCCCATCAAAACCAGCTGGACTCCACCCCGTTATGTTCTGAGCAT
GTCTGAAGAGCGACATGAGCGCGTGCGGAAGAAATACCACATCCTGGTGGA
GGGAGACGGTATCCCACCACCCATCAAGAGCTTCAAGGAAATGAAGTTTCCT
GCAGCCATCCTGAGAGGCCTGAAGAAGAAAGGCATTCACCACCCAACACCC
ATTCAGATCCAGGGCATCCCCACCATTCTATCTGGCCGTGACATGATAGGCAT
CGCTTTCACGGGTTCAGGCAAGACACTGGTGTTCACGTTGCCCGTCATCATGT
TCTGCCTGGAACAAGAGAAGAGGTTACCCTTCTCAAAGCGCGAGGGGCCCTA
TGGACTCATCATCTGCCCCTCGCGGGAGCTGGCCCGGCAGACCCATGGCATC
CTGGAGTACTACTGCCGCCTGCTGCAGGAGGACAGCTCACCACTCCTGCGCT
GCGCCCTCTGCATTGGGGGCATGTCCGTGAAAGAGCAGATGGAGACCATCCG
ACACGGTGTACACATGATGGTGGCCACCCCGGGGCGCCTCATGGATTTGCTG
CAGAAGAAGATGGTCAGCCTAGACATCTGTCGCTACCTGGCCCTGGACGAGG
CTGACCGCATGATCGACATGGGCTTCGAGGGTGACATCCGTACCATCTTCTCC
TACTTCAAGGGCCAGCGACAGACCCTGCTCTTCAGTGCCACCATGCCGAAGA
AGATTCAGAACTTTGCTAAGAGTGCCCTTGTAAAGCCTGTGACCATCAATGTG
GGGCGCGCTGGGGCTGCCAGCCTGGATGTCATCCAGGAGGTAGAATATGTGA
AGGAGGAGGCCAAGATGGTGTACCTGCTCGAGTGCCTGCAGAAGACACCCCC
GCCTGTACTCATCTTTGCAGAGAAGAAGGCAGACGTGGACGCCATCCACGAG
TACCTGCTGCTCAAGGGGGTTGAGGCCGTAGCCATCCATGGGGGCAAAGACC
AGGAGGAACGGACTAAGGCCATCGAGGCATTCCGGGAGGGCAAGAAGGATG
TCCTAGTAGCCACAGACGTTGCCTCCAAGGGCCTGGACTTCCCTGCCATCCAG
CACGTCATCAATTATGACATGCCAGAGGAGATTGAGAACTATGTACACCGGA
TTGGCCGCACCGGGCGCTCGGGAAACACAGGCATCGCCACTACCTTCATCAA
CAAAGCGTGTGATGAGTCAGTGCTGATGGACCTCAAAGCGCTGCTGCTAGAA
GCCAAGCAGAAGGTGCCGCCCGTGCTGCAGGTGCTGCATTGCGGGGATGAGT
CCATGCTGGACATTGGAGGAGAGCGCGGCTGTGCCTTCTGCGGGGGCCTGGG
TCATCGGATCACTGACTGCCCCAAACTCGAGGCTATGCAGACCAAGCAGGTC
AGCAACATCGGTCGCAAGGACTACCTGGCCCACAGCTCCATGGACTTCTGA
Human DDX41 Protein Sequence, Variant 1 (SEQ ID NO: 69)
MEESEPERKRARTDEVPAGGSRSEAEDEDDEDYVPYVPLRQRRQLLLQKLLQRR
RKGAAEEEQQDSGSEPRGDEDDIPLGPQSNVSLLDQHQHLKEKAEARKESAKEK
QLKEEEKILESVAEGRALMSVKEMAKGITYDDPIKTSWTPPRYVLSMSEERHERV
RKKYHILVEGDGIPPPIKSFKEMKFPAAILRGLKKKGIHHPTPIQIQGIPTILSGRDM
IGIAFTGSGKTLVFTLPVIMFCLEQEKRLPFSKREGPYGLIICPSRELARQTHGILEY
YCRLLQEDSSPLLRCALCIGGMSVKEQMETIRHGVHMMVATPGRLMDLLQKKM
VSLDICRYLALDEADRMIDMGFEGDIRTIFSYFKGQRQTLLFSATMPKKIQNFAKS
ALVKPVTINVGRAGAASLDVIQEVEYVKEEAKMVYLLECLQKTPPPVLIFAEKK
ADVDAIHEYLLLKGVEAVAIHGGKDQEERTKAIEAFREGKKDVLVATDVASKGL
DFPAIQHVINYDMPEEIENYVHRIGRTGRSGNTGIATTFINKACDESVLMDLKALL
LEAKQKVPPVLQVLHCGDESMLDIGGERGCAFCGGLGHRITDCPKLEAMQTKQ
VSNIGRKDYLAHSSMDF
Human DDX41 cDNA Sequence, Variant 2 (SEQ ID NO: 70)
ATGTCAGTGAAGGAGATGGCTAAGGGCATTACGTATGATGACCCCATCAAAA
CCAGCTGGACTCCACCCCGTTATGTTCTGAGCATGTCTGAAGAGCGACATGA
GCGCGTGCGGAAGAAATACCACATCCTGGTGGAGGGAGACGGTATCCCACC
ACCCATCAAGAGCTTCAAGGAAATGAAGTTTCCTGCAGCCATCCTGAGAGGC
CTGAAGAAGAAAGGCATTCACCACCCAACACCCATTCAGATCCAGGGCATCC
CCACCATTCTATCTGGCCGTGACATGATAGGCATCGCTTTCACGGGTTCAGGC
AAGACACTGGTGTTCACGTTGCCCGTCATCATGTTCTGCCTGGAACAAGAGA
AGAGGTTACCCTTCTCAAAGCGCGAGGGGCCCTATGGACTCATCATCTGCCC
CTCGCGGGAGCTGGCCCGGCAGACCCATGGCATCCTGGAGTACTACTGCCGC
CTGCTGCAGGAGGACAGCTCACCACTCCTGCGCTGCGCCCTCTGCATTGGGG
GCATGTCCGTGAAAGAGCAGATGGAGACCATCCGACACGGTGTACACATGAT
GGTGGCCACCCCGGGGCGCCTCATGGATTTGCTGCAGAAGAAGATGGTCAGC
CTAGACATCTGTCGCTACCTGGCCCTGGACGAGGCTGACCGCATGATCGACA
TGGGCTTCGAGGGTGACATCCGTACCATCTTCTCCTACTTCAAGGGCCAGCGA
CAGACCCTGCTCTTCAGTGCCACCATGCCGAAGAAGATTCAGAACTTTGCTA
AGAGTGCCCTTGTAAAGCCTGTGACCATCAATGTGGGGCGCGCTGGGGCTGC
CAGCCTGGATGTCATCCAGGAGGTAGAATATGTGAAGGAGGAGGCCAAGAT
GGTGTACCTGCTCGAGTGCCTGCAGAAGACACCCCCGCCTGTACTCATCTTTG
CAGAGAAGAAGGCAGACGTGGACGCCATCCACGAGTACCTGCTGCTCAAGG
GGGTTGAGGCCGTAGCCATCCATGGGGGCAAAGACCAGGAGGAACGGACTA
AGGCCATCGAGGCATTCCGGGAGGGCAAGAAGGATGTCCTAGTAGCCACAG
ACGTTGCCTCCAAGGGCCTGGACTTCCCTGCCATCCAGCACGTCATCAATTAT
GACATGCCAGAGGAGATTGAGAACTATGTACACCGGATTGGCCGCACCGGGC
GCTCGGGAAACACAGGCATCGCCACTACCTTCATCAACAAAGCGTGTGATGA
GTCAGTGCTGATGGACCTCAAAGCGCTGCTGCTAGAAGCCAAGCAGAAGGTG
CCGCCCGTGCTGCAGGTGCTGCATTGCGGGGATGAGTCCATGCTGGACATTG
GAGGAGAGCGCGGCTGTGCCTTCTGCGGGGGCCTGGGTCATCGGATCACTGA
CTGCCCCAAACTCGAGGCTATGCAGACCAAGCAGGTCAGCAACATCGGTCGC
AAGGACTACCTGGCCCACAGCTCCATGGACTTCTGA
Human DDX41 Protein Sequence, Variant 2 (SEQ ID NO: 71)
MSVKEMAKGITYDDPIKTSWTPPRYVLSMSEERHERVRKKYHILVEGDGIPPPIK
SFKEMKFPAAILRGLKKKGIHHPTPIQIQGIPTILSGRDMIGIAFTGSGKTLVFTLPV
IMFCLEQEKRLPFSKREGPYGLIICPSRELARQTHGILEYYCRLLQEDSSPLLRCAL
CIGGMSVKEQMETIRHGVHMMVATPGRLMDLLQKKMVSLDICRYLALDEADR
MIDMGFEGDIRTIFSYFKGQRQTLLFSATMPKKIQNFAKSALVKPVTINVGRAGA
ASLDVIQEVEYVKEEAKMVYLLECLQKTPPPVLIFAEKKADVDAIHEYLLLKGVE
AVAIHGGKDQEERTKAIEAFREGKKDVLVATDVASKGLDFPAIQHVINYDMPEEI
ENYVHRIGRTGRSGNTGIATTFINKACDESVLMDLKALLLEAKQKVPPVLQVLH
CGDESMLDIGGERGCAFCGGLGHRITDCPKLEAMQTKQVSNIGRKDYLAHSSMD
F
Human EXO1 cDNA Sequence, Variant 1 (SEQ ID NO: 72)
ATGGGGATACAGGGATTGCTACAATTTATCAAAGAAGCTTCAGAACCCATCC
ATGTGAGGAAGTATAAAGGGCAGGTAGTAGCTGTGGATACATATTGCTGGCT
TCACAAAGGAGCTATTGCTTGTGCTGAAAAACTAGCCAAAGGTGAACCTACT
GATAGGTATGTAGGATTTTGTATGAAATTTGTAAATATGTTACTATCTCATGG
GATCAAGCCTATTCTCGTATTTGATGGATGTACTTTACCTTCTAAAAAGGAAG
TAGAGAGATCTAGAAGAGAAAGACGACAAGCCAATCTTCTTAAGGGAAAGC
AACTTCTTCGTGAGGGGAAAGTCTCGGAAGCTCGAGAGTGTTTCACCCGGTC
TATCAATATCACACATGCCATGGCCCACAAAGTAATTAAAGCTGCCCGGTCT
CAGGGGGTAGATTGCCTCGTGGCTCCCTATGAAGCTGATGCGCAGTTGGCCT
ATCTTAACAAAGCGGGAATTGTGCAAGCCATAATTACAGAGGACTCGGATCT
CCTAGCTTTTGGCTGTAAAAAGGTAATTTTAAAGATGGACCAGTTTGGAAAT
GGACTTGAAATTGATCAAGCTCGGCTAGGAATGTGCAGACAGCTTGGGGATG
TATTCACGGAAGAGAAGTTTCGTTACATGTGTATTCTTTCAGGTTGTGACTAC
CTGTCATCACTGCGTGGGATTGGATTAGCAAAGGCATGCAAAGTCCTAAGAC
TAGCCAATAATCCAGATATAGTAAAGGTTATCAAGAAAATTGGACATTATCT
CAAGATGAATATCACGGTACCAGAGGATTACATCAACGGGTTTATTCGGGCC
AACAATACCTTCCTCTATCAGCTAGTTTTTGATCCCATCAAAAGGAAACTTAT
TCCTCTGAACGCCTATGAAGATGATGTTGATCCTGAAACACTAAGCTACGCTG
GGCAATATGTTGATGATTCCATAGCTCTTCAAATAGCACTTGGAAATAAAGA
TATAAATACTTTTGAACAGATCGATGACTACAATCCAGACACTGCTATGCCTG
CCCATTCAAGAAGTCATAGTTGGGATGACAAAACATGTCAAAAGTCAGCTAA
TGTTAGCAGCATTTGGCATAGGAATTACTCTCCCAGACCAGAGTCGGGTACT
GTTTCAGATGCCCCACAATTGAAGGAAAATCCAAGTACTGTGGGAGTGGAAC
GAGTGATTAGTACTAAAGGGTTAAATCTCCCAAGGAAATCATCCATTGTGAA
AAGACCAAGAAGTGCAGAGCTGTCAGAAGATGACCTGTTGAGTCAGTATTCT
CTTTCATTTACGAAGAAGACCAAGAAAAATAGCTCTGAAGGCAATAAATCAT
TGAGCTTTTCTGAAGTGTTTGTGCCTGACCTGGTAAATGGACCTACTAACAAA
AAGAGTGTAAGCACTCCACCTAGGACGAGAAATAAATTTGCAACATTTTTAC
AAAGGAAAAATGAAGAAAGTGGTGCAGTTGTGGTTCCAGGGACCAGAAGCA
GGTTTTTTTGCAGTTCAGATTCTACTGACTGTGTATCAAACAAAGTGAGCATC
CAGCCTCTGGATGAAACTGCTGTCACAGATAAAGAGAACAATCTGCATGAAT
CAGAGTATGGAGACCAAGAAGGCAAGAGACTGGTTGACACAGATGTAGCAC
GTAATTCAAGTGATGACATTCCGAATAATCATATTCCAGGTGATCATATTCCA
GACAAGGCAACAGTGTTTACAGATGAAGAGTCCTACTCTTTTGAGAGCAGCA
AATTTACAAGGACCATTTCACCACCCACTTTGGGAACACTAAGAAGTTGTTTT
AGTTGGTCTGGAGGTCTTGGAGATTTTTCAAGAACGCCGAGCCCCTCTCCAAG
CACAGCATTGCAGCAGTTCCGAAGAAAGAGCGATTCCCCCACCTCTTTGCCT
GAGAATAATATGTCTGATGTGTCGCAGTTAAAGAGCGAGGAGTCCAGTGACG
ATGAGTCTCATCCCTTACGAGAAGAGGCATGTTCTTCACAGTCCCAGGAAAG
TGGAGAATTCTCACTGCAGAGTTCAAATGCATCAAAGCTTTCTCAGTGCTCTA
GTAAGGACTCTGATTCAGAGGAATCTGATTGCAATATTAAGTTACTTGACAGT
CAAAGTGACCAGACCTCCAAGCTACGTTTATCTCATTTCTCAAAAAAAGACA
CACCTCTAAGGAACAAGGTTCCTGGGCTATATAAGTCCAGTTCTGCAGACTCT
CTTTCTACAACCAAGATCAAACCTCTAGGACCTGCCAGAGCCAGTGGGCTGA
GCAAGAAGCCGGCAAGCATCCAGAAGAGAAAGCATCATAATGCCGAGAACA
AGCCGGGGTTACAGATCAAACTCAATGAGCTCTGGAAAAACTTTGGATTTAA
AAAAGATTCTGAAAAGCTTCCTCCTTGTAAGAAACCCCTGTCCCCAGTCAGA
GATAACATCCAACTAACTCCAGAAGCGGAAGAGGATATATTTAACAAACCTG
AATGTGGCCGTGTTCAAAGAGCAATATTCCAGTAA
Human EXO1 Protein Sequence, Variant 1 (SEQ ID NO: 73)
MGIQGLLQFIKEASEPIHVRKYKGQVVAVDTYCWLHKGAIACAEKLAKGEPTDR
YVGFCMKFVNMLLSHGIKPILVFDGCTLPSKKEVERSRRERRQANLLKGKQLLR
EGKVSEARECFTRSINITHAMAHKVIKAARSQGVDCLVAPYEADAQLAYLNKAG
IVQAIITEDSDLLAFGCKKVILKMDQFGNGLEIDQARLGMCRQLGDVFTEEKFRY
MCILSGCDYLSSLRGIGLAKACKVLRLANNPDIVKVIKKIGHYLKMNITVPEDYIN
GFIRANNTFLYQLVFDPIKRKLIPLNAYEDDVDPETLSYAGQYVDDSIALQIALGN
KDINTFEQIDDYNPDTAMPAHSRSHSWDDKTCQKSANVSSIWHRNYSPRPESGT
VSDAPQLKENPSTVGVERVISTKGLNLPRKSSIVKRPRSAELSEDDLLSQYSLSFT
KKTKKNSSEGNKSLSFSEVFVPDLVNGPTNKKSVSTPPRTRNKFATFLQRKNEES
GAVVVPGTRSRFFCSSDSTDCVSNKVSIQPLDETAVTDKENNLHESEYGDQEGK
RLVDTDVARNSSDDIPNNHIPGDHIPDKATVFTDEESYSFESSKFTRTISPPTLGTL
RSCFSWSGGLGDFSRTPSPSPSTALQQFRRKSDSPTSLPENNMSDVSQLKSEESSD
DESHPLREEACSSQSQESGEFSLQSSNASKLSQCSSKDSDSEESDCNIKLLDSQSD
QTSKLRLSHFSKKDTPLRNKVPGLYKSSSADSLSTTKIKPLGPARASGLSKKPASI
QKRKHHNAENKPGLQIKLNELWKNFGFKKDSEKLPPCKKPLSPVRDNIQLTPEA
EEDIFNKPECGRVQRAIFQ
Human EXO cDNA Sequence, Variant 2 (SEQ ID NO: 74)
ATGGGGATACAGGGATTGCTACAATTTATCAAAGAAGCTTCAGAACCCATCC
ATGTGAGGAAGTATAAAGGGCAGGTAGTAGCTGTGGATACATATTGCTGGCT
TCACAAAGGAGCTATTGCTTGTGCTGAAAAACTAGCCAAAGGTGAACCTACT
GATAGGTATGTAGGATTTTGTATGAAATTTGTAAATATGTTACTATCTCATGG
GATCAAGCCTATTCTCGTATTTGATGGATGTACTTTACCTTCTAAAAAGGAAG
TAGAGAGATCTAGAAGAGAAAGACGACAAGCCAATCTTCTTAAGGGAAAGC
AACTTCTTCGTGAGGGGAAAGTCTCGGAAGCTCGAGAGTGTTTCACCCGGTC
TATCAATATCACACATGCCATGGCCCACAAAGTAATTAAAGCTGCCCGGTCT
CAGGGGGTAGATTGCCTCGTGGCTCCCTATGAAGCTGATGCGCAGTTGGCCT
ATCTTAACAAAGCGGGAATTGTGCAAGCCATAATTACAGAGGACTCGGATCT
CCTAGCTTTTGGCTGTAAAAAGGTAATTTTAAAGATGGACCAGTTTGGAAAT
GGACTTGAAATTGATCAAGCTCGGCTAGGAATGTGCAGACAGCTTGGGGATG
TATTCACGGAAGAGAAGTTTCGTTACATGTGTATTCTTTCAGGTTGTGACTAC
CTGTCATCACTGCGTGGGATTGGATTAGCAAAGGCATGCAAAGTCCTAAGAC
TAGCCAATAATCCAGATATAGTAAAGGTTATCAAGAAAATTGGACATTATCT
CAAGATGAATATCACGGTACCAGAGGATTACATCAACGGGTTTATTCGGGCC
AACAATACCTTCCTCTATCAGCTAGTTTTTGATCCCATCAAAAGGAAACTTAT
TCCTCTGAACGCCTATGAAGATGATGTTGATCCTGAAACACTAAGCTACGCTG
GGCAATATGTTGATGATTCCATAGCTCTTCAAATAGCACTTGGAAATAAAGA
TATAAATACTTTTGAACAGATCGATGACTACAATCCAGACACTGCTATGCCTG
CCCATTCAAGAAGTCATAGTTGGGATGACAAAACATGTCAAAAGTCAGCTAA
TGTTAGCAGCATTTGGCATAGGAATTACTCTCCCAGACCAGAGTCGGGTACT
GTTTCAGATGCCCCACAATTGAAGGAAAATCCAAGTACTGTGGGAGTGGAAC
GAGTGATTAGTACTAAAGGGTTAAATCTCCCAAGGAAATCATCCATTGTGAA
AAGACCAAGAAGTGAGCTGTCAGAAGATGACCTGTTGAGTCAGTATTCTCTT
TCATTTACGAAGAAGACCAAGAAAAATAGCTCTGAAGGCAATAAATCATTGA
GCTTTTCTGAAGTGTTTGTGCCTGACCTGGTAAATGGACCTACTAACAAAAAG
AGTGTAAGCACTCCACCTAGGACGAGAAATAAATTTGCAACATTTTTACAAA
GGAAAAATGAAGAAAGTGGTGCAGTTGTGGTTCCAGGGACCAGAAGCAGGT
TTTTTTGCAGTTCAGATTCTACTGACTGTGTATCAAACAAAGTGAGCATCCAG
CCTCTGGATGAAACTGCTGTCACAGATAAAGAGAACAATCTGCATGAATCAG
AGTATGGAGACCAAGAAGGCAAGAGACTGGTTGACACAGATGTAGCACGTA
ATTCAAGTGATGACATTCCGAATAATCATATTCCAGGTGATCATATTCCAGAC
AAGGCAACAGTGTTTACAGATGAAGAGTCCTACTCTTTTGAGAGCAGCAAAT
TTACAAGGACCATTTCACCACCCACTTTGGGAACACTAAGAAGTTGTTTTAGT
TGGTCTGGAGGTCTTGGAGATTTTTCAAGAACGCCGAGCCCCTCTCCAAGCAC
AGCATTGCAGCAGTTCCGAAGAAAGAGCGATTCCCCCACCTCTTTGCCTGAG
AATAATATGTCTGATGTGTCGCAGTTAAAGAGCGAGGAGTCCAGTGACGATG
AGTCTCATCCCTTACGAGAAGAGGCATGTTCTTCACAGTCCCAGGAAAGTGG
AGAATTCTCACTGCAGAGTTCAAATGCATCAAAGCTTTCTCAGTGCTCTAGTA
AGGACTCTGATTCAGAGGAATCTGATTGCAATATTAAGTTACTTGACAGTCA
AAGTGACCAGACCTCCAAGCTACGTTTATCTCATTTCTCAAAAAAAGACACA
CCTCTAAGGAACAAGGTTCCTGGGCTATATAAGTCCAGTTCTGCAGACTCTCT
TTCTACAACCAAGATCAAACCTCTAGGACCTGCCAGAGCCAGTGGGCTGAGC
AAGAAGCCGGCAAGCATCCAGAAGAGAAAGCATCATAATGCCGAGAACAAG
CCGGGGTTACAGATCAAACTCAATGAGCTCTGGAAAAACTTTGGATTTAAAA
AAGATTCTGAAAAGCTTCCTCCTTGTAAGAAACCCCTGTCCCCAGTCAGAGAT
AACATCCAACTAACTCCAGAAGCGGAAGAGGATATATTTAACAAACCTGAAT
GTGGCCGTGTTCAAAGAGCAATATTCCAGTAA
Human EXO Protein Sequence, Variant 2 (SEQ ID NO: 75)
MGIQGLLQFIKEASEPIHVRKYKGQVVAVDTYCWLHKGAIACAEKLAKGEPTDR
YVGFCMKFVNMLLSHGIKPILVFDGCTLPSKKEVERSRRERRQANLLKGKQLLR
EGKVSEARECFTRSINITHAMAHKVIKAARSQGVDCLVAPYEADAQLAYLNKAG
IVQAIITEDSDLLAFGCKKVILKMDQFGNGLEIDQARLGMCRQLGDVFTEEKFRY
MCILSGCDYLSSLRGIGLAKACKVLRLANNPDIVKVIKKIGHYLKMNITVPEDYIN
GFIRANNTFLYQLVFDPIKRKLIPLNAYEDDVDPETLSYAGQYVDDSIALQIALGN
KDINTFEQIDDYNPDTAMPAHSRSHSWDDKTCQKSANVSSIWHRNYSPRPESGT
VSDAPQLKENPSTVGVERVISTKGLNLPRKSSIVKRPRSELSEDDLLSQYSLSFTK
KTKKNSSEGNKSLSFSEVFVPDLVNGPTNKKSVSTPPRTRNKFATFLQRKNEESG
AVVVPGTRSRFFCSSDSTDCVSNKVSIQPLDETAVTDKENNLHESEYGDQEGKRL
VDTDVARNSSDDIPNNHIPGDHIPDKATVFTDEESYSFESSKFTRTISPPTLGTLRS
CFSWSGGLGDFSRTPSPSPSTALQQFRRKSDSPTSLPENNMSDVSQLKSEESSDDE
SHPLREEACSSQSQESGEFSLQSSNASKLSQCSSKDSDSEESDCNIKLLDSQSDQTS
KLRLSHFSKKDTPLRNKVPGLYKSSSADSLSTTKIKPLGPARASGLSKKPASIQKR
KHHNAENKPGLQIKLNELWKNFGFKKDSEKLPPCKKPLSPVRDNIQLTPEAEEDI
FNKPECGRVQRAIFQ
Human EXO cDNA Sequence, Variant 3 (SEQ ID NO: 76)
ATGGGGATACAGGGATTGCTACAATTTATCAAAGAAGCTTCAGAACCCATCC
ATGTGAGGAAGTATAAAGGGCAGGTAGTAGCTGTGGATACATATTGCTGGCT
TCACAAAGGAGCTATTGCTTGTGCTGAAAAACTAGCCAAAGGTGAACCTACT
GATAGGTATGTAGGATTTTGTATGAAATTTGTAAATATGTTACTATCTCATGG
GATCAAGCCTATTCTCGTATTTGATGGATGTACTTTACCTTCTAAAAAGGAAG
TAGAGAGATCTAGAAGAGAAAGACGACAAGCCAATCTTCTTAAGGGAAAGC
AACTTCTTCGTGAGGGGAAAGTCTCGGAAGCTCGAGAGTGTTTCACCCGGTC
TATCAATATCACACATGCCATGGCCCACAAAGTAATTAAAGCTGCCCGGTCT
CAGGGGGTAGATTGCCTCGTGGCTCCCTATGAAGCTGATGCGCAGTTGGCCT
ATCTTAACAAAGCGGGAATTGTGCAAGCCATAATTACAGAGGACTCGGATCT
CCTAGCTTTTGGCTGTAAAAAGGTAATTTTAAAGATGGACCAGTTTGGAAAT
GGACTTGAAATTGATCAAGCTCGGCTAGGAATGTGCAGACAGCTTGGGGATG
TATTCACGGAAGAGAAGTTTCGTTACATGTGTATTCTTTCAGGTTGTGACTAC
CTGTCATCACTGCGTGGGATTGGATTAGCAAAGGCATGCAAAGTCCTAAGAC
TAGCCAATAATCCAGATATAGTAAAGGTTATCAAGAAAATTGGACATTATCT
CAAGATGAATATCACGGTACCAGAGGATTACATCAACGGGTTTATTCGGGCC
AACAATACCTTCCTCTATCAGCTAGTTTTTGATCCCATCAAAAGGAAACTTAT
TCCTCTGAACGCCTATGAAGATGATGTTGATCCTGAAACACTAAGCTACGCTG
GGCAATATGTTGATGATTCCATAGCTCTTCAAATAGCACTTGGAAATAAAGA
TATAAATACTTTTGAACAGATCGATGACTACAATCCAGACACTGCTATGCCTG
CCCATTCAAGAAGTCATAGTTGGGATGACAAAACATGTCAAAAGTCAGCTAA
TGTTAGCAGCATTTGGCATAGGAATTACTCTCCCAGACCAGAGTCGGGTACT
GTTTCAGATGCCCCACAATTGAAGGAAAATCCAAGTACTGTGGGAGTGGAAC
GAGTGATTAGTACTAAAGGGTTAAATCTCCCAAGGAAATCATCCATTGTGAA
AAGACCAAGAAGTGCAGAGCTGTCAGAAGATGACCTGTTGAGTCAGTATTCT
CTTTCATTTACGAAGAAGACCAAGAAAAATAGCTCTGAAGGCAATAAATCAT
TGAGCTTTTCTGAAGTGTTTGTGCCTGACCTGGTAAATGGACCTACTAACAAA
AAGAGTGTAAGCACTCCACCTAGGACGAGAAATAAATTTGCAACATTTTTAC
AAAGGAAAAATGAAGAAAGTGGTGCAGTTGTGGTTCCAGGGACCAGAAGCA
GGTTTTTTTGCAGTTCAGATTCTACTGACTGTGTATCAAACAAAGTGAGCATC
CAGCCTCTGGATGAAACTGCTGTCACAGATAAAGAGAACAATCTGCATGAAT
CAGAGTATGGAGACCAAGAAGGCAAGAGACTGGTTGACACAGATGTAGCAC
GTAATTCAAGTGATGACATTCCGAATAATCATATTCCAGGTGATCATATTCCA
GACAAGGCAACAGTGTTTACAGATGAAGAGTCCTACTCTTTTGAGAGCAGCA
AATTTACAAGGACCATTICACCACCCACTTTGGGAACACTAAGAAGTTGTTTT
AGTTGGTCTGGAGGTCTTGGAGATTTTTCAAGAACGCCGAGCCCCTCTCCAAG
CACAGCATTGCAGCAGTTCCGAAGAAAGAGCGATTCCCCCACCTCTTTGCCT
GAGAATAATATGTCTGATGTGTCGCAGTTAAAGAGCGAGGAGTCCAGTGACG
ATGAGTCTCATCCCTTACGAGAAGAGGCATGTTCTTCACAGTCCCAGGAAAG
TGGAGAATTCTCACTGCAGAGTTCAAATGCATCAAAGCTTTCTCAGTGCTCTA
GTAAGGACTCTGATTCAGAGGAATCTGATTGCAATATTAAGTTACTTGACAGT
CAAAGTGACCAGACCTCCAAGCTACGTTTATCTCATTTCTCAAAAAAAGACA
CACCTCTAAGGAACAAGGTTCCTGGGCTATATAAGTCCAGTTCTGCAGACTCT
CTTTCTACAACCAAGATCAAACCTCTAGGACCTGCCAGAGCCAGTGGGCTGA
GCAAGAAGCCGGCAAGCATCCAGAAGAGAAAGCATCATAATGCCGAGAACA
AGCCGGGGTTACAGATCAAACTCAATGAGCTCTGGAAAAACTTTGGATTTAA
AAAATTCTGA
Human EXO Protein Sequence, Variant 3 (SEQ ID NO: 77)
MGIQGLLQFIKEASEPIHVRKYKGQVVAVDTYCWLHKGAIACAEKLAKGEPTDR
YVGFCMKFVNMLLSHGIKPILVFDGCTLPSKKEVERSRRERRQANLLKGKQLLR
EGKVSEARECFTRSINITHAMAHKVIKAARSQGVDCLVAPYEADAQLAYLNKAG
IVQAIITEDSDLLAFGCKKVILKMDQFGNGLEIDQARLGMCRQLGDVFTEEKFRY
MCILSGCDYLSSLRGIGLAKACKVLRLANNPDIVKVIKKIGHYLKMNITVPEDYIN
GFIRANNTFLYQLVFDPIKRKLIPLNAYEDDVDPETLSYAGQYVDDSIALQIALGN
KDINTFEQIDDYNPDTAMPAHSRSHSWDDKTCQKSANVSSIWHRNYSPRPESGT
VSDAPQLKENPSTVGVERVISTKGLNLPRKSSIVKRPRSAELSEDDLLSQYSLSFT
KKTKKNSSEGNKSLSFSEVFVPDLVNGPTNKKSVSTPPRTRNKFATFLQRKNEES
GAVVVPGTRSRFFCSSDSTDCVSNKVSIQPLDETAVTDKENNLHESEYGDQEGK
RLVDTDVARNSSDDIPNNHIPGDHIPDKATVFTDEESYSFESSKFTRTISPPTLGTL
RSCFSWSGGLGDFSRTPSPSPSTALQQFRRKSDSPTSLPENNMSDVSQLKSEESSD
DESHPLREEACSSQSQESGEFSLQSSNASKLSQCSSKDSDSEESDCNIKLLDSQSD
QTSKLRLSHFSKKDTPLRNKVPGLYKSSSADSLSTTKIKPLGPARASGLSKKPASI
QKRKHHNAENKPGLQIKLNELWKNFGFKKF
Human DNA2 cDNA Sequence (SEQ ID NO: 78)
ATGGAGCAGCTGAACGAACTGGAGCTGCTGATGGAGAAGAGTTTTTGGGAGG
AGGCGGAGCTGCCGGCGGAGCTATTTCAGAAGAAAGTGGTAGCTTCCTTTCC
AAGAACAGTTCTGAGCACAGGAATGGATAACCGGTACCTGGTGTTGGCAGTC
AATACTGTACAGAACAAAGAGGGAAACTGTGAAAAGCGCCTGGTCATCACTG
CTTCACAGTCACTAGAAAATAAAGAACTATGCATCCTTAGGAATGACTGGTG
TTCTGTTCCAGTAGAGCCAGGAGATATCATTCATTTGGAGGGAGACTGCACA
TCTGACACTTGGATAATAGATAAAGATTTTGGATATTTGATTCTGTATCCAGA
CATGCTGATTTCTGGCACCAGCATAGCCAGTAGTATTCGATGTATGAGAAGA
GCTGTCCTGAGTGAAACTTTTAGGAGCTCTGATCCAGCCACACGCCAAATGCT
AATTGGTACGGTTCTCCATGAGGTGTTTCAAAAAGCCATAAATAATAGCTTTG
CCCCAGAAAAGCTACAAGAACTTGCTTTTCAAACAATTCAAGAAATAAGACA
TTTGAAGGAAATGTACCGCTTAAATCTAAGTCAAGATGAAATAAAACAAGAA
GTAGAGGACTATCTTCCTTCGTTTTGTAAATGGGCAGGAGATTTCATGCATAA
AAACACTTCGACTGACTTCCCTCAGATGCAGCTCTCTCTGCCAAGTGATAATA
GTAAGGATAATTCAACATGTAACATTGAAGTCGTGAAACCAATGGATATTGA
AGAAAGCATTTGGTCCCCTAGGTTTGGATTGAAAGGCAAAATAGATGTTACA
GTTGGTGTGAAAATACATCGAGGGTATAAAACAAAATACAAGATAATGCCGC
TGGAACTTAAAACTGGCAAAGAATCAAATTCTATTGAACACCGTAGTCAGGT
TGTTCTGTACACTCTACTAAGCCAAGAGAGAAGAGCTGATCCAGAGGCTGGC
TTGCTTCTCTACCTCAAGACTGGTCAGATGTACCCTGTGCCTGCCAACCATCT
AGATAAAAGAGAATTATTAAAGCTAAGAAACCAGATGGCATTCTCATTGTTT
CACCGTATTAGCAAATCTGCTACTAGACAGAAGACACAGCTTGCTTCTTTGCC
ACAAATAATTGAGGAAGAGAAAACTTGTAAATATTGTTCACAAATTGGCAAT
TGTGCTCTTTATAGCAGAGCAGTTGAACAACAGATGGATTGTAGTTCAGTCCC
AATTGTGATGCTGCCCAAAATAGAAGAAGAAACCCAGCATCTGAAGCAAAC
ACACTTAGAATATTTCAGCCTTTGGTGTCTAATGTTAACCCTGGAGTCACAAT
CGAAGGATAATAAAAAGAATCACCAAAATATCTGGCTAATGCCTGCTTCGGA
AATGGAGAAGAGTGGCAGTTGCATTGGAAACCTGATTAGAATGGAACATGTA
AAGATAGTTTGTGATGGGCAATATTTACATAATTTCCAATGTAAACATGGTGC
CATACCTGTCACAAATCTAATGGCAGGTGACAGAGTTATTGTAAGTGGAGAA
GAAAGGTCACTGTTTGCTTTGTCTAGAGGATATGTGAAGGAGATTAACATGA
CAACAGTAACTTGTTTATTAGACAGAAACTTGTCGGTCCTTCCAGAATCAACT
TTGTTCAGATTAGACCAAGAAGAAAAAAATTGTGATATAGATACCCCATTAG
GAAATCTTTCCAAATTGATGGAAAACACGTTTGTCAGCAAAAAACTTCGAGA
TTTAATTATTGACTTTCGTGAACCTCAGTTTATATCCTACCTTAGTTCTGTTCT
TCCACATGATGCAAAGGATACAGTTGCCTGCATTCTAAAGGGTTTGAATAAG
CCTCAGAGGCAAGCGATGAAAAAGGTACTTCTTTCAAAAGACTACACACTCA
TCGTGGGTATGCCTGGGACAGGAAAAACAACTACGATATGTACTCTCGTAAG
AATTCTCTACGCCTGTGGTTTTAGCGTTTTGTTGACCAGCTATACACACTCTGC
TGTTGACAATATTCTTTTGAAGTTAGCCAAGTTTAAAATAGGATTTTTGCGTT
TGGGTCAGATTCAGAAGGTTCATCCAGCTATCCAGCAATTTACAGAGCAAGA
AATTTGCAGATCAAAGTCCATTAAATCCTTAGCTCTTCTAGAAGAACTCTACA
ATAGTCAACTTATAGTTGCAACAACATGTATGGGAATAAACCATCCAATATTT
TCCCGTAAAATTTTTGATTTTTGTATTGTGGATGAAGCCTCTCAAATTAGCCA
ACCAATTTGTCTGGGCCCCCTTTTTTTTTCACGGAGATTTGTGTTAGTGGGGG
ACCATCAGCAGCTTCCTCCCCTGGTGCTAAACCGTGAAGCAAGAGCTCTTGG
CATGAGTGAAAGCTTATTCAAGAGGCTGGAGCAGAATAAGAGTGCTGTTGTA
CAGTTAACCGTGCAGTACAGAATGAACAGTAAAATTATGTCCTTAAGTAATA
AGCTGACCTATGAGGGCAAGCTGGAGTGTGGATCAGACAAAGTGGCCAATGC
AGTGATAAACCTACGTCACTTTAAAGATGTGAAGCTGGAACTGGAATTTTAT
GCTGACTATTCTGATAATCCTTGGTTGATGGGAGTATTTGAACCCAACAATCC
TGTTTGTTTCCTTAATACAGACAAGGTTCCAGCGCCAGAACAAGTTGAAAAA
GGTGGTGTGAGCAATGTAACAGAAGCCAAACTCATAGTTTTCCTAACCTCCA
TTTTTGTTAAGGCTGGATGCAGTCCCTCTGATATTGGTATTATTGCACCGTAC
AGGCAGCAATTAAAGATCATCAATGATTTATTGGCACGTTCTATTGGGATGGT
CGAAGTTAATACAGTAGACAAATACCAAGGAAGGGACAAAAGTATTGTCCTA
GTATCTTTTGTTAGAAGTAATAAGGATGGAACTGTTGGTGAACTCTTGAAAG
ATTGGCGACGTCTTAATGTTGCTATAACCAGAGCCAAACATAAACTGATTCTT
CTGGGGTGTGTGCCCTCACTAAATTGCTATCCTCCTTTGGAGAAGCTGCTTAA
TCATTTAAACTCAGAAAAATTAATCATTGATCTTCCATCAAGAGAACATGAA
AGTCTTTGCCACATATTGGGTGACTTTCAAAGAGAATAA
Human DNA2 Protein Sequence (SEQ ID NO: 79)
MEQLNELELLMEKSFWEEAELPAELFQKKVVASFPRTVLSTGMDNRYLVLAVN
TVQNKEGNCEKRLVITASQSLENKELCILRNDWCSVPVEPGDIIHLEGDCTSDTWI
IDKDFGYLILYPDMLISGTSIASSIRCMRRAVLSETFRSSDPATRQMLIGTVLHEVF
QKAINNSFAPEKLQELAFQTIQEIRHLKEMYRLNLSQDEIKQEVEDYLPSFCKWA
GDFMHKNTSTDFPQMQLSLPSDNSKDNSTCNIEVVKPMDIEESIWSPRFGLKGKI
DVTVGVKIHRGYKTKYKIMPLELKTGKESNSIEHRSQVVLYTLLSQERRADPEAG
LLLYLKTGQMYPVPANHLDKRELLKLRNQMAFSLFHRISKSATRQKTQLASLPQI
IEEEKTCKYCSQIGNCALYSRAVEQQMDCSSVPIVMLPKIEEETQHLKQTHLEYFS
LWCLMLTLESQSKDNKKNHQNIWLMPASEMEKSGSCIGNLIRMEHVKIVCDGQ
YLHNFQCKHGAIPVTNLMAGDRVIVSGEERSLFALSRGYVKEINMTTVTCLLDR
NLSVLPESTLFRLDQEEKNCDIDTPLGNLSKLMENTFVSKKLRDLIIDFREPQFISY
LSSVLPHDAKDTVACILKGLNKPQRQAMKKVLLSKDYTLIVGMPGTGKTTTICT
LVRILYACGFSVLLTSYTHSAVDNILLKLAKFKIGFLRLGQIQKVHPAIQQFTEQEI
CRSKSIKSLALLEELYNSQLIVATTCMGINHPIFSRKIFDFCIVDEASQISQPICLGPL
FFSRRFVLVGDHQQLPPLVLNREARALGMSESLFKRLEQNKSAVVQLTVQYRM
NSKIMSLSNKLTYEGKLECGSDKVANAVINLRHFKDVKLELEFYADYSDNPWLM
GVFEPNNPVCFLNTDKVPAPEQVEKGGVSNVTEAKLIVFLTSIFVKAGCSPSDIGII
APYRQQLKIINDLLARSIGMVEVNTVDKYQGRDKSIVLVSFVRSNKDGTVGELLK
DWRRLNVAITRAKHKLILLGCVPSLNCYPPLEKLLNHLNSEKLIIDLPSREHESLC
HILGDFQRE
Human RBBP8 cDNA Sequence, Variant 1 (SEQ ID NO: 80)
ATGAACATCTCGGGAAGCAGCTGTGGAAGCCCTAACTCTGCAGATACATCTA
GTGACTTTAAGGACCTTTGGACAAAACTAAAAGAATGTCATGATAGAGAAGT
ACAAGGTTTACAAGTAAAAGTAACCAAGCTAAAACAGGAACGAATCTTAGAT
GCACAAAGACTAGAAGAATTCTTCACCAAAAATCAACAGCTGAGGGAACAG
CAGAAAGTCCTTCATGAAACCATTAAAGTTTTAGAAGATCGGTTAAGAGCAG
GCTTATGTGATCGCTGTGCAGTAACTGAAGAACATATGCGGAAAAAACAGCA
AGAGTTTGAAAATATCCGGCAGCAGAATCTTAAACTTATTACAGAACTTATG
AATGAAAGGAATACTCTACAGGAAGAAAATAAAAAGCTTTCTGAACAACTCC
AGCAGAAAATTGAGAATGATCAACAGCATCAAGCAGCTGAGCTTGAATGTGA
GGAAGACGTTATTCCAGATTCACCGATAACAGCCTTCTCATTTTCTGGCGTTA
ACCGGCTACGAAGAAAGGAGAACCCCCATGTCCGATACATAGAACAAACAC
ATACTAAATTGGAGCACTCTGTGTGTGCAAATGAAATGAGAAAAGTTTCCAA
GTCTTCAACTCATCCACAACATAATCCTAATGAAAATGAAATTCTAGTAGCTG
ACACTTATGACCAAAGTCAATCTCCAATGGCCAAAGCACATGGAACAAGCAG
CTATACCCCTGATAAGTCATCTTTTAATTTAGCTACAGTTGTTGCTGAAACAC
TTGGACTTGGTGTTCAAGAAGAATCTGAAACTCAAGGTCCCATGAGCCCCCTT
GGTGATGAGCTCTACCACTGTCTGGAAGGAAATCACAAGAAACAGCCTTTTG
AGGAATCTACAAGAAATACTGAAGATAGTTTAAGATTTTCAGATTCTACTTCA
AAGACTCCTCCTCAAGAAGAATTACCTACTCGAGTGTCATCTCCTGTATTTGG
AGCTACCTCTAGTATCAAAAGTGGTTTAGATTTGAATACAAGTTTGTCCCCTT
CTCTTTTACAGCCTGGGAAAAAAAAACATCTGAAAACACTCCCTTTTAGCAA
CACTTGTATATCTAGATTAGAAAAAACTAGATCAAAATCTGAAGATAGTGCC
CTTTTCACACATCACAGTCTTGGGTCTGAAGTGAACAAGATCATTATCCAGTC
ATCTAATAAACAGATACTTATAAATAAAAATATAAGTGAATCCCTAGGTGAA
CAGAATAGGACTGAGTACGGTAAAGATTCTAACACTGATAAACATTTGGAGC
CCCTGAAATCATTGGGAGGCCGAACATCCAAAAGGAAGAAAACTGAGGAAG
AAAGTGAACATGAAGTAAGCTGCCCCCAAGCTTCTTTTGATAAAGAAAATGC
TTTCCCTTTTCCAATGGATAATCAGTTTTCCATGAATGGAGACTGTGTGATGG
ATAAACCTCTGGATCTGTCTGATCGATTTTCAGCTATTCAGCGTCAAGAGAAA
AGCCAAGGAAGTGAGACTTCTAAAAACAAATTTAGGCAAGTGACTCTTTATG
AGGCTTTGAAGACCATTCCAAAGGGCTTTTCCTCAAGCCGTAAGGCCTCAGA
TGGCAACTGCACGTTGCCCAAAGATTCCCCAGGGGAGCCCTGTTCACAGGAA
TGCATCATCCTTCAGCCCTTGAATAAATGCTCTCCAGACAATAAACCATCATT
ACAAATAAAAGAAGAAAATGCTGTCTTTAAAATTCCTCTACGTCCACGTGAA
AGTTTGGAGACTGAGAATGTTTTAGATGACATAAAGAGTGCTGGTTCTCATG
AGCCAATAAAAATACAAACCAGGTCAGACCATGGAGGATGTGAACTTGCATC
AGTTCTTCAGTTAAATCCATGTAGAACTGGTAAAATAAAGTCTCTACAAAAC
AACCAAGATGTATCCTTTGAAAATATCCAGTGGAGTATAGATCCGGGAGCAG
ACCTTTCTCAGTATAAAATGGATGTTACTGTAATAGATACAAAGGATGGCAG
TCAGTCAAAATTAGGAGGAGAGACAGTGGACATGGACTGTACATTGGTTAGT
GAAACCGTTCTCTTAAAAATGAAGAAGCAAGAGCAGAAGGGAGAAAAAAGT
TCAAATGAAGAAAGAAAAATGAATGATAGCTTGGAAGATATGTTTGATCGGA
CAACACATGAAGAGTATGAATCCTGTTTGGCAGACAGTTTCTCCCAAGCAGC
AGATGAAGAGGAGGAATTGTCTACTGCCACAAAGAAACTACACACTCATGGT
GATAAACAAGACAAAGTCAAGCAGAAAGCGTTTGTGGAGCCGTATTTTAAAG
GTGATGAAAGAGAGACTAGCTTGCAAAATTTTCCTCATATTGAGGTGGTTCG
GAAAAAAGAGGAGAGAAGAAAACTGCTTGGGCACACGTGTAAGGAATGTGA
AATTTATTATGCAGATATGCCAGCAGAAGAAAGAGAAAAGAAATTGGCTTCC
TGCTCAAGACACCGATTCCGCTACATTCCACCCAACACACCAGAGAATTTTTG
GGAAGTTGGTTTTCCTTCCACTCAGACTTGTATGGAAAGAGGTTATATTAAGG
AAGATCTTGATCCTTGTCCTCGTCCAAAAAGACGTCAGCCTTACAACGCAATA
TTTTCTCCAAAAGGCAAGGAGCAGAAGACATAG
Human RBBP8 Protein Sequence, Variant 1 (SEQ ID NO: 81)
MNISGSSCGSPNSADTSSDFKDLWTKLKECHDREVQGLQVKVTKLKQERILDAQ
RLEEFFTKNQQLREQQKVLHETIKVLEDRLRAGLCDRCAVTEEHMRKKQQEFEN
IRQQNLKLITELMNERNTLQEENKKLSEQLQQKIENDQQHQAAELECEEDVIPDS
PITAFSFSGVNRLRRKENPHVRYIEQTHTKLEHSVCANEMRKVSKSSTHPQHNPN
ENEILVADTYDQSQSPMAKAHGTSSYTPDKSSFNLATVVAETLGLGVQEESETQ
GPMSPLGDELYHCLEGNHKKQPFEESTRNTEDSLRFSDSTSKTPPQEELPTRVSSP
VFGATSSIKSGLDLNTSLSPSLLQPGKKKHLKTLPFSNTCISRLEKTRSKSEDSALF
THHSLGSEVNKIIIQSSNKQILINKNISESLGEQNRTEYGKDSNTDKHLEPLKSLGG
RTSKRKKTEEESEHEVSCPQASFDKENAFPFPMDNQFSMNGDCVMDKPLDLSDR
FSAIQRQEKSQGSETSKNKFRQVTLYEALKTIPKGFSSSRKASDGNCTLPKDSPGE
PCSQECIILQPLNKCSPDNKPSLQIKEENAVFKIPLRPRESLETENVLDDIKSAGSHE
PIKIQTRSDHGGCELASVLQLNPCRTGKIKSLQNNQDVSFENIQWSIDPGADLSQY
KMDVTVIDTKDGSQSKLGGETVDMDCTLVSETVLLKMKKQEQKGEKSSNEERK
MNDSLEDMFDRTTHEEYESCLADSFSQAADEEEELSTATKKLHTHGDKQDKVK
QKAFVEPYFKGDERETSLQNFPHIEVVRKKEERRKLLGHTCKECEIYYADMPAEE
REKKLASCSRHRFRYIPPNTPENFWEVGFPSTQTCMERGYIKEDLDPCPRPKRRQ
PYNAIFSPKGKEQKT
Human RBBP8 cDNA Sequence, Variant 2 (SEQ ID NO: 82)
ATGAACATCTCGGGAAGCAGCTGTGGAAGCCCTAACTCTGCAGATACATCTA
GTGACTTTAAGGACCTTTGGACAAAACTAAAAGAATGTCATGATAGAGAAGT
ACAAGGTTTACAAGTAAAAGTAACCAAGCTAAAACAGGAACGAATCTTAGAT
GCACAAAGACTAGAAGAATTCTTCACCAAAAATCAACAGCTGAGGGAACAG
CAGAAAGTCCTTCATGAAACCATTAAAGTTTTAGAAGATCGGTTAAGAGCAG
GCTTATGTGATCGCTGTGCAGTAACTGAAGAACATATGCGGAAAAAACAGCA
AGAGTTTGAAAATATCCGGCAGCAGAATCTTAAACTTATTACAGAACTTATG
AATGAAAGGAATACTCTACAGGAAGAAAATAAAAAGCTTTCTGAACAACTCC
AGCAGAAAATTGAGAATGATCAACAGCATCAAGCAGCTGAGCTTGAATGTGA
GGAAGACGTTATTCCAGATTCACCGATAACAGCCTTCTCATTTTCTGGCGTTA
ACCGGCTACGAAGAAAGGAGAACCCCCATGTCCGATACATAGAACAAACAC
ATACTAAATTGGAGCACTCTGTGTGTGCAAATGAAATGAGAAAAGTTTCCAA
GTCTTCAACTCATCCACAACATAATCCTAATGAAAATGAAATTCTAGTAGCTG
ACACTTATGACCAAAGTCAATCTCCAATGGCCAAAGCACATGGAACAAGCAG
CTATACCCCTGATAAGTCATCTTTTAATTTAGCTACAGTTGTTGCTGAAACAC
TTGGACTTGGTGTTCAAGAAGAATCTGAAACTCAAGGTCCCATGAGCCCCCTT
GGTGATGAGCTCTACCACTGTCTGGAAGGAAATCACAAGAAACAGCCTTTTG
AGGAATCTACAAGAAATACTGAAGATAGTTTAAGATTTTCAGATTCTACTTCA
AAGACTCCTCCTCAAGAAGAATTACCTACTCGAGTGTCATCTCCTGTATTTGG
AGCTACCTCTAGTATCAAAAGTGGTTTAGATTTGAATACAAGTTTGTCCCCTT
CTCTTTTACAGCCTGGGAAAAAAAAACATCTGAAAACACTCCCTTTTAGCAA
CACTTGTATATCTAGATTAGAAAAAACTAGATCAAAATCTGAAGATAGTGCC
CTTTTCACACATCACAGTCTTGGGTCTGAAGTGAACAAGATCATTATCCAGTC
ATCTAATAAACAGATACTTATAAATAAAAATATAAGTGAATCCCTAGGTGAA
CAGAATAGGACTGAGTACGGTAAAGATTCTAACACTGATAAACATTTGGAGC
CCCTGAAATCATTGGGAGGCCGAACATCCAAAAGGAAGAAAACTGAGGAAG
AAAGTGAACATGAAGTAAGCTGCCCCCAAGCTTCTTTTGATAAAGAAAATGC
TTTCCCTTTTCCAATGGATAATCAGTTTTCCATGAATGGAGACTGTGTGATGG
ATAAACCTCTGGATCTGTCTGATCGATTTTCAGCTATTCAGCGTCAAGAGAAA
AGCCAAGGAAGTGAGACTTCTAAAAACAAATTTAGGCAAGTGACTCTTTATG
AGGCTTTGAAGACCATTCCAAAGGGCTTTTCCTCAAGCCGTAAGGCCTCAGA
TGGCAACTGCACGTTGCCCAAAGATTCCCCAGGGGAGCCCTGTTCACAGGAA
TGCATCATCCTTCAGCCCTTGAATAAATGCTCTCCAGACAATAAACCATCATT
ACAAATAAAAGAAGAAAATGCTGTCTTTAAAATTCCTCTACGTCCACGTGAA
AGTTTGGAGACTGAGAATGTTTTAGATGACATAAAGAGTGCTGGTTCTCATG
AGCCAATAAAAATACAAACCAGGTCAGACCATGGAGGATGTGAACTTGCATC
AGTTCTTCAGTTAAATCCATGTAGAACTGGTAAAATAAAGTCTCTACAAAAC
AACCAAGATGTATCCTTTGAAAATATCCAGTGGAGTATAGATCCGGGAGCAG
ACCTTTCTCAGTATAAAATGGATGTTACTGTAATAGATACAAAGGATGGCAG
TCAGTCAAAATTAGGAGGAGAGACAGTGGACATGGACTGTACATTGGTTAGT
GAAACCGTTCTCTTAAAAATGAAGAAGCAAGAGCAGAAGGGAGAAAAAAGT
TCAAATGAAGAAAGAAAAATGAATGATAGCTTGGAAGATATGTTTGATCGGA
CAACACATGAAGAGTATGAATCCTGTTTGGCAGACAGTTTCTCCCAAGCAGC
AGATGAAGAGGAGGAATTGTCTACTGCCACAAAGAAACTACACACTCATGGT
GATAAACAAGACAAAGTCAAGCAGAAAGCGTTTGTGGAGCCGTATTTTAAAG
GTGATGAAAGTATTATGCAGATATGCCAGCAGAAGAAAGAGAAAAGAAATT
GGCTTCCTGCTCAAGACACCGATTCCGCTACATTCCACCCAACACACCAGAG
AATTTTTGGGAAGTTGGTTTTCCTTCCACTCAGACTTGTATGGAAAGAGGTTA
TATTAAGGAAGATCTTGATCCTTGTCCTCGTCCAAAAAGACGTCAGCCTTACA
ACGCAATATTTTCTCCAAAAGGCAAGGAGCAGAAGACATAGACGTTGA
Human RBBP8 Protein Sequence, Variant 2 (SEQ ID NO: 83)
MNISGSSCGSPNSADTSSDFKDLWTKLKECHDREVQGLQVKVTKLKQERILDAQ
RLEEFFTKNQQLREQQKVLHETIKVLEDRLRAGLCDRCAVTEEHMRKKQQEFEN
IRQQNLKLITELMNERNTLQEENKKLSEQLQQKIENDQQHQAAELECEEDVIPDS
PITAFSFSGVNRLRRKENPHVRYIEQTHTKLEHSVCANEMRKVSKSSTHPQHNPN
ENEILVADTYDQSQSPMAKAHGTSSYTPDKSSFNLATVVAETLGLGVQEESETQ
GPMSPLGDELYHCLEGNHKKQPFEESTRNTEDSLRFSDSTSKTPPQEELPTRVSSP
VFGATSSIKSGLDLNTSLSPSLLQPGKKKHLKTLPFSNTCISRLEKTRSKSEDSALF
THHSLGSEVNKIIIQSSNKQILINKNISESLGEQNRTEYGKDSNTDKHLEPLKSLGG
RTSKRKKTEEESEHEVSCPQASFDKENAFPFPMDNQFSMNGDCVMDKPLDLSDR
FSAIQRQEKSQGSETSKNKFRQVTLYEALKTIPKGFSSSRKASDGNCTLPKDSPGE
PCSQECIILQPLNKCSPDNKPSLQIKEENAVFKIPLRPRESLETENVLDDIKSAGSHE
PIKIQTRSDHGGCELASVLQLNPCRTGKIKSLQNNQDVSFENIQWSIDPGADLSQY
KMDVTVIDTKDGSQSKLGGETVDMDCTLVSETVLLKMKKQEQKGEKSSNEERK
MNDSLEDMFDRTTHEEYESCLADSFSQAADEEEELSTATKKLHTHGDKQDKVK
QKAFVEPYFKGDESIMQICQQKKEKRNWLPAQDTDSATFHPTHQRIFGKLVFLPL
RLVWKEVILRKILILVLVQKDVSLTTQYFLQKARSRRHRR
Human MRE11 cDNA Sequence, Variant 1 (SEQ ID NO: 84)
ATGAGTACTGCAGATGCACTTGATGATGAAAACACATTTAAAATATTAGTTG
CAACAGATATTCATCTTGGATTTATGGAGAAAGATGCAGTCAGAGGAAATGA
TACGTTTGTAACACTCGATGAAATTTTAAGACTTGCCCAGGAAAATGAAGTG
GATTTTATTTTGTTAGGTGGTGATCTTTTTCATGAAAATAAGCCCTCAAGGAA
AACATTACATACCTGCCTCGAGTTATTAAGAAAATATTGTATGGGTGATCGGC
CTGTCCAGTTTGAAATTCTCAGTGATCAGTCAGTCAACTTTGGTTTTAGTAAG
TTTCCATGGGTGAACTATCAAGATGGCAACCTCAACATTTCAATTCCAGTGTT
TAGTATTCATGGCAATCATGACGATCCCACAGGGGCAGATGCACTTTGTGCCT
TGGACATTTTAAGTTGTGCTGGATTTGTAAATCACTTTGGACGTTCAATGTCT
GTGGAGAAGATAGACATTAGTCCGGTTTTGCTTCAAAAAGGAAGCACAAAGA
TTGCGCTATATGGTTTAGGATCCATTCCAGATGAAAGGCTCTATCGAATGTTT
GTCAATAAAAAAGTAACAATGTTGAGACCAAAGGAAGATGAGAACTCTTGGT
TTAACTTATTTGTGATTCATCAGAACAGGAGTAAACATGGAAGTACTAACTTC
ATTCCAGAACAATTTTTGGATGACTTCATTGATCTTGTTATCTGGGGCCATGA
ACATGAGTGTAAAATAGCTCCAACCAAAAATGAACAACAGCTGTTTTATATC
TCACAACCTGGAAGCTCAGTGGTTACTTCTCTTTCCCCAGGAGAAGCTGTAAA
GAAACATGTTGGTTTGCTGCGTATTAAAGGGAGGAAGATGAATATGCATAAA
ATTCCTCTTCACACAGTGCGGCAGTTTTTCATGGAGGATATTGTTCTAGCTAA
TCATCCAGACATTTTTAACCCAGATAATCCTAAAGTAACCCAAGCCATACAA
AGCTTCTGTTTGGAGAAGATTGAAGAAATGCTTGAAAATGCTGAACGGGAAC
GTCTGGGTAATTCTCACCAGCCAGAGAAGCCTCTTGTACGACTGCGAGTGGA
CTATAGTGGAGGTTTTGAACCTTTCAGTGTTCTTCGCTTTAGCCAGAAATTTG
TGGATCGGGTAGCTAATCCAAAAGACATTATCCATTTTTTCAGGCATAGAGA
ACAAAAGGAAAAAACAGGAGAAGAGATCAACTTTGGGAAACTTATCACAAA
GCCTTCAGAAGGAACAACTTTAAGGGTAGAAGATCTTGTAAAACAGTACTTT
CAAACCGCAGAGAAGAATGTGCAGCTCTCACTGCTAACAGAAAGAGGGATG
GGTGAAGCAGTACAAGAATTTGTGGACAAGGAGGAGAAAGATGCCATTGAG
GAATTAGTGAAATACCAGTTGGAAAAAACACAGCGATTTCTTAAAGAACGTC
ATATTGATGCCCTCGAAGACAAAATCGATGAGGAGGTACGTCGTTTCAGAGA
AACCAGACAAAAAAATACTAATGAAGAAGATGATGAAGTCCGTGAGGCTAT
GACCAGGGCCAGAGCACTCAGATCTCAGTCAGAGGAGTCTGCTTCTGCCTTT
AGTGCTGATGACCTTATGAGTATAGATTTAGCAGAACAGATGGCTAATGACT
CTGATGATAGCATCTCAGCAGCAACCAACAAAGGAAGAGGCCGAGGAAGAG
GTCGAAGAGGTGGAAGAGGGCAGAATTCAGCATCGAGAGGAGGGTCTCAAA
GAGGAAGAGCAGACACTGGTCTGGAGACTTCTACCCGTAGCAGGAACTCAAA
GACTGCTGTGTCAGCATCTAGAAATATGTCTATTATAGATGCCTTTAAATCTA
CAAGACAGCAGCCTTCCCGAAATGTCACTACTAAGAATTATTCAGAGGTGAT
TGAGGTAGATGAATCAGATGTGGAAGAAGACATTTTTCCTACCACTTCAAAG
ACAGATCAAAGGTGGTCCAGCACATCATCCAGCAAAATCATGTCCCAGAGTC
AAGTATCGAAAGGGGTTGATTTTGAATCAAGTGAGGATGATGATGATGATCC
TTTTATGAACACTAGTTCTTTAAGAAGAAATAGAAGATAA
Human MRE11 Protein Sequence, Variant 1 (SEQ ID NO: 85)
MSTADALDDENTFKILVATDIHLGFMEKDAVRGNDTFVTLDEILRLAQENEVDFI
LLGGDLFHENKPSRKTLHTCLELLRKYCMGDRPVQFEILSDQSVNFGFSKFPWV
NYQDGNLNISIPVFSIHGNHDDPTGADALCALDILSCAGFVNHFGRSMSVEKIDIS
PVLLQKGSTKIALYGLGSIPDERLYRMFVNKKVTMLRPKEDENSWFNLFVIHQN
RSKHGSTNFIPEQFLDDFIDLVIWGHEHECKIAPTKNEQQLFYISQPGSSVVTSLSP
GEAVKKHVGLLRIKGRKMNMHKIPLHTVRQFFMEDIVLANHPDIFNPDNPKVTQ
AIQSFCLEKIEEMLENAERERLGNSHQPEKPLVRLRVDYSGGFEPFSVLRFSQKFV
DRVANPKDIIHFFRHREQKEKTGEEINFGKLITKPSEGTTLRVEDLVKQYFQTAEK
NVQLSLLTERGMGEAVQEFVDKEEKDAIEELVKYQLEKTQRFLKERHIDALEDKI
DEEVRRFRETRQKNTNEEDDEVREAMTRARALRSQSEESASAFSADDLMSIDLA
EQMANDSDDSISAATNKGRGRGRGRRGGRGQNSASRGGSQRGRADTGLETSTR
SRNSKTAVSASRNMSIIDAFKSTRQQPSRNVTTKNYSEVIEVDESDVEEDIFPTTS
KTDQRWSSTSSSKIMSQSQVSKGVDFESSEDDDDDPFMNTSSLRRNRR
Human MRE11 cDNA Sequence, Variant 2 (SEQ ID NO: 86)
ATGAGTACTGCAGATGCACTTGATGATGAAAACACATTTAAAATATTAGTTG
CAACAGATATTCATCTTGGATTTATGGAGAAAGATGCAGTCAGAGGAAATGA
TACGTTTGTAACACTCGATGAAATTTTAAGACTTGCCCAGGAAAATGAAGTG
GATTTTATTTTGTTAGGTGGTGATCTTTTTCATGAAAATAAGCCCTCAAGGAA
AACATTACATACCTGCCTCGAGTTATTAAGAAAATATTGTATGGGTGATCGGC
CTGTCCAGTTTGAAATTCTCAGTGATCAGTCAGTCAACTTTGGTTTTAGTAAG
TTTCCATGGGTGAACTATCAAGATGGCAACCTCAACATTTCAATTCCAGTGTT
TAGTATTCATGGCAATCATGACGATCCCACAGGGGCAGATGCACTTTGTGCCT
TGGACATTTTAAGTTGTGCTGGATTTGTAAATCACTTTGGACGTTCAATGTCT
GTGGAGAAGATAGACATTAGTCCGGTTTTGCTTCAAAAAGGAAGCACAAAGA
TTGCGCTATATGGTTTAGGATCCATTCCAGATGAAAGGCTCTATCGAATGTTT
GTCAATAAAAAAGTAACAATGTTGAGACCAAAGGAAGATGAGAACTCTTGGT
TTAACTTATTTGTGATTCATCAGAACAGGAGTAAACATGGAAGTACTAACTTC
ATTCCAGAACAATTTTTGGATGACTTCATTGATCTTGTTATCTGGGGCCATGA
ACATGAGTGTAAAATAGCTCCAACCAAAAATGAACAACAGCTGTTTTATATC
TCACAACCTGGAAGCTCAGTGGTTACTTCTCTTTCCCCAGGAGAAGCTGTAAA
GAAACATGTTGGTTTGCTGCGTATTAAAGGGAGGAAGATGAATATGCATAAA
ATTCCTCTTCACACAGTGCGGCAGTTTTTCATGGAGGATATTGTTCTAGCTAA
TCATCCAGACATTTTTAACCCAGATAATCCTAAAGTAACCCAAGCCATACAA
AGCTTCTGTTTGGAGAAGATTGAAGAAATGCTTGAAAATGCTGAACGGGAAC
GTCTGGGTAATTCTCACCAGCCAGAGAAGCCTCTTGTACGACTGCGAGTGGA
CTATAGTGGAGGTTTTGAACCTTTCAGTGTTCTTCGCTTTAGCCAGAAATTTG
TGGATCGGGTAGCTAATCCAAAAGACATTATCCATTTTTTCAGGCATAGAGA
ACAAAAGGAAAAAACAGGAGAAGAGATCAACTTTGGGAAACTTATCACAAA
GCCTTCAGAAGGAACAACTTTAAGGGTAGAAGATCTTGTAAAACAGTACTTT
CAAACCGCAGAGAAGAATGTGCAGCTCTCACTGCTAACAGAAAGAGGGATG
GGTGAAGCAGTACAAGAATTTGTGGACAAGGAGGAGAAAGATGCCATTGAG
GAATTAGTGAAATACCAGTTGGAAAAAACACAGCGATTTCTTAAAGAACGTC
ATATTGATGCCCTCGAAGACAAAATCGATGAGGAGGTACGTCGTTTCAGAGA
AACCAGACAAAAAAATACTAATGAAGAAGATGATGAAGTCCGTGAGGCTAT
GACCAGGGCCAGAGCACTCAGATCTCAGTCAGAGGAGTCTGCTTCTGCCTTT
AGTGCTGATGACCTTATGAGTATAGATTTAGCAGAACAGATGGCTAATGACT
CTGATGATAGCATCTCAGCAGCAACCAACAAAGGAAGAGGCCGAGGAAGAG
GTCGAAGAGGTGGAAGAGGGCAGAATTCAGCATCGAGAGGAGGGTCTCAAA
GAGGAAGAGCCTTTAAATCTACAAGACAGCAGCCTTCCCGAAATGTCACTAC
TAAGAATTATTCAGAGGTGATTGAGGTAGATGAATCAGATGTGGAAGAAGAC
ATTTTTCCTACCACTTCAAAGACAGATCAAAGGTGGTCCAGCACATCATCCAG
CAAAATCATGTCCCAGAGTCAAGTATCGAAAGGGGTTGATTTTGAATCAAGT
GAGGATGATGATGATGATCCTTTTATGAACACTAGTTCTTTAAGAAGAAATA
GAAGATAA
Human MRE11 Protein Sequence, Variant 2 (SEQ ID NO: 87)
MSTADALDDENTFKILVATDIHLGFMEKDAVRGNDTFVTLDEILRLAQENEVDFI
LLGGDLFHENKPSRKTLHTCLELLRKYCMGDRPVQFEILSDQSVNFGFSKFPWV
NYQDGNLNISIPVFSIHGNHDDPTGADALCALDILSCAGFVNHFGRSMSVEKIDIS
PVLLQKGSTKIALYGLGSIPDERLYRMFVNKKVTMLRPKEDENSWFNLFVIHQN
RSKHGSTNFIPEQFLDDFIDLVIWGHEHECKIAPTKNEQQLFYISQPGSSVVTSLSP
GEAVKKHVGLLRIKGRKMNMHKIPLHTVRQFFMEDIVLANHPDIFNPDNPKVTQ
AIQSFCLEKIEEMLENAERERLGNSHQPEKPLVRLRVDYSGGFEPFSVLRFSQKFV
DRVANPKDIIHFFRHREQKEKTGEEINFGKLITKPSEGTTLRVEDLVKQYFQTAEK
NVQLSLLTERGMGEAVQEFVDKEEKDAIEELVKYQLEKTQRFLKERHIDALEDKI
DEEVRRFRETRQKNTNEEDDEVREAMTRARALRSQSEESASAFSADDLMSIDLA
EQMANDSDDSISAATNKGRGRGRGRRGGRGQNSASRGGSQRGRAFKSTRQQPS
RNVTTKNYSEVIEVDESDVEEDIFPTTSKTDQRWSSTSSSKIMSQSQVSKGVDFES
SEDDDDDPFMNTSSLRRNRR
Human MRE11 cDNA Sequence, Variant 3 (SEQ ID NO: 88)
ATGAGTACTGCAGATGCACTTGATGATGAAAACACATTTAAAATATTAGTTG
CAACAGATATTCATCTTGGATTTATGGAGAAAGATGCAGTCAGAGGAAATGA
TACGTTTGTAACACTCGATGAAATTTTAAGACTTGCCCAGGAAAATGAAGTG
GATTTTATTTTGTTAGGTGGTGATCTTTTTCATGAAAATAAGCCCTCAAGGAA
AACATTACATACCTGCCTCGAGTTATTAAGAAAATATTGTATGGGTGATCGGC
CTGTCCAGTTTGAAATTCTCAGTGATCAGTCAGTCAACTTTGGTTTTAGTAAG
TTTCCATGGGTGAACTATCAAGATGGCAACCTCAACATTTCAATTCCAGTGTT
TAGTATTCATGGCAATCATGACGATCCCACAGGGGCAGATGCACTTTGTGCCT
TGGACATTTTAAGTTGTGCTGGATTTGTAAATCACTTTGGACGTTCAATGTCT
GTGGAGAAGATAGACATTAGTCCGGTTTTGCTTCAAAAAGGAAGCACAAAGA
TTGCGCTATATGGTTTAGGATCCATTCCAGATGAAAGGCTCTATCGAATGTTT
GTCAATAAAAAAGTAACAATGTTGAGACCAAAGGAAGATGAGAACTCTTGGT
TTAACTTATTTGTGATTCATCAGAACAGGAGTAAACATGGAAGTACTAACTTC
ATTCCAGAACAATTTTTGGATGACTTCATTGATCTTGTTATCTGGGGCCATGA
ACATGAGTGTAAAATAGCTCCAACCAAAAATGAACAACAGCTGTTTTATATC
TCACAACCTGGAAGCTCAGTGGTTACTTCTCTTTCCCCAGGAGAAGCTGTAAA
GAAACATGTTGGTTTGCTGCGTATTAAAGGGAGGAAGATGAATATGCATAAA
ATTCCTCTTCACACAGTGCGGCAGTTTTTCATGGAGGATATTGTTCTAGCTAA
TCATCCAGACATTTTTAACCCAGATAATCCTAAAGTAACCCAAGCCATACAA
AGCTTCTGTTTGGAGAAGATTGAAGAAATGCTTGAAAATGCTGAACGGGAAC
GTCTGGGTAATTCTCACCAGCCAGAGAAGCCTCTTGTACGACTGCGAGTGGA
CTATAGTGGAGGTTTTGAACCTTTCAGTGTTCTTCGCTTTAGCCAGAAATTTG
TGGATCGGGTAGCTAATCCAAAAGACATTATCCATTTTTTCAGGCATAGAGA
ACAAAAGGAAAAAACAGGAGAAGAGATCAACTTTGGGAAACTTATCACAAA
GCCTTCAGAAGGAACAACTTTAAGGGTAGAAGATCTTGTAAAACAGTACTTT
CAAACCGCAGAGAAGAATGTGCAGCTCTCACTGCTAACAGAAAGAGGGATG
GGTGAAGCAGTACAAGAATTTGTGGACAAGGAGGAGAAAGATGCCATTGAG
GAATTAGTGAAATACCAGTTGGAAAAAACACAGCGATTTCTTAAAGAACGTC
ATATTGATGCCCTCGAAGACAAAATCGATGAGGAGGTACGTCGTTTCAGAGA
AACCAGACAAAAAAATACTAATGAAGAAGATGATGAAGTCCGTGAGGCTAT
GACCAGGGCCAGAGCACTCAGATCTCAGTCAGAGGAGTCTGCTTCTGCCTTT
AGTGCTGATGACCTTATGAGTATAGATTTAGCAGAACAGATGGCTAATGACT
CTGATGATAGCATCTCAGCAGCAACCAACAAAGGAAGAGGCCGAGGAAGAG
GTCGAAGAGGTGGAAGAGGGCAGAATTCAGCATCGAGAGGAGGGTCTCAAA
GAGGAAGAGACACTGGTCTGGAGACTTCTACCCGTAGCAGGAACTCAAAGAC
TGCTGTGTCAGCATCTAGAAATATGTCTATTATAGATGCCTTTAAATCTACAA
GACAGCAGCCTTCCCGAAATGTCACTACTAAGAATTATTCAGAGGTGATTGA
GGTAGATGAATCAGATGTGGAAGAAGACATTTTTCCTACCACTTCAAAGACA
GATCAAAGGTGGTCCAGCACATCATCCAGCAAAATCATGTCCCAGAGTCAAG
TATCGAAAGGGGTTGATTTTGAATCAAGTGAGGATGATGATGATGATCCTTTT
ATGAACACTAGTTCTTTAAGAAGAAATAGAAGATAA
Human MRE11 Protein Sequence, Variant 3 (SEQ ID NO: 89)
MSTADALDDENTFKILVATDIHLGFMEKDAVRGNDTFVTLDEILRLAQENEVDFI
LLGGDLFHENKPSRKTLHTCLELLRKYCMGDRPVQFEILSDQSVNFGFSKFPWV
NYQDGNLNISIPVFSIHGNHDDPTGADALCALDILSCAGFVNHFGRSMSVEKIDIS
PVLLQKGSTKIALYGLGSIPDERLYRMFVNKKVTMLRPKEDENSWFNLFVIHQN
RSKHGSTNFIPEQFLDDFIDLVIWGHEHECKIAPTKNEQQLFYISQPGSSVVTSLSP
GEAVKKHVGLLRIKGRKMNMHKIPLHTVRQFFMEDIVLANHPDIFNPDNPKVTQ
AIQSFCLEKIEEMLENAERERLGNSHQPEKPLVRLRVDYSGGFEPFSVLRFSQKFV
DRVANPKDIIHFFRHREQKEKTGEEINFGKLITKPSEGTTLRVEDLVKQYFQTAEK
NVQLSLLTERGMGEAVQEFVDKEEKDAIEELVKYQLEKTQRFLKERHIDALEDKI
DEEVRRFRETRQKNTNEEDDEVREAMTRARALRSQSEESASAFSADDLMSIDLA
EQMANDSDDSISAATNKGRGRGRGRRGGRGQNSASRGGSQRGRDTGLETSTRS
RNSKTAVSASRNMSIIDAFKSTRQQPSRNVTTKNYSEVIEVDESDVEEDIFPTTSK
TDQRWSSTSSSKIMSQSQVSKGVDFESSEDDDDDPFMNTSSLRRNRR
Wildtype Human ATM (SEQ ID NO: 90)
MSLVLNDLLICCRQLEHDRATERKKEVEKFKRLIRDPETIKHLDRHSDSKQGKYLNWDAV
FRFLQKYIQKETECLRIAKPNVSASTQASRQKKMQEISSLVKYFIKCANRRAPRLKCQEL
LNYIMDTVKDSSNGAIYGADCSNILLKDILSVRKYWCEISQQQWLELFSVYFRLYLKPSQ
DVHRVLVARIIHAVTKGCCSQTDGLNSKELDFFSKAIQCARQEKSSSGLNHILAALTIFL
KTLAVNFRIRVCELGDEILPTLLYIWTQHRLNDSLKEVIIELFQLQIYIHHPKGAKTQEK
GAYESTKWRSILYNLYDLLVNEISHIGSRGKYSSGFRNIAVKENLIELMADICHQVFNED
TRSLEISQSYTTTQRESSDYSVPCKRKKIELGWEVIKDHLQKSQNDFDLVPWLQIATQLI
SKYPASLPNCELSPLLMILSQLLPQQRHGERTPYVLRCLTEVALCQDKRSNLESSQKSDL
LKLWNKIWCITFRGISSEQIQAENFGLLGAIIQGSLVEVDREFWKLFTGSACRPSCPAVC
CLTLALTTSIVPGTVKMGIEQNMCEVNRSFSLKESIMKWLLFYQLEGDLENSTEVPPILH
SNFPHLVLEKILVSLTMKNCKAAMNFFQSVPECEHHQKDKEELSFSEVEELFLQTTFDKM
DFLTIVRECGIEKHQSSIGFSVHQNLKESLDRCLLGLSEQLLNNYSSEITNSETLVRCSR
LLVGVLGCYCYMGVIAEEEAYKSELFQKAKSLMQCAGESITLFKNKTNEEFRIGSLRNMM
QLCTRCLSNCTKKSPNKIASGFFLRLLTSKLMNDIADICKSLASFIKKPFDRGEVESMED
DTNGNLMEVEDQSSMNLFNDYPDSSVSDANEPGESQSTIGAINPLAEEYLSKQDLLFLDM
LKFLCLCVTTAQTNTVSFRAADIRRKLLMLIDSSTLEPTKSLHLHMYLMLLKELPGEEYP
LPMEDVLELLKPLSNVCSLYRRDQDVCKTILNHVLHVVKNLGQSNMDSENTRDAQGQFLT
VIGAFWHLTKERKYIFSVRMALVNCLKTLLEADPYSKWAILNVMGKDFPVNEVFTQFLAD
NHHQVRMLAAESINRLFQDTKGDSSRLLKALPLKLQQTAFENAYLKAQEGMREMSHSAEN
PETLDEIYNRKSVLLTLIAVVLSCSPICEKQALFALCKSVKENGLEPHLVKKVLEKVSET
FGYRRLEDFMASHLDYLVLEWLNLQDTEYNLSSFPFILLNYTNIEDFYRSCYKVLIPHLV
IRSHFDEVKSIANQIQEDWKSLLTDCFPKILVNILPYFAYEGTRDSGMAQQRETATKVYD
MLKSENLLGKQIDHLFISNLPEIVVELLMTLHEPANSSASQSTDLCDFSGDLDPAPNPPH
FPSHVIKATFAYISNCHKTKLKSILEILSKSPDSYQKILLAICEQAAETNNVYKKHRILK
IYHLFVSLLLKDIKSGLGGAWAFVLRDVIYTLIHYINQRPSCIMDVSLRSFSLCCDLLSQ
VCQTAVTYCKDALENHLHVIVGTLIPLVYEQVEVQKQVLDLLKYLVIDNKDNENLYITIK
LLDPFPDHVVFKDLRITQQKIKYSRGPFSLLEEINHFLSVSVYDALPLTRLEGLKDLRRQ
LELHKDQMVDIMRASQDNPQDGIMVKLVVNLLQLSKMAINHTGEKEVLEAVGSCLGEVGP
IDFSTIAIQHSKDASYTKALKLFEDKELQWTFIMLTYLNNTLVEDCVKVRSAAVTCLKNI
LATKTGHSFWEIYKMTTDPMLAYLQPERTSRKKFLEVPREDKENPFEGLDDINLWIPLSE
NHDIWIKTLTCAFLDSGGTKCEILQLLKPMCEVKTDFCQTVLPYLIHDILLQDTNESWRN
LLSTHVQGFFTSCLRHFSQTSRSTTPANLDSESEHFFRCCLDKKSQRTMLAVVDYMRRQK
RPSSGTIFNDAFWLDLNYLEVAKVAQSCAAHFTALLYAEIYADKKSMDDQEKRSLAFEEG
SQSTTISSLSEKSKEETGISLQDLLLEIYRSIGEPDSLYGCGGGKMLQPITRLRTYEHEA
MWGKALVTYDLETAIPSSTRQAGIIQALQNLGLCHILSVYLKGLDYENKDWCPELEELHY
QAAWRNMQWDHCTSVSKEVEGTSYHESLYNALQSLRDREFSTFYESLKYARVKEVEEMCK
RSLESVYSLYPTLSRLQAIGELESIGELFSRSVTHRQLSEVYIKWQKHSQLLKDSDFSFQ
EPIMALRTVILEILMEKEMDNSQRECIKDILTKHLVELSILARTFKNTQLPERAIFQIKQ
YNSVSCGVSEWQLEEAQVFWAKKEQSLALSILKQMIKKLDASCAANNPSLKLTYTECLRV
CGNWLAETCLENPAVIMQTYLEKAVEVAGNYDGESSDELRNGKMKAFLSLARFSDTQYQR
IENYMKSSEFENKQALLKRAKEEVGLLREHKIQTNRYTVKVQRELELDELALRALKEDRK
RFLCKAVENYINCLLSGEEHDMWVFRLCSLWLENSGVSEVNGMMKRDGMKIPTYKFLPLM
YQLAARMGTKMMGGLGFHEVLNNLISRISMDHPHHTLFIILALANANRDEFLTKPEVARR
SRITKNVPKQSSQLDEDRTEAANRIICTIRSRRPQMVRSVEALCDAYIILANLDATQWKT
QRKGINIPADQPITKLKNLEDVVVPTMEIKVDHTGEYGNLVTIQSFKAEFRLAGGVNLPK
IIDCVGSDGKERRQLVKGRDDLRQDAVMQQVFQMCNTLLQRNTETRKRKLTICTYKVVPL
SQRSGVLEWCTGTVPIGEFLVNNEDGAHKRYRPNDFSAFQCQKKMMEVQKKSFEEKYEVF
MDVCQNFQPVFRYFCMEKFLDPAIWFEKRLAYTRSVATSSIVGYILGLGDRHVQNILINE
QSAELVHIDLGVAFEQGKILPTPETVPFRLTRDIVDGMGITGVEGVFRRCCEKTMEVMRN
SQETLLTIVEVLLYDPLFDWTMNPLKALYLQQRPEDETELHPTLNADDQECKRNLSDIDQ
SFNKVAERVLMRLQEKLKGVEEGTVLSVGGQVNLLIQQAIDPKNLSRLFPGWKAWV
Wildtype Human ATM cDNA (SEQ ID NO: 91)
aggtagctgc gtggctaacg gagaaaagaa gccgtggccg cgggaggagg cgagaggagt
cgggatctgc gctgcagcca ccgccgcggt tgatactact ttgaccttcc gagtgcagtg
acagtgatgt gtgttctgaa attgtgaacc atgagtctag tacttaatga tctgcttatc
tgctgccgtc aactagaaca tgatagagct acagaacgaa agaaagaagt tgagaaattt
aagcgcctga ttcgagatcc tgaaacaatt aaacatctag atcggcattc agattccaaa
caaggaaaat atttgaattg ggatgctgtt tttagatttt tacagaaata tattcagaaa
gaaacagaat gtctgagaat agcaaaacca aatgtatcag cctcaacaca agcctccagg
cagaaaaaga tgcaggaaat cagtagtttg gtcaaatact tcatcaaatg tgcaaacaga
agagcaccta ggctaaaatg tcaagaactc ttaaattata tcatggatac agtgaaagat
tcatctaatg gtgctattta cggagctgat tgtagcaaca tactactcaa agacattctt
tctgtgagaa aatactggtg tgaaatatct cagcaacagt ggttagaatt gttctctgtg
tacttcaggc tctatctgaa accttcacaa gatgttcata gagttttagt ggctagaata
attcatgctg ttaccaaagg atgctgttct cagactgacg gattaaattc caaatttttg
gacttttttt ccaaggctat tcagtgtgcg agacaagaaa agagctcttc aggtctaaat
catatcttag cagctcttac tatcttcctc aagactttgg ctgtcaactt tcgaattcga
gtgtgtgaat taggagatga aattcttccc actttgcttt atatttggac tcaacatagg
cttaatgatt ctttaaaaga agtcattatt gaattatttc aactgcaaat ttatatccat
catccgaaag gagccaaaac ccaagaaaaa ggtgcttatg aatcaacaaa atggagaagt
attttataca acttatatga tctgctagtg aatgagataa gtcatatagg aagtagagga
aagtattctt caggatttcg taatattgcc gtcaaagaaa atttgattga attgatggca
gatatctgtc accaggtttt taatgaagat accagatcct tggagatttc tcaatcttac
actactacac aaagagaatc tagtgattac agtgtccctt gcaaaaggaa gaaaatagaa
ctaggctggg aagtaataaa agatcacctt cagaagtcac agaatgattt tgatcttgtg
ccttggctac agattgcaac ccaattaata tcaaagtatc ctgcaagttt acctaactgt
gagctgtctc cattactgat gatactatct cagcttctac cccaacagcg acatggggaa
cgtacaccat atgtgttacg atgccttacg gaagttgcat tgtgtcaaga caagaggtca
aacctagaaa gctcacaaaa gtcagattta ttaaaactct ggaataaaat ttggtgtatt
acctttcgtg gtataagttc tgagcaaata caagctgaaa actttggctt acttggagcc
ataattcagg gtagtttagt tgaggttgac agagaattct ggaagttatt tactgggtca
gcctgcagac cttcatgtcc tgcagtatgc tgtttgactt tggcactgac caccagtata
gttccaggaa cggtaaaaat gggaatagag caaaatatgt gtgaagtaaa tagaagcttt
tctttaaagg aatcaataat gaaatggctc ttattctatc agttagaggg tgacttagaa
aatagcacag aagtgcctcc aattcttcac agtaattttc ctcatcttgt actggagaaa
attcttgtga gtctcactat gaaaaactgt aaagctgcaa tgaatttttt ccaaagcgtg
ccagaatgtg aacaccacca aaaagataaa gaagaacttt cattctcaga agtagaagaa
ctatttcttc agacaacttt tgacaagatg gactttttaa ccattgtgag agaatgtggt
atagaaaagc accagtccag tattggcttc tctgtccacc agaatctcaa ggaatcactg
gatcgctgtc ttctgggatt atcagaacag cttctgaata attactcatc tgagattaca
aattcagaaa ctcttgtccg gtgttcacgt cttttggtgg gtgtccttgg ctgctactgt
tacatgggtg taatagctga agaggaagca tataagtcag aattattcca gaaagccaag
tctctaatgc aatgtgcagg agaaagtatc actctgttta aaaataagac aaatgaggaa
ttcagaattg gttccttgag aaatatgatg cagctatgta cacgttgctt gagcaactgt
accaagaaga gtccaaataa gattgcatct ggctttttcc tgcgattgtt aacatcaaag
ctaatgaatg acattgcaga tatttgtaaa agtttagcat ccttcatcaa aaagccattt
gaccgtggag aagtagaatc aatggaagat gatactaatg gaaatctaat ggaggtggag
gatcagtcat ccatgaatct atttaacgat taccctgata gtagtgttag tgatgcaaac
gaacctggag agagccaaag taccataggt gccattaatc ctttagctga agaatatctg
tcaaagcaag atctactttt cttagacatg ctcaagttct tgtgtttgtg tgtaactact
gctcagacca atactgtgtc ctttagggca gctgatattc ggaggaaatt gttaatgtta
attgattcta gcacgctaga acctaccaaa tccctccacc tgcatatgta tctaatgctt
ttaaaggagc ttcctggaga agagtacccc ttgccaatgg aagatgttct tgaacttctg
aaaccactat ccaatgtgtg ttctttgtat cgtcgtgacc aagatgtttg taaaactatt
ttaaaccatg tccttcatgt agtgaaaaac ctaggtcaaa gcaatatgga ctctgagaac
acaagggatg ctcaaggaca gtttcttaca gtaattggag cattttggca tctaacaaag
gagaggaaat atatattctc tgtaagaatg gccctagtaa attgccttaa aactttgctt
gaggctgatc cttattcaaa atgggccatt cttaatgtaa tgggaaaaga ctttcctgta
aatgaagtat ttacacaatt tcttgctgac aatcatcacc aagttcgcat gttggctgca
gagtcaatca atagattgtt ccaggacacg aagggagatt cttccaggtt actgaaagca
cttcctttga agcttcagca aacagctttt gaaaatgcat acttgaaagc tcaggaagga
atgagagaaa tgtcccatag tgctgagaac cctgaaactt tggatgaaat ttataataga
aaatctgttt tactgacgtt gatagctgtg gttttatcct gtagccctat ctgcgaaaaa
caggctttgt ttgccctgtg taaatctgtg aaagagaatg gattagaacc tcaccttgtg
aaaaaggttt tagagaaagt ttctgaaact tttggatata gacgtttaga agactttatg
gcatctcatt tagattatct ggttttggaa tggctaaatc ttcaagatac tgaatacaac
ttatcttctt ttccttttat tttattaaac tacacaaata ttgaggattt ctatagatct
tgttataagg ttttgattcc acatctggtg attagaagtc attttgatga ggtgaagtcc
attgctaatc agattcaaga ggactggaaa agtcttctaa cagactgctt tccaaagatt
cttgtaaata ttcttcctta ttttgcctat gagggtacca gagacagtgg gatggcacag
caaagagaga ctgctaccaa ggtctatgat atgcttaaaa gtgaaaactt attgggaaaa
cagattgatc acttattcat tagtaattta ccagagattg tggtggagtt attgatgacg
ttacatgagc cagcaaattc tagtgccagt cagagcactg acctctgtga cttttcaggg
gatttggatc ctgctcctaa tccacctcat tttccatcgc atgtgattaa agcaacattt
gcctatatca gcaattgtca taaaaccaag ttaaaaagca ttttagaaat tctttccaaa
agccctgatt cctatcagaa aattcttctt gccatatgtg agcaagcagc tgaaacaaat
aatgtttata agaagcacag aattcttaaa atatatcacc tgtttgttag tttattactg
aaagatataa aaagtggctt aggaggagct tgggcctttg ttcttcgaga cgttatttat
actttgattc actatatcaa ccaaaggcct tcttgtatca tggatgtgtc attacgtagc
ttctcccttt gttgtgactt attaagtcag gtttgccaga cagccgtgac ttactgtaag
gatgctctag aaaaccatct tcatgttatt gttggtacac ttatacccct tgtgtatgag
caggtggagg ttcagaaaca ggtattggac ttgttgaaat acttagtgat agataacaag
gataatgaaa acctctatat cacgattaag cttttagatc cttttcctga ccatgttgtt
tttaaggatt tgcgtattac tcagcaaaaa atcaaataca gtagaggacc cttttcactc
ttggaggaaa ttaaccattt tctctcagta agtgtttatg atgcacttcc attgacaaga
cttgaaggac taaaggatct tcgaagacaa ctggaactac ataaagatca gatggtggac
attatgagag cttctcagga taatccgcaa gatgggatta tggtgaaact agttgtcaat
ttgttgcagt tatccaagat ggcaataaac cacactggtg aaaaagaagt tctagaggct
gttggaagct gcttgggaga agtgggtcct atagatttct ctaccatagc tatacaacat
agtaaagatg catcttatac caaggccctt aagttatttg aagataaaga acttcagtgg
accttcataa tgctgaccta cctgaataac acactggtag aagattgtgt caaagttcga
tcagcagctg ttacctgttt gaaaaacatt ttagccacaa agactggaca tagtttctgg
gagatttata agatgacaac agatccaatg ctggcctatc tacagccttt tagaacatca
agaaaaaagt ttttagaagt acccagattt gacaaagaaa acccttttga aggcctggat
gatataaatc tgtggattcc tctaagtgaa aatcatgaca tttggataaa gacactgact
tgtgcttttt tggacagtgg aggcacaaaa tgtgaaattc ttcaattatt aaagccaatg
tgtgaagtga aaactgactt ttgtcagact gtacttccat acttgattca tgatatttta
ctccaagata caaatgaatc atggagaaat ctgctttcta cacatgttca gggatttttc
accagctgtc ttcgacactt ctcgcaaacg agccgatcca caacccctgc aaacttggat
tcagagtcag agcacttttt ccgatgctgt ttggataaaa aatcacaaag aacaatgctt
gctgttgtgg actacatgag aagacaaaag agaccttctt caggaacaat ttttaatgat
gctttctggc tggatttaaa ttatctagaa gttgccaagg tagctcagtc ttgtgctgct
cactttacag ctttactcta tgcagaaatc tatgcagata agaaaagtat ggatgatcaa
gagaaaagaa gtcttgcatt tgaagaagga agccagagta caactatttc tagcttgagt
gaaaaaagta aagaagaaac tggaataagt ttacaggatc ttctcttaga aatctacaga
agtatagggg agccagatag tttgtatggc tgtggtggag ggaagatgtt acaacccatt
actagactac gaacatatga acacgaagca atgtggggca aagccctagt aacatatgac
ctcgaaacag caatcccctc atcaacacgc caggcaggaa tcattcaggc cttgcagaat
ttgggactct gccatattct ttccgtctat ttaaaaggat tggattatga aaataaagac
tggtgtcctg aactagaaga acttcattac caagcagcat ggaggaatat gcagtgggac
cattgcactt ccgtcagcaa agaagtagaa ggaaccagtt accatgaatc attgtacaat
gctctacaat ctctaagaga cagagaattc tctacatttt atgaaagtct caaatatgcc
agagtaaaag aagtggaaga gatgtgtaag cgcagccttg agtctgtgta ttcgctctat
cccacactta gcaggttgca ggccattgga gagctggaaa gcattgggga gcttttctca
agatcagtca cacatagaca actctctgaa gtatatatta agtggcagaa acactcccag
cttctcaagg acagtgattt tagttttcag gagcctatca tggctctacg cacagtcatt
ttggagatcc tgatggaaaa ggaaatggac aactcacaaa gagaatgtat taaggacatt
ctcaccaaac accttgtaga actctctata ctggccagaa ctttcaagaa cactcagctc
cctgaaaggg caatatttca aattaaacag tacaattcag ttagctgtgg agtctctgag
tggcagctgg aagaagcaca agtattctgg gcaaaaaagg agcagagtct tgccctgagt
attctcaagc aaatgatcaa gaagttggat gccagctgtg cagcgaacaa tcccagccta
aaacttacat acacagaatg tctgagggtt tgtggcaact ggttagcaga aacgtgctta
gaaaatcctg cggtcatcat gcagacctat ctagaaaagg cagtagaagt tgctggaaat
tatgatggag aaagtagtga tgagctaaga aatggaaaaa tgaaggcatt tctctcatta
gcccggtttt cagatactca ataccaaaga attgaaaact acatgaaatc atcggaattt
gaaaacaagc aagctctcct gaaaagagcc aaagaggaag taggtctcct tagggaacat
aaaattcaga caaacagata cacagtaaag gttcagcgag agctggagtt ggatgaatta
gccctgcgtg cactgaaaga ggatcgtaaa cgcttcttat gtaaagcagt tgaaaattat
atcaactgct tattaagtgg agaagaacat gatatgtggg tattccgact ttgttccctc
tggcttgaaa attctggagt ttctgaagtc aatggcatga tgaagagaga cggaatgaag
attccaacat ataaattttt gcctcttatg taccaattgg ctgctagaat ggggaccaag
atgatgggag gcctaggatt tcatgaagtc ctcaataatc taatctctag aatttcaatg
gatcaccccc atcacacttt gtttattata ctggccttag caaatgcaaa cagagatgaa
tttctgacta aaccagaggt agccagaaga agcagaataa ctaaaaatgt gcctaaacaa
agctctcagc ttgatgagga tcgaacagag gctgcaaata gaataatatg tactatcaga
agtaggagac ctcagatggt cagaagtgtt gaggcacttt gtgatgctta tattatatta
gcaaacttag atgccactca gtggaagact cagagaaaag gcataaatat tccagcagac
cagccaatta ctaaacttaa gaatttagaa gatgttgttg tccctactat ggaaattaag
gtggaccaca caggagaata tggaaatctg gtgactatac agtcatttaa agcagaattt
cgcttagcag gaggtgtaaa tttaccaaaa ataatagatt gtgtaggttc cgatggcaag
gagaggagac agcttgttaa gggccgtgat gacctgagac aagatgctgt catgcaacag
gtcttccaga tgtgtaatac attactgcag agaaacacgg aaactaggaa gaggaaatta
actatctgta cttataaggt ggttcccctc tctcagcgaa gtggtgttct tgaatggtgc
acaggaactg tccccattgg tgaatttctt gttaacaatg aagatggtgc tcataaaaga
tacaggccaa atgatttcag tgcctttcag tgccaaaaga aaatgatgga ggtgcaaaaa
aagtcttttg aagagaaata tgaagtcttc atggatgttt gccaaaattt tcaaccagtt
ttccgttact tctgcatgga aaaattcttg gatccagcta tttggtttga gaagcgattg
gcttatacgc gcagtgtagc tacttcttct attgttggtt acatacttgg acttggtgat
agacatgtac agaatatctt gataaatgag cagtcagcag aacttgtaca tatagatcta
ggtgttgctt ttgaacaggg caaaatcctt cctactcctg agacagttcc ttttagactc
accagagata ttgtggatgg catgggcatt acgggtgttg aaggtgtctt cagaagatgc
tgtgagaaaa ccatggaagt gatgagaaac tctcaggaaa ctctgttaac cattgtagag
gtccttctat atgatccact ctttgactgg accatgaatc ctttgaaagc tttgtattta
cagcagaggc cggaagatga aactgagctt caccctactc tgaatgcaga tgaccaagaa
tgcaaacgaa atctcagtga tattgaccag agtttcaaca aagtagctga acgtgtctta
atgagactac aagagaaact gaaaggagtg gaagaaggca ctgtgctcag tgttggtgga
caagtgaatt tgctcataca gcaggccata gaccccaaaa atctcagccg acttttccca
ggatggaaag cttgggtgtg atcttcagta tatgaattac cctttcattc agcctttaga
aattatattt tagcctttat ttttaacctg ccaacatact ttaagtaggg attaatattt
aagtgaacta ttgtgggttt ttttgaatgt tggttttaat acttgattta atcaccactc
aaaaatgttt tgatggtctt aaggaacatc tctgctttca ctctttagaa ataatggtca
ttcgggctgg gcgcagcggc tcacgcctgt aatcccagca ctttgggagg ccgaggtgag
cggatcacaa ggtcaggagt tcgagaccag cctggccaag agaccagcct ggccagtatg
gtgaaaccct gtctctacta aaaatacaaa aattagccga gcatggtggc gggcacctgt
aatcccagct actcgagagg ctgaggcagg agaatctctt gaacctggga ggtgaaggtt
gctgtgggcc aaaatcatgc cattgcactc cagcctgggt gacaagagcg aaactccatc
tcaaaaaaaa aaaaaaaaaa acagaaacgt atttggattt ttcctagtaa gatcactcag
tgttactaaa taatgaagtt gttatggaga acaaatttca aagacacagt tagtgtagtt
actatttttt taagtgtgta ttaaaacttc tcattctatt ctctttatct tttaagccct
tctgtactgt ccatgtatgt tatctttctg tgataacttc atagattgcc ttctagttca
tgaattctct tgtcagatgt atataatctc ttttacccta tccattgggc ttcttctttc
agaaattgtt tttcatttct aattatgcat catttttcag atctctgttt cttgatgtca
tttttaatgt ttttttaatg ttttttatgt cactaattat tttaaatgtc tgtacttgat
agacactgta atagttctat taaatttagt tcctgctgtt tatatctgtt gatttttgta
tttgataggc tgttcatcca gttttgtctt tttgaaaagt gagtttattt tcagcaaggc
tttatctatg ggaatcttga gtgtctgttt atgtcatatt cccagggctg ttgctgcaca
caagcccatt cttattttaa tttcttggct ttagggtttc catacctgaa gtgtagcata
aatactgata ggagatttcc caggccaagg caaacacact tcctcctcat ctccttgtgc
tagtgggcag aatatttgat tgatgccttt ttcactgaga gtataagctt ccatgtgtcc
cacctttatg gcaggggtgg aaggaggtac atttaattcc cactgcctgc ctttggcaag
ccctgggttc tttgctcccc atatagatgt ctaagctaaa agccgtgggt taatgagact
ggcaaattgt tccaggacag ctacagcatc agctcacata ttcacctctc tggtttttca
ttcccctcat ttttttctga gacagagtct tgctctgtca cccaggctgg agtgcagtgg
catgatctca gctcactgaa acctctgcct cctgggttca agcaattctc ctgcctcagc
ctcccgagta gctgggacta caggcgtgtg ccaacacgcc cggctaattt tttgtatttt
tattagagac ggagtttcac cgtgttagcc aggatggtct cgatcgcttg acctcgtgat
ccaccctcct cggcctccca aagtgctggg attacaggtg tgagccaccg cgcccggcct
cattcccctc atttttgacc gtaaggattt cccctttctt gtaagttctg ctatgtattt
aaaagaatgt tttctacatt ttatccagca tttctctgtg ttctgttgga agggaagggc
ttaggtatct agtttgatac ataggtagaa gtggaacatt tctctgtccc ccagctgtca
tcatataaga taaacatcag ataaaaagcc acctgaaagt aaaactactg actcgtgtat
tagtgagtat aatctcttct ccatccttag gaaaatgttc atcccagctg cggagattaa
caaatgggtg attgagcttt ctcctcgtat ttggaccttg aaggttatat aaattttttt
cttatgaaga gttggcattt ctttttattg ccaatggcag gcactcattc atatttgatc
tcctcacctt cccctcccct aaaaccaatc tccagaactt tttggactat aaatttcttg
gtttgacttc tggagaactg ttcagaatat tactttgcat ttcaaattac aaacttacct
tggtgtatct ttttcttaca agctgcctaa atgaatattt ggtatatatt ggtagtttta
ttactatagt aaatcaagga aatgcagtaa acttaaaatg tctttaagaa agccctgaaa
tcttcatggg tgaaattaga aattatcaac tagataatag tatagataaa tgaatttgta
gctaattctt gctagttgtt gcatccagag agctttgaat aacatcatta atctactctt
tagccttgca tggtatgcta tgaggctcct gttctgttca agtattctaa tcaatggctt
tgaaaagttt atcaaattta catacagatc acaagcctag gagaaataac taattcacag
atgacagaat taagattata aaagattttt tttttgtaat tttagtagag acagggttgc
cattgtattc cagccttggc gacagagcaa gactctgcct caaaaaaaaa aaaaaaaagg
ttttggcaag ctggaactct ttctgcaaat gactaagata gaaaactgcc aaggacaaat
gaggagtagt tagattttga aaatattaat catagaatag ttgttgtatg ctaagtcact
gacccatatt atgtacagca tttctgatct ttactttgca agattagtga tactatccca
atacactgct ggagaaatca gaatttggag aaataagttg tccaaggcaa gaagatagta
aattataagt acaagtgtaa tatggacagt atctaacttg aaaagatttc aggcgaaaag
aatctggggt ttgccagtca gttgctcaaa aggtcaatga aaaccaaata gtgaagctat
cagagaagct aataaattat agactgcttg aacagttgtg tccagattaa gggagataat
agctttccca ccctactttg tgcaggtcat acctccccaa agtgtttacc taatcagtag
gttcacaaac tcttggtcat tatagtatat gcctaaaatg tatgcactta ggaatgctaa
aaatttaaat atggtctaaa gcaaataaaa gcaaagagga aaaactttgg acagcgtaaa
gactagaata gtcttttaaa aagaaagcca gtatattggt ttgaaatata gagatgtgtc
ccaatttcaa gtattttaat tgcaccttaa tgaaattatc tattttctat agattttagt
actattgaat gtattacttt actgttacct gaatttatta taaagtgttt ttgaataaat
aattctaaaa gcata

What is claimed is:

1. A method of treating a subject in need thereof, the method comprising:
(a) identifying a subject having a cancer cell having decreased ATM level and/or activity in a tumor sample obtained from the subject as compared to a reference level; and
(b) administering a treatment comprising a therapeutically effective amount of a STING antagonist or a cGAS inhibitor, or a pharmaceutically acceptable salt, solvate, or co-crystal thereof to the identified subject.

2. A method of treating a subject in need thereof, the method comprising administering a treatment comprising a therapeutically effective amount of a STING antagonist or a cGAS inhibitor, or a pharmaceutically acceptable salt, solvate, or co-crystal thereof to a subject identified as having a cancer cell having decreased ATM level and/or activity in a tumor sample obtained from the subject as compared to a reference level.

3. A method of selecting a treatment for a subject in need thereof, the method comprising:
(a) identifying a subject having a cancer cell having decreased ATM level and/or activity in a tumor sample obtained from the subject as compared to a reference level; and
(b) selecting for the identified subject a treatment comprising a therapeutically effective amount of a STING antagonist or a cGAS inhibitor, or a pharmaceutically acceptable salt, solvate, or co-crystal thereof.

4. A method of selecting a treatment for a subject in need thereof, the method comprising selecting a treatment comprising a therapeutically effective amount of a STING antagonist or cGAS inhibitor, or a pharmaceutically acceptable salt, solvate, or co-crystal thereof for a subject identified as having a cancer cell having decreased ATM level and/or activity in a tumor sample obtained from the subject as compared to a reference level.

5. A method of selecting a subject for treatment, the method comprising:
(a) identifying a subject having a cancer cell having decreased ATM level and/or activity in a tumor sample obtained from the subject as compared to a reference level; and
(b) selecting the identified subject for treatment with a therapeutically effective amount of a STING antagonist or a cGAS inhibitor, or a pharmaceutically acceptable salt, solvate, or co-crystal thereof.

6. A method of selecting a subject for participation in a clinical trial, the method comprising:
(a) identifying a subject having a cancer cell having decreased ATM level and/or activity in a tumor sample obtained from the subject as compared to a reference level; and
(b) selecting the identified subject for participation in a clinical trial that comprises administration of a treatment comprising a therapeutically effective amount of a STING antagonist or a cGAS inhibitor, or a pharmaceutically acceptable salt, solvate, or co-crystal thereof.

7. A method of selecting a subject for participation in a clinical trial, the method comprising selecting a subject identified as having a cancer cell having decreased ATM level and/or activity in a tumor sample obtained from the subject as compared to a reference level, for participation in a clinical trial that comprises administration of a treatment comprising a therapeutically effective amount of a STING antagonist or a cGAS inhibitor, or a pharmaceutically acceptable salt, solvate, or co-crystal thereof.

8. A method of predicting a subject's responsiveness to a STING antagonist or cGAS inhibitor, the method comprising:
(a) determining that a subject has a cancer cell having decreased ATM level and/or activity in a tumor sample obtained from the subject as compared to a reference level; and
(b) identifying that the subject determined to have decreased ATM expression and/or activity in a tumor sample obtained from the subject as compared to a reference level, in step (a) has an increased likelihood of being responsive to treatment with a STING antagonist or a cGAS inhibitor.

9. A method of predicting a subject's responsiveness to a STING antagonist or cGAS inhibitor, the method comprising identifying a subject determined to have a cancer cell having decreased ATM level and/or activity in a tumor sample obtained from the subject as compared to a reference level, as having an increased likelihood of being responsive to treatment with a STING antagonist or a cGAS inhibitor.

10. The method of any one of claims 1-9, wherein the subject is identified as having a cancer cell having both (i) decreased ATM level and/or activity and (ii) increased cGAS/STING signaling pathway activity, as compared to a reference level; and
optionally wherein the subject is identified as having an elevated level of cGAMP in a serum or tumor sample obtained from the subject as compared to a reference level.

11. The method of any one of claims 1-10, wherein the decreased ATM level and/or activity is a result of loss of one or both alleles of an ATM gene in the subject.

12. The method of any one of claims 1-10, wherein the decreased ATM level and/or activity is a result of a mutation in one or both alleles of an ATM gene in the subject.

13. The method of claim 3 or 4, further comprising administering the selected treatment to the subject.

14. The method of claim 8 or 9, further comprising administering a therapeutically effective amount of a STING antagonist or a cGAS inhibitor to a subject identified as having an increased likelihood of being responsive to treatment with a STING antagonist or a cGAS inhibitor.

15. The method of any one of claims 1-14, wherein the subject has been diagnosed or identified as having a cancer, such as a cancer is selected from the group consisting of: renal clear cell carcinoma, kidney renal papillary cell carcinoma, chromophobe renal cell carcinoma, uveal melanoma, tongue squamous cell carcinoma, breast cancer, osteosarcoma, and skin cancer.

16. The method of any one of claims 1-15, wherein the STING antagonist is a compound of any one of Formulas I-XXIV or Formulas M1-M6, or a pharmaceutically acceptable salt, solvate, or co-crystal thereof.

17. The method of any one of claims 1-15, wherein the STING antagonist or the cGAS inhibitor is a compound selected from the group consisting of the compounds in Tables C1-C2, or a pharmaceutically acceptable salt, solvate, or co-crystal thereof.
